SEQUENCE LISTING (26 sequences)

SEQ ID NO: 1 - DNA, 36 bp, Artificial sequence
  Sense primer for MET6-1
gacgacgacaagatggttcaatcttccgtcttaggt

SEQ ID NO: 2 - DNA, 35 bp, Artificial sequence
  Anti-sense primer for MET6-1
gaggagaagcccggttaagaagattcggatctagc

SEQ ID NO: 3 - DNA, 32 bp, Artificial sequence
  Sense primer for MET6-2
gacgacgacaagatgataccaacgatccaaag

SEQ ID NO: 4 - DNA, 35 bp, Artificial sequence
  Anti-sense primer for MET6-2
gaggagaagcccggttagtatttagctctgaattc

SEQ ID NO: 5 - DNA, 51 bp, Artificial sequence
  Sense primer for RBT4
gacgacgacaagatgaagttttctcaagttgccactactgctgctgccatt

SEQ ID NO: 6 - DNA, 45 bp, Artificial sequence
  Anti-sense primer for RBT4
gaggagaagcccggtaataacaccagagttctgtaaaagtcggta

SEQ ID NO: 7 - DNA, 51 bp, Artificial sequence
  Sense primer for IPF9162
gacgacgacaagatgaaaaaaaggttagttttgtttgatgattctgatgat

SEQ ID NO: 8 - DNA, 57 bp, Artificial sequence
  Anti-sense primer for IPF9162
gaggagaagcccggtaatttatcaatttacatatagtgctcaaaatggacctgtcaa

SEQ ID NO: 9 - DNA, 47 bp, Artificial sequence
  Sense primer for CAR1
gacgacgacaagatgtcatcaattcaatataaatatcatccagacaa

SEQ ID NO: 10 - DNA, 46 bp, Artificial sequence
  Anti-sense primer for CAR1
gaggagaagcccggtaatgttatttcaaactgggttacgtgtagat

SEQ ID NO: 11 - DNA, 36 bp, Artificial sequence
  Sense primer for GAP1
gacgacgacaagatggctattaaaattggtattaac

SEQ ID NO: 12 - DNA, 35 bp, Artificial sequence
  Anti-sense primer for GAP1
gaggagaagcccggttaagcagaagctttagcaac

SEQ ID NO: 13 - DNA, 36 bp, Artificial sequence
  Sense primer for ENO1
gacgacgacaagatgtcttacgccactaaaatccac

SEQ ID NO: 14 - DNA, 38 bp, Artificial sequence
  Anti-sense primer for ENO1
gaggagaagcccggttacaattgagaagccttttggaa

SEQ ID NO: 15 - DNA, 36 bp, Artificial sequence
  Sense primer for BGL2
gacgacgacaagatgcaaatcaaattcttgactact

SEQ ID NO: 16 - DNA, 38 bp, Artificial sequence
  Anti-sense primer for BGL2
gaggagaagcccggttagttgaatttacagtcaattga

SEQ ID NO: 17 - DNA, 36 bp, Artificial sequence
  Sense primer for FBA1
gacgacgacaagatggctcctccagcagttttaagt

SEQ ID NO: 18 - DNA, 38 bp, Artificial sequence
  Anti-sense primer for FBA1
gaggagaagcccggttacaattgtcctttggtgtggaa

SEQ ID NO: 19 - DNA, 35 bp, Artificial sequence
  Sense primer for MUC1-1
gacgacgacaagatgtcattttgggacaacaacaa

SEQ ID NO: 20 - DNA, 37 bp, Artificial sequence
  Anti-sense primer for MUC1-1
gaggagaagcccggttaggttgagttattggttaaaa

SEQ ID NO: 21 - DNA, 36 bp, Artificial sequence
  Sense primer for MUC1-2
gacgacgacaagatggagtatatcgcatcttggtgt

SEQ ID NO: 22 - DNA, 38 bp, Artificial sequence
  Anti-sense primer for MUC1-2
gaggagaagcccggttattcatgtggcattgctcgata

SEQ ID NO: 23 - DNA, 36 bp, Artificial sequence
  Sense primer for PGK1-1
gacgacgacaagatgtcattatctaacaaattatca

SEQ ID NO: 24 - DNA, 33 bp, Artificial sequence
  Anti-sense primer for PGK1-1
gaggagaagcccggttaaccaccaccaacaatc

SEQ ID NO: 25 - DNA, 33 bp, Artificial sequence
  Sense primer for PGK1-2
gacgacgacaagatggccttcactttcaagaaa

SEQ ID NO: 26 - DNA, 35 bp, Artificial sequence
  Anti-sense primer for PGK1-2
gaggagaagcccggttagtttttgttggaaagagc

(19) United States
(12) Patent Application Publication
(10) Pub. No.: US 2010/0119533 A1
(43) Pub. Date: May 13, 2010

(54) Polynucleotides and Polypeptides Identified by IVIAT Screening and Methods of Use

(76) Inventors: Cornelius Joseph Clancy, Pittsburgh, PA (US); Minh-Hong Thi Nguyen, Gainesville, FL (US); Shaoji Cheng, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/529,919

(22) PCT Filed: Mar. 7, 2008
(86) PCT No.: PCT/US08/56236

§ 371 Date: Jan. 18, 2010

Related U.S. Application Data
(60) Provisional application No. 60/893,537, filed on Mar. 7, 2007.
(60) Provisional application No. 60/972,413, filed on Sep. 14, 2007.

(51) Int. Cl. A61K 39/00; C40B 40/10; C07K 14/00; C07K 7/00; C40B 40/06; C07H 21/04; C40B 30/04; C07K 16/00; C12Q 1/68

(52) U.S. Cl. 424/185.1; 506/18; 530/350; 530/300; 506/16; 536/23.1; 506/9; 530/387.9; 435/6

(57) ABSTRACT

The Candida and Aspergillus polypeptides of the invention have been found to be immunogenic and are useful as diagnostic test antigens. The polypeptide antigens of the subject invention can provide the basis of a diagnostic assay that would allow the rapid, in-house, laboratory diagnosis of infection with Candida and/or Aspergillus using a sample (e.g., serum, plasma, or whole blood) from an infected human or animal. Additionally, the subject invention provides methods of detecting the presence of Candida albicans and/or Aspergillus fumigatus in biological or environmental samples utilizing antibodies provided by the subject invention. Furthermore, the use of single antigens or, more preferably, one or more groups (sets) of antigens of the invention in the diagnosis of these important diseases offers many advantages including enhanced test specificity, ease of testing and consistency of results using synthetically or recombinantly produced test antigens instead of cultured, whole organisms. In one embodiment, the group of antigens comprises or consists of one or more polypeptides (e.g., one, two, three, or four or more antigens) selected from among SET1 (chromatin regulatory protein), ENO1 (enolase I), PGK1-2 (phosphoglycerate kinease), and MUC1-2 (cell surface glycoprotein). In another embodiment, the group of antigens comprises or consists of one or more polypeptides (e.g., one, two, three, four, five, six, or seven or more polypeptides) selected from among SET1, ENO1, FBA1, PGK1-1, PGK1-2, MUC1-2, and BGL2. In another embodiment, the group of antigens comprises or consists of one or more polypeptides (e.g., one, two, three, four, five, six, seven, eight, nine, ten, eleven, twelve, thirteen, fourteen, or fifteen or more polypeptides) selected from among MET6-1, MET6-2, NOT5, RBT4, IPF9162, CAR1, GAP1, SET1, ENO1, FBA1, PGK1-1, PGK1-2, MUC1-1, MUC1-2, and BGL2.

### CROSS-REFERENCE TO RELATED APPLICATIONS

This application claims the benefit of U.S. Provisional Application Ser. No. 60/893,537, filed Mar. 7, 2007, and Ser. No. 60/972,413, filed Sep. 14, 2007, the disclosures of which are hereby incorporated by reference in their entireties, including all figures, tables and amino acid or nucleic acid, sequences.

### GOVERNMENT SUPPORT

The subject matter of this application has been supported by research grants from the National Institute of Dental and Craniofacial Research (NIH/NIDCR) under grant numbers 1RO1DE13980-01 and 1R21DE015069-01 A1 and the National Institute of Allergy and Infectious Diseases (NIH/NIAID) under grant number PO1A1061537-01. Accordingly, the government has certain rights in this invention.

### BACKGROUND OF THE INVENTION

Candida spp. are major causes of systemic infections among hospitalized patients in the developed world, particularly among immunosuppressed patients. Candidemia, for example, is the fourth most common bloodstream infection in the United States and is associated with attributable mortality as high as 40-50% despite treatment with antifungal agents (Edmond, M B et al. Clin Infect Dis., 1999; 29:239-44; Gudlaugsson, O et al. Clin Infect Dis., 2003; 37:1172-7). The management of systemic candidiasis is complicated by the limitations of current diagnostic tests. Blood cultures, generally taken as the gold standard for the diagnosis of candidemia, are negative in up to 50% of autopsy-proven cases of disseminated candidiasis (Berenguer, J et al. Diagn Microbiol Infect Dis., 1993; 17:103-9). Moreover, blood cultures that are positive often become so late in the disease course (Ellepola, A N and Morrison, C J J Microbiol., 2005; 43:65-84; Morris, A J et al. J Clin Microbiol., 1996; 34:1583-5), which delays appropriate therapy. Such delays in the institution of an antifungal agent are associated with significantly increased mortality rates among patients with candidemia (Morrell, M et al. Antimicrob Agents Chemother., 2005; 49:3640-5; Garey, K W et al. Clin Infect Dis., 2006 Jul 1; 43:25-31). Cultures of deep tissue sites are even more problematic, and require invasive sampling procedures, which are difficult to perform and are often contra-indicated in patients at high-risk for systemic candidiasis. Because of these shortcomings, there is great interest in non-culture based diagnostic tests.

Investigators have assessed a wide range of potential diagnostic markers, including the detection of candidal nucleic acids, metabolites such as D-arabinitol, cell wall components such as β-D-glucan, and antigens such as secreted aspartyl proteinase (Sap), enolase and mannan (Ellepola, A N and Morrison, C J J Microbiol., 2005; 43:65-84). Despite individual reports of reasonable diagnostic yield for each of these tests, none have been broadly validated or accepted in widespread clinical practice. There has been less enthusiasm for antibody detection as a diagnostic strategy because of concerns for false negative tests in immunocompromised patients and false-positive tests resulting from prior exposure to commensal Candida spp. (Quindos, G et al. Rev Iberoam Micol., 2004; 21(1)). Nevertheless, recent studies detecting antibodies against Sap (Na B K and Song C Y Clin Diagn Lab Immunol., 1999; 6:924-9; Morrison C J et al. Clin Diagn Lab Immunol., 2003; 10:835-48; Yang Q et al. Mycoses, 2007; 50:165-71), enolase (van Deventer, A J et al. Microbiol Immunol., 1996; 40:125-31; Mitsutake, K et al. J Clin Microbiol., 1996; 1918-21; Lain, A et al. Clin Vaccine Immunol., 2007; 14:318-9), hyphal wall protein 1 (Lain, A et al. Clin Vaccine Immunol., 2007; 14:318-9), mannan (Sendid, B et al. J Clin Microbiol., 1999; 37:1510-7; Yera, H et al. Eur J Clin Microbiol Infect Dis., 2001; 20:864-70), a 52 kDa metalloprotein (El Moudni, B et al. Clin Diagn Lab Immunol., 1998; 5:823-5), and a C. albicans germ-tube antigen (CTGA) (Quindos, G et al. Rev Iberoam Micol., 2004; 21(1); Lain, A et al. Clin Vaccine Immunol., 2007; 14:318-9) have reported sensitivities and specificities that are consistent with other diagnostic markers, even among highly immunocompromised hosts like stem cell transplant and liver transplant recipients (Hoppe, J E et al. Mycoses, 1995; 38:41-9; Klingspor, L et al. Acta Paediatr., 1995; 84:424-8; Navarro, D et al. Eur J Clin Microbiol Infect Dis., 1993; 12:839-46; Tollemar, J et al. Scand J Infect Dis., 1989; 21:205-12; Villalba, R. et al. Eur J Clin Microbiol Infect Dis., 1993; 12:347-9). Moreover, various combinations of antibody test with an antigen test have been shown to be superior to either test alone in diagnosing systemic candidiasis (Sendid, B et al. J Med Microbial., 2002; 51:433-42; Sendid, B et al. J Clin Microbial., 2004; 42:164-71; Pazos, C et al. Rev Iberoam Micol., 2006; 23:209-15).

Despite shortcomings, cultures of blood or sterile sites remain the gold-standard for diagnosing systemic candidiasis. Alternative diagnostic markers including antibody detection have been developed, but none have been widely accepted in clinical practice. Clearly, much work remains to be done in developing diagnostic markers; antibody detection strategies merit exploration as part of these endeavors.

In Vivo Induced Antigen Technology (IVIAT) is a technique that identifies pathogen antigens that are immunogenic and expressed in vivo during human infection (Rollins et al., Cell. Microbial., 2005, 7(1):1-9; Richardson et al., J. Med. Microbial., 2005, 54:497-5-4; John et al., Infection and Immunity, 2005, 73(5):2665-2679; Hang et al., PNAS, 2003, 100(14):8508-8513; International Publication No. WO 01/11081 (Progulskefox et al.)). IVIAT is complementary to other techniques that identify genes and their products expressed in vivo. Genes and gene pathways identified by IVIAT can play a role in virulence or pathogenesis during human infection, and may be appropriate for inclusion in therapeutic, vaccine, or diagnostic applications.

### BRIEF SUMMARY OF THE INVENTION

Selected genes expressed during the course of infection encode immunogenic proteins that represent targets for novel diagnostic tests. The present inventors identified over 60 immunogenic C. albicans proteins using an antibody-based screening method. In Vivo Induced Antigen Technology (IVIAT) was used to identify immunogenic Candida and Aspergillus proteins that may be targets for novel therapeutics, diagnostics, and vaccines (Handfield M. et al., Trends Microbial., 2000, 8:336-339, which is incorporated herein by reference in its entirety).

In brief, sera were pooled from 24 HIV-infected patients with active candidiasis. Serum from a patient was also collected after he had recovered from IA. The present inventors repeatedly adsorbed the sera against Candida albicans or Aspergillus fumigatus cells and cell lysates. Using ELISA, it was demonstrated that repeated rounds of adsorption reduced overall anti-Candida and anti-Aspergillus antibody titers. The adsorbed sera was then used to screen C. albicans and A. fumigatus genomic DNA expression libraries. After identifying colonies that were consistently reactive with antibodies in the sera, genes encoding the immunogenic proteins were identified by searching genome sequencing databases. To date, the present inventors have identified 69 C. albicans and 14 A. fumigatus genes and their corresponding proteins (see Tables 7-8, which lists C. albicans and A. fumigatus genes and proteins, including GenBank accession numbers). A. fumigatus screening in on-going. Furthermore, the C. albicans and A. fumigatus nucleic acids and polypeptides, and the pathogenic conditions associated with the nucleic acids and/or polypeptides (such as disseminated candidiasis, oropharyngeal candidiasis, vulvovaginal candidiasis, etc.), referenced in Raman et al., Molecular Microbiology, 60(3):697-709; Cheng et al., Infection and Immunity, 2005, 73(11):7190-7197; Badrane et al., Microbiology, 2005, 151:2923-2931; Nguyen et al., Med. Mycol., 2004, 42(4):293-304; Cheng et al., Molecular Microbiology, 2003, 48(5):1275-1288; and Cheng et al., Infection and Immunity, 2003, 71(19):6101-6103, are incorporated herein by reference.

Since the C. albicans and A. fumigatus genes and proteins identified by the present inventors' screening are reactive with antibodies from humans infected with the organisms, it is expected that they are promising targets for novel therapeutics, diagnostics, and vaccines.

The Candida and Aspergillus polypeptides of the invention have been found to be immunogenic and are useful as diagnostic test antigens. The polypeptide antigens of the subject invention can provide the basis of a diagnostic assay that would allow the rapid, in-house, laboratory diagnosis of infection with Candida and/or Aspergillus using a sample (e.g., adsorbed or unadsorbed serum, plasma, or whole blood) from an infected human or animal. Additionally, the subject invention provides methods of detecting the presence of Candida albicans and/or Aspergillus fumigatus in biological or environmental samples utilizing antibodies provided by the subject invention. Furthermore, the use of single antigens or, more preferably, one or more groups of antigens of the invention in the diagnosis of these important diseases offers many advantages including enhanced test specificity, ease of testing and consistency of results using synthetically or recombinantly produced test antigens instead of cultured, whole organisms. In one embodiment of each aspect of the invention, the one or more antigens (e.g., one, two, three, four, five, or six or more antigens) is among those polypeptides disclosed in Tables 3, 4, 5, 6, 7, 8, 9, 12, 13, 14, 15, 16, 17 or 18 herein, or among those polypeptides encoded by the nucleic acids disclosed in Tables 3, 4, 5, 6, 7, 8, 9, 12, 13, 14, 15, 16, or 17 herein. In another embodiment, the one or more antigens (e.g., one, two, three, four, five, or six or more antigens) are from C. albicans and selected from the group among Set1p, Rbt4p, Met6p, BGl2p, Gap1, Bgl2, Car1, Enol1, Fba1, IPF9162, PGK1, and Muc1. In another embodiment, the one or more antigens (e.g., one, two, three, or four or more antigens) are from C. albicans and selected from among Set1p, Rbt4p, Met6p, and BGl2p. In another embodiment, the one or more antigens (e.g., one, two, three, four, or five or more antigens) are from C. albicans and selected from among Set1p, Rbt4p, Met6p, BGl2p, and Gap1. In another embodiment, the one or more antigens (e.g., one, two, three, or four or more antigens) are from C. albicans and selected from among Car1, Enol1, Fba1, and IPF9162. In another embodiment, the antigen is from C. albicans and is PGK1. In another embodiment, the antigen is from C. albicans and is Muc1.

The inventors have used a human antibody-based screening strategy to identify C. albicans genes that encode immunogenic proteins, including previously uncharacterized virulence factors (Cheng, S et al. Mol Microbiol., 2003; 48:1275-88; Nguyen, M H et al. Med Mycol., 2004; 42:293-304). 12 proteins were chosen to study as targets for antibody detection assays. These proteins can be classified into four groups according to their cellular localizations and functions: classic cell wall proteins; glycolytic enzymes localized to the cell wall; intracellular proteins localized to cell wall; and intracellular proteins, likely not localized to cell wall (Table 9). The objectives were to determine if serum antibody responses against any of the recombinant antigens could reliably distinguish patients with systemic candidiasis from un-infected controls. The inventors also sought to derive a predictive model for systemic candidiasis that considered antibody responses against multiple antigens.

In this study, ELISA was used to measure serum antibody responses against 15 recombinant Candida albicans antigens among patients with systemic candidiasis due to a variety of Candida spp. (n=60) and un-infected controls (n=24). IgG responses were better than IgM in differentiating patients with systemic candidiasis from controls. The inventors tested a prediction model including serum IgG responses against all 15 antigens, and identified patients with systemic candidiasis with an error rate of 3.7%, sensitivity of 96.6% and specificity of 95.6%. Furthermore, a subset of 4 antigens (SET1, ENO1, PGK1-2 and MUC1-2) identified through backwards elimination and canonical correlation analyses performed as accurately as the full panel. Using that simplified model, systemic candidiasis could be predicted in a test sample of 32 patients with 100% sensitivity and 87.5% specificity. These findings show that detection of antibodies against a panel of candidal antigens represents an advance in the diagnosis of systemic candidiasis. Accordingly, in another embodiment, the one or more antigens (e.g., one, two, three, or four or more antigens) are from C. albicans and selected from among SET1 (chromatin regulatory protein), ENO1 (enolase I), PGK1-2 (phosphoglycerate kinease), and MUC1-2 (cell surface glycoprotein). In another embodiment, the antigen is from C. albicans and is SET1. In another embodiment, the antigen is from C. albicans and is ENO1. In another embodiment, the antigen is from C. albicans and is PGK1-2. In another embodiment, the antigen is from C. albicans and is MUC1-2.

In another embodiment, the antigen is from C. albicans and is one or more polypeptides (e.g., one, two, three, four, five, six, seven, eight, nine, ten, eleven, twelve, thirteen, fourteen, or fifteen or more polypeptides) selected from among METE-1, MET6-2, NOT5, RBT4, IPF9162, CAR1, GAP1, SET1, ENO1, FBA1, PGK1-1, PGK1-2, MUC1-1, MUC1-2, and BGL2.

In another embodiment, the antigen is from C. albicans and is one or more polypeptides (e.g., one, two, three, four, five, six, or seven or more polypeptides) selected from among SET1, ENO1, FBA1, PGK1-1, PGK1-2, MUC1-2, and BGL2.

### BRIEF DESCRIPTION OF THE DRAWINGS

FIG. 1 shows a pie chart of portals of entry of fungemia.

FIGS. 2A-2F show representative IgG responses against specific Candida proteins.

FIGS. 3A and 3B are graphs showing IgM and IgG titers, respectively, against specific proteins among patients with systemic candidiasis and controls.

### BRIEF DESCRIPTION OF THE SEQUENCES

SEQ ID NO: 1 is the sense primer for MET6-1.

SEQ ID NO: 2 is the anti-sense primer for MET6-1.

SEQ ID NO: 3 is the sense primer for MET6-2.

SEQ ID NO: 4 is the anti-sense primer for MET6-2.

SEQ ID NO: 5 is the sense primer for RBT4.

SEQ ID NO: 6 is the anti-sense primer for RBT4.

SEQ ID NO: 7 is the sense primer for IPF9162.

SEQ ID NO: 8 is the anti-sense primer for IPF9162.

SEQ ID NO: 9 is the sense primer for CAR1.

SEQ ID NO: 10 is the anti-sense primer for CAR1.

SEQ ID NO: 11 is the sense primer for GAP1.

SEQ ID NO: 12 is the anti-sense primer for GAP1.

SEQ ID NO: 13 is the sense primer for ENO1.

SEQ ID NO: 14 is the anti-sense primer for ENO1.

SEQ ID NO: 15 is the sense primer for BGL2.

SEQ ID NO: 16 is the anti-sense primer for BGL2.

SEQ ID NO: 17 is the sense primer for FBA1.

SEQ ID NO: 18 is the anti-sense primer for FBA1.

SEQ ID NO: 19 is the sense primer for MUC1-1.

SEQ ID NO: 20 is the anti-sense primer for MUC1-1.

SEQ ID NO: 21 is the sense primer for MUC1-2.

SEQ ID NO: 22 is the anti-sense primer for MUC1-2.

SEQ ID NO: 23 is the sense primer for PGK1-1.

SEQ ID NO: 24 is the anti-sense primer for PGK1-1.

SEQ ID NO: 25 is the sense primer for PGK1-2.

SEQ ID NO: 26 is the anti-sense primer for PGK1-2.

### DETAILED DESCRIPTION OF THE INVENTION

In Vivo Induced Antigen Technology (IVIAT) was used to identify immunogenic Candida and Aspergillus proteins that may be targets for novel therapeutics, diagnostics, and vaccines (Handfield M. et al., Trends Microbiol., 2000, 8:336-339, which is incorporated herein by reference in its entirety).

The subject invention provides:

a) one or more:

1) isolated, purified, and/or recombinant polypeptides comprising those listed in Tables 3, 4, 5, 6, 7, 8, 9, 12, 13, 14, 15, 16, 17 or 18 disclosed herein;

2) isolated, purified, and/or recombinant polypeptides (e.g., one, two, three, or four or more polypeptides) selected from among SET1 (chromatin regulatory protein), ENO1 (enolase I), PGK1-2 (phosphoglycerate kinease), and MUC1-2 (cell surface glycoprotein); or

3) one or more polypeptides (e.g., one, two, three, four, five, six, seven, eight, nine, ten, eleven, twelve, thirteen, fourteen, or fifteen or more polypeptides) selected from among MET6-1, METE-2, NOT5, RBT4, IPF9162, CAR1, GAP1, SET1, ENO1, FBA1, PGK1-1, PGK1-2, MUC1-1, MUC1-2, and BGL2; or

4) one or more polypeptides (e.g., one, two, three, four, five, six, or seven or more polypeptides) selected from among SET1, ENO1, FBA1, PGK1-1, PGK1-2, MUC1-2, and BGL2; or

5) variant polypeptides having at least about 20% to 99.99% identity, preferably at least 60% to 99.99% identity to the polypeptide of listed in the tables disclosed herein and which have at least one of the activities associated with the polypeptides listed in tables 7-8 disclosed herein;

6) a fragment of the polypeptide(s) listed in Tables 3, 4, 5, 6, 7, 8, 9, 12, 13, 14, 15, 16, 17 or 18 disclosed herein, or a variant polypeptide, wherein the polypeptide fragment or fragment of the variant polypeptide has substantially the same activity as the full length polypeptides listed in Tables 3, 4, 5, 6, 7, 8, 9, 12, 13, 14, 15, 16, 17 or 18 disclosed herein;

7) a multimeric polypeptide construct comprising a series of repeating elements that are, optionally, joined together by linker elements, wherein said repeating elements are selected from one, or more, of the polypeptides listed in Tables 3, 4, 5, 6, 7, 8, 9, 12, 13, 14, 15, 16, 17 or 18 disclosed herein;

8) an epitope of a polypeptide selected from those listed in Tables 3, 4, 5, 6, 7, 8, 9, 12, 13, 14, 15, 16, 17 or 18 disclosed herein;

9) a multi-epitope construct comprising at least one epitope as set forth herein; or

10) a polypeptide according to embodiments a(1), a(2), a(3), a(4), a(5), a(6), a(7), a(8), or a(9) that further comprises a heterologous polypeptide sequence;

b) a composition comprising a carrier and a polypeptide as set forth in a(1), a(2), a(3), a(4), a(5), a(6), a(7), a(8), a(9), or a(10) wherein said carrier is an adjuvant or a pharmaceutically acceptable excipient;

c) methods of detecting the presence of antibodies in a subject infected with Candida and/or Aspergillus (e.g., Candida albicans and/or Aspergillus fumigatus) comprising contacting a biological sample with a polypeptide or polypeptides as set forth in a(1), a(2), a(3), a(4), a(5), a(6), a(7), a(8), a(9), or a(10) and detecting the presence of an antigen/antibody complex; and

d) an improvement in methods of diagnosing or detecting an Candida and/or Aspergillus (e.g., Candida albicans and/or Aspergillus fumigatus) Candida and/or Aspergillus (e.g., Candida albicans and/or Aspergillus fumigatus) infection in a subject, wherein the improvement comprises the use of an isolated, purified, and/or recombinant polypeptide as set forth in a(1), a(2), a(3), a(4), a(5), a(6), a(7), a(8), a(9), or a(10) in an immunoassay for the detection or diagnosis of an Candida and/or Aspergillus infection.

In one embodiment of each of the aforementioned aspects of the invention a)-d), the one or more polypeptides (e.g., one, two, three, four, five, or six or more polypeptides) is among those polypeptides disclosed in. Tables 3, 4, 5, 6, 7, 8, 9, 12, 13, 14, 15, 16, 17 or 18 herein, or among those polypeptides encoded by the nucleic acids disclosed in Tables 3, 4, 5, 6, 7, 8, 9, 12, 13, 14, 15, 16, or 17 herein. In another embodiment, the one or more polypeptides (e.g., one, two, three, four, five, or six or more polypeptides) are from C. albicans and selected from the group consisting of Set1p, Rbt4p, Met6p, BGl2p, Gap1, Bgl2, Car1, Enol1, Fba1, IPF9162, PGK1, and Muc1. In another embodiment, the one or more polypeptides (e.g., one, two, three, or four polypeptides) are from C. albicans and selected from the group consisting of Set1p, Rbt4p, Met6p, and BGl2p. In another embodiment, the one or more polypeptides (e.g., one, two, three, four, or five polypeptides) are from C. albicans and selected from the group consisting of Set1p, Rbt4p, Met6p, BGl2p, and Gap1. In another embodiment, the one or more polypeptides (e.g., one, two, three, or four polypeptides) are from C. albicans and selected from the group consisting of Car1, Enol1, Fba1, and IPF9162. In another embodiment, the polypeptide is from C. albicans and is PGK1. In another embodiment, the polypeptide is from C. albicans and is Muc1. The disclosed polypeptides and variants therof contain one or more eptitopes that are specifically bound by antibodies in adsorbed or unadsorbed serum.

In another embodiment, the one or more antigens (e.g., one, two, three, or four or more antigens) are from C. albicans and selected from among SET1 (chromatin regulatory protein), ENO1 (enolase I), PGK1-2 (phosphoglycerate kinease), and MUC1-2 (cell surface glycoprotein). In another embodiment, the antigen is from C. albicans and is SET1. In another embodiment, the antigen is from C. albicans and is ENO1. In another embodiment, the antigen is from C. albicans and is PGK1-2. In another embodiment, the antigen is from C. albicans and is MUC1-2.

In another embodiment, the antigen is from C. albicans and is one or more polypeptides (e.g., one, two, three, four, five, six, seven, eight, nine, ten, eleven, twelve, thirteen, fourteen, or fifteen or more polypeptides) selected from among METE-1, MET6-2, NOT5, RBT4, IPF9162, CAR1, GAP1, SET1, ENO1, FBA1, PGK1-1, PGK1-2, MUC1-1, MUC1-2, and BGL2.

In another embodiment, the antigen is from C. albicans and is one or more polypeptides (e.g., one, two, three, four, five, six, or seven or more polypeptides) selected from among SET1, ENO1, FBA1, PGK1-1, PGK1-2, MUC1-2, and BGL2.

In the context of the instant invention, the terms “oligopeptide”, “polypeptide”, “peptide” and “protein” can be used interchangeably to refer to a chain of two or more amino acids; however, it should be understood that the invention does not relate to the polypeptides in natural form, that is to say that they are not in their natural environment but that the polypeptides may have been isolated or obtained by purification from natural sources or obtained from host cells prepared by genetic manipulation (e.g., the polypeptides, or fragments thereof, are recombinantly produced by host cells, or by chemical synthesis). Polypeptides according to the instant invention may also contain non-natural amino acids, as will be described below. The terms “oligopeptide”, “polypeptide”, “peptide” and “protein” are also used, in the instant specification, to designate a series of residues, typically L-amino acids, connected one to the other, typically by peptide bonds between the a-amino and carboxyl groups of adjacent amino acids. Linker elements can be joined to the polypeptides of the subject invention through peptide bonds or via chemical bonds (e.g., heterobifunctional chemical linker elements) as set forth below. Additionally, the terms “amino acid(s)” and “residue(s)” can be used interchangeably.

Thus, the subject invention provides polypeptides comprising those listed in Tables 3, 4, 5, 6, 7, 8, 9, 12, 13, 14, 15, 16, 17 or 18 disclosed herein and/or polypeptide fragments of those polypeptides. In some embodiments of the subject invention, polypeptide fragments of the subject invention are epitopes that are bound by antibodies or T-cell receptors are designated “epitopes”; in the context of the subject invention, “epitopes” are considered to be a subset of the invention designated as “fragments of invention”.

Polypeptide fragments (and/or epitopes) according to the subject invention, usually comprise a contiguous span of or at least 3, 4, 5, 6, 7, 8, 9, 10, 11, 12, 13, 14, 15, 16, 17, 18, 19, 20, 21 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, and up to one amino acid less than the full length polypeptides of the invention.

Polypeptide fragments of the subject invention can be any integer in length from at least 3, preferably 4, and more preferably 5 consecutive amino acids to 1 amino acid less than a full length polypeptide of those listed in Tables 3, 4, 5, 6, 7, 8, 9, 12, 13, 14, 15, 16, 17 or 18 disclosed herein. The term “integer” is used herein in its mathematical sense and thus representative integers include, for example: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, and so on.

Each polypeptide fragment of the subject invention can also be described in terms of its N-terminal and C-terminal positions. For example, combinations of N-terminal to C-terminal fragments of 6 contiguous amino acids to 1 amino acid less than the full length polypeptide are included in the present invention. Thus, for example, a 6 consecutive amino acid fragment could occupy positions selected from the group consisting of 1-6, 2-7, 3-8, 4-9, 5-10, 6-11, 7-12, 8-13, 9-14, 10-15, 11-16, 12-17, 13-18, 14-19, 15-20, 16-21, 17-22, 18-23, 19-24, 20-25, 21-26, 22-27, 23-28, 24-29, 25-30, 26-31, 27-32, 28-33, 29-34, 30-35, 31-36, 32-37, 33-38, 34-39, 35-40, 36-41, 37-42, 38-43, 39-44, 40-45, 41-46, 42-47, 43-48, 44-49, 45-50, 46-51, 47-52, 48-53, 49-54, 50-55, 51-56, 52-57, 53-58, 54-59, 55-60, 56-61, 57-62, 58-63, 59-64, 60-65, 61-66, 62-67, 63-68, 64-69, 65-70, 66-71, 67-72, 68-73, and 69-74.

Fragments, as described herein, can be obtained by cleaving the polypeptides of the invention with a proteolytic enzyme (such as trypsin, chymotrypsin, or collagenase) or with a chemical reagent, such as cyanogen bromide (CNBr). Alternatively, polypeptide fragments can be generated in a highly acidic environment, for example at pH 2.5 or by other means (see, e.g., Kolaskar, A S and Tongaonkar, P C [1990], FEBS Letters 276: 172-174; Parker, J M R, Guo, D and Hodges, R S [1986] Biochemistry 25: 5425-5432; and/or Saha, S and Raghava, G. P. S. [2006] Proteins 65(1):40-48). Such polypeptide fragments may be equally well prepared by chemical synthesis or using hosts transformed with an expression vector according to the invention. The transformed host cells contain a nucleic acid, allowing the expression of these fragments, under the control of appropriate elements for regulation and/or expression of the polypeptide fragments.

In certain preferred embodiments, fragments of the polypeptides disclosed herein retain at least one property or activity of the full-length polypeptide from which the fragments are derived. Thus, fragments of the polypeptide have one or more of the following properties or activities: a) the ability to: 1) specifically bind to adsorbed or unadsorbed antibodies specific for the full length polypeptide; and/or 2) specifically bind antibodies found in an animal or human infected with Candida and/or Aspergillus; b) the ability to bind to, and activate T-cell receptors (CTL (cytotoxic T-lymphocyte) and/or HTL (helper T-lymphocyte receptors)) in the context of MHC Class I or Class II antigen that are isolated or derived from an animal or human infected with Candida and/or Aspergillus; 3) the ability to induce an immune response in an animal or human; and/or 4) the ability to induce a protective immune response in an animal or human against Candida and/or Aspergillus.

The polypeptides, and fragments thereof, may further comprise linker elements (L) that facilitate the attachment of the fragments to other molecules, amino acids, or polypeptide sequences. The linkers can also be used to attach the polypeptides, or fragments thereof, to solid support matrices for use in affinity purification protocols. Non-limiting examples of “linkers” suitable for the practice of the invention include chemical linkers (such as those sold by Pierce, Rockford, Ill.), or peptides that allow for the connection combinations of polypeptides (see, for example, linkers such as those disclosed in U.S. Pat. Nos. 6,121,424, 5,843,464, 5,750,352, and 5,990,275, hereby incorporated by reference in their entirety).

In other embodiments, the linker element (L) can amino acid sequences. In other embodiments, the peptide linker has one or more of the following characteristics: a) it allows for the free rotation of the polypeptides that it links (relative to each other); b) it is resistant or susceptible to digestion (cleavage) by proteases; and c) it does not interact with the polypeptides it joins together. In various embodiments, a multimeric construct according to the subject invention includes a peptide linker and the peptide linker is 5 to 60 amino acids in length. More preferably, the peptide linker is 10 to 30, amino acids in length; even more preferably, the peptide linker is 10 to 20 amino acids in length. In some embodiments, the peptide linker is 17 amino acids in length.

Peptide linkers suitable for use in the subject invention are made up of amino acids selected from the group consisting of Gly, Ser, Asn, Thr and Ala. Preferably, the peptide linker includes a Gly-Ser element. In a preferred embodiment, the peptide linker comprises (Ser-Gly-Gly-Gly-Gly)_{y} wherein y is 1, 2, 3, 4, 5, 6, 7, or 8. Other embodiments provide for a peptide linker comprising ((Ser-Gly-Gly-Gly-Gly)_{y}-Ser-Pro). In certain preferred embodiments, y is a value of 3, 4, or 5. In other preferred embodiment, the peptide linker comprises (Ser-Ser-Ser-Ser-Gly)_{y} or ((Ser-Ser-Ser-Ser-Gly)_{y}-Ser-Pro), wherein y is 1, 2, 3, 4, 5, 6, 7, or 8. In certain preferred embodiments, y is a value of 3, 4, or 5. Where cleavable linker elements are desired, one or more cleavable linker sequences such as Factor Xa or enterokinase (Invitrogen, San Diego Calif.) can be used alone or in combination with the aforementioned linkers

Multimeric constructs of the subject invention typically comprise a series of repeating elements, optionally interspersed with other elements. As would be appreciated by one skilled in the art, the order in which the repeating elements occur in the multimeric polypeptide is not critical and any arrangement of the repeating elements as set forth herein can be provided by the subject invention. Thus, a “multimeric construct” according to the subject invention can provide a multimeric polypeptide comprising a series of polypeptides, polypeptide fragments, or epitopes that are, optionally, joined together by linker elements (either chemical linker elements or amino acid linker elements).

A “variant polypeptide” (or polypeptide variant) is to be understood to designate polypeptides exhibiting, in relation to the natural polypeptide, certain modifications. These modifications can include a deletion, addition, or substitution of at least one amino acid, a truncation, an extension, a chimeric fusion, a mutation, or polypeptides exhibiting post-translational modifications. Among these homologous variant polypeptides, are those comprising amino acid sequences exhibiting between at least (or at least about) 20.00% to 99.99% (inclusive) identity to the full length, native, or naturally occurring polypeptide are another aspect of the invention. The aforementioned range of percent identity is to be taken as including, and providing written description and support for, any fractional percentage, in intervals of 0.01%, between 20.00% and, up to, including 99.99%. These percentages are purely statistical and differences between two polypeptide sequences can be distributed randomly and over the entire sequence length. Thus, variant polypeptides can have 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity with the polypeptide sequences of the instant invention. In a preferred embodiment, a variant or modified polypeptide exhibits at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity to one of those listed in Tables 3, 4, 5, 6, 7, 8, 9, 12, 13, 14, 15, 16, 17 or 18 disclosed herein. Typically, the percent identity is calculated with reference to the full-length, native, and/or naturally occurring polypeptide. In all instances, variant polypeptides retain at least one of the activities associated with the native polypeptide. In some embodiments, variant polypeptides retain at least 2, and preferably all of the activities associated with the native polypeptide.

Variant polypeptides can also comprise one or more heterologous polypeptide sequences (e.g., tags that facilitate purification of the polypeptides of the invention (see, for example, U.S. Pat. No. 6,342,362, hereby incorporated by reference in its entirety; Altendorf et al. [1999-WWW, 2000] “Structure and Function of the Fₒ Complex of the ATP Synthase from Escherichia Coli,” J. of Experimental Biology 203:19-28, The Co. of Biologists, Ltd., G. B.; Baneyx [1999] “Recombinant Protein Expression in Escherichia coli,” Biotechnology 10:411-21, Elsevier Science Ltd.; Eihauer et al. [2001] “The FLAG™ Peptide, a Versatile Fusion Tag for the Purification of Recombinant Proteins,” J. Biochem Biophys Methods 49:455-65; Jones et al. [1995] J. Chromatography 707:3-22; Jones et al. [1995] “Current Trends in Molecular Recognition and Bioseparation,” J. of Chromatography A. 707:3-22, Elsevier Science B. V.; Margolin [2000] “Green Fluorescent Protein as a Reporter for Macromolecular Localization in Bacterial Cells,” Methods 20:62-72, Academic Press; Puig et al. [2001] “The Tandem Affinity Purification (TAP) Method: A General Procedure of Protein Complex Purification,” Methods 24:218-29, Academic Press; Sassenfeld [1990] “Engineering Proteins for Purification,” TibTech 8:88-93; Sheibani [1999] “Prokaryotic Gene Fusion Expression Systems and Their Use in Structural and Functional Studies of Proteins,” Prep. Biochem. & Biotechnol. 29(1):77-90, Marcel Dekker, Inc.; Skerra et al. [1999] “Applications of a Peptide Ligand for Streptavidin: the Strep-tag”, Biomolecular Engineering 16:79-86, Elsevier Science, B. V.; Smith [1998] “Cookbook for Eukaryotic Protein Expression: Yeast, Insect, and Plant Expression Systems,” The Scientist 12(22):20; Smyth et al. [200] “Eukaryotic Expression and Purification of Recombinant Extracellular Matrix Proteins Carrying the Strep II Tag”, Methods in Molecular Biology, 139:49-57; Unger [1997] “Show Me the Money: Prokaryotic Expression Vectors and Purification Systems,” The Scientist 11(17):20, each of which is hereby incorporated by reference in their entireties), or commercially available tags from vendors such as such as STRATAGENE (La Jolla, Calif.), NOVAGEN (Madison, Wis.), QIAGEN, Inc., (Valencia, Calif.), or InVitrogen (San Diego, Calif.).

In other embodiments, polypeptides of the subject invention can be fused to heterologous polypeptide sequences that have adjuvant activity (a polypeptide adjuvant). Non-limiting examples of such polypeptides include heat shock proteins (hsp) (see, for example, U.S. Pat. No. 6,524,825, the disclosure of which is hereby incorporated by reference in its entirety).

Also included within the scope of the subject invention are at least one or more polypeptide fragments that are an “epitope” or which contain one or more epitope. In the context of the subject invention, an the term “epitope” is used to designate a series of residues, typically L-amino acids, connected one to the other, typically by peptide bonds between the α-amino and carboxyl groups of adjacent amino acids. The preferred CTL (or CD8⁺ T cell)-inducing peptides of the invention are 13 residues or less in length and usually consist of between about 8 and about 11 residues (e.g., 8, 9, 10 or 11 residues), preferably 9 or 10 residues. The preferred HTL (or CD4⁺ T cell)-inducing peptides are less than about 50 residues in length and usually consist of between about 6 and about 30 residues, more usually between about 12 and 25 (e.g., 12, 13, 14, 15, 16, 17, 18, 19, 20, 21, 22, 23, 24 or 25), and often between about 15 and 20 residues (e.g., 15, 16, 17, 18, 19 or 20).

The nomenclature used to describe peptide compounds follows the conventional practice wherein the amino group is presented to the left (the N-terminus) and the carboxyl group to the right (the C-terminus) of each amino acid residue. The L-form of an amino acid residue is represented by a capital single letter or a capital first letter of a three-letter symbol, and the D-form, for those amino acids having D-forms, is represented by a lower case single letter or a lower case three letter symbol. Glycine has no asymmetric carbon atom and is simply referred to as “Gly” or G. Symbols for the amino acids are as follows: (Single Letter Symbol; Three Letter Symbol Amino Acid) A; Ala; Alanine: C; Cys; Cysteine: D; Asp; Aspartic Acid: E; Glu; Glutamic Acid: F; Phe; Phenylalanine: G; Gly; Glycine: H; His; Histidine: I; Ile; Isoleucine: K; Lys; Lysine: L; Leu; Leucine: M; Met; Methionine: N; Asn; Asparagine: P; Pro; Proline: Q; Gln; Glutamine: R; Arg; Arginine: S; Ser; Serine: T; Thr; Threonine: V; Val; Valine: W; Trp; Tryptophan: Y; Tyr; Tyrosine. Amino acid “chemical characteristics” are defined as: Aromatic (F, W, Y); Aliphatic-hydrophobic (L, I, V, M); Small polar (S, T, C); Large polar (Q, N); Acidic (D, E); Basic (R, H, K); Non-polar: (P, A, G) Proline; Alanine; and Glycine. By way of example, amino acid substitutions can be carried out without resulting in a substantial modification of the associated activity (or activities) of the corresponding modified polypeptides; for example, the replacement of leucine with valine or isoleucine, of aspartic acid with glutamic acid, of glutamine with asparagine, of arginine with lysine, and the like, the reverse substitutions can be performed without substantial modification of the biological activity of the polypeptides.

In order to extend the life of the polypeptides according to the invention, it may be advantageous to use non-natural amino acids, for example in the D-form, or alternatively amino acid analogs, for example sulfur-containing forms of amino acids in the production of “variant polypeptides”. Alternative means for increasing the life of polypeptides can also be used in the practice of the instant invention. For example, polypeptides of the invention, and fragments thereof, can be recombinantly modified to include elements that increase the plasma, or serum half-life of the polypeptides of the invention. These elements include, and are not limited to, antibody constant regions (see for example, U.S. Pat. No. 5,565,335, hereby incorporated by reference in its entirety, including all references cited therein), or other elements such as those disclosed in U.S. Pat. Nos. 6,319,691, 6,277,375, or 5,643,570, each of which is incorporated by reference in its entirety, including all references cited within each respective patent. Alternatively, the polynucleotides and genes of the instant invention can be recombinantly fused to elements, well known to the skilled artisan, that are useful in the preparation of immunogenic constructs for the purposes of vaccine formulation.

The subject invention also provides biologically active fragments of a polypeptide according to the invention and includes those peptides capable of eliciting an immune response directed against Candida and/or Aspergillus, the immune response providing components (B-cells, antibodies, and/or or components of the cellular immune response (e.g., helper, cytotoxic, and/or suppressor T-cells)) reactive with the fragment of the polypeptide; the intact, full length, unmodified polypeptide disclosed herein; or both a fragment of a polypeptide and the intact, full length, unmodified polypeptides disclosed herein.

The subject application also provides a composition comprising at least one isolated, recombinant, or purified polypeptide as set forth herein and at least one additional component. In various aspects of the invention, the additional component is a solid support (for example, microtiter wells, magnetic beads, non-magnetic beads, agarose beads, glass, cellulose, plastics, polyethylene, polypropylene, polyester, nitrocellulose, nylon, or polysulfone) and/or a pharmaceutically acceptable excipient or adjuvant known to those skilled in the art. In some aspects of the invention, the solid support provides an array of polypeptides of the subject invention or an array of polypeptides comprising combinations of various polypeptides of the subject invention. Compositions of the subject invention can also comprise additional antigens of interest.

In one embodiment, the subject invention provides methods for eliciting an immune response in a subject comprising the administration of compositions comprising polypeptides according to the subject invention to a subject in amounts sufficient to induce an immune response in the subject. In some embodiments, a “protective” or “therapeutic immune response” is induced in the subject. A “protective immune response” or “therapeutic immune response” refers to a CTL (or CD8⁺ T cell) and/or an HTL (or CD4⁺ T cell), and/or an antibody response to an antigen derived from an infectious agent or a tumor antigen, which in some way prevents or at least partially arrests disease symptoms, side effects or progression. The protective immune response may also include an antibody response that has been facilitated by the stimulation of helper T cells (or CD4⁺ T cells). Additional methods of inducing an immune response in an individual are taught in U.S. Pat. No. 6,419,931, hereby incorporated by reference in its entirety. The term CTL can be used interchangeably with CD8⁺ T-cell(s) and the term HTL can be used interchangeably with CD4⁺ T-cell(s) throughout the subject application.

The terms “individual” and “subject” are used interchangeably to include mammals, such as apes, chimpanzees, orangutans, humans, monkeys or domesticated animals (pets) such as dogs, cats, guinea pigs, hamsters, Vietnamese pot-bellied pigs, rabbits, ferrets, cows, horses, goats and sheep. In a preferred embodiment, the methods of inducing an immune response contemplated herein are practiced on humans.

The composition administered to the subject may, optionally, contain an adjuvant and may be delivered in any manner known in the art for the delivery of immunogen to a subject. Compositions may also be formulated in any carriers, including for example, pharmaceutically acceptable carriers such as those described in E. W. Martin's Remington's Pharmaceutical Science, Mack Publishing Company, Easton, Pa. In preferred embodiments, compositions may be formulated in incomplete Freund's adjuvant, complete Freund's adjuvant, or alum.

In other embodiments, the subject invention provides for diagnostic assays based upon Western blot formats or standard immunoassays known to the skilled artisan. For example, antibody-based assays such as enzyme linked immunosorbent assays (ELISAs), radioimmunoassays (RIAs), lateral flow assays, reversible flow chromatographic binding assay (see, for example, U.S. Pat. No. 5,726,010, which is hereby incorporated by reference in its entirety), immunochromatographic strip assays, automated flow assays, and assays utilizing peptide- or antibody-containing biosensors may be employed for the detection of: 1) the polypeptides, and fragments thereof, provided by the subject invention; or 2) antibodies that bind to the polypeptides or fragments thereof, provided by the subject invention. Such assays and methods for conducting the assays are well-known in the art and the methods may test biological samples (e.g., serum, plasma, or blood) or environmental samples (e.g., soil, food, water) qualitatively (presence or absence of polypeptide) or quantitatively (comparison of a sample against a standard curve prepared using a polypeptide of the subject invention) for the presence of: a) one or more polypeptide of the subject invention, or 2) antibodies that bind to polypeptides of the subject invention. The detection of antibodies in adsorbed or unadsorbed samples derived from an individual using the polypeptides (and/or combinations thereof) disclosed in this application is an indication that the individual may have an active infection.

The subject invention provides a method of detecting a Candida and/or Aspergillus in a biological sample from a subject, comprising assessing the presence of a polypeptide or nucleic acid molecule encoding the polypeptide, in the sample; wherein the presence of the nucleic acid molecule or polypeptide is indicative of the presence of Candida and/or Aspergillus or an active infection by these organisms (e.g., is a marker associated with infection by one or both of these organisms). In one embodiment, the polypeptide is one or more polypeptides (e.g., one, two, three, or four or more antigens) selected from among SET1 (chromatin regulatory protein), ENO1 (enolase I), PGK1-2 (phosphoglycerate kinease), and MUC1-2 (cell surface glycoprotein).

In one embodiment, the subject invention provides a method of detecting a Candida and/or Aspergillus polypeptide, variant, or fragment of said polypeptide or variant (e.g., to detect an infection or monitor a known infection), comprising contacting a sample with an antibody that specifically binds to: 1) a polypeptide, or fragment thereof, or 2) a variant, or a fragment thereof, and detecting the presence of an antibody-antigen complex. Alternatively, the subject invention provides a method of detecting antibodies to Candida and/or Aspergillus comprising contacting a sample from a subject with: 1) a polypeptide of the subject invention, or fragment thereof, or 2) a variant of the subject invention, or a fragment thereof, and detecting the presence of an antibody-antigen complex. A sample can comprise a blood, serum, or tissue sample from an individual infected by Candida and/or Aspergillus. Alternatively, a sample can comprise culture medium in which polypeptides of the subject invention (or fragments thereof) are expressed or transformed host cells (lysed or intact cells) expressing polypeptides (or fragments thereof) that are provided by the subject invention.

In one embodiment of the method of detecting a Candida and/or Aspergillus polypeptide, variant, or fragment, the one or more polypeptides (e.g., one, two, three, four, five, or six or more polypeptides) is among those polypeptides disclosed in Tables 3, 4, 5, 6, 7, 8, 9, 12, 13, 14, 15, 16, 17 or 18 herein, or among those polypeptides encoded by the nucleic acids disclosed in Tables 3, 4, 5, 6, 7, 8, 9, 12, 13, 14, 15, 16, or 17 herein. In another embodiment, the one or more polypeptides (e.g., one, two, three, four, five, or six or more polypeptides) are from C. albicans and selected from the group consisting of Set1p, Rbt4p, Met6p, BGl2p, Gap1, Bgl2, Carl, Enol1, Fba1, IPF9162, PGK1, and Muc1. In another embodiment, the one or more polypeptides (e.g., one, two, three, or four polypeptides) are from C. albicans and selected from the group consisting of Set1p, Rbt4p, Met6p, and BGl2p. In another embodiment, the one or more polypeptides (e.g., one, two, three, four, or five polypeptides) are from C. albicans and selected from the group consisting of Set1p, Rbt4p, Met6p, BGl2p, and Gap1. In another embodiment, the one or more polypeptides (e.g., one, two, three, or four polypeptides) are from C. albicans and selected from the group consisting of Car1, Enol1, Fba1, and IPF9162. In another embodiment, the polypeptide is from C. albicans and is PGK1. In another embodiment, the polypeptide is from C. albicans and is Muc1.

In another embodiment, the one or more antigens (e.g., one, two, three, or four or more antigens) are from C. albicans and selected from among SET1 (chromatin regulatory protein), ENO1 (enolase I), PGK1-2 (phosphoglycerate kinease), and MUC1-2 (cell surface glycoprotein). In another embodiment, the antigen is from C. albicans and is SET1. In another embodiment, the antigen is from C. albicans and is ENO1. In another embodiment, the antigen is from C. albicans and is PGK1-2. In another embodiment, the antigen is from C. albicans and is MUC1-2.

The Candida or Aspergillus infection detected or monitored using the methods of the invention can be of various types, such as invasive aspergillosis (IA), disseminated candidiasis (DC), systemic candidiasis, oropharyngeal infection (OPC), vulvovaginal infection (VVC), allergic bronchopulmonary aspergillosis (ABPA), aspergilloma or chronic pulmonary aspergillosis, aspergillus sinusitis, invasive aspergillosis, etc. Optionally, the detection method further includes diagnosing the subject as suffering from the infection. Optionally, the detection methods of the invention further include evaluating the subject for clinical symptoms of infection. Optionally, the detection methods of the invention further include administering a treatment appropriate for the infection (e.g., with an antifungal agent). The detection methods of the invention may be used to monitor an infection and to predict the subject's responsiveness to treatment based on the profile of Candida or Aspergillus nucleic acids/polypeptides detected in a biological sample from the subject (such as whole blood, serum, plasma, aspirate, saliva, urine, discharge, etc.).

Typically, the antibody-based assays can be considered to be of four general types: direct binding assays, sandwich assays, competition assays, and displacement assays. In a direct binding assay, either the antibody or antigen is labeled, and there is a means of measuring the number of complexes formed. In a sandwich assay, the formation of a complex of at least three components (e.g., antibody-antigen-antibody) is measured. In a competition assay, labeled antigen and unlabelled antigen compete for binding to the antibody, and either the bound or the free component is measured. In a displacement assay, the labeled antigen is pre-bound to the antibody, and a change in signal is measured as the unlabelled antigen displaces the bound, labeled antigen from the receptor.

Lateral flow assays can be conducted according to the teachings of U.S. Pat. No. 5,712,170 and the references cited therein. U.S. Pat. No. 5,712,170 and the references cited therein are hereby incorporated by reference in their entireties. Displacement assays and flow immunosensors useful for carrying out displacement assays are described in: (1) Kusterbeck et al., “Antibody-Based Biosensor for Continuous Monitoring”, in Biosensor Technology, R. P. Buck et al., eds., Marcel Dekker, N.Y. pp. 345-350 (1990); Kusterbeck et al., “A Continuous Flow Immunoassay for Rapid and Sensitive Detection of Small Molecules”, Journal of Immunological Methods, vol. 135, pp. 191-197 (1990); Ligler et al., “Drug Detection Using the Flow Immunosensor”, in Biosensor Design and Application, J. Findley et al., eds., American Chemical Society Press, pp. 73-80 (1992); and Ogert et al., “Detection of Cocaine Using the Flow Immunosensor”, Analytical Letters, vol. 25, pp. 1999-2019 (1992), all of which are incorporated herein by reference in their entireties. Displacement assays and flow immunosensors are also described in U.S. Pat. No. 5,183,740, which is also incorporated herein by reference in its entirety. The displacement immunoassay, unlike most of the competitive immunoassays used to detect small molecules, can generate a positive signal with increasing antigen concentration. One aspect of the invention allows for the exclusion of Western blots as a diagnostic assay, particularly where the Western blot is a screen of whole cell lysates of Candida and/or Aspergillus, or related organisms, against immune serum of infected individuals. In another aspect of the invention, peptide, or polypeptide, based diagnostic assays utilize Candida and/or Aspergillus polypeptides that have been produced either by chemical peptide synthesis or by recombinant methodologies.

The subject invention also provides methods of binding an antibody to a polypeptide of the subject invention comprising contacting a sample containing an antibody with a polypeptide under conditions that allow for the formation of an antibody-antigen complex. These methods can further comprise the step of detecting the formation of said antibody-antigen complex. In various aspects of this method, an immunoassay is conducted for the detection of Candida and/or Aspergillus. Non-limiting examples of such immunoassays include enzyme linked immunosorbent assays (ELISAs), radioimmunoassays (RIAs), lateral flow assays, immunochromatographic strip assays, automated flow assays, Western blots, immunoprecipitation assays, reversible flow chromatographic binding assays, agglutination assays, and biosensors. Additional aspects of the invention provide for the use of an array of polypeptides when conducted the aforementioned methods of detection (the array can comprise polypeptides of the same or different sequence as well as polypeptides from one or more other organisms.

The subject invention also concerns antibodies that bind to polypeptides of the invention. Antibodies that are immunospecific for the polypeptides as set forth herein are specifically contemplated. In various embodiments, antibodies that do not cross-react with other proteins are also specifically contemplated. The antibodies of the subject invention can be prepared using standard materials and methods known in the art (see, for example, Monoclonal Antibodies: Principles and Practice, 1983; Monoclonal Hybridoma Antibodies: Techniques and Applications, 1982; Selected Methods in Cellular Immunology, 1980; Immunological Methods, Vol. II, 1981; Practical Immunology, and Kohler et al. [1975] Nature 256:495). These antibodies can further comprise one or more additional components, such as a solid support, a carrier or pharmaceutically acceptable excipient, or a label.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity, particularly neutralizing activity. Antibody fragments compromise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multi-specific antibodies formed from antibody fragments.

The term monoclonal antibody as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. [1975] Nature 256: 495, or may be made by recombinant DNA methods (see, e.g., U.S. Pat. No. 4,816,567). The monoclonal antibodies may also be isolated from phage antibody libraries using the techniques described in Clackson et al. [1991] Nature 352: 624-628 and Marks et al. [1991] J. Mol. Biol. 222: 581-597, for example.

The monoclonal antibodies described herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. [1984] Proc. Natl. Acad Sci. USA 81: 6851-6855). Also included are humanized antibodies, such as those taught in U.S. Pat. Nos. 6,407,213 or 6,417,337 which are hereby incorporated by reference in their entirety.

“Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies [1994] Vol. 113:269-315, Rosenburg and Moore eds. Springer-Verlag, New York.

The term diabodies refers to small antibody fragments with two antigen-binding sites, which fragments comprise a heavy chain variable domain (V_{H}) connected to a light chain variable domain (V_{L}) in the same polypeptide chain (V_{H}-V_{L}). Diabodies are described more fully in, for example, EP 404,097; WO 93/11161; and Hollinger et al. [1993] Proc. Natl. Acad. Sci. USA 90: 6444-6448. The term linear antibodies refers to the antibodies described in Zapata et al. [1995] Protein Eng. 8(10):1057-1062.

An isolated antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The terms “comprising”, “consisting of and “consisting essentially of are defined according to their standard meaning. The terms may be substituted for one another throughout the instant application in order to attach the specific meaning associated with each term. The phrases “isolated” or “biologically pure” refer to material that is substantially or essentially free from components which normally accompany the material as it is found in its native state. Thus, isolated peptides in accordance with the invention preferably do not contain materials normally associated with the peptides in their in situ environment. “Link” or “join” refers to any method known in the art for functionally connecting peptides, including, without limitation, recombinant fusion, covalent bonding, disulfide bonding, ionic bonding, hydrogen bonding, and electrostatic bonding.

The subject invention also provides isolated, recombinant, and/or purified polynucleotide sequences comprising:

a) a polynucleotide sequence encoding a polypeptide as set forth in Tables 3, 4, 5, 6, 7, 8, 9, 12, 13, 14, 15, 16, 17 or 18 disclosed herein;

b) a polynucleotide sequence having at least about 20% to 99.99% identity to a polynucleotide sequence encoding a polypeptide set forth in Tables 3, 4, 5, 6, 7, 8, 9, 12, 13, 14, 15, 16, 17 or 18 disclosed herein, wherein said polynucleotide encodes a polypeptide having at least one of the activities of the native polypeptide;

c) a polynucleotide sequence (a coding sequence) set forth in Tables 3, 4, 5, 6, 7, 8, 9, 12, 13, 14, 15, 16, 17 or 18 disclosed herein;

d) a polynucleotide sequence having at least about 20% to 99.99% identity to the polynucleotide sequence of (a), (b), or (c);

e) a polynucleotide that is complementary to the polynucleotides set forth in (a), (b), (c), or (d);

f) a genetic construct comprising a polynucleotide sequence as set forth in (a), (b), (c), (d), or (e);

g) a vector comprising a polynucleotide or genetic construct as set forth in (a), (b), (c), (d), (e), or (f);

h) a host cell comprising a vector as set forth in (g);

i) a polynucleotide that hybridizes under low, intermediate or high stringency with a polynucleotide sequence as set forth in (a), (b), (c), (d), (e), (f), or (g); or

j) a probe comprising a polynucleotide according to (a), (b), (c), (d), (e), (f), or (g) and, optionally, a label or marker.

In one embodiment of each of the aforementioned aspects of the invention a)-n), the one or more polynucleotides (e.g., one, two, three, four, five, or six or more polynucleotides) is among those disclosed Tables 3, 4, 5, 6, 7, 8, 9, 12, 13, 14, 15, 16, or 17 herein. In another embodiment, the one or more polynucleotides (e.g., one, two, three, four, five, or six or more polynucleotides) are from C. albicans and selected from the group consisting of Set1p, Rbt4p, Met6p, BGl2p, Gap1, Bgl2, Car1, Enol1, Fba1, IPF9162, PGK1, and Muc1. In another embodiment, the one or more polynucleotides (e.g., one, two, three, or four polynucleotides) are from C. albicans and selected from the group consisting of Set1p, Rbt4p, Met6p, and BGl2p. In another embodiment, the one or more polynucleotides (e.g., one, two, three, four, or five polynucleotides) are from C. albicans and selected from the group consisting of Set1p, Rbt4p, Met6p, BGl2p, and Gap1. In another embodiment, the one or more polynucleotides (e.g., one, two, three, or four antigens) are from C. albicans and selected from the group consisting of Car1, Enol1, Fba1, and IPF9162. In another embodiment, the polynucleotide is from C. albicans and is PGK1. In another embodiment, the polynucleotide is from C. albicans and is Muc1.

In another embodiment, the one or more antigens (e.g., one, two, three, or four or more antigens) are from C. albicans and selected from among SET1 (chromatin regulatory protein), ENO1 (enolase I), PGK1-2 (phosphoglycerate kinease), and MUC1-2 (cell surface glycoprotein). In another embodiment, the antigen is from C. albicans and is SET1. In another embodiment, the antigen is from C. albicans and is ENO1. In another embodiment, the antigen is from C. albicans and is PGK1-2. In another embodiment, the antigen is from C. albicans and is MUC1-2.

The terms “nucleotide sequence”, “polynucleotide” and “nucleic acid” can be used interchangeably and are understood to mean, according to the present invention, either a double-stranded DNA, a single-stranded DNA or products of transcription of the said DNAs (e.g., RNA molecules). It should also be understood that the present invention does not relate to genomic polynucleotide sequences in their natural environment or natural state. The nucleic acid, polynucleotide, or nucleotide sequences of the invention can be isolated, purified (or partially purified), by separation methods including, but not limited to, ion-exchange chromatography, molecular size exclusion chromatography, or by genetic engineering methods such as amplification, subtractive hybridization, cloning, subcloning or chemical synthesis, or combinations of these genetic engineering methods.

A homologous polynucleotide or polypeptide sequence, for the purposes of the present invention, encompasses a sequence having a percentage identity with the polynucleotide or polypeptide sequences, set forth herein, of between at least (or at least about) 20.00% to 99.99% (inclusive). The aforementioned range of percent identity is to be taken as including, and providing written description and support for, any fractional percentage, in intervals of 0.01%, between 20.00% and, up to, including 99.99%. These percentages are purely statistical and differences between two nucleic acid sequences can be distributed randomly and over the entire sequence length. For example, homologous sequences can exhibit a percent identity of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent with the sequences of the instant invention. Typically, the percent identity is calculated with reference to the full length, native, and/or naturally occurring polynucleotide. The terms “identical” or percent “identity”, in the context of two or more polynucleotide or polypeptide sequences, refer to two or more sequences or subsequences that are the same or have a specified percentage of amino acid residues that are the same, when compared and aligned for maximum correspondence over a comparison window, as measured using a sequence comparison algorithm or by manual alignment and visual inspection.

Both protein and nucleic acid sequence homologies may be evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman, 1988, Proc. Natl. Acad. Sci. USA 85(8):2444-2448; Altschul et al., 1990, J. Mol. Biol. 215(3):403-410; Thompson et al., 1994, Nucleic Acids Res. 22(2):4673-4680; Higgins et al., 1996, Methods Enzymol. 266:383-402; Altschul et al., 1990, J, Mol. Biol. 215(3):403-410; Altschul et al., 1993, Nature Genetics 3:266-272). Sequence comparisons are, typically, conducted using default parameters provided by the vendor or using those parameters set forth in the above-identified references, which are hereby incorporated by reference in their entireties.

A “complementary” polynucleotide sequence, as used herein, generally refers to a sequence arising from the hydrogen bonding between a particular purine and a particular pyrimidine in double-stranded nucleic acid molecules (DNA-DNA, DNA-RNA, or RNA-RNA). The major specific pairings are guanine with cytosine and adenine with thymine or uracil. A “complementary” polynucleotide sequence may also be referred to as an “antisense” polynucleotide sequence or an “antisense sequence”.

Sequence homology and sequence identity can also be determined by hybridization studies under high stringency, intermediate stringency, and/or low stringency. Various degrees of stringency of hybridization can be employed. The more severe the conditions, the greater the complementarity that is required for duplex formation. Severity of conditions can be controlled by temperature, probe concentration, probe length, ionic strength, time, and the like. Preferably, hybridization is conducted under low, intermediate, or high stringency conditions by techniques well known in the art, as described, for example, in Keller, G. H., M. M. Manak [1987] DNA Probes, Stockton Press, New York, N.Y., pp. 169-170.

For example, hybridization of immobilized DNA on Southern blots with ³²P-labeled gene-specific probes can be performed by standard methods (Maniatis et al. [1982] Molecular Cloning: A Laboratory Manual, Cold Spring Harbor Laboratory, New York). In general, hybridization and subsequent washes can be carried out under intermediate to high stringency conditions that allow for detection of target sequences with homology to the exemplified polynucleotide sequence. For double-stranded DNA gene probes, hybridization can be carried out overnight at 20-25° C. below the melting temperature (Tₘ) of the DNA hybrid in 6× SSPE, 5× Denhardt's solution, 0.1% SDS, 0.1 mg/ml denatured DNA. The melting temperature is described by the following formula (Beltz et al. [1983] Methods of Enzymology, R. Wu, L. Grossman and K. Moldave [eds.] Academic Press, New York 100:266-285).

Tm=81.5° C+16.6 Log[Na⁺]0.41 (% G+C)-0.61 (% formamide)-600/length of duplex in base pairs.

Washes are typically carried out as follows:

(1) twice at room temperature for 15 minutes in 1× SSPE, 0.1% SDS (low stringency wash);

(2) once at Tₘ−20° C. for 15 minutes in 0.2× SSPE, 0.1% SDS (intermediate stringency wash).

For oligonucleotide probes, hybridization can be carried out overnight at 10-20° C. below the melting temperature (Tₘ) of the hybrid in 6× SSPE, 5× Denhardt's solution, 0.1% SDS, 0.1 mg/ml denatured DNA. Tₘ for oligonucleotide probes can be determined by the following formula:

Tₘ(° C.)=2(number T/A base pairs)⁺4(number G/C base pairs) (Suggs et al. [1981] ICN-UCLA Symp. Dev. Biol. Using Purified Genes, D. D. Brown [ed.], Academic Press, New York, 23:683-693).

Washes can be carried out as follows:

(1) twice at room temperature for 15 minutes 1× SSPE, 0.1% SDS (low stringency wash);

(2) once at the hybridization temperature for 15 minutes in 1× SSPE, 0.1% SDS (intermediate stringency wash).

In general, salt and/or temperature can be altered to change stringency. With a labeled DNA fragment >70 or so bases in length, the following conditions can be used:
Low: 1 or 2× SSPE, room temperature
Low: 1 or 2× SSPE, 42° C.
Intermediate: 0.2× or 1× SSPE, 65° C.
High: 0.1× SSPE, 65° C.

By way of another non-limiting example, procedures using conditions of high stringency can also be performed as follows: Pre-hybridization of filters containing DNA is carried out for 8 hours to overnight at 65° C. in buffer composed of 6× SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C., the preferred hybridization temperature, in pre-hybridization mixture containing 100 μg/ml denatured salmon sperm DNA and 5-20×10⁶ cpm of ³²P-labeled probe. Alternatively, the hybridization step can be performed at 65° C. in the presence of SSC buffer, 1× SSC corresponding to 0.15M NaCl and 0.05 M Na citrate. Subsequently, filter washes can be done at 37° C. for 1 hour in a solution containing 2× SSC, 0.01% PVP, 0.01% Ficoll, and 0.01% BSA, followed by a wash in 0.1× SSC at 50° C. for 45 minutes. Alternatively, filter washes can be performed in a solution containing 2× SSC and 0.1% SDS, or 0.5× SSC and 0.1% SDS, or 0.1× SSC and 0.1% SDS at 68° C. for 15 minute intervals. Following the wash steps, the hybridized probes are detectable by autoradiography. Other conditions of high stringency which may be used are well known in the art and as cited in Sambrook et al., 1989, Molecular Cloning, A Laboratory Manual, Second Edition, Cold Spring Harbor Press, N.Y., pp. 9.47-9.57; and Ausubel et al., 1989, Current Protocols in Molecular Biology, Green Publishing Associates and Wiley Interscience, N.Y. are incorporated herein in their entirety.

Another non-limiting example of procedures using conditions of intermediate stringency are as follows: Filters containing DNA are pre-hybridized, and then hybridized at a temperature of 60° C. in the presence of a 5× SSC buffer and labeled probe. Subsequently, filters washes are performed in a solution containing 2× SSC at 50° C. and the hybridized probes are detectable by autoradiography. Other conditions of intermediate stringency which may be used are well known in the art and as cited in Sambrook et al., 1989, Molecular Cloning, A Laboratory Manual, Second Edition, Cold Spring Harbor Press, N.Y., pp. 9.47-9.57; and Ausubel et al., 1989, Current Protocols in Molecular Biology, Green Publishing Associates and Wiley Interscience, N.Y. are incorporated herein in their entirety.

Duplex formation and stability depend on substantial complementarity between the two strands of a hybrid and, as noted above, a certain degree of mismatch can be tolerated. Therefore, the probe sequences of the subject invention include mutations (both single and multiple), deletions, insertions of the described sequences, and combinations thereof, wherein said mutations, insertions and deletions permit formation of stable hybrids with the target polynucleotide of interest. Mutations, insertions and deletions can be produced in a given polynucleotide sequence in many ways, and these methods are known to an ordinarily skilled artisan. Other methods may become known in the future.

It is also well known in the art that restriction enzymes can be used to obtain functional fragments of the subject DNA sequences. For example, Bal31 exonuclease can be conveniently used for time-controlled limited digestion of DNA (commonly referred to as “erase-a-base” procedures). See, for example, Maniatis et al. [1982] Molecular Cloning: A Laboratory Manual, Cold Spring Harbor Laboratory, New York; Wei et al. [1983] J. Biol. Chem. 258:13006-13512.

The present invention further comprises fragments of the polynucleotide sequences of the instant invention. Representative fragments of the polynucleotide sequences according to the invention will be understood to mean any nucleotide fragment having at least 5 successive nucleotides, preferably at least 12 successive nucleotides, and still more preferably at least 15, 18, or at least 20 successive nucleotides of the sequence from which it is derived. The upper limit for such fragments is the total number of nucleotides found in the full-length sequence encoding a particular polypeptide. The term “successive” can be interchanged with the term “consecutive” or the phrase “contiguous span”. Thus, in some embodiments, a polynucleotide fragment may be referred to as “a contiguous span of at least X nucleotides, wherein X is any integer value beginning with 5; the upper limit for such fragments is one nucleotide less than the total number of nucleotides found in the full-length sequence encoding a particular polypeptide.

In some embodiments, the subject invention includes those fragments capable of hybridizing under various conditions of stringency conditions (e.g., high or intermediate or low stringency) with a nucleotide sequence according to the invention; fragments that hybridize with a nucleotide sequence of the subject invention can be, optionally, labeled as set forth below.

The subject invention provides, in one embodiment, methods for the identification of the presence of nucleic acids according to the subject invention in transformed host cells or in cells isolated from an individual suspected of being infected by Candida and/or Aspergillus. In these varied embodiments, the invention provides for the detection of nucleic acids in a sample (obtained from the individual or from a cell culture) comprising contacting a sample with a nucleic acid (polynucleotide) of the subject invention (such as an RNA, mRNA, DNA, cDNA, or other nucleic acid). In a preferred embodiment, the polynucleotide is a probe that is, optionally, labeled and used in the detection system. Many methods for detection of nucleic acids exist and any suitable method for detection is encompassed by the instant invention. Typical assay formats utilizing nucleic acid hybridization include, and are not limited to, 1) nuclear run-on assay, 2) slot blot assay, 3) northern blot assay (Alwine, et al., Proc. Natl. Acad. Sci. 74:5350), 4) magnetic particle separation, 5) nucleic acid or DNA chips, 6) reverse Northern blot assay, 7) dot blot assay, 8) in situ hybridization, 9) RNase protection assay (Melton, et al., Nuc. Acids Res. 12:7035 and as described in the 1998 catalog of Ambion, Inc., Austin, Tex.), 10) ligase chain reaction, 11) polymerase chain reaction (PCR), 12) reverse transcriptase (RT)-PCR (Berchtold, et al., Nuc. Acids. Res. 17:453), 13) differential display RT-PCR (DDRT-PCR) or other suitable combinations of techniques and assays. Labels suitable for use in these detection methodologies include, and are not limited to 1) radioactive labels, 2) enzyme labels, 3) chemiluminescent labels, 4) fluorescent labels, 5) magnetic labels, or other suitable labels, including those set forth below. These methodologies and labels are well known in the art and widely available to the skilled artisan. Likewise, methods of incorporating labels into the nucleic acids are also well known to the skilled artisan.

Thus, the subject invention also provides detection probes (e.g., fragments of the disclosed polynucleotide sequences) for hybridization with a target sequence or the amplicon generated from the target sequence. Such a detection probe will comprise a contiguous/consecutive span of at least 8, 9, 10, 11, 12, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 nucleotides. Labeled probes or primers are labeled with a radioactive compound or with another type of label as set forth above (e.g., 1) radioactive labels, 2) enzyme labels, 3) chemiluminescent labels, 4) fluorescent labels, or 5) magnetic labels). Alternatively, non-labeled nucleotide sequences may be used directly as probes or primers; however, the sequences are generally labeled with a radioactive element (³²P, ³⁵S, ³H, ¹²⁵I) or with a molecule such as biotin, acetylaminofluorene, digoxigenin, 5-bromo-deoxyuridine, or fluorescein to provide probes that can be used in numerous applications.

Polynucleotides of the subject invention can also be used for the qualitative and quantitative analysis of gene expression using arrays or polynucleotides that are attached to a solid support. As used herein, the term array means a one-, two-, or multi-dimensional arrangement of full length polynucleotides or polynucleotides of sufficient length to permit specific detection of gene expression. Preferably, the fragments are at least 15 nucleotides in length. More preferably, the fragments are at least 100 nucleotides in length. More preferably, the fragments are more than 100 nucleotides in length. In some embodiments the fragments may be more than 500 nucleotides in length.

For example, quantitative analysis of gene expression may be performed with full-length polynucleotides of the subject invention, or fragments thereof, in a complementary DNA microarray as described by Schena et al. (Science 270:467-470, 1995; Proc. Natl. Acad. Sci. U.S.A. 93:10614-10619, 1996). Polynucleotides, or fragments thereof, are amplified by PCR and arrayed onto silylated microscope slides. Printed arrays are incubated in a humid chamber to allow rehydration of the array elements and rinsed, once in 0.2% SDS for 1 minute, twice in water for 1 minute and once for 5 minutes in sodium borohydride solution. The arrays are submerged in water for 2 minutes at 95° C., transferred into 0.2% SDS for 1 minute, rinsed twice with water, air dried and stored in the dark at 25° C.

mRNA is isolated from a biological sample and probes are prepared by a single round of reverse transcription. Probes are hybridized to 1 cm² microarrays under a 14×14 mm glass coverslip for 6-12 hours at 60° C. Arrays are washed for 5 minutes at 25° C. in low stringency wash buffer (1× SSC/0.2% SDS), then for 10 minutes at room temperature in high stringency wash buffer (0.1× SSC/0.2% SDS). Arrays are scanned in 0.1× SSC using a fluorescence laser scanning device fitted with a custom filter set. Accurate differential expression measurements are obtained by taking the average of the ratios of two independent hybridizations.

Quantitative analysis of the polynucleotides present in a biological sample can also be performed in complementary DNA arrays as described by Pietu et al. (Genome Research 6:492-503, 1996). The polynucleotides of the invention, or fragments thereof, are PCR amplified and spotted on membranes. Then, mRNAs originating from biological samples derived from various tissues or cells are labeled with radioactive nucleotides. After hybridization and washing in controlled conditions, the hybridized mRNAs are detected by phospho-imaging or autoradiography. Duplicate experiments are performed and a quantitative analysis of differentially expressed mRNAs is then performed.

Alternatively, the polynucleotide sequences of to the invention may also be used in analytical systems, such as DNA chips. DNA chips and their uses are well known in the art and (see for example, U.S. Pat. Nos. 5,561,071; 5,753,439; 6,214,545; Schena et al., BioEssays, 1996, 18:427-431; Bianchi et al., Clin. Diagn. Virol., 1997, 8:199-208; each of which is hereby incorporated by reference in their entireties) and/or are provided by commercial vendors such as Affymetrix, Inc. (Santa Clara, Calif.). In addition, the nucleic acid sequences of the subject invention can be used as molecular weight markers in nucleic acid analysis procedures.

The subject invention also provides for modified nucleotide sequences. Modified nucleic acid sequences will be understood to mean any nucleotide sequence that has been modified, according to techniques well known to persons skilled in the art, and exhibiting modifications in relation to the native, naturally occurring nucleotide sequences.

The subject invention also provides genetic constructs comprising: a) a polynucleotide sequence encoding a polypeptide set forth in Tables 3, 4, 5, 6, 7, 8, 9, 12, 13, 14, 15, 16, 17 or 18 disclosed herein, or a fragment thereof; b) a polynucleotide sequence having at least about 20% to 99.99% identity to a polynucleotide sequence encoding a native polypeptide, or a fragment of the native polypeptide, wherein the polynucleotide encodes a polypeptide having at least one of the activities or a polypeptide of the native full length polypeptide, or a fragment thereof; c) a polynucleotide sequence encoding a fragment of a polypeptide listed in Tables 3, 4, 5, 6, 7, 8, 9, 12, 13, 14, 15, 16, 17 or 18 disclosed herein, wherein said fragment has at least one of the activities of the polypeptide; d) a polynucleotide sequence listed in Tables 3, 4, 5, 6, 7, 8, 9, 12, 13, 14, 15, 16 or 17 disclosed herein; e) a polynucleotide sequence having at least about 20% to 99.99% identity to the polynucleotide sequence listed in Tables 3, 4, 5, 6, 7, 8, 9, 12, 13, 14, 15, 16, or 17; f) a polynucleotide sequence encoding a fragment of a variant polypeptide as set forth in (e); g) a polynucleotide sequence encoding a multimeric construct; or h) a polynucleotide that is complementary to the polynucleotides set forth in (a), (b), (c), (d), (e), (f), or (g). Genetic constructs of the subject invention can also contain additional regulatory elements such as promoters and enhancers and, optionally, selectable markers.

Also within the scope of the subject instant invention are vectors or expression cassettes containing genetic constructs as set forth herein or polynucleotides encoding the polypeptides, set forth supra, operably linked to regulatory elements. The vectors and expression cassettes may contain additional transcriptional control sequences as well. The vectors and expression cassettes may further comprise selectable markers. The expression cassette may contain at least one additional gene, operably linked to control elements, to be co-transformed into the organism. Alternatively, the additional gene(s) and control element(s) can be provided on multiple expression cassettes. Such expression cassettes are provided with a plurality of restriction sites for insertion of the sequences of the invention to be under the transcriptional regulation of the regulatory regions. The expression cassette(s) may additionally contain selectable marker genes operably linked to control elements.

The expression cassette will include in the 5′-3′ direction of transcription, a transcriptional and translational initiation region, a DNA sequence of the invention, and a transcriptional and translational termination regions. The transcriptional initiation region, the promoter, may be native or analogous, or foreign or heterologous, to the host cell. Additionally, the promoter may be the natural sequence or alternatively a synthetic sequence. By “foreign” is intended that the transcriptional initiation region is not found in the native plant into which the transcriptional initiation region is introduced. As used herein, a chimeric gene comprises a coding sequence operably linked to a transcriptional initiation region that is heterologous to the coding sequence.

Another aspect of the invention provides vectors for the cloning and/or the expression of a polynucleotide sequence taught herein. Vectors of this invention, including vaccine vectors, can also comprise elements necessary to allow the expression and/or the secretion of the said nucleotide sequences in a given host cell. The vector can contain a promoter, signals for initiation and for termination of translation, as well as appropriate regions for regulation of transcription. In certain embodiments, the vectors can be stably maintained in the host cell and can, optionally, contain signal sequences directing the secretion of translated protein. These different elements are chosen according to the host cell used. Vectors can integrate into the host genome or, optionally, be autonomously-replicating vectors.

The subject invention also provides for the expression of a polypeptide, peptide, fragment, or variant encoded by a polynucleotide sequence disclosed herein comprising the culture of a host cell transformed with a polynucleotide of the subject invention under conditions that allow for the expression of the polypeptide and, optionally, recovering the expressed polypeptide.

The disclosed polynucleotide sequences can also be regulated by a second nucleic acid sequence so that the protein or peptide is expressed in a host transformed with the recombinant DNA molecule. For example, expression of a protein or peptide may be controlled by any promoter/enhancer element known in the art. Promoters which may be used to control expression include, but are not limited to, the CMV-IE promoter, the SV40 early promoter region (Bernoist and Chambon Nature, 1981, 290:304-310), the promoter contained in the 3′ long terminal repeat of Rous sarcoma virus (Yamamoto et al., 1980, Cell 22:787-797), the herpes simplex thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); prokaryotic vectors containing promoters such as the β-lactamase promoter (Villa-Kamaroff et al., 1978, Proc. Natl. Acad. Sci. U.S.A. 75:3727-3731), or the tac promoter (DeBoer et al., 1983, Proc. Natl. Acad. Sci. U.S.A. 80:21-25); see also “Useful proteins from recombinant bacteria” in Scientific American, 1980, 242:74-94; plant expression vectors comprising the nopaline synthetase promoter region (Herrera-Estrella et al., 1983, Nature 303:209-213) or the cauliflower mosaic virus 35S RNA promoter (Gardner et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or fungi such as the Gal 4 promoter, the ADC (alcohol dehydrogenase) promoter, PGK (phosphoglycerol kinase) promoter, and/or the alkaline phosphatase promoter.

The vectors according to the invention are, for example, vectors of plasmid or viral origin. In a specific embodiment, a vector is used that comprises a promoter operably linked to a protein or peptide-encoding nucleic acid sequence contained within the disclosed polynucleotide sequences, one or more origins of replication, and, optionally, one or more selectable markers (e.g., an antibiotic resistance gene). Expression vectors comprise regulatory sequences that control gene expression, including gene expression in a desired host cell. Exemplary vectors for the expression of the polypeptides of the invention include the pET-type plasmid vectors (Promega) or pBAD plasmid vectors (Invitrogen) or those provided in the examples below. Furthermore, the vectors according to the invention are useful for transforming host cells so as to clone or express the polynucleotide sequences of the invention.

The invention also encompasses the host cells transformed by a vector according to the invention. These cells may be obtained by introducing into host cells a nucleotide sequence inserted into a vector as defined above, and then culturing the said cells under conditions allowing the replication and/or the expression of the polynucleotide sequences of the subject invention.

The host cell may be chosen from eukaryotic or prokaryotic systems, such as for example bacterial cells, (Gram negative or Gram positive), yeast cells (for example, Saccharomyces cereviseae or Pichia pastoris), animal cells (such as Chinese hamster ovary (CHO) cells), plant cells, and/or insect cells using baculovirus vectors. In some embodiments, the host cells for expression of the polypeptides include, and are not limited to, those taught in U.S. Pat. Nos. 6,319,691, 6,277,375, 5,643,570, or 5,565,335, each of which is incorporated by reference in its entirety, including all references cited within each respective patent.

Furthermore, a host cell strain may be chosen which modulates the expression of the inserted sequences, or modifies and processes the gene product in the specific fashion desired. Expression from certain promoters can be elevated in the presence of certain inducers; thus, expression of the genetically engineered polypeptide may be controlled. Furthermore, different host cells have characteristic and specific mechanisms for the translational and post-translational processing and modification (e.g., glycosylation, phosphorylation) of proteins. Appropriate cell lines or host systems can be chosen to ensure the desired modification and processing of the foreign protein expressed. For example, expression in a bacterial system can be used to produce an unglycosylated core protein product. Expression in yeast will produce a glycosylated product. Expression in mammalian cells can be used to ensure “native” glycosylation of a heterologous protein. Furthermore, different vector/host expression systems may effect processing reactions to different extents.

The subject invention also concerns novel compositions that can be employed to elicit an immune response or a protective immune response. In this aspect of the invention, an amount of a composition comprising recombinant DNA or mRNA encoding a polynucleotide of the subject invention sufficient to elicit an immune response or protective immune response is administered to an individual. Signal sequences may be deleted from the nucleic acid encoding an antigen of interest and the individual may be monitored for the induction of an immune response according to methods known in the art. A “protective immune response” or “therapeutic immune response” refers to a CTL (or CD8⁺ T cell) and/or an HTL (or CD4³⁰ T cell) response to an antigen that, in some way, prevents or at least partially arrests disease symptoms, side effects or progression. The immune response may also include an antibody response that has been facilitated by the stimulation of helper T cells.

In another embodiment, the subject invention further comprises the administration of polynucleotide vaccines in conjunction with a polypeptide antigen, or composition thereof, of the invention. In a preferred embodiment, the antigen is the polypeptide that is encoded by the polynucleotide administered as the polynucleotide vaccine. As a particularly preferred embodiment, the polypeptide antigen is administered as a booster subsequent to the initial administration of the polynucleotide vaccine.

A further embodiment of the subject invention provides for the induction of an immune response to the Candida and/or Aspergillus antigens disclosed herein using a “prime-boost” vaccination regimen known to those skilled in the art. In this aspect of the invention, a DNA vaccine or polypeptide antigen of the subject invention is administered to a subject in an amount sufficient to “prime” the immune response of the subject. The immune response of the subject is then “boosted” via the administration of: 1) one or a combination of: a peptide, polypeptide, and/or full length polypeptide antigen of the subject invention (optionally in conjunction with a immunostimulatory molecule and/or an adjuvant); or 2) a viral vector that contains nucleic acid encoding one, or more, of the same or, optionally, different, antigens, multi-epitope constructs, and/or peptide antigens set forth herein. In some alternative embodiments of the invention, a gene encoding an immunostimulatory molecule may be incorporated into the viral vector used to “boost the immune response of the individual. Exemplary immunostimulatory molecules include, and are not limited to, IL-1, IL-2, IL-3, IL-4, IL-5, IL-6, IL-7, IL-8, IL-9, IL-10, IL-11, IL-15, Il-16, Il-18, IL-23, IL-24, erythropoietin, G-CSF, M-CSF, platelet derived growth factor (PDGF), MSF, FLT-3 ligand, EGF, fibroblast growth factor (FGF; e.g., aFGF (FGF-1)), bFGF (FGF-2), FGF-3, FGF-4, FGF-5, FGF-6, or FGF-7), insulin-like growth factors (e.g., IGF-1, IGF-2); vascular endothelial growth factor (VEGF); interferons (e.g., IFN-γ, IFN-α, IFN-β); leukemia inhibitory factor (LIF); ciliary neurotrophic factor (CNTF); oncostatin M; stem cell factor (SCF); transforming growth factors (e.g., TGF-a, TGF-β1, TGF-β1, TGF-β1), or chemokines (such as, but not limited to, BCA-1/BLC-1, BRAK/Kec, CXCL16, CXCR3, ENA-78/LIX, Eotaxin-1, Eotaxin-2/MPIF-2, Exodus-2/SLC, Fractalkine/Neurotactin, GROalpha/MGSA, HCC-1, 1-TAC, Lymphotactin/ATAC/SCM, MCP-1/MCAF, MCP-3, MCP-4, MDC/STCP-1, ABCD-1, MIP-1α, MIP-1β, MIP-2α/GROβ, MIP-3α/Exodus/LARC, MIP-3β/Exodus-3/ELC, MIP-4/PARC/DC-CK1, PF-4, RANTES, SDF1α, TARC, or TECK). Genes encoding these immunostimulatory molecules are known to those skilled in the art and coding sequences may be obtained from a variety of sources, including various patents databases, publicly available databases (such as the nucleic acid and protein databases found at the National Library of Medicine or the European Molecular Biology Laboratory), the scientific literature, or scientific literature cited in catalogs produced by companies such as Genzyme, Inc., R&D Systems, Inc, or InvivoGen, Inc. [see, for example, the 1995 Cytokine Research Products catalog, Genzyme Diagnostics, Genzyme Corporation, Cambridge Mass.; 2002 or 1995 Catalog of R&D Systems, Inc (Minneapolis, Minn.); or 2002 Catalog of InvivoGen, Inc (San Diego, Calif.) each of which is incorporated by reference in its entirety, including all references cited therein].

Methods of introducing DNA vaccines into individuals are well-known to the skilled artisan. For example, DNA can be injected into skeletal muscle or other somatic tissues (e.g., intramuscular injection). Cationic liposomes or biolistic devices, such as a gene gun, can be used to deliver DNA vaccines. Alternatively, iontophoresis and other means for transdermal transmission can be used for the introduction of DNA vaccines into an individual.

Viral vectors for use in the subject invention can have a portion of the viral genome is deleted to introduce new genes without destroying infectivity of the virus. The viral vector of the present invention is, typically, a non-pathogenic virus. At the option of the practitioner, the viral vector can be selected so as to infect a specific cell type, such as professional antigen presenting cells (e.g., macrophage or dendritic cells). Alternatively, a viral vector can be selected that is able to infect any cell in the individual. Exemplary viral vectors suitable for use in the present invention include, but are not limited to poxvirus such as vaccinia virus, avipox virus, fowlpox virus, a highly attenuated vaccinia virus (such as Ankara or MVA [Modified Vaccinia Ankara]), retrovirus, adenovirus, baculovirus and the like. In a preferred embodiment, the viral vector is Ankara or MVA.

General strategies for construction of vaccinia virus expression vectors are known in the art (see, for example, Smith and Moss Bio Techniques November/December, 306-312, 1984; U.S. Pat. No. 4,738,846 (hereby incorporated by reference in its entirety). Sutter and Moss (Proc. Natl. Acad. Sci U.S.A. 89:10847-10851, 1992) and Sutter et al. (Vaccine, 12(11):1032-40, 1994) disclose the construction and use as a vector, a non-replicating recombinant Ankara virus (MVA) which can be used as a viral vector in the present invention.

Compositions comprising the subject polynucleotides can include appropriate nucleic acid vaccine vectors (plasmids), which are commercially available (e.g., Vical, San Diego, Calif.) or other nucleic acid vectors (plasmids), which are also commercially available (e.g., Valenti, Burlingame, Calif.). Alternatively, compositions comprising viral vectors and polynucleotides according to the subject invention are provided by the subject invention. In addition, the compositions can include a pharmaceutically acceptable carrier, e.g., saline. The pharmaceutically acceptable carriers are well known in the art and also are commercially available. For example, such acceptable carriers are described in E. W. Martin's Remington's Pharmaceutical Science, Mack Publishing Company, Easton, Pa.

The subject invention also provides an assay that comprises the use of polynucleotides, as set forth herein, for the detection of Candida and/or Aspergillus. Some aspects of the invention provide for a method that comprises contacting a sample comprising a population of polynucleotides with a second population of polynucleotides under conditions that allow for the formation of an hybridization complex, wherein said second population of polynucleotides comprises polynucleotides that encode at least one polypeptide that is listed in Tables 3, 4, 5, 6, 7, 8, 9, 12, 13, 14, 15, 16, 17 or 18 disclosed herein; d) fragments of polynucleotides; e) a polypeptide as set forth in Tables 3, 4, 5, 6, 7, 8, 9, 12, 13, 14, 15, 16, 17 or 18 disclosed herein; f) a variant polypeptide of such native polypeptide, wherein the variant polypeptide specifically binds to an antibody that specifically binds to a polypeptide listed in Tables 3, 4, 5, 6, 7, 8, 9, 12, 13, 14, 15, 16, 17 or 18 disclosed herein; g) a variant polypeptide fragment of those listed in Tables 3, 4, 5, 6, 7, 8, 9, 12, 13, 14, 15, 16, 17 or 18 disclosed herein, wherein the variant polypeptide fragment specifically binds to an antibody that specifically binds to a polypeptide disclosed in Tables 3, 4, 5, 6, 7, 8, 9, 12, 13, 14, 15, 16, 17 or 18 disclosed herein or a fragment of those polypeptides; h) a variant of a polypeptide as set forth in Tables 3, 4, 5, 6, 7, 8, 9, 12, 13, 14, 15, 16, 17 or 18 disclosed herein, wherein the variant polypeptide specifically binds to an antibody that specifically binds to a polypeptide listed in Tables 3, 4, 5, 6, 7, 8, 9, 12, 13, 14, 15, 16, 17 or 18 disclosed herein; i) a heterologous polypeptide fused, in frame, to a polypeptide comprising one of those listed in Tables 3, 4, 5, 6, 7, 8, 9, 12, 13, 14, 15, 16, 17 or 18 disclosed herein; and j) mixtures of polypeptides as set forth in a), b), c), d), e), 1), g), h), or i). The method can further comprise the step of detecting the hybridization complex and the second population of polynucleotides can be an array of polynucleotides or the same or different sequence if desired.

In one embodiment of each of the aforementioned aspects of the invention a)-i), the one or more polypeptides (e.g., one, two, three, four, five, or six or more polypeptides) is among those polypeptides disclosed in Tables 3, 4, 5, 6, 7, 8, 9, 12, 13, 14, 15, 16, 17 or 18 herein, or among those polypeptides encoded by the nucleic acids disclosed in Tables 3, 4, 5, 6, 7, 8, 9, 12, 13, 14, 15, 16, or 17 herein. In another embodiment, the one or more polypeptides (e.g., one, two, three, four, five, or six or more polypeptides) are from C. albicans and selected from the group consisting of Set1p, Rbt4p, Met6p, BGl2p, Gap1, Bgl2, Car1, Enol1, Fba1, IPF9162, PGK1, and Muc1. In another embodiment, the one or more polypeptides (e.g., one, two, three, or four polypeptides) are from C. albicans and selected from the group consisting of Set1p, Rbt4p, Met6p, and BGl2p. In another embodiment, the one or more polypeptides (e.g., one, two, three, four, or five polypeptides) are from C. albicans and selected from the group consisting of Set1p, Rbt4p, Met6p, BGl2p, and Gap1. In another embodiment, the one or more polypeptides (e.g., one, two, three, or four polypeptides) are from C. albicans and selected from the group consisting of Car1, Enol1, Fba1, and 1PF9162. In another embodiment, the polypeptide is from C. albicans and is PGK1. In another embodiment, the polypeptide is from C. albicans and is Muc1.

The terms “comprising”, “consisting of”, and “consisting essentially of are defined according to their standard meaning and may he substituted for one another throughout the instant application in order to attach the specific meaning associated with each term.

As used in this specification and the appended claims, the singular forms “a”, “an”, and “the” include plural reference unless the context clearly dictates otherwise. Thus, for example, a reference to “a polypeptide” includes more than one such polypeptide, and the like. Reference to “an antigen” includes more than one such antigen. Reference to “a cell” includes more than one such cell. Reference to “a polynucleotide” includes more than one such polynucleotide.

### Exemplified Embodiments

The invention includes, but is not limited to, the following embodiments:
Embodiment 1. An isolated, recombinant, or purified polypeptide comprising

(a) an amino acid sequence listed in Tables 3, 4, 5, 6, 7, 8, 9, 12, 13, 14, 15, 16, 17 or 18 disclosed herein; or

(b) fragments of (a); or

(c) a polypeptide listed in Tables 3, 4, 5, 6, 7, 8, 9, 12, 13, 14, 15, 16, 17 or 18 disclosed herein; or

(d) one or more polypeptides (e.g., one, two, three, or four or more polypeptides) selected from among SET1 (chromatin regulatory protein), ENO1 (enolase I), PGK1-2 (phosphoglycerate kinease), and MUC1-2 (cell surface glycoprotein); or

(e) one or more polypeptides (e.g., one, two, three, four, five, six, seven, eight, nine, ten, eleven, twelve, thirteen, fourteen, or fifteen or more polypeptides) selected from among METE-1, MET6-2, NOT5, RBT4, IPF9162, CAR1, GAP1, SET1, ENO1, FBA1, PGK1-1, PGK1-2, MUC1-1, MUC1-2, and BGL2; or

(f) one or more polypeptides (e.g., one, two, three, four, five, six, or seven or more polypeptides) selected from among SET1, ENO1, FBA1, PGK1-1, PGK1-2, MUC1-2, and BGL2; or

(g) a variant of a polypeptide of (a), (b), (c), (d), (e), or (f), wherein said variant polypeptide specifically binds to an antibody that specifically binds to a polypeptide of (a), (b), (c), (d), (e), or (f); or

(h) a fragment of a polypeptide of (c), (d), (e), or (f), wherein said fragment specifically binds to an antibody that specifically binds to a polypeptide of (c), (d), (e), or (f), or a fragment of (c), (d), (e), or (f); or

(i) a heterologous polypeptide fused, in frame, to a polypeptide comprising the polypeptide of (a), (b), (c), (d), (e), or (f); or

(j) a multimeric construct comprising a polypeptide of (a), (b), (c), (d), (e), or (f); or a fragment or variant of (a), (b), (c), (d), (e), (f), (g), (h), or (i).
Embodiment 2. A composition comprising at least one isolated or purified polypeptide according to embodiment 1, or a isolated polynucleotide encoding the polypeptide; and an additional component.
Embodiment 3. The composition according to embodiment 2, wherein said additional component is a solid support, and wherein said polypeptide or said encoding polynucleotide is immobilized on said support.
Embodiment 4. The composition according to embodiment 3, wherein said solid support is selected from the group consisting of microtiter wells, magnetic beads, non-magnetic beads, agarose beads, glass, cellulose, plastics, polyethylene, polypropylene, polyester, nitrocellulose, nylon, and polysulfone.
Embodiment 5. The composition according to embodiment 2, wherein said additional component is a pharmaceutically acceptable excipient.
Embodiment 6. The composition according to embodiment 3 or 4, wherein said solid support provides an array of polypeptides or encoding polynucleotides, and wherein said array of polypeptides is selected from among the polypeptides listed in Tables 3, 4, 5, 6, 7, 8, 9, 12, 13, 14, 15, 16, 17 or 18 disclosed herein, or a fragment or variant thereof.
Embodiment 7. The composition according to any one of embodiments 2-5, wherein said polypeptide is one or more polypeptides (e.g., one, two, three, or four or more antigens) selected from among SET1 (chromatin regulatory protein), ENO1 (enolase I), PGK1-2 (phosphoglycerate kinease), and MUC1-2 (cell surface glycoprotein).
Embodiment 8. The composition according to any one of embodiments 2-5, wherein said polypeptide is one or more polypeptides (e.g., one, two, three, four, five, six, seven, eight, nine, ten, eleven, twelve, thirteen, fourteen, or fifteen or more polypeptides) selected from among METE-1, MET6-2, NOT5, RBT4, IPF9162, CAR1, GAP1, SET1, ENO1, FBA1, PGK1-1, PGK1-2, MUC1-1, MUC1-2, and BGL2.
Embodiment 9. The composition according to any one of embodiments 2-5, wherein said polypeptide is one or more polypeptides (e.g., one, two, three, four, five, six, or seven or more polypeptides) selected from among SET1, ENO1, FBA1, PGK1-1, PGK1-2, MUC1-2, and BGL2.
Embodiment 10. The composition according to any of embodiments 2-9, further comprising an additional antigen of interest.
Embodiment 11. A method of binding an antibody to a polypeptide comprising contacting a sample containing an antibody with a polypeptide under conditions that allow for the formation of an antibody-antigen complex, wherein said polypeptide is selected from the group consisting of the polypeptides listed in Tables 3, 4, 5, 6, 7, 8, 9, 12, 13, 14, 15, 16, 17 or 18 disclosed herein, or a fragment or variant thereof, wherein said sample containing an antibody is an adsorbed or nonadsorbed sample.
Embodiment 12. The method according to embodiment 11, wherein said polypeptide is one or more polypeptides (e.g., one, two, three, or four or more antigens) selected from among SET1 (chromatin regulatory protein), ENO1 (enolase I), PGK1-2 (phosphoglycerate kinease), and MUC1-2 (cell surface glycoprotein).
Embodiment 13. The method according to embodiment 11, wherein said polypeptide is one or more polypeptides (e.g., one, two, three, four, five, six, seven, eight, nine, ten, eleven, twelve, thirteen, fourteen, or fifteen or more polypeptides) selected from among MET6-1, MET6-2, NOT5, RBT4, 1PF9162, CAR1, GAP1, SET1, ENO1, FBA1, PGK1-1, PGK1-2, MUC1-1, MUC1-2, and BGL2.
Embodiment 14. The method according to embodiment 11, wherein said polypeptide is one or more polypeptides (e.g., one, two, three, four, five, six, or seven or more polypeptides) selected from among SET1, ENO1, FBA1, PGK1-1 PGK1-2, MUC1-2, and BGL2.
Embodiment 15. The method according to any of embodiments 11-14, further comprising the step of detecting the formation of said antibody-antigen complex.
Embodiment 16. The method according to any of embodiments 11-15, wherein said method is an immunoassay.
Embodiment 17. The method according to embodiment 16, wherein said immunoassay is selected from the group consisting of enzyme linked immunosorbent assays (ELISAs), radioimmunoassays (RIAs), lateral flow assays, immunochromatographic strip assays, automated flow assays, Western blots, immunoprecipitation assays, reversible flow chromatographic binding assays, agglutination assays, and biosensors.
Embodiment 18. The method according to any of embodiments 11-17, wherein said method is performed using an array of polypeptides.
Embodiment 19. The method according to embodiment 18, wherein said array comprises or consists of SET1 (chromatin regulatory protein), ENO1 (enolase I), PGK1-2 (phosphoglycerate kinease), and MUC1-2 (cell surface glycoprotein).
Embodiment 20. The method according to embodiment 18, wherein said array comprises or consists of METE-1, METE-2, NOT5, RBT4, IPF9162, CAR1, GAP1, SET1, ENO1, FBA1, PGK1-1, PGK1-2, MUC1-1, MUC1-2, and BGL2.
Embodiment 21. The method according to embodiment 18, wherein said comprises or consists of SET1, ENO1, FBA1, PGK1-1, PGK1-2, MUC1-2, and BGL2.
Embodiment 22. The method according to embodiment 18, wherein said array of polypeptides comprises the same polypeptide.
Embodiment 23. The method according to any of embodiments 18-21, wherein said array of polypeptides further comprises isolated polypeptides from other organisms of interest.
Embodiment 24. An isolated or purified polynucleotide comprising a nucleic acid sequence encoding a polypeptide of embodiment 1, or a fragment or variant thereof
Embodiment 25. An antibody that specifically binds to a polypeptide of embodiment 1, or a fragment or variant thereof.
Embodiment 26. The antibody according to embodiment 25, further comprising an additional component.
Embodiment 27. The antibody according to embodiment 26, wherein said additional component is a solid support.
Embodiment 28. The antibody according to embodiment 26, wherein said additional component is a carrier.
Embodiment 29. The antibody according to embodiment 28, wherein said carrier is a pharmaceutically acceptable excipient.
Embodiment 30. The antibody according to embodiment 26, wherein said additional component is a label.
Embodiment 31. A host cell comprising a polynucleotide according to embodiment 24.
Embodiment 32. A method of hybridizing polynucleotides comprising contacting a sample comprising a population of polynucleotides with a second population of polynucleotides under conditions that allow for the formation of an hybridization complex, wherein said second population of polynucleotides comprises polynucleotides that encode at least one polypeptide that is selected from among:

(a) an amino acid sequence listed in Tables 3, 4, 5, 6, 7, 8, 9, 12, 13, 14, 15, 16, 17 or 18 disclosed herein; or

(b) a fragment of (a); or

(c) a polypeptide listed in Tables 3, 4, 5, 6, 7, 8, 9, 12, 13, 14, 15, 16, 17 or 18 disclosed herein; or

(d) one or more polypeptides (e.g., one, two, three, or four or more polypeptides) selected from among SET1 (chromatin regulatory protein), ENO1 (enolase I), PGK1-2 (phosphoglycerate kinease), and MUC1-2 (cell surface glycoprotein); or

(e) one or more polypeptides (e.g., one, two, three, four, five, six, seven, eight, nine, ten, eleven, twelve, thirteen, fourteen, or fifteen or more polypeptides) selected from among MET6-1, MET6-2, NOT5, RBT4, IPF9162, CAR1, GAP1, SET1, ENO1, FBA1, PGK1-1, PGK1-2, MUC1-1, MUC1-2, and BGL2; or

(f) one or more polypeptides (e.g., one, two, three, four, five, six, or seven or more polypeptides) selected from among SET1, ENO1, FBA1, PGK1-1, PGK1-2, MUC1-2, and BGL2; or

(g) a variant of a polypeptide of (a), (b), (c), (d), (e), or (f), wherein said variant polypeptide specifically binds to an antibody that specifically binds to a polypeptide of (a), (b), (c), (d), (e), or (f); or

(h) a fragment of a polypeptide of (c), (d), (e), or (f), wherein said fragment specifically binds to an antibody that specifically binds to a polypeptide of (c), (d), (e), or (f), or a fragment of (c), (d), (e), or (f); or

(i) a heterologous polypeptide fused, in frame, to a polypeptide comprising the polypeptide of (a), (b), (c), (d), (e), (f), (g), or (h); or

(j) a multimeric construct comprising a polypeptide of (a), (b), (c), (d), (e), (f), (g), (h), or (i); or a fragment or variant of (c), (d), (c), or (f).
Embodiment 33. The method according to embodiment 32, further comprising the step of detecting the hybridization complex.
Embodiment 34. The method according to embodiment 32, wherein said second population of polynucleotides is an array of polynucleotides or the same or different sequence.
Embodiment 35. A method of inducing an immune response in a subject, comprising administering an effective amount of:

(1) a polypeptide antigen comprising:
(a) an amino acid sequence listed in Tables 3, 4, 5, 6, 7, 8, 9, 12, 13, 14, 15, 16, 17 or 18 disclosed herein; or
(b) a fragment of (a); or
(c) a polypeptide listed in Tables 3, 4, 5, 6, 7, 8, 9, 12, 13, 14, 15, 16, 17 or 18 disclosed herein; or
(d) one or more polypeptides (e.g., one, two, three, or four or more polypeptides) selected from among SET1 (chromatin regulatory protein), ENO1 (enolase I), PGK1-2 (phosphoglycerate kinease), and MUC1-2 (cell surface glycoprotein); or
(e) one or more polypeptides (e.g., one, two, three, four, five, six, seven, eight, nine, ten, eleven, twelve, thirteen, fourteen, or fifteen or more polypeptides) selected from among MET6-1, MET6-2, NOT5, RBT4, IPF9162, CAR1, GAP1, SET1, ENO1, FBA1, PGK1-1, PGK1-2, MUC1-1, MUC1-2, and BGL2; or
(f) one or more polypeptides (e.g., one, two, three, four, five, six, or seven or more polypeptides) selected from among SET1, ENO1, FBA1, PGK1-1, PGK1-2, MUC1-2, and BGL2; or
(g) a variant of a polypeptide of (a), (b), (c), (d), (e), or (f), wherein said variant polypeptide specifically binds to an antibody that specifically binds to a polypeptide of (a), (b), (c), (d), (e), or (f); or
(h) a fragment of a polypeptide of (c), (d), (e), or (f), wherein said fragment specifically binds to an antibody that specifically binds to a polypeptide of (c), (d), (e), or (1), or a fragment of (c), (d), (e), or (f); or
(i) a heterologous polypeptide fused, in frame, to a polypeptide comprising the polypeptide of (a), (b), (c), (d), (e), (f), (g), or (h); or
(j) a multimeric construct comprising a polypeptide of (c), (d), (e), or (f); or a fragment or variant of (a), (b), (c), (d), (e), (f), (g), (h), or (i); or

(2) a polynucleotide encoding at least one polypeptide antigen that is selected from (1).
Embodiment 36. The method according to embodiment 35, wherein the subject is immunocompromised.
Embodiment 37. A method for diagnosing or monitoring a Candida or Aspergillus infection in a subject, the method comprising:

(a) providing a gene expression profile obtained from a biological sample of the subject, wherein the expression profile comprises a plurality of Candida or Aspergillus genes that are expressed at the protein level; and

(b) comparing the subject's gene expression profile to a reference gene expression profile.
Embodiment 38. The method according to embodiment 37, wherein the reference gene expression profile is obtained from a normal, healthy individual, or from an infected individual.
Embodiment 39. The method according to embodiment 37, wherein the reference gene expression profile is contained within a database.
Embodiment 40. The method according to embodiment 37, wherein said comparing is carried out using a computer algorithm.
Embodiment 41. The method according to embodiment 37, wherein said method further comprises preparing the patient's gene expression profile.
Embodiment 42. The method according to embodiment 37, wherein said method further comprises:

(c) providing a gene expression profile obtained from a biological sample from the subject after the subject has undergone a treatment regimen for Candida or Aspergillus infection; and

(d) comparing the subject's post-treatment gene expression profile to the reference gene expression profile, to monitor the subject's response to the treatment regimen.
Embodiment 43. The method according to embodiment 37, wherein said method further comprises:

(c) providing a diagnosis of Candida or Aspergillus infection to the patient.
Embodiment 44. The method according to embodiment 33, wherein the plurality of Candida or Aspergillus genes is listed in Tables 3, 4, 5, 6, 7, 8, 9, 12, 13, 14, 15, 16, or 17 disclosed herein.
Embodiment 45. The method according to embodiment 37, wherein the plurality of Candida or Aspergillus genes comprises one or more polypeptides (e.g., one, two, three, or four or more polypeptides) selected from among SET1 (chromatin regulatory protein), ENO1 (enolase I), PGK1-2 (phosphoglycerate kinease), and MUC1-2 (cell surface glycoprotein).
Embodiment 46. The method according to embodiment 37, wherein the plurality of Candida or Aspergillus genes comprises one or more polypeptides (e.g., one, two, three, four, five, six, seven, eight, nine, ten, eleven, twelve, thirteen, fourteen, or fifteen or more polypeptides) selected from among MET6-1, METE-2, NOT5, RBT4, IPF9162, CAR1, GAP1, SET1, ENO1, FBA1, PGK1-1, PGK1-2, MUC1-1, MUC1-2, and BGL2.
Embodiment 47. The method according to embodiment 37, wherein the plurality of Candida or Aspergillus genes comprises one or more polypeptides (e.g., one, two, three, four, five, six, or seven or more polypeptides) selected from among SET1, ENO1, FBA1, PGK1-1, PGK1-2, MUC1-2, and BGL2.
Embodiment 48. The method according to embodiment 37, wherein the subject's gene expression profile further comprises one or more genes endogenous to the subject.
Embodiment 49. The method according to embodiment 37, wherein the subject is immunocompromised at the time the biological sample is obtained from the subject.
Embodiment 50. The method according to embodiment 37, further comprising administering an anti-fungal agent to the subject.

### Materials and Methods

Definitions. Outcomes were classified as systemic candidiasis or controls. Systemic candidiasis was defined as recovery of Candida sp. from blood or a sterile site. Controls were defined as non-immunocompromised patients hospitalized at the Shands Teaching Hospital at the University of Florida (STH-UF) who did not have any clinical or microbiologic evidence of candida infection. Predictor variables were the titers of antibodies against specific antigens.

Study population. Over the period of thirty-six months, sera from 68 patients with systemic candidiasis was collected, including 66 patients with candidemia and 2 patients with deep-seated candidiasis (one with candida peritonitis related to chronic peritoneal dialysis, and one with biopsy-proven candida pneumonia). Patients were sub-classified into pre-term and newborn infants (<6 months old), immunocompromised hosts and burn victims, as well as by portal of entry. Descriptive data for the patients are provided in Table 11. The median time from the onset of infection to serum collection was 2 days. Seven patients died, and four of the survivors had infections that persisted for over 2 weeks. Sera from 24 hospitalized patients who had no evidence of candidiasis were also collected as controls.

Collection of sera. Patients at STH-UF were identified on the day of positive blood or sterile site cultures for Candida sp. Controls were identified by the Infectious Diseases consultation service at STH-UF. After informed consent was obtained in accordance with procedures approved by the National Institutes of Health and the UF Institutional Review Board, sera were collected and stored at −70° C. in the repository at the UF Mycology Research Unit. For patients with candidiasis, sera were obtained from the earliest possible date on or after the date that the first positive cultures were drawn. In all cases, this was within 7 days of the first positive culture.

Enzyme-linked immunosorbent assay (ELISA). Antibody titers were evaluated for a set of twelve antigens that were identified using In Vivo Induced Antigen Technology (MET6, SET1, GAP1, ENO1, NOT5, BGL2, FBA1, MUC1, CAR1, RBT4, IPF9162 and PGK1) (Cheng, S et al. Mol Microbiol., 2003; 48:1275-88). Whole or partial DNA sequences of the genes encoding the antigens were amplified by polymerase chain reaction using the primers listed in Table 10. Two fragments of MET6, PGK1 and MUC1 were amplified, resulting in a total of 15 DNA sequences. The resulting PCR products were cloned into the plasmid pET30 using an EK/LIC cloning kit (EMD Biosciences, Inc.). All inserts were confirmed by DNA sequencing. Each plasmid was transformed into E. coli BL21(DE3) (Novagen). Expression of the recombinant proteins was induced by isopropyl-β-d-thiogalactopyranoside (IPTG). The recombinant proteins were purified from cell-free supernatants by chromatography on Ni²⁺-NTA-agarose as previously described (Cheng, S et al. Infect Immun., 2005; 73:7190-7).

96-well flat-bottom microtiter plates were coated with purified recombinant proteins in carbonate buffer (pH 9.6) at a concentration of 0.5 ug per well for 1 h at 37° C. (Cheng, S et al. Infect Immun., 2005; 73:7190-7). The plates were washed in PBS with 0.1% Tween-20 (PBS-T), and blocked with 0.25% gelatin in PBS-T for 1 h at 37° C. The wells were again washed with PBS-T. Serially diluted serum specimens were added to each well, and the plate incubated for 1 h at 37° C. The plates were washed, and peroxidase-conjugated goat anti-human immunoglobulin IgM (1:5,000 dilution) or IgG (1:25,000 dilution) in PBS-T was added. After an 1-hour incubation at 37° C., the plates was washed and developed with a o-phenylenediamine in citrate buffer and 5% hydrogen peroxidase. The developing solution was stopped with 1 M phosphoric acid. The optical densities (ODs) were determined using a spectrophotometer at 450 nm. Background was defined in wells coated with the protein to which the secondary antibody was added but the primary antibody was not. The reactive titer was defined as the inverse of the greatest dilution at which the OD was two-fold greater than background. All serum samples were tested in duplicate. In addition to wells lacking the primary antibody, wells that were not coated with protein were further included as negative controls.

Statistical analyses. Statistical significance was set at 0.05. The antibody titers for individual proteins were first logₑ-transformed to approximate normal distribution prior to data analysis. Multicollinearity among the predictor variables was assessed using collinearity diagnostics in SAS PROC REG. Means and standard errors for each predictor were calculated for each outcome variable.

Potentially significant predictor variables for the discriminant model were identified by backward elimination analysis using the STEPDISC procedure and by canonical correlation analysis using the CANCORR procedure in SAS/STAT. In the backward elimination analysis, the predictor variables chosen to leave the model were based on the significance level of an F test from an analysis of covariance. In the canonical analysis, standardized canonical coefficients, which reflect the relative contribution of each predictor variable to the power of discriminating between the two outcomes, were generated; the variables with highest absolute values were included in the discriminant model.

The DISCRIM procedure in SAS/STAT was used to identify the smallest subset of predictor variables that best discriminate the two outcomes. The performance of this discriminant analysis was evaluated by estimating the error rate (probability of misclassification of outcome). Finally, linear regression analysis using PROC REG in SAS was performed to generate the predicted function for the best set of predictors that were identified. The prediction score takes the form of y=a+β₁x₁+β₂x₂+ . . . +βₙxₙ, where a is the constant where the regression line intercepts the y axis and β is a regression coefficient.

All patents, patent applications, provisional applications, and publications referred to or cited herein, supra or infra, are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

### Antibody Responses Against Candida Proteins Among Patients with Candidiasis

The present inventors measured antibody responses against immunogenic Candida proteins among human patients with candidemia, oropharyngeal candidiasis, and healthy controls. The patient population and underlying conditions are described in Tables 1 and 2, respectively.

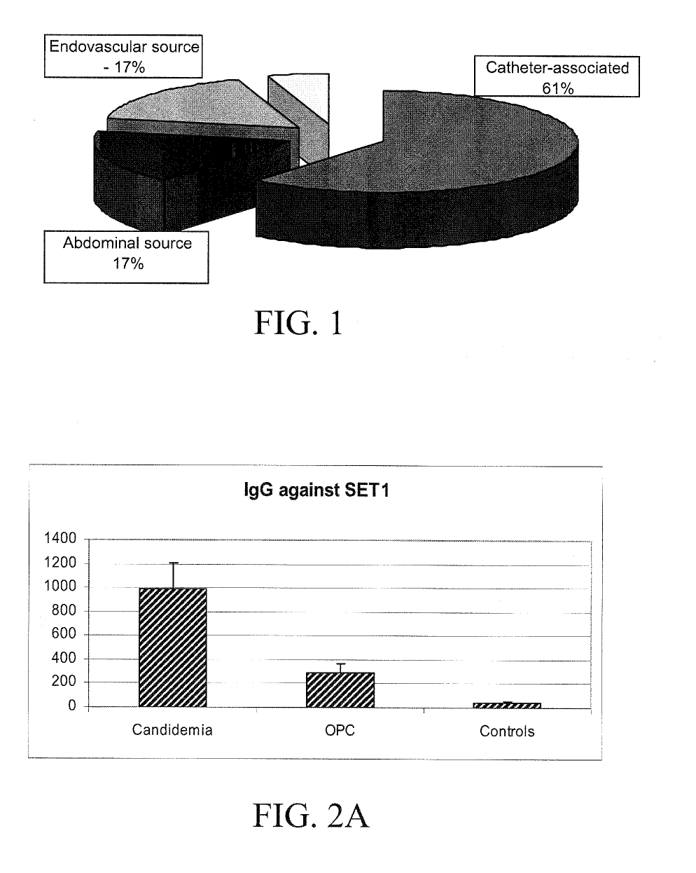

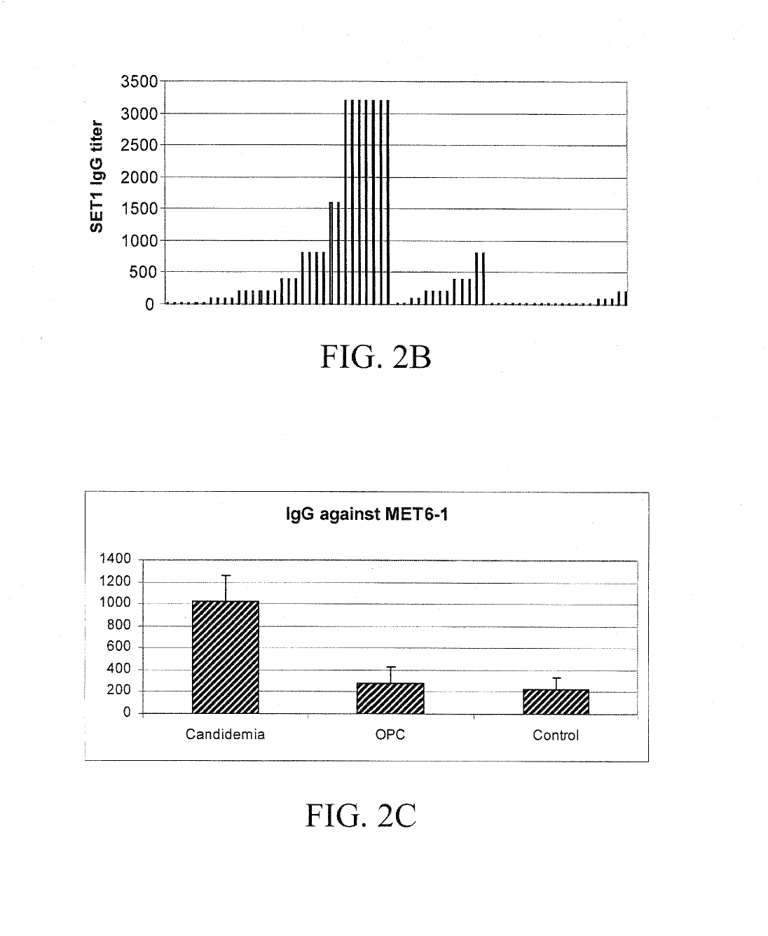

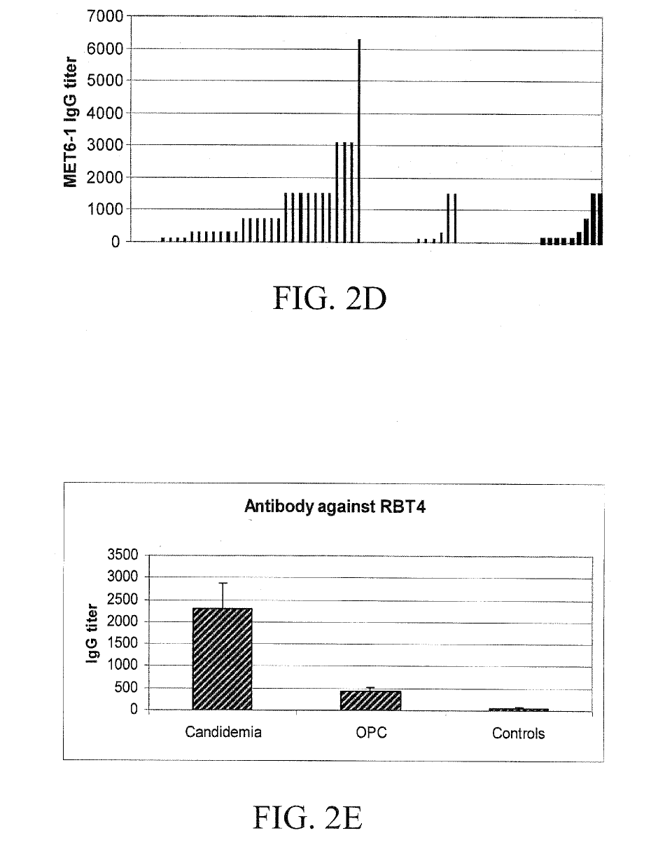

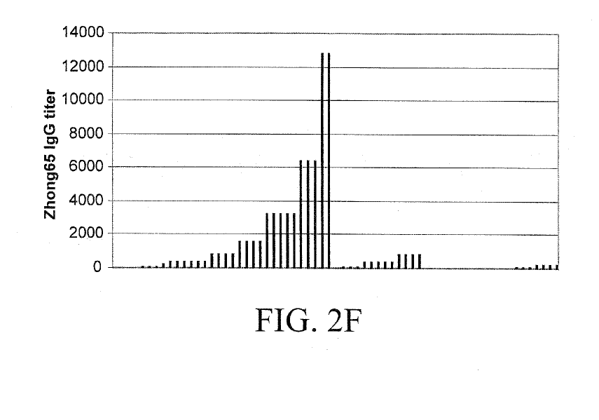

Sera were collected within 2 weeks of diagnosis from patients with candidemia, OPC and healthy controls as part of the University of Florida Mycology Research Unit. Recombinant C. albicans proteins Bgl2p, Eno1p, Fba1p, Gap1p, Pgk2p, Mep6p, Set1p, Rbt4p, Car1p, Muc1p and IPF9162p were purified. Antibody titers were measured against recombinant proteins by ELISA. Results are shown in Tables 3-6 and FIGS. 1 and 2A-2F.

TABLE 1
Patient population

| Types of patients | Number of patients |
|---|---|
| Patients with C. albicans candidemia | 32 patients |
| Patients with oropharyngeal candidiasis (OPC) | 13 patients |
| Hospitalized patients with no evidence of | 20 patients |
| candidiasis (controls) | |

TABLE 2
Underlying conditions

| Types of patients | Percent of patients |
|---|---|
| Burn or trauma patients | 28% |
| Patients receiving TPN | 17% |
| Patients undergoing GI surgery | 17% |
| Patients with diabetes mellitus and vascular diseases | 17% |
| Neonates | 11% |
| Other underlying illness | 17% |

There were no differences in IgM and IgA responses to proteins between the 3 groups. Total immunoglobulin and IgG responses, however, did differ between the groups. FIGS. 2A-2F show representative IgG responses against specific proteins.

TABLE 3
Three types of IgG responses

| Type of response | Proteins |
|---|---|
| Specific to DC | Rbt4, Met6, Set1, Gap1, Bgl2 |
| Specific to DC and OPC | Car1, Enol1, Fba1, IPF9162 |
| Non-specific | PGK1 |
| Low immunogenicity | Muc1 |

TABLE 4
IgG response against Set1p

| Antibody against Set1p | Rate |
|---|---|
| Sensitivity | 81.2% |
| Specificity | 70% |
| PPV | 81.2% |
| NPV | 70%* |

TABLE 5
IgG response against Met6

| Antibody against Met6 | Rate |
|---|---|
| Sensitivity | 87.5% |
| Specificity | 55% |
| PPV | 75.7% |
| NPV | 73.3%* |

TABLE 6
IgG response against Rbt4p

| Antibody against Rbt4p | Rate |
|---|---|
| Sensitivity | 87.5% |
| Specificity | 40.7% |
| PPV | 63% |
| NPV | 50.1%* |

*All 4 neonates had undetected antibody

TABLE 7
C. albicans genes and proteins identified by IVIAT screening

| | | | Function | |
|---|---|---|---|---|
| | | | deduced | |
| | | Known | from | |
| CandidaDB | Accession | function | homologous | |
| designation | No. | in C. albicans | protein in S. cerevisiae | Description |
| Transcription | | | | |
| Factor/Regulation | | | | |
| Dimorphism: | | | | |
| RBF1 | XM_714028; | Yes | | DNA-binding protein; |
| | XM_435193 | | | transcription factor |
| | | | | involves in yeast- |
| | | | | hyphae transition |
| CPP1 | | Yes | | Mitogen-activated |
| | | | | protein kinase |
| | | | | phosphatase; suppresses |
| | | | | hyphal growth |
| CST20 | U73457 | Yes | | Regulator leads to the |
| | | | | coordinate control of |
| | | | | hyphal development |
| CHK1 | | Yes | | Histidine kinase signal |
| | | | | transduction; essential |
| | | | | for hyphal |
| | | | | development; regulates |
| | | | | cell wall mannan and |
| | | | | glucan synthesis |
| CAP1 | U95611 | Yes | | Adenylate cyclase- |
| | | | | associated protein |
| | | | | regulates adenylate |
| | | | | cyclase activity |
| CDC24 | AY208122 | Yes | | GTP/GDP exchange |
| | | | | factor for CDC42; |
| | | | | contains RhoGEF |
| | | | | domain (2E-43) |
| | | | | involved in regulation |
| | | | | of various cellular |
| | | | | processes |
| NOT5 | XM_712551; | No | NOT5 | *Subunit of the CCR4- |
| | XM_436807 | | (1E-17) | NOT complex, a global |
| | | | | transcriptional regulator |
| IPF11281 | AB084519 | No | GPR1 | *G-protein coupled |
| | | | (1E-29) | receptor; regulates both |
| | | | | pseudohyphal and |
| | | | | invasive growth by a |
| | | | | cAMP-dependent |
| | | | | mechanism. |
| Metabolism: | | | | |
| PPR1 | | No | PPR1 | *Transcription factor |
| | | | (2E-68) | regulating pyrimidine |
| | | | | pathway (+ regulator of |
| | | | | URA1 and URA3) |
| IPF3598 | | No | SIP3 | *Activator of Snflp |
| | | | (2E-78) | protein kinase involved |
| | | | | in signal transduction, |
| | | | | filamentous growth and |
| | | | | cellular response to |
| | | | | nitrogen starvation. |
| | | | | Has a PH domain which |
| | | | | is found in eukaryotic |
| | | | | signaling pathway, a |
| | | | | constituent of |
| | | | | cytoskeleton |
| IPF9385 | | No | PHO2 | *Homeobox-domain |
| | | | (2E-30) | containing transcription |
| | | | | factor; involves in |
| | | | | regulation of phosphate |
| | | | | metabolism |
| Cell cycle: | | | | |
| IPF9413 | XM_707295; | No | CLG1 | *Cyclin dependent |
| | XM_442263 | | (4.5E-14) | protein kinase |
| | | | | holoenzyme complex, |
| | | | | regulates acid |
| | | | | phosphatase gene |
| | | | | expression; involves |
| | | | | with cell growth and |
| | | | | division |
| Unknown target: | | | | |
| SPT6 | XM_710094 | No | SPT6 | *DNA-dependent |
| | | | (E-140) | regulation of |
| | | | | transcription. |
| SET1 | XM_713878; | Yes | SET1 | *Chromatin-mediated |
| | XM_435267 | | (2E-72) | gene regulation. |
| SAS3 | XM_713115; | | SAS3 | *Histone |
| | XM_436111 | | (1E-90) | acetyltransferase, |
| | | | | silencing protein. |
| RPC53 | | No | RPC53 | *DNA-directed RNA |
| | | | (5E-11) | polymerase III; |
| | | | | transcription from Pol |
| | | | | III promoter |
| IPF2140 | XM_710153 | No | CAF40 | *CCR-NOT complex, |
| | | | (7E-92) | CCR4 Associated |
| | | | | Factor; regulates |
| | | | | transcription from Pol II |
| | | | | promoter. |
| IPF19724 | XM_715832; | No | TBF1 | *Transcription factor |
| | XM_433240 | | (2E-43) | involves in loss of |
| | | | | chromatin silencing. |
| IPF11711 | XM_710225; | No | TOM1 | *E3 ubiquitin protein |
| | XM_439087 | | (4.5e-250) | ligase required for |
| | | | | G2/M transition. |
| | | | | Contains a yeast hect- |
| | | | | domain protein which |
| | | | | mediates transcriptional |
| | | | | regulation |
| IPF1009 | | No | RFX1 | *Transcription factor |
| | | | (1.4e-7) | regulating a wide |
| | | | | variety of processes; |
| | | | | involves in DNA |
| | | | | damage response, signal |
| | | | | transduction resulting in |
| | | | | cell cycle arrest |
| IPF2971 | | No | | Unknown function. |
| | | | | Contains a cyclin |
| | | | | domain that regulates |
| | | | | cyclin-dependent |
| | | | | kinases |
| IPF1798 | | No | No | Unknown function. |
| | | | | Contains Fungal |
| | | | | specific transcription |
| | | | | factor domain found in |
| | | | | transcription activator |
| | | | | xInR, yeast regulatory |
| | | | | protein GAL4, and |
| | | | | other transcription |
| | | | | proteins regulating |
| | | | | cellular and metabolic |
| | | | | processes |
| IPF4805 | | No | | Unknown function. |
| | | | | Contains a zinc-finger |
| | | | | transcriptional factor |
| | | | | with low homology to |
| | | | | scNFL11, and a SAP |
| | | | | domain found in diverse |
| | | | | nuclear proteins |
| | | | | involved in |
| | | | | chromosomal |
| | | | | organization |
| STRESS | | | | |
| RESPONSE & | | | | |
| ADAPTATION | | | | |
| Stress response: | | | | |
| RBT 4 | XM_713699; | Yes | | Filament-specific gene, |
| | XM_435487 | | | but has no role in |
| | | | | morphology transition. |
| | | | | Encodes PR |
| | | | | (pathogenesis-related) |
| | | | | protein that is |
| | | | | synthesized during |
| | | | | infection, stress-related |
| | | | | responses, serum |
| | | | | treatment of |
| | | | | filamentous cells, and |
| | | | | depletion of TUP1. |
| IPF5761 | | No | FMO | *A flavin-containing |
| | | | (4E-32) | monooxygenase |
| | | | | involved in oxidation of |
| | | | | biologic thiols; is vital |
| | | | | for yeast response to |
| | | | | reductive stress. |
| IPF1428 | XM_713923; | No | BUL2 | *Contains the N- |
| | XM_435312 | | (8E-18) | terminus of scBUL1. |
| | | | | Essential for growth in |
| | | | | various stress |
| | | | | conditions. |
| Nutrient | | | | |
| sensor/transport: | | | | |
| MEP2 | | No | MEP2 | *An ammonium |
| | | | (1E-121) | transport affecting |
| | | | | pseudohyphal growth. |
| IPF11281 | XM_715449; | Yes | | Proline transport helper |
| (PTH1) | XM_433638 | | | [Ref]. Also similar to |
| | | | | scGpr1p, a G-protein- |
| | | | | coupled receptor at |
| | | | | plasma membrane; |
| | | | | interacts in two-hybrid |
| | | | | system with Gpa2p |
| | | | | involving in cell growth |
| | | | | and maintenance, |
| | | | | pseudohyphal growth, |
| | | | | signal transduction and |
| | | | | sporulation. |
| ENA22 | | No | ENA5 | *Sodium ion transport |
| | | | (0.0) | (P-type ATPases) |
| | | | | member of a |
| | | | | superfamily cation |
| | | | | transport enzyme, |
| | | | | mediates membrane |
| | | | | flux of all cations. |
| MDL1 | XM_713187; | | MDL1 | *ABC transporter |
| | XM_436186 | | (3E-145) | involves in the export |
| | | | | of peptides from the |
| | | | | mitochondrial matrix; |
| | | | | regulates cellular |
| | | | | resistance to oxidative |
| | | | | stress. |
| ALP1 | XM_708849; | | ALP1 | *Basic amino acid |
| | XM_440625 | | (2E-70) | transporter, involved in |
| | | | | uptake of cationic |
| | | | | amino acids |
| DNA repair: | | | | |
| RAD23 | | No | RAD23 | *Nucleotide excision |
| | | | (2E-24) | repair protein |
| | | | | (ubiquitin-like protein). |
| | | | | Also plays a role in |
| | | | | negative regulation of |
| | | | | protein catabolism; |
| | | | | nucleotide-excision |
| | | | | repair; DNA damage |
| | | | | recognition. |
| RFA1 | XM_714446; | No | RFA1 | *DNA replication |
| | XM_434861 | | (1E-102) | factor A, required for |
| | | | | DNA-damage repair. |
| IPF9141 | | No | CTF4 | *Chromatin-associated |
| | | | 1E-60 | protein involves in |
| | | | | DNA repair, DNA |
| | | | | dependent DNA |
| | | | | replication, sister |
| | | | | chromatid cohesion, |
| | | | | replicative cell aging. |
| IPF19872 | XM_712512; | No | | Unknown function. |
| | XM_436767 | | | Has DNA-binding |
| | | | | protein C1D domain |
| | | | | (3E-05) involved in |
| | | | | regulation of double- |
| | | | | strand break repair |
| METABOLISM | | | | |
| LPD1 | XM_707241; | No | LPD1 | *Dihydrolipoamide |
| | XM_442283 | | (1E-111) | dehydrogenase: |
| | | | | involved in acetyl-CoA |
| | | | | biosynthesis from |
| | | | | pyruvate and amino |
| | | | | acid catabolism. |
| PDB1 | | No | PDB1 | pyruvate dehydrogenase |
| | | | (7E-92) | involved in pyruvate |
| | | | | metabolism |
| MDH12 | | No | MDH12 | *Mitochondrial malate |
| | | | (2E-51) | dehydrogenase involves |
| | | | | in NADH regeneration, |
| | | | | fatty acid oxidation, |
| | | | | glyoxylate cycle, and |
| | | | | malate metabolism |
| CAR1 | XM_716750; | No | CAR1 | *Arginase involved in |
| | XM_432299 | | (3E-59) | arginine catabolism to |
| | | | | ornithine. |
| IPF6881 | | No | S. pombe | Function deduced by |
| | | | (3E-30) | homology to S. pombe |
| | | | | phosphatidyl synthase. |
| | | | | Contains NagD |
| | | | | domains with predicted |
| | | | | sugar phosphatases of |
| | | | | the HAD superfamily |
| | | | | involved in |
| | | | | carbohydrate transport |
| | | | | and metabolism. |
| IPF4258 | | No | | Unknown function. |
| | | | | Contains a eukaryotic- |
| | | | | specific Acyl CoA |
| | | | | binding protein domain |
| | | | | (6E-13) involved in |
| | | | | lipid transport and |
| | | | | metabolism. |
| IPF7489 | | | YOR171C | Contains LCB5 domain |
| | | | (1E-5) | sphingosine kinase and |
| | | | | diacylglycerol kinase |
| | | | | involved in lipid |
| | | | | metabolism. |
| HOST- | | | | |
| PATHOGEN | | | | |
| INTERACTION | | | | |
| Cell wall | | | | |
| structure, | | | | |
| organization and | | | | |
| biosynthesis: | | | | |
| MYO5 | XM_705894; | Yes | MYO5 | *Involved in cell wall |
| | XM_443689 | | (0.0) | organization and |
| | | | | biogenesis, endocytosis, |
| | | | | exocytosis, polar |
| | | | | budding, response to |
| | | | | osmotic stress, and |
| | | | | salinity response. |
| | | | | Required for Candida |
| | | | | hyphal formation. |
| PRAI | | Yes | | Cell wall protein with a |
| | | | | role in pH-regulation, |
| | | | | temperature dependent |
| | | | | morphogenesis |
| AMYG2 | XM_711719; | Yes | ROT2 | Glucoamylase. Also |
| | XM_437664 | | (1E-34) | similar to sc ROT2 |
| | | | | involved in cell wall |
| | | | | biosynthesis (glucosyl |
| | | | | hydrolase enzyme of |
| | | | | carbohydrate |
| | | | | metabolism) |
| LP19 | | No | MHP1 | *Cell wall |
| | | | (2E-75) | organization and |
| | | | | biogenesis; microtubule |
| | | | | stabilization |
| ALG5 | | | ALG5 | *Mannosyltransferase, |
| | | | (6E-95) | involved in asparagine- |
| | | | | linked glycosylation in |
| | | | | the endoplasmic |
| | | | | reticulum. |
| Adherence to host | | | | |
| cell/Flocculation | | | | |
| HWP1 | AY445062 | Yes | | Hyphal wall |
| | | | | protein required for |
| | | | | normal hyphal |
| | | | | formation. |
| ALS10 | XM_705343; | Yes | C. albicans | Function deduced from |
| | XM_444273 | | ALS3 | homology with C. albicans |
| | | | | ALS3, an |
| | | | | agglutinin-like protein. |
| IPF5185 | | No | FLO1 | *Cell wall protein |
| | | | (3E-7) | involved in |
| | | | | flocculation; binds to |
| | | | | mannose chains on the |
| | | | | surface of other cells. |
| IPF10919 | | No | FLO1 | *Cell wall protein |
| | | | (1E-14) | involved in |
| | | | | flocculation; binds to |
| | | | | mannose chains on the |
| | | | | surface of other cells. |
| IPF15911 | | No | MUC1 | *Cell surface flocculin |
| | | | (2E-10) | with structure similar to |
| | | | | serine/threonine-rich |
| | | | | GPI-anchored cell wall |
| | | | | protein. |
| Hydrolytic | | | | |
| enzyme: | | | | |
| PLB4.5f | | Yes | PLB3 | Function deduced by |
| | | | (3e-41) | homology to PLB3 (3E- |
| | | | | 41): phospholipase B |
| | | | | involved in |
| | | | | phosphatidylserine |
| | | | | catabolism and |
| | | | | phosphoinositide |
| | | | | metabolism. |
| OTHER CELL | | | | |
| STRUCTURES | | | | |
| PCT1 | | | PCT1 | *Cholinephosphate |
| | | | (6E-78) | cytidylyltransferase |
| | | | | involved in |
| | | | | phosphatidylcholine |
| | | | | biosynthesis and CDP- |
| | | | | choline pathway. |
| COX11 | XM_706452; | | COX11 | *Mitochondrial protein |
| | XM_443117 | | (3E-78) | required for assembly |
| | | | | of active cytochrome c |
| | | | | oxidase, the terminal |
| | | | | electron acceptor of the |
| | | | | respiratory chain in |
| | | | | mitochondria |
| KEL1 | | | KEL1 | *Involved in cell fusion |
| | | | (4E-77) | and morphology; |
| | | | | localizes to regions of |
| | | | | polarized growth. |

In addition, further screening has identified the following (which are included as Table 7):
GAP1 (XM_715518; XM_433708), IRS4 (XM_707661; XM_441886), INP51 (XM_709454; XM_439936), SET1, SET2 (XM_709308; XM_440093), DOT1 (XM_710974; XM_438330), ENO1 (XM_706790; XM_442766), BGL2 (XM_717544; XM_431403), FBA1, MUC1, IPF9162, BUR2, PGK1 (XM_706231; XM_443352)

TABLE 8
A. fumigatus genes and proteins identified by IVIAT screening

| | A. fumigatus clone | Function |
|---|---|---|
| C17.3 | Aspergillus fumigatus | Putative serine-threonine protein kinase with |
| | Afu2g10620 | homology to S. cerevisiae YPK1 and YPK2, which |
| | | are required for receptor-mediated endocytosis and |
| | | are involved in the cell integrity signaling pathway |
| W11 | Garden petunia | Catalyzes final step in ethylene biosynthesis |
| | ACC oxidase I (32%) | |
| W12 | Aspergillus nidulans | Hypothetical protein containing NmrA-like domain, |
| | AN8970.2 (31%) | which is part of a system controlling nitrogen |
| | | metabolite repression |
| W10 | Aspergillus nidulans | Hypothetical protein containing an E3-ubiquitin |
| | AN2162.2 (56%) | protein ligase domain |
| W6 | Aspergillus nidulans | Hypothetical protein containing a guanine |
| | AN6709.2 (47%) | nucleotide exchange factor domain. Similar |
| | | domains are found in yeast proteins regulating |
| | | vesicle trafficking in endocytosis and exocytosis |
| W3 | Aspergillus nidulans | Hypothetical protein containing a WD domain. |
| | AN7704.2 (69%) | Proteins containing WD domains regulate diverse |
| | | processes in eukaryotes (often in a species-specific |
| | | manner) and are especially prevalent in chromatin |
| | | modification and transcriptional mechanisms |
| A01 | Saccharomyces | tRNA C-5 methyltransferase |
| | cerevisiae YBL024w | |
| B01 | Aspergillus fumigatus | Putative DNA-directed RNA polymerase |
| | CAF32099 | |
| C16.3 | Aspergillus nidulans | Hypothetical protein containing a putative |
| | AN7465.2 | cohesion complex protein domain |
| C17.9 | Aspergillus nidulans | Hypothetical protein with homology to S. cerevisiae |
| | AN4541.2 | NNTI, which encodes nicotinamide N- |
| | | methyltransferase (involved in rDNA silencing) |
| C19.5 | Aspergillus nidulans | Hypothetical protein, putative acetyl transferase |
| | AN3628.2 | |
| C20.5 | Aspergillus nidulans | Hypothetical protein containing a conserved GTP |
| | AN EAA58968 | binding protein domain |
| D04 | Aspergillus nidulans | Hypothetical protein, contains a clavamic acid |
| | AN2960.2 | synthetase (CAS)-like domain |
| D06 | Aspergillus nidulans | Hypothetical protein, contains a formyl transferase RING |
| | AN5922.2 | domain (Zinc binding) |

TABLE 9
Classifications of proteins used as targets for antibody detection

| | Protein | | | |
|---|---|---|---|---|
| Classification | name | Protein description | Note | Reference |
| classic cell | BGL2 | glucan 1,3-β- | protein reported by our | Pitarch, A et |
| wall proteins | | glucosidase | lab as well as other | al. Mol Cell |
| | | | groups to elicit higher | Proteomics, |
| | | | antibody responses | 2006; 5: 79-96 |
| | | | among patients with | |
| | | | systemic candidiasis | |
| | | | than un-infected | |
| | | | controls | |
| | MUC1 | cell surface | Protein not been | (our lab) |
| | | glycoprotein | previously reported to | |
| | | | be immunogenic | |
| glycolytic | ENO1 | enolase I | protein reported by our | van |
| enzymes | | | lab as well as other | Deventer, AJ |
| localized to | | | groups to elicit higher | et al. |
| the cell wall | | | antibody responses | Microbiol |
| | | | among patients with | Immunol., |
| | | | systemic candidiasis | 1996; |
| | | | than un-infected | 40: 125-31; |
| | | | controls | Mitsutake, K |
| | | | | et al. J |
| | | | | Clin Lab |
| | | | | Anal., 1994; |
| | | | | 8: 207-10; |
| | | | | Mitsutake, K |
| | | | | et al. J |
| | | | | Clin |
| | | | | Microbiol., |
| | | | | 1996; 1918-21 |
| | FBA1 | FBA1 | protein reported by our | Pitarch, A et |
| | | | lab as well as other | al. Mol Cell |
| | | | groups to elicit higher | Proteomics, |
| | | | antibody responses | 2006; 5: 79-96 |
| | | | among patients with | |
| | | | systemic candidiasis | |
| | | | than un-infected | |
| | | | controls | |
| | GAP1 | glyceraldehyde-3- | protein reported by our | Pitarch, A et |
| | | phosphate | lab as well as other | al. Mol Cell |
| | | dehydrogenase | groups to elicit higher | Proteomics, |
| | | | antibody responses | 2006; 5: 79-96 |
| | | | among patients with | |
| | | | systemic candidiasis | |
| | | | than un-infected | |
| | | | controls | |
| | PGK1 | phosphoglycerate | protein reported by our | Pitarch, A et |
| | | kinase | lab as well as other | al. Mol Cell |
| | | | groups to elicit higher | Proteomics, |
| | | | antibody responses | 2006; 5: 79-96 |
| | | | among patients with | |
| | | | systemic candidiasis | |
| | | | than un-infected | |
| | | | controls | |
| intracellular | NOT5 | a member of the | Protein previously | Cheng, S et |
| proteins | | transcription | shown by our labs to | al. Mol |
| localized to | | regulatory CCR4- | contribute to virulence | Microbiol., |
| cell wall | | NOT complex | and to elicit higher | 2003; |
| | | | antibody responses | 48: 1275-88 |
| | | | among patients with | |
| | | | systemic candidiasis | |
| | | | than un-infected | |
| | | | controls | |
| | MET6 | 5- | protein reported by our | Pitarch, A et |
| | | methyltetrahydropteroyltri- | lab as well as other | al. Mol Cell |
| | | glutamate | groups to elicit higher | Proteomics, |
| | | homocysteine | antibody responses | 2006; 5: 79-96 |
| | | methyltransferase | among patients with | |
| | | | systemic candidiasis | |
| | | | than un-infected | |
| | | | controls | |
| intracellular | CAR1 | arginase | Protein not been | Our lab |
| proteins, | | | previously reported to | |
| likely not | | | be immunogenic | |
| localized to | | | | |
| cell wall | | | | |
| | RBT4 | repressor of TUP1 | Protein not been | Our lab |
| | | | previously reported to | |
| | | | be immunogenic | |
| | SET1 | chromatin | Protein previously | Raman, SB |
| | | regulatory protein | shown by our labs to | et al. Mol |
| | | | contribute to virulence | Microbiol., |
| | | | and to elicit higher | 2006; |
| | | | antibody responses | 60: 697-709 |
| | | | among patients with | |
| | | | systemic candidiasis | |
| | | | than un-infected | |
| | | | controls | |
| | IPF11897 | protein of | Protein not been | Our lab |
| | | unknown function. | previously reported to | |
| | | | be immunogenic | |

TABLE 10
Primers used for cloning of antigens

| | | Anti-sense primers (3′ | Length of antigen |
|---|---|---|---|
| Antigens | Sense primers (5′→ 3′) | → 5′) | (amino acids) |
| MET6-1 | 5′GACGACGACAAGAT | 5′GAGGAGAAGCCC | 400 aa |
| | GGTTCAATCTTCCGTC | GGTTAAGAAGATTC | (position 1-400 aa) |
| | TTAGGT (SEQ ID NO: 1) | GGATCTAGC (SEQ ID | |
| | | NO: 2) | |
| MET6-2 | 5′GACGACGACAAGAT | 5′GAGGAGAAGCCC | 367 aa |
| | GATACCAACGATCCAA | GGTTAGTATTTAGC | (position 401-768) |
| | AG (SEQ ID NO: 3) | TCTGAATTC (SEQ ID | |
| | | NO: 4) | |
| RBT4 | 5′GACGACGACAAGAT | 5′GAGGAGAAGCCC | 359 aa |
| | GAAGTTTTCTCAAGTT | GGTAATAACACCAG | (entire gene) |
| | GCCACTACTGCTGCTG | AGTTCTGTAAAAGT | |
| | CCATT (SEQ ID NO: 5) | CGGTA (SEQ ID NO: | |
| | | 6) | |
| IPF9162 | 5′GACGACGACAAGAT | 5′GAGGAGAAGCCC | 272 aa |
| | GAAAAAAAGGTTAGT | GGTAATTTATCAAT | (entire gene) |
| | TTTGTTTGATGATTCT | TTACATATAGTGCT | |
| | GATGAT (SEQ ID NO: 7) | CAAAATGGACCTGT | |
| | | CAA (SEQ ID NO: 8) | |
| CAR1 | 5′GACGACGACAAGAT | 5′GAGGAGAAGCCC | 318 aa |
| | GTCATCAATTCAATAT | GGTAATGTTATTTC | (entire gene) |
| | AAATATCATCCAGACA | AAACTGGGTTACGT | |
| | A (SEQ ID NO: 9) | GTAGAT (SEQ ID NO: | |
| | | 10) | |
| GAP1 | 5′GACGACGACAAGAT | 5′GAGGAGAAGCCC | 336 aa |
| | GGCTATTAAAATT | GGTTAAGCAGAAGC | (entire gene) |
| | GGTATTAAC (SEQ ID | TTTAGCAAC (SEQ ID | |
| | NO: 11) | NO: 12) | |
| ENO1 | 5′GACGACGACAAGAT | 5′GAGGAGAAGCCC | 441 aa |
| | GTCTTACGCCACTAAA | GGTTACAATTGAGA | (entire gene) |
| | ATCCAC (SEQ ID NO: | AGCCTTTTGGAA | |
| | 13) | (SEQ ID NO: 14) | |
| BGL2 | 5′GACGACGACAAGAT | 5′GAGGAGAAGCCC | 309 aa |
| | GCAAATCAAATTCTTG | GGTTAGTTGAATTT | (entire gene) |
| | ACTACT (SEQ ID NO: | ACAGTCAATTGA | |
| | 15) | (SEQ ID NO: 16) | |
| FBA1 | 5′GACGACGACAAGAT | 5′GAGGAGAAGCCC | 360 aa |
| | GGCTCCTCCAGCAGTT | GGTTACAATTGTCC | (entire gene) |
| | TTAAGT (SEQ ID NO: | TTTGGTGTGGAA | |
| | 17) | (SEQ ID NO: 18 | |
| MUC1-1 | 5′GACGACGACAAGAT | 5′GAGGAGAAGCCC | 306 aa |
| | GTCATTTTGGGACAAC | GGTTAGGTTGAGTT | (position 1-306) |
| | AACAA (SEQ ID NO: 19) | ATTGGTTAAAA (SEQ | |
| | | ID NO: 20) | |
| MUC1-2 | 5′GACGACGACAAGAT | 5′GAGGAGAAGCCC | 153 aa |
| | GGAGTATATCGCATCT | GGTTATTCATGTGG | (position 735-888) |
| | TGGTGT (SEQ ID NO: | CATTGCTCGATA | |
| | 21) | (SEQ ID NO: 22) | |
| PGK1-1 | CA-EXP-FOR | CAPKG1-F1-REV | 238 aa |
| | 5′GACGACGACAAGAT | 5′GAGGAGAAGCCC | (position 1-238) |
| | GTCATTATCTAACAAA | GGTTAACCACCACC | |
| | TTATCA (SEQ ID NO: | AACAATC (SEQ ID | |
| | 23) | NO: 24) | |
| PGK1-2 | 5′GACGACGACAAGAT | 5′GAGGAGAAGCCC | 180 aa |
| | GGCCTTCACTTTCAAG | GGTTAGTTTTTGTTG | (position 239-438) |
| | AAA (SEQ ID NO: 25) | GAAAGAGC (SEQ ID | |
| | | NO: 26) | |

TABLE 11
Descriptive data of patients with systemic candidiasis

| Characteristics | Number of patients |
|---|---|
| Age: | |
| Median (range) | 51.5 years (3 days to 81 years) |
| Age < 6 month old | 8 patients |
| Immunocompromised | 12 patients¹ |
| Burn victims | 8 patients |
| Portal of entry: | |
| Catheter | 33 patients |
| Abdominal | 21 patients |
| Wound | 6 patients |
| Complications: | |
| Endocarditis | 2 patients |
| Mediastinitis | 2 patients |
| Vascular graft infection | 2 patients |
| Candida spp.: | |
| Candida albicans | 28 patients² |
| Non-C. albicans | |
| C. glabrata | 19 patients |
| C. parapsilosis | 10 patients³ |
| C. tropicalis | 5 patients |
| C. krusei | 4 patients |
| C. lusitaniae | 1 patient |
| unspeciated | 1 patient |

¹three bone marrow transplant recipients, 5 solid transplant recipients, and 1 both BMT and SOT recipient, 2 patients with hematologic malignancy on chemotherapy, and one patient with systemic lupus on high dose steroid.
²three patients were co-infected with C. glabrata, C. parapsilosis, C. tropicalis.
³a patient was co-infected with C. guilliermondii

TABLE 12
Performance tests of antibody against specific proteins

| | | | Likelihood | |
|---|---|---|---|---|
| | Sensitivity | Specificity | ratio | p-value |
| MET6-1 | 65% (39/60) | 83.3% (20/24) | 3.9 | <0.0001 |
| MET6-2 | 60% (36/60) | 54.2% (13/24) | 1.3 | NS |
| NOT5 | 85% (51/60) | 87.5% (21/24) | 6.8 | <0.0001 |
| SET1 | 98.3% (59/60) | 66.7% (16/24) | 3.0 | <0.0001 |
| RBT4 | 98.3% (59/60) | 62.5% (15/24) | 2.6 | <0.0001 |
| IPF9162 | 96.7% (58/60) | 50% (12/24) | 1.9 | <0.0001 |
| CAR1 | 90% (54/60) | 75% (18/24) | 3.6 | <0.0001 |
| GAP1 | 91.7% (55/60) | 87.5% (21/24) | 7.3 | <0.0001 |
| ENO1 | 98.3% (59/60) | 70.8% (17/24) | 3.4 | <0.0001 |
| BGL2 | 95% (57/60) | 75% (18/24) | 3.8 | <0.0001 |
| FBA1 | 93.3% (56/60) | 91.7% (22/24) | 11.2 | <0.0001 |
| MUC1-1 | 96.7% (58/60) | 50% (12/24) | 1.9 | <0.0001 |
| MUC1-2 | 86.7% (52/60) | 54.2% (13/24) | 1.9 | 0.0002 |
| PGK1-1 | 72.9% (43/59)* | 56.5% (13/23)* | 1.7 | 0.02 |
| PGK1-2 | 62.7% (37/59)* | 52.2% (12/23)* | 1.3 | NS |

*one patient from the systemic candidiasis group and one from the control group did not have sufficient sera to perform antibody response to PGK1-1 and PGK1-2.

TABLE 13
Rank order of predictors identified using canonical correlation analysis

| Antibodies to specific antigens | Standardized canonical coefficients |
|---|---|
| SET1 | 0.574 |
| MUC1-2 | −0.324 |
| FBA1 | 0.318 |
| PGK-1 | 0.309 |
| PGK-2 | −0.302 |
| BGL2 | −0.298 |
| ENO1 | 0.272 |
| IPF9162 | 0.196 |
| NOT5 | 0.100 |
| RBT4 | 0.080 |
| MET6-1 | 0.058 |
| GAP1 | −0.021 |
| CAR1 | 0.017 |
| MUC1-1 | −0.018 |
| MET6-2 | −0.001 |

TABLE 14
Performance of the full model as well as with subsets of predictors
chosen by backward elimination and canonical analyses

| Model | Error | Sensitivity | Specificity |
|---|---|---|---|
| Full model (with 15 | 3.7% (3/82)* | 96.6% (57/59)* | 95.6% (22/23)* |
| predictors) | | | |
| SET1, ENO1, FBA1, | 3.7% (3/82)* | 96.6% (57/59)* | 95.6% (22/23)* |
| PGK1-1, PGK1-2, | | | |
| MUC1-2, BGL2 | | | |
| SET1, ENO1, PGK1-2, | 3.7% (3/82)* | 96.6% (57/59)* | 95.6% (22/23)* |
| MUC1-2 | | | |
| SET1, ENO1, MUC1-2 | 4.8% (4/84) | 95.0% (57/60) | 95.8% (23/24) |

*2 patients (one from the systemic candidiasis group and one from the control group) had limited serum; antibody titers were sufficient to test only against 14 predictor variables (all predictors except PGK1).

TABLE 15
Serum IgG responses against specific antigens

| | | Standard error Mean | |
|---|---|---|---|
| | Mean of log₂titer ± | of log₂titer ± | |
| | standard error of | standard error of | |
| Predictor variable | patients with DC | control patients | P-value |
| BGL2 | 8.69 ± 0.27 | 4.32 ± 0.36 | <0.0001 |
| PGK1-1 | 7.93 ± 0.27 | 2.20 ± 0.46 | 0.01 |
| PKG1-2 | 7.49 ± 0.26 | 6.52 ± 0.43 | 0.05 |
| CAR1 | 8.40 ± 0.28 | 4.28 ± 0.35 | <0.0001 |
| ENO1 | 9.07 ± 0.26 | 4.46 ± 0.38 | <0.0001 |
| FBA1 | 8.49 ± 0.29 | 3.60 ± 0.19 | <0.0001 |
| 7GAP1 | 8.38 ± 0.29 | 3.74 ± 0.23 | <0.0001 |
| IPF9162 | 8.92 ± 0.22 | 5.14 ± 0.39 | <0.0001 |
| MET6-1 | 8.67 ± 0.28 | 6.28 ± 0.50 | 0.0002 |
| MET6-2 | 9.60 ± 0.21 | 8.57 ± 0.48 | 0.02 |
| MUC1-1 | 8.72 ± 0.23 | 5.86 ± 0.56 | <0.0001 |
| MUC1-2 | 8.69 ± 0.24 | 7.02 ± 0.52 | 0.001 |
| NOT5 | 8.18 ± 0.37 | 3.74 ± 0.23 | <0.0001 |
| RBT4 | 9.42 ± 0.27 | 4.78 ± 0.40 | <0.0001 |
| SET1 | 9.16 ± 0.22 | 4.51 ± 0.36 | <0.0001 |

TABLE 16
Backward selection procedure to identify variables best
distinguishing class membership. The table lists the order
by which the variables were removed.

| | | | | | | Average |
|---|---|---|---|---|---|---|
| | Number | | | | | squared |
| | of | Variables | Partial | | | canonical |
| Step | variables | removed | R² | F value | P-value | correlation |
| 0 | 15 | | | | | 0.7345 |
| 1 | 14 | MET6-F2 | 0.0000 | 0.00 | 0.99 | 0.7345 |
| 2 | 13 | CAR1 | 0.0001 | 0.01 | 0.93 | 0.7344 |
| 3 | 12 | GAP1 | 0.0001 | 0.01 | 0.93 | 0.7344 |
| 4 | 11 | MUC1-F1 | 0.0002 | 0.01 | 0.92 | 0.7344 |
| 5 | 10 | MET6-F1 | 0.0016 | 0.11 | 0.74 | 0.7340 |
| 6 | 9 | RBT4 | 0.0024 | 0.17 | 0.68 | 0.7333 |
| 7 | 8 | NOT5 | 0.0169 | 1.24 | 0.27 | 0.7288 |
| 8 | 7 | BGL2 | 0.0206 | 1.53 | 0.22 | 0.7230 |
| 9 | 6 | FBA1 | 0.0160 | 1.20 | 0.28 | 0.7185 |
| 10 | 5 | IPF | 0.0412 | 3.23 | 0.08 | 0.7064 |
| 11 | 4 | PKG1-F1 | 0.0497 | 3.98 | 0.05 | 0.6911 |
| 12 | 3 | PKG1-F2 | 0.0218 | 1.72 | 0.19 | 0.6819 |
| 13 | 2 | MUC1-F2 | 0.0869 | 7.42 | 0.008 | 0.6541 |
| 14 | 1 | ENOL | 0.1428 | 13.16 | 0.0005 | 0.560 |
| 15 | 0 | SET | 0.5965 | 118.28 | <0.0001 | |

The F-values denote the distances between the two outcomes in the multivariate model.

The F-test of significance (p-value) denotes significance level of the discriminant function as a whole at each elimination step. (the difference of specific predictor between the two outcome means at each elimination step).

TABLE 17
Weights contributed by the specific antibodies in predicting class
memberships

| Predictors | Standardized canonical coefficient |
|---|---|
| SET1 | 1.29 |
| ENOL | 0.911 |
| MUC1-F2 | −0.39 |
| PKG1-F2 | −0.20 |

TABLE 18

1. RBF1
MSSNKNQSDLNIPTNSASLKQKQRQQLGIKSEIGASTSDVYDPQVASYLSAGDSPSQFANTALHHSNSVSYS
ASAAAAAAELQHRAELQRRQQQLQQQELQHQQEQLQQYRQAQAQAQAQAQAQAQAQREHQQLQHAYQQQQQL
HQLGQLSQQLAQPHLSQHEHVRDALTTDEFDTNEDLRSRYIENEIVKTFNSKAELVHFVKNELGPEERCKIV
INSSKPKAVYFQCERSGSFRTTVKDATKRQRIAYTKRNKCAYRLVANLYPNEKDQKRKNKPDEPGHNEENSR
ISEMWVLRMINPQHNHAPDPINKKKRQKTSRTLVEKPINKPHHHHLLQQEQQQQQQQQQQQQQQQQQQQQQQ
HNANSQAQQQAAQLQQQMQQQLQASGLPTTPNYSELLGQLGQLSQQQSQQQQLHHIPQQRQRTQSQQSQQQP
QQTPHGLDQPDAAVIAAIEASAAAAVASQGSPNVTAAAVAALQHTQGNEHDAQQQQDRGGNNGGAIDSNVDP
SLDPNVDPNVQAHDHSHGLRNSYGKRSGFL*

| Rank | Sequence | Start position | Score |
|---|---|---|---|
| 1 | HEHVRDALTTDEFDTN | 162 | 0.93 |
| 2 | GGAIDSNVDPSLDPNV | 495 | 0.92 |
| 3 | DRGGNNGGAIDSNVDP | 489 | 0.90 |
| 4 | SGLPTTPNYSELLGQL | 385 | 0.88 |
| 4 | AYTKRNKCAYRLVANL | 249 | 0.88 |
| 5 | LVANLYPNEKDQKRKN | 260 | 0.87 |
| 6 | ASYLSAGDSPSQFANT | 46 | 0.86 |
| 7 | EIGASTSDVYDPQVAS | 32 | 0.84 |
| 7 | HNHAPDPINKKKRQKT | 302 | 0.84 |
| 8 | QKRKNKPDEPGHNEEN | 271 | 0.83 |
| 9 | QQMQQQLQASGLPTTP | 376 | 0.82 |
| 10 | EKPINKPHHHHLLQQE | 323 | 0.81 |
| 10 | TKRQRIAYTKRNKCAY | 243 | 0.81 |
| 11 | DSPSQFANTALHHSNS | 53 | 0.80 |
| 11 | DPNVQAHDHSHGLRNS | 511 | 0.80 |
| 11 | RQKTSRTLVEKPINKP | 314 | 0.80 |
| 12 | QQQQQQQQQHNANSQA | 352 | 0.79 |
| 12 | CERSGSFRTTVKDATK | 229 | 0.79 |
| 12 | VHFVKNELGPEERCKI | 200 | 0.79 |
| 13 | AAAAAAELQHRAELQR | 75 | 0.77 |
| 13 | ALQHTQGNEHDAQQQQ | 473 | 0.77 |
| 14 | QRTQSQQSQQQPQQTP | 421 | 0.76 |
| 14 | WVLRMINPQHNHAPDP | 293 | 0.76 |
| 14 | CKIVINSSKPKAVYFQ | 213 | 0.76 |
| 14 | YRQAQAQAQAQAQAQA | 110 | 0.76 |
| 15 | KQRQQLGIKSEIGAST | 22 | 0.75 |
| 15 | EFDTNEDLRSRYIENE | 173 | 0.75 |

| Start | End | Max_score_pos | Sequence |
|---|---|---|---|
| 255 | 264 | 259 | KCAYRLVANL |
| 197 | 206 | 200 | AELVHFVKNE |
| 222 | 232 | 226 | PKAVYFQCERS |
| 327 | 338 | 332 | NKPHHNHLLQQE |
| 463 | 476 | 471 | SPNVTAAAVAALQH |
| 37 | 52 | 46 | TSDVYDPQVASYLSAG |
| 442 | 461 | 447 | PDAAVIAAIEASAAAAVASQ |
| 212 | 219 | 216 | RCKIVINS |
| 131 | 168 | 156 | HQQLQHAYQQQQQLHQLGQLSQQLAQPHLSQHEHVRDA |
| 60 | 87 | 72 | NTALHHSNSVSYSASAAAAAAELQHRAE |
| 393 | 419 | 414 | YSELLGQLGQLSQQQSQQQQLHHIPQQ |
| 511 | 521 | 513 | DPNVQAHDHSH |
| 319 | 325 | 323 | RTLVEKP |
| 379 | 388 | 385 | QQQLQASGLP |
| 368 | 377 | 371 | QQQAAQLQQQ |
| 91 | 126 | 97 | RQQQLQQQELQHQQEQLQQYRQAQAQAQAQAQAQAQ |
| 427 | 440 | 439 | QSQQQPQQTPHGLD |
| 342 | 358 | 358 | QQQQQQQQQQQQQQQQQ |

2. CPP1
MTTPLSSYSTTVTNHHPTFSFESLNSISSNNSTRNNQSNSVNSLLYFNSSGSSMVSSSSDAAPTSISTTTTS
TTSMTDASANADNQQVYTITKEDSINDINQKEQNSFSIQPNQTPTMLPTSSYTLQRPPGLHEYTSSISSISS
TSSNSTSTPVSPALINYSPKHSRKPNSLNLNRNMKNLSLNLHDSTNGYTSPLPKSTNSNQSRGNFIMDSPSK
KSTPVNRIGNNNGNDYINATLLQTPSITQTPTMPPPLSLAQGPPSSVGSESVYKFPPISNACLNYSAGDSDS
EVESMSMKQSAKNTIIPPMAPPFALQSKSSPLSTPPRLHSPLGVDRGLPISMSPIQSSLNQKFNNIALQTPL
NSSFSINNDEATNFNNKNNKNNNNNSTATTTITNTILSTPQNVRYNSKKFHPPEELQESTSINAYPNGPKNV
LNNLIYLYSDPVQGKIDINKFDLVINVAKECDNMSLQYMNQVPNQREYVYIPWSHNSNISKDLFQITNKIDQ
FFTNGRKILIHCQCGVSRSACVVVAFYMKKFQLGVNEAYELLKNGDQKYIDACDRICPNMNLIFELMEFGDK
LNNNEISTQQLLMNSPPTINL*

| Rank | Sequence | Start position | Score |
|---|---|---|---|
| 1 | MNLIFELMEFGDKLNN | 564 | 0.95 |
| 1 | QKYIDACDRICPNMNL | 551 | 0.95 |
| 2 | PALINYSPKHSRKPNS | 156 | 0.94 |
| 3 | PTSISTTTTSTTSMTD | 63 | 0.93 |
| 4 | SSSSDAAPTSISTTTT | 56 | 0.92 |
| 5 | ESVYKFPPISNACLNY | 266 | 0.91 |
| 6 | SPSKKSTPVNRIGNNN | 213 | 0.89 |
| 7 | STSINAYPNGPKNVLN | 419 | 0.88 |
| 7 | AGDSDSEVESMSMKQS | 283 | 0.88 |
| 8 | NSTATTTITNTILSTP | 385 | 0.87 |
| 8 | SISSISSTSSNSTSTP | 138 | 0.87 |
| 9 | SINNDEATNFNNKNNK | 365 | 0.86 |
| 10 | TQTPTMPPPLSLAQGP | 244 | 0.85 |
| 10 | YINATLLQTPSITQTP | 232 | 0.85 |
| 10 | PKHSRKPNSLNLNRNM | 163 | 0.85 |
| 11 | TNTILSTPQNVRYNSK | 393 | 0.84 |
| 11 | SMKQSAKNTIIPPMAP | 294 | 0.84 |
| 11 | PPISNACLNYSAGDSD | 272 | 0.84 |
| 11 | TTVTNHHPTFSFESLN | 10 | 0.84 |
| 12 | TSTTSMTDASANADNQ | 71 | 0.83 |
| 12 | QYMNQVPNQREYVYIP | 469 | 0.83 |
| 13 | PEELQESTSINAYPNG | 413 | 0.82 |
| 13 | SFSIQPNQTPTMLPTS | 107 | 0.82 |
| 14 | LGVNEAYELLKNGDQK | 537 | 0.81 |
| 14 | VVVAFYMKKFQLGVNE | 526 | 0.81 |
| 14 | GVDRGLPISMSPIQSS | 331 | 0.81 |
| 15 | GVSRSACVVVAFYMKK | 519 | 0.80 |
| 15 | LIHCQCGVSRSACVVV | 513 | 0.80 |
| 15 | QREYVYIPWSHNSNIS | 477 | 0.80 |
| 15 | PPRLHSPLGVDRGLPI | 323 | 0.80 |
| 15 | NSISSNNSTRNNQSNS | 25 | 0.80 |
| 16 | SGSSMVSSSSDAAPTS | 50 | 0.79 |
| 16 | KECDNMSLQYMNQVPN | 461 | 0.79 |
| 16 | VNRIGNNNGNDYINAT | 221 | 0.79 |
| 17 | QVYTITKEDSINDINQ | 87 | 0.78 |
| 17 | LMEFGDKLNNNEISTQ | 570 | 0.78 |
| 17 | VQGKIDINKFDLVINV | 444 | 0.78 |
| 17 | NQTPTMLPTSSYTLQR | 113 | 0.78 |
| 18 | KNTIIPPMAPPFALQS | 300 | 0.77 |
| 18 | DSTNGYTSPLPKSTNS | 187 | 0.77 |
| 18 | TSSYTLQRPPGLHEYT | 121 | 0.77 |
| 18 | NQKEQNSFSIQPNQTP | 101 | 0.77 |
| 19 | DLVINVAKECDNMSLQ | 454 | 0.76 |
| 19 | NSKKFHPPEELQESTS | 406 | 0.76 |
| 19 | LQSKSSPLSTPPRLHS | 313 | 0.76 |
| 19 | SSNSTSTPVSPALINY | 146 | 0.76 |

| Start | End | Max_score_pos | Sequence |
|---|---|---|---|
| 511 | 534 | 528 | KILIHCQCGVSRSACVVVAFYMKK |
| 151 | 164 | 156 | STPVSPALINYSPK |
| 305 | 348 | 329 | PPMAPPFALQSKSSPLSTPPRLHSPLGVDRGLPISMSPIQSSLN |
| 453 | 462 | 458 | FDLVINVAKE |
| 41 | 48 | 44 | VNSLLYFN |
| 433 | 447 | 441 | LNNLIYLYSDPVQGK |
| 250 | 282 | 271 | PPPLSLAQGPPSSVGSESVYKFPPISNACLNYS |
| 553 | 560 | 559 | YIDACDRI |
| 477 | 486 | 483 | QREYVYIPWS |
| 356 | 363 | 357 | LQTPLNSS |
| 234 | 242 | 240 | NATLLQTPS |
| 584 | 590 | 585 | TQQLLMN |
| 86 | 91 | 89 | QQVYTI |
| 5 | 15 | 6 | LSSYSTTVTNH |
| 397 | 403 | 400 | LSTPQNV |
| 121 | 144 | 129 | TSSYTLQRPPGLHEYTSSISSISS |
| 193 | 198 | 195 | TSPLPK |
| 17 | 26 | 18 | PTFSFESLNS |
| 52 | 60 | 58 | SSMVSSSSD |
| 493 | 499 | 494 | KDLFQIT |

3. CST20
MSILSENNPTQTSITDPNESSHLHNPELNSGTRVASGPGPGPEVESTPLAPPTEVMNTTSANTSSLSLGSPM
HEKIKQFDQDEVDTGETNDRTIESGSSDIDDSQQSHNNNNNNNNNESNPESSEADDEKTQGMPPRMPGTFNV
KGLHQGDDSDNEKQYTELTKSINKRTSKDSYSPGTLESPGTLNALETNNVSPAVIEEEQHTSSLEDLSLSLQ
HQNENARLSAPRSAPPQVSTSKTSSFHDMSSVISSSTSVHKIPSNPTSTRGSHLSSYKSTLDPGKPAQAAAP
PPPEIDIDNLLTKSELDSETDTLSSATNSPNLLRNDTLQGIPTRDDENIDDSPRQLSQNTSATSRNTSGTST
STVVKNSRSGTSKLTSTSTAHNQTAAITPIIPSHNKFHQQVINTNSTNSSSSLEPLGVGINSNSSPKNGKKR
KSGSKVRGVFSSMFGKNKSTSSSSSSNSGSNSHSQEVNIKISTPFNAKHLAHVGIDDNGSYTGLPIEWERLL
SASGITKKEQQQHPQAVMDIVAFYQDTSENPDDAAFKKFHFDNNKSSSSGWSNENTPPATPGGSNSGSGGGG
GGAPSSPHRTPPSSIIEKNNVEQKVITPSQSMPTKTESKQSENQHPHEDNATQYTPRTPTSHVQEGQFIPSR
PAPKPPSTPLSSMSVSHKTPSSQSLPRSDSQSDIRSSTPKSHQDISPSKIKIRSISSKSLKSMRSRKSGDKF
THIAPAPPPPSLPSIPKSKSHSASLSSQLRPATNGSTTAPIPASAAFGGENNALPRQRINEFKAHRAPPPPP
SASPAPPVPPAPPANLLSEQTSEIPQQRTAPSQALADVTAPTNIYEIQQTKYQEAQQKLREKKARELEEIQR
LREKNERQNRQQETGQNNADTASGGSNIAPPVPVPNKKPPSGSGGGRDAKQAALIAQKKREEKKRKNLQIIA
KLKTICNPGDPNELYVDLVKIGQGASGGVFLAHDVRDKSNIVAIKQMNLEQQPKKELIINEILVMKGSSHPN
IVNFIDSYLLKGDLWVIMEYMEGGSLTDIVTHSVMTEGQIGVVCRETLKGLKFLHSKGVIHRDIKSDNILLN
MDGNIKITDFGFCAQINEINSKRITMVGTPYWMAPEIVSRKEYGPKVDVWSLGIMIIEMLEGEPPYLNETPL
RALYLIATNGTPKLKDPESLSYDIRKFLAWCLQVDFNKRADADELLHDNFITECDDVSSLSPLVKIARLKKM
SESD*

| Rank | Sequence | Start position | Score |
|---|---|---|---|
| 1 | QTSITDPNESSHLHNP | 11 | 0.98 |
| 2 | GGSNIAPPVPVPNKKP | 888 | 0.97 |
| 3 | SSDIDDSQQSHNNNNN | 98 | 0.96 |
| 4 | NATQYTPRTPTSHVQE | 626 | 0.95 |
| 5 | LKTICNPGDPNELYVD | 938 | 0.94 |
| 6 | APEIVSRKEYGPKVDV | 1114 | 0.93 |
| 7 | QSENQHPHEDNATQYT | 616 | 0.92 |
| 7 | SGGGGGGAPSSPHRTP | 572 | 0.92 |
| 7 | PESSEADDEKTQGMPP | 121 | 0.92 |
| 7 | KGVIHRDIKSDNILLN | 1065 | 0.92 |
| 7 | LWVIMEYMEGGSLTDI | 1022 | 0.92 |
| 8 | LEEIQRLREKNERQNR | 859 | 0.91 |
| 8 | SRKSGDKFTHIAPAPP | 713 | 0.91 |
| 8 | GQFIPSRPAPKPPSTP | 642 | 0.91 |
| 8 | DTLQGIPTRDDENIDD | 324 | 0.91 |
| 9 | EILVMKGSSHPNIVNF | 997 | 0.90 |
| 9 | KSNIVAIKQMNLEQQP | 974 | 0.90 |
| 9 | ALIAQKKREEKKRKNL | 917 | 0.90 |
| 9 | KPPSGSGGGRDAKQAA | 902 | 0.90 |
| 9 | PQQRTAPSQALADVTA | 817 | 0.90 |
| 9 | ALPRQRINEFKAHRAP | 773 | 0.90 |
| 9 | LSSYKSTLDPGKPAQA | 270 | 0.90 |
| 9 | PGTLESPGTLNALETN | 177 | 0.90 |
| 9 | FLAWCLQVDFNKRADA | 1179 | 0.90 |
| 10 | HKTPSSQSLPRSDSQS | 665 | 0.89 |
| 10 | SSPHRTPPSSIIEKNN | 581 | 0.89 |
| 10 | KRTSKDSYSPGTLESP | 168 | 0.89 |
| 10 | EKTQGMPPRMPGTFNV | 129 | 0.89 |
| 10 | IMIIEMLEGEPPYLNE | 1134 | 0.89 |
| 10 | ITMVGTPYWMAPEIVS | 1104 | 0.89 |
| 11 | EQKVITPSQSMPTKTE | 598 | 0.88 |
| 11 | SSSSGWSNENTPPATP | 550 | 0.88 |
| 11 | GTRVASGPGPGPEVES | 31 | 0.88 |
| 12 | TAPIPASAAFGGENNA | 758 | 0.87 |
| 12 | PKPPSTPLSSMSVSHK | 651 | 0.87 |
| 12 | LGVGINSNSSPKNGKK | 416 | 0.87 |
| 12 | DTLSSATNSPNLLRND | 309 | 0.87 |
| 12 | DVSSLSPLVKIARLKK | 1208 | 0.87 |
| 13 | THIAPAPPPPSLPSIP | 721 | 0.86 |
| 13 | MSSVISSSTSVHKIPS | 245 | 0.86 |
| 14 | AHRAPPPPPSASPAPP | 784 | 0.85 |
| 14 | SSIIEKNNVEQKVITP | 589 | 0.85 |
| 14 | AITPIIPSHNKFHQQV | 386 | 0.85 |
| 14 | SGTSKLTSTSTAHNQT | 369 | 0.85 |
| 14 | STRGSHLSSYKSTLDP | 264 | 0.85 |
| 15 | QNRQQETGQNNADTAS | 872 | 0.84 |
| 15 | AHVGIDDNGSYTGLPI | 483 | 0.84 |
| 15 | SRNTSGTSTSTVVKNS | 352 | 0.84 |
| 15 | DENIDDSPRQLSQNTS | 334 | 0.84 |
| 15 | KGLHQGDDSDNEKQYT | 145 | 0.84 |
| 16 | DIVAFYQDTSENPDDA | 523 | 0.83 |
| 16 | SGSNSHSQEVNIKIST | 460 | 0.83 |
| 16 | PEVESTPLAPPTEVMN | 42 | 0.83 |
| 16 | STVVKNSRSGTSKLTS | 361 | 0.83 |
| 17 | GGVFLAHDVRDKSNIV | 963 | 0.82 |
| 17 | GETNDRTIESGSSDID | 87 | 0.82 |
| 17 | PPPSASPAPPVPPAPP | 790 | 0.82 |
| 17 | AFGGENNALPRQRINE | 766 | 0.82 |
| 17 | NKSTSSSSSSNSGSNS | 449 | 0.82 |
| 18 | DDAAFKKFHFDNNKSS | 536 | 0.81 |
| 18 | GKPAQAAAPPPPEIDI | 280 | 0.81 |
| 18 | THSVMTEGQIGVVCRE | 1039 | 0.81 |
| 19 | SQSDIRSSTPKSHQDI | 678 | 0.80 |
| 19 | PRTPTSHVQEGQFIPS | 632 | 0.80 |
| 19 | EKQYTELTKSINKRTS | 156 | 0.80 |
| 20 | QDEVDTGETNDRTIES | 81 | 0.79 |
| 20 | TSSLEDLSLSLQHQNE | 205 | 0.79 |
| 21 | EQTSEIPQQRTAPSQA | 811 | 0.78 |
| 21 | SPRQLSQNTSATSRNT | 340 | 0.78 |
| 22 | SSMSVSHKTPSSQSLP | 659 | 0.77 |
| 22 | EVNIKISTPFNAKHLA | 468 | 0.77 |
| 22 | PRMPGTFNVKGLHQGD | 136 | 0.77 |
| 22 | GVVCRETLKGLKFLHS | 1049 | 0.77 |
| 23 | LPSIPKSKSHSASLSS | 732 | 0.76 |
| 23 | QSLPRSDSQSDIRSST | 671 | 0.76 |
| 23 | KVRGVFSSMFGKNKST | 437 | 0.76 |
| 23 | SSTSVHKIPSNPTSTR | 251 | 0.76 |
| 23 | NARLSAPRSAPPQVST | 221 | 0.76 |
| 23 | ESLSYDIRKFLAWCLQ | 1170 | 0.76 |
| 24 | KIKQFDQDEVDTGETN | 75 | 0.75 |
| 24 | SSLSLGSPMHEKIKQF | 64 | 0.75 |
| 24 | ASGITKKEQQQHPQAV | 506 | 0.75 |
| 24 | SSSSLEPLGVGINSNS | 409 | 0.75 |
| 24 | LYLIATNGTPKLKDPE | 1155 | 0.75 |

| Start | End | Max_score_pos | Sequence |
|---|---|---|---|
| 1171 | 1187 | 1183 | SLSYDIRKFLAWCLQVD |
| 949 | 958 | 953 | ELYVDLVKIG |
| 892 | 901 | 895 | IAPPVPVPNK |
| 962 | 972 | 968 | SGGVFLAHDVR |
| 1047 | 1055 | 1049 | QIGVVCRET |
| 1203 | 1221 | 1215 | ITECDDVSSLSPLVKIARL |
| 1152 | 1159 | 1155 | LRALYLIA |
| 479 | 487 | 484 | AKHLAHVGI |
| 195 | 201 | 197 | SPAVIEE |
| 205 | 217 | 214 | TSSLEDLSLSLQH |
| 932 | 944 | 935 | LQIIAKLKTICNP |
| 1035 | 1041 | 1039 | TDIVTHS |
| 1007 | 1027 | 1015 | PNIVNFIDSYLLKGDLWVIME |
| 785 | 811 | 801 | HRAPPPPPSASPAPPVPPAPPANLLSE |
| 516 | 529 | 524 | QHPQAVMDIVAFYQ |
| 1124 | 1135 | 1129 | GPKVDVWSLGIM |
| 995 | 1003 | 997 | INEILVMKG |
| 1058 | 1071 | 1065 | GLKFLHSKGVIHRD |
| 1090 | 1096 | 1093 | FGFCAQI |
| 386 | 403 | 399 | AITPIIPSHNKFHQQVIN |
| 412 | 419 | 415 | SLEPLGVG |
| 245 | 259 | 257 | MSSVISSSTSVHKIP |
| 823 | 831 | 827 | PSQALADVT |
| 438 | 444 | 441 | VRGVFSS |
| 915 | 921 | 918 | QAALIAQ |
| 598 | 604 | 600 | EQKVITP |
| 721 | 750 | 732 | THIAPAPPPPSLPSIPKSKSHSASLSSQLR |
| 224 | 237 | 232 | LSAPRSAPPQVSTS |
| 45 | 53 | 52 | ESTPLAPPT |
| 636 | 668 | 660 | TSHVQEGQFIPSRPAPKPPSTPLSSMSVSHKTP |
| 360 | 366 | 362 | TSTVVKN |
| 976 | 982 | 980 | NIVAIKQ |
| 759 | 766 | 764 | APIPASAA |
| 267 | 276 | 271 | GSHLSSYKST |
| 63 | 70 | 68 | TSSLSLGS |
| 282 | 293 | 288 | PAQAAAPPPPEI |
| 670 | 676 | 671 | SQSLPRS |
| 20 | 26 | 25 | SSHLHNP |
| 1114 | 1121 | 1117 | APEIVSRK |
| 690 | 707 | 705 | HQDISPSKIKIRSISSKS |
| 581 | 591 | 589 | SSPHRTPPSSI |
| 466 | 472 | 472 | SQEVNIK |
| 1143 | 1149 | 1149 | EPPYLNE |
| 836 | 842 | 839 | IYEIQQT |

4. CHK1
MSMNFFNSSEPARDHKPDQEKETVMTTEHYEFERPDVKAIRNFKFFRSDETETKKGPNLHISDLSPLESQSV
PPSALSLNHSIIPDQYERRQDTPDPIHTPEISLSDYLYDQTLSPQGFDNSRENFNIHKTIASLFEDNSSVVS
QESTDDTKTTLSSETCDSFSLNNASYLTNINFVQNHLQYLSQNVLGNRTSNSLPPSSSSQIDFDASNLTPDS
IPGYILNKKLGSVHQSTDSVYNAIKIPQNEEYNCCTKASASQNPTNLNSKVIVRLSPNIFQNLSLSRFLNEW
YILSGKHSSKEHQIWSNESLTNEYVQDKTIPTFDKESARFRPTLPINIPGILYPQEIINFCVNSHDYPLEHP
SQSTDQKRFAMVYQDNDYKTFKELSMFTLHELQTRQGSYSSNESRRKSSSGFNIGVNATTTEAGSLESFSNL
MQNHHLGATSTNGDPFHSKLAKFEYGVSKSPMKLIEILTDIMRVVETISVIHELGFVHNGLTSSNLLKSEKN
VRDIKITGWGFAFSFTENCSQGYRNKHLAQVQDLIPYMAPEVLAITNSVVDYRSDFYSLGVIMYELVLGILP
FKNSNPQKLIRMHTFENPIAPSALAPGWISEKLSGVIMKLLEKHPHNRYTDCHSLLHDLIEVKNMYISKLLD
SGETIPNSNLNLSDRQYYLTKENLLHPEKMGITPVLGLKESFIGRRDFLQNVTEVYNNSKNGIDLLFISGES
GRGKTIILQDLRAAAVLKQDFYYSWKFSFFGADTHVYRFLVEGVQKIITQILNSSEEIQNTWRDVILTHIPI
DLSILFYLIPELKVLLGKKYTSIYKHKIGMGMLKRSFKEDQTSRLEIKLRQILKEFFKLVAKQGLSIFLDDV
QWCSEESWRLLCDVLDFDSSGEVRESYNIKIVVCYALNADHLENVNIEHKKISFCQYAKQSHLNLREFSIPH
IPLEDAIEFLCEPYTRSHDHECNSKKSDVIANLNCTNEYQQNTCKVIPSIIQELYQSSEGNVLLLIFLTRMT
KLSGKVPFQRFSVKNSYLYDHLSNSNYGTTRKEILTNYLNMGTNSDTRALLKVAALISNGSGFFFSDLIVAT
DLPMAEAFQLLQICIHSRIIVPTSTYYKIPMDLIASDQTPFDLTDDNIWKLATLCSYKFYHDSICTHIIKEL
NASGEFKELSRLCGLRFYNTITKERLLNIGGYLQMATHFRNSYEVAGPEENEKYVEVLVQAGRYAISTYNMK
LSQWFFNVVGELVYNLDSKTQLKSVLTIAENHFNSREFEQCLSVVENAQRKFGFDRLIFSIQIVRCKIELGD
YDEAHRIAIECLKELGVPLDDDDEYTSENSLETCLGKIPLSVADIRGILKIKRCKNSRTLLMYQLISELIVL
FKLQGKDKVRRFLTAYAMSQIHTQGSSPYCAVILIDFAQSFVNETTTSGMLKAKELSIVMLSLINRAPEISL
SYVQSIYEYYFSCHAVFFESIEKMSDLIHPGNASSHCTRSSYYSSFHLIVNVSKIFFSCMNGESFKMFSTFK
CKSYLTGDPQMPEMDNFLYDSEMLLAGHSELNEFMRKYQSFNQTSVGKFCYYLIVLLVMSREHRFDEAADLV
LKVLEDLSEKLPVSFLHHQYYLICGKVFAYHQTKTPESEEQVERILARQFERYELWASTNKPTLLPRYLLLS
TYKQIRENHVDKLEILDSFEEALQTAHKFHNVYDMCWINLECARWLISINQKRHRISRMVKQGLKILRSLEL
NNHLRLAEFEFDEYIEDEDHRNKWAGLTNNPTLDTVTTWQQQNMPDKVSPCNDKQLVHGKQFGKKEFDSHLL
RLHFDGQYTGLDLNSAIRECLAISEALDENSILTKLMASAIKYSGATYGVIVTKKNQETPFLRTIGSQHNIH
TLNNMPISDDICPAQLIRHVLHTGETVNKAHDHIGFANKFENEYFQTTDKKYSVVCLPLKSSLGLFGALYLE
GSDGDFGHEDLFNERKCDLLQLFCTQAAVALGKERLLLQMELAKMAAEDATDEKASFLANMSHEIRTPFNSL
LSFAIFLLDTKLDSTQREYVEAIQSSAMITLNIIDGILAFSKIEHGSFTLENAPFSLNDCIETAIQVSGETI
LNDQIELVFCNNCPEIEFVVGDLTRFRQIVINLVGNAIKFTTKGHVLISCDSRKITDDRFEINVSVEDSGIG
ISKKSQNKVFGAFSQVDGSARREYGGSGLGLAISKKLTELMGGTIRFESEEGIGTTFYVSVIMDAKEYSSPP
FSLNKKCLIYSQHCLTAKSISNMLNYFGSTVKVTNQKSEFSTSVQANDIIFVDRGMEPDVSCKTKIIPIDPK
PFKRNKLISILKEQPSLPTKVFGNNKSNLSKQYPLRILLAEDNLLNYKVCLKHLDKLGYKADHAKDGVVVLD
KCKELLEKDEKYDVILMDIQMPRKDGITATRDLKTLFHTQKKESWLPVIVALTANVAGDDKKRCLEEGMFDF
ITKPILPDELRRILTKVGETVNM*

| Rank | Sequence | Start position | Score |
|---|---|---|---|
| 1 | TETKKGPNLHISDLSP | 51 | 0.96 |
| 2 | LMDIQMPRKDGITATR | 2392 | 0.93 |
| 2 | SVHQSTDSVYNAIKIP | 228 | 0.93 |
| 2 | GGTIRFESEEGIGTTF | 2202 | 0.93 |
| 3 | FLCEPYTRSHDHECNS | 945 | 0.92 |
| 3 | IGMGMLKRSFKEDQTS | 820 | 0.92 |
| 3 | EHYEFERPDVKAIRNF | 28 | 0.92 |
| 4 | HSSKEHQIWSNESLTN | 295 | 0.91 |
| 4 | SDLIVATDLPMAEAFQ | 1074 | 0.91 |
| 5 | HSIIPDQYERRQDTPD | 81 | 0.90 |
| 5 | PPSSSSQIDFDASNLT | 198 | 0.90 |
| 5 | VILIDFAQSFVNETTT | 1400 | 0.90 |
| 5 | PEISLSDYLYDQTLSP | 101 | 0.90 |
| 6 | CSEESWRLLCDVLDFD | 867 | 0.89 |
| 6 | ESRRKSSSGFNIGVNA | 403 | 0.89 |
| 6 | CLAISEALDENSILTK | 1820 | 0.89 |
| 7 | KESFIGRRDFLQNVTE | 687 | 0.88 |
| 7 | RQGSYSSNESRRKSSS | 395 | 0.88 |
| 7 | AKEYSSPPFSLNKKCL | 2225 | 0.88 |
| 7 | FDEYIEDEDHRNKWAG | 1739 | 0.88 |
| 7 | KSYLTGDPQMPEMDNF | 1514 | 0.88 |
| 7 | CTRSSYYSSFHLIVNV | 1477 | 0.88 |
| 8 | PGWISEKLSGVIMKLL | 602 | 0.87 |
| 8 | QKLIRMHTFENPIAPS | 583 | 0.87 |
| 8 | KETVMTTEHYEFERPD | 21 | 0.87 |
| 8 | AFSKIEHGSFTLENAP | 2055 | 0.87 |
| 8 | QREYVEAIQSSAMITL | 2032 | 0.87 |
| 8 | TWQQQNMPDKVSPCND | 1766 | 0.87 |
| 8 | NSYEVAGPEENEKYVE | 1193 | 0.87 |
| 8 | DLIASDQTPFDLTDDN | 1112 | 0.87 |
| 8 | PTSTYYKIPMDLIASD | 1102 | 0.87 |
| 9 | HDHECNSKKSDVIANL | 954 | 0.86 |
| 9 | HDLIEVKNMYISKLLD | 633 | 0.86 |
| 9 | DLIPYMAPEVLAITNS | 537 | 0.86 |
| 9 | HPSQSTDQKRFAMVYQ | 359 | 0.86 |
| 9 | DKTIPTFDKESARFRP | 315 | 0.86 |
| 9 | VSVIMDAKEYSSPPFS | 2219 | 0.86 |
| 9 | DSRKITDDRFEINVSV | 2139 | 0.86 |
| 9 | HDHIGFANKFENEYFQ | 1903 | 0.86 |
| 9 | HRNKWAGLTNNPTLDT | 1748 | 0.86 |
| 9 | PGNASSHCTRSSYYSS | 1470 | 0.86 |
| 9 | VQSIYEYYFSCHAVFF | 1443 | 0.86 |
| 9 | RFSVKNSYLYDHLSNS | 1018 | 0.86 |
| 10 | LHPEKMGITPVLGLKE | 673 | 0.85 |
| 10 | QPSLPTKVFGNNKSNL | 2318 | 0.85 |
| 10 | EGIGTTFYVSVIMDAK | 2211 | 0.85 |
| 10 | DGDFGHEDLFNERKCD | 1947 | 0.85 |
| 10 | AQLIRHVLHTGETVNK | 1886 | 0.85 |
| 10 | SAIKYSGATYGVIVTK | 1839 | 0.85 |
| 10 | HGKQFGKKEFDSHLLR | 1786 | 0.85 |
| 10 | AGHSELNEFMRKYQSF | 1538 | 0.85 |
| 11 | VNSHDYPLEHPSQSTD | 350 | 0.84 |
| 11 | NEEYNCCTKASASQNP | 245 | 0.84 |
| 11 | KKRCLEEGMFDFITKP | 2437 | 0.84 |
| 11 | LISINQKRHRISRMVK | 1702 | 0.84 |
| 11 | ASTNKPTLLPRYLLLS | 1641 | 0.84 |
| 11 | SVLTIAENHFNSREFE | 1248 | 0.84 |
| 12 | SSEEIQNTWRDVILTH | 774 | 0.83 |
| 12 | SGRGKTIILQDLRAAA | 720 | 0.83 |
| 12 | LSDRQYYLTKENLLHP | 660 | 0.83 |
| 12 | IPGILYPQEIINFCVN | 336 | 0.83 |
| 12 | KELLEKDEKYDVILMD | 2379 | 0.83 |
| 12 | YNAIKIPQNEEYNCCT | 237 | 0.83 |
| 12 | KIIPIDPKPFKRNKLI | 2297 | 0.83 |
| 12 | IIFVDRGMEPDVSCKT | 2281 | 0.83 |
| 12 | SVVENAQRKFGFDRLI | 1267 | 0.83 |
| 12 | QAGRYAISTYNMKLSQ | 1212 | 0.83 |
| 12 | ATLCSYKFYHDSICTH | 1132 | 0.83 |
| 12 | TLSPQGFDNSRENFNI | 113 | 0.83 |
| 12 | TRKEILTNYLNMGTNS | 1038 | 0.83 |
| 13 | PSIIQELYQSSEGNVL | 984 | 0.82 |
| 13 | SSEPARDHKPDQEKET | 8 | 0.82 |
| 13 | EVLAITNSVVDYRSDF | 545 | 0.82 |
| 13 | NCSQGYRNKHLAQVQD | 522 | 0.82 |
| 13 | GSARREYGGSGLGLAI | 2178 | 0.82 |
| 13 | KGHVLISCDSRKITDD | 2131 | 0.82 |
| 13 | LYLEGSDGDFGHEDLF | 1941 | 0.82 |
| 14 | EFSIPHIPLEDAIEFL | 931 | 0.81 |
| 14 | KIITQILNSSEEIQNT | 766 | 0.81 |
| 14 | DQKRFAMVYQDNDYKT | 365 | 0.81 |
| 14 | RFLNEWYILSGKHSSK | 283 | 0.81 |
| 14 | AFSQVDGSARREYGGS | 2172 | 0.81 |
| 14 | ERYELWASTNKPTLLP | 1635 | 0.81 |
| 14 | TLSSETCDSFSLNNAS | 154 | 0.81 |
| 15 | LGFVHNGLTSSNLLKS | 486 | 0.80 |
| 15 | TISVIHELGFVHNGLT | 479 | 0.80 |
| 15 | GATSTNGDPFHSKLAK | 439 | 0.80 |
| 15 | VKAIRNFKFFRSDETE | 37 | 0.80 |
| 15 | GYKADHAKDGVVVLDK | 2362 | 0.80 |
| 15 | SFLANMSHEIRTPFNS | 2000 | 0.80 |
| 15 | FFSCMNGESFKMFSTF | 1496 | 0.80 |
| 15 | SVVSQESTDDTKTTLS | 141 | 0.80 |
| 15 | AMSQIHTQGSSPYCAV | 1385 | 0.80 |
| 15 | GGYLQMATHFRNSYEV | 1182 | 0.80 |
| 15 | FQLLQICIHSRIIVPT | 1088 | 0.80 |
| 16 | YERRQDTPDPIHTPEI | 88 | 0.79 |
| 16 | NSKVIVRLSPNIFQNL | 264 | 0.79 |
| 16 | LPVIVALTANVAGDDK | 2422 | 0.79 |
| 16 | VERILARQFERYELWA | 1626 | 0.79 |
| 16 | HKTIASLFEDNSSVVS | 129 | 0.79 |
| 17 | VRDIKITGWGFAFSFT | 505 | 0.78 |
| 17 | YGVSKSPMKLIEILTD | 457 | 0.78 |
| 17 | EFEQCLSVVENAQRKF | 1261 | 0.78 |
| 18 | DFLQNVTEVYNNSKNG | 695 | 0.77 |
| 18 | MYELVLGILPFKNSNP | 567 | 0.77 |
| 18 | RFRPTLPINIPGILYP | 327 | 0.77 |
| 18 | TPDSIPGYILNKKLGS | 213 | 0.77 |
| 18 | FHNVYDMCWINLECAR | 1685 | 0.77 |
| 18 | SFVNETTTSGMLKAKE | 1408 | 0.77 |
| 19 | PQEIINFCVNSHDYPL | 342 | 0.76 |
| 19 | HQTKTPESEEQVERIL | 1615 | 0.76 |
| 19 | SGMLKAKELSIVMLSL | 1416 | 0.76 |
| 19 | RCKIELGDYDEAHRIA | 1289 | 0.76 |
| 20 | SGEVRESYNIKIVVCY | 884 | 0.75 |
| 20 | EFVVGDLTRFRQIVIN | 2105 | 0.75 |
| 20 | KFENEYFQTTDKKYSV | 1911 | 0.75 |
| 20 | ENSILTKLMASAIKYS | 1829 | 0.75 |
| 20 | GVPLDDDDEYTSENSL | 1312 | 0.75 |
| 20 | DYLYDQTLSPQGFDNS | 107 | 0.75 |

| Start | End | Max_score_pos | Sequence |
|---|---|---|---|
| 1558 | 1572 | 1567 | VGKFCYYLIVLLVMS |
| 892 | 904 | 898 | NIKIVVCYALNAD |
| 1923 | 1944 | 1928 | KYSVVCLPLKSSLGLFGALYLE |
| 996 | 1005 | 1001 | GNVLLLIFLT |
| 2369 | 2381 | 2375 | KDGVVVLDKCKEL |
| 1394 | 1410 | 1400 | SSPYCAVILIDFAQSFV |
| 2421 | 2431 | 2425 | WLPVIVALTAN |
| 1206 | 1219 | 1209 | YVEVLVQAGRYAIS |
| 1087 | 1118 | 1093 | AFQLLQICIHSRIIVPTSTYYKIPMDLIASDQ |
| 1580 | 1617 | 1585 | AADLVLKVLEDLSEKLPVSFLHHQYYLICG |
| | | | KVFAYHQT |
| 1356 | 1371 | 1368 | LLMYQLISELIVLFKL |
| 1262 | 1271 | 1266 | FEQCLSVVEN |
| 1226 | 1240 | 1235 | SQWFFNVVGELVYNL |
| 873 | 881 | 876 | RLLCDVLDF |
| 2349 | 2366 | 2355 | LNYKVCLKHLDKLGYKAD |
| 752 | 773 | 758 | ADTHVYRFLVEGVQKIITQILN |
| 1437 | 1459 | 1453 | EISLSYVQSIYEYYFSCHAVFFE |
| 530 | 577 | 573 | KHLAQVQDLIPYMAPEVLAITNSVVDYRSDFYSLGVIMY |
| | | | ELVLGILPF |
| 1056 | 1066 | 1061 | RALLKVAALIS |
| 2131 | 2141 | 2135 | KGHVLISCDSR |
| 1961 | 1985 | 1966 | CDLLQLFCTQAAVALGKERLLLQME |
| 784 | 811 | 797 | DVILTHIPIDLSILFYLIPELKVLLGKK |
| 2090 | 2101 | 2095 | NDQIELVFCNNC |
| 1474 | 1499 | 1489 | SSHCTRSSYYSSFHLIVNVSKIFFSC |
| 1881 | 1896 | 1891 | DDICPAQLIRHVLHTG |
| 264 | 274 | 270 | NSKVIVRLSPN |
| 2114 | 2125 | 2119 | FRQIVINLVGNA |
| 2228 | 2250 | 2242 | YSSPPFSLNKKCLIYSQHCLTAK |
| 977 | 993 | 983 | QNTCKVIPSIIQELYQS |
| 1646 | 1659 | 1651 | PTLLPRYLLLSTYK |
| 2216 | 2224 | 2219 | TFYVSVIMD |
| 449 | 492 | 479 | HSKLAKFEYGVSKSPMKLIEILTDIMRVVETISVIHELG |
| | | | FVHNG |
| 1131 | 1152 | 1134 | LATLCSYKFYHDSICTHIIKEL |
| 1421 | 1434 | 1429 | AKELSIVMLSLINR |
| 625 | 641 | 630 | YTDCHSLLHDLIEVKNM |
| 2334 | 2346 | 2340 | SKQYPLRILLAED |
| 1279 | 1295 | 1287 | DRLIFSIQIVRCKIELG |
| 1302 | 1316 | 1310 | RIAIECLKELGVPLD |
| 725 | 747 | 734 | TIILQDLRAAAVLKQDFYYSWKF |
| 1327 | 1350 | 1333 | LETCLGKIPLSVADIRGILKIKRC |
| 1833 | 1854 | 1849 | LTKLMASAIKYSGATYGVIVTK |
| 1796 | 1806 | 1801 | DSHLLRLHFDG |
| 1161 | 1168 | 1164 | LSRLCGLR |
| 2014 | 2027 | 2019 | NSLLSFAIFLLDTK |
| 914 | 950 | 919 | KKISFCQYAKQSHLNLREFSIPHIPLEDAIEFLCEPY |
| 1245 | 1253 | 1249 | QLKSVLTIA |
| 680 | 689 | 683 | ITPVLGLKES |
| 1071 | 1082 | 1079 | FFFSDLIVATDL |
| 58 | 86 | 74 | NLHISDLSPLESQSVPPSALSLNHSIIPD |
| 177 | 188 | 186 | VQNHLQYLSQNV |
| 329 | 360 | 347 | RPTLPINIPGILYPQEIINFCVNSHDYPLEHP |
| 963 | 969 | 968 | SDVIANL |
| 1817 | 1826 | 1823 | IRECLAISEA |
| 1011 | 1030 | 1027 | SGKVPFQRFSVKNSYLYDHL |
| 2103 | 2111 | 2104 | EIEFVVGDL |
| 248 | 255 | 253 | YNCCTKAS |
| 1510 | 1519 | 1514 | TFKCKSYLTG |
| 711 | 717 | 714 | IDLLFIS |
| 838 | 868 | 854 | EIKLRQILKEFFKLVAKQGLSIFLDDVQWCS |
| 1685 | 1705 | 1695 | FHNVYDMCWINLECARWLISI |
| 276 | 284 | 282 | FQNLSLSRF |
| 1775 | 1788 | 1776 | KVSPCNDKQLVHGK |
| 2291 | 2304 | 2295 | DVSCKTKIIPIDPK |
| 1717 | 1726 | 1723 | KQGLKILRSL |
| 96 | 117 | 107 | DPIHTPEISLSDYLYDQTLSPQ |
| 1666 | 1674 | 1669 | VDKLEILDS |
| 2311 | 2325 | 2313 | LISILKEQPSLPTKV |
| 596 | 603 | 597 | APSALAPG |
| 140 | 146 | 143 | SSVVSQE |
| 217 | 242 | 219 | IPGYILNKKLGSVHQSTDSVYNAIKI |
| 607 | 620 | 614 | EKLSGVIMKLLEKH |
| 2050 | 2059 | 2056 | IDGILAFSKI |
| 2150 | 2157 | 2151 | INVSVEDS |
| 2388 | 2394 | 2393 | YDVILMD |
| 2033 | 2042 | 2038 | REYVEAIQSS |
| 643 | 649 | 645 | ISKLLDS |
| 2188 | 2197 | 2194 | GLGLAISKKL |
| 1627 | 1633 | 1628 | ERILARQ |
| 2259 | 2266 | 2262 | FGSTVKVT |
| 697 | 703 | 700 | LQNVTEV |
| 2067 | 2086 | 2074 | ENAPFSLNDCIETAIQVSGE |
| 157 | 163 | 162 | SETCDSF |
| 2449 | 2457 | 2451 | ITKPILPDE |
| 2167 | 2178 | 2172 | NKVFGAFSQVDG |
| 2273 | 2285 | 2275 | STSVQANDIIFVD |
| 1731 | 1737 | 1734 | HLRLAEF |
| 287 | 294 | 292 | EWYILSGK |
| 386 | 393 | 390 | MFTLHELQ |
| 2410 | 2415 | 2412 | KTLFHT |
| 1378 | 1392 | 1380 | RRFLTAYAMSQIHTQ |
| 813 | 818 | 817 | TSIYKH |
| 196 | 207 | 199 | SLPPSSSSQIDF |
| 129 | 135 | 132 | HKTIASL |
| 1530 | 1540 | 1539 | LYDSEMLLAGH |
| 2459 | 2468 | 2463 | RRILTKVGET |
| 1677 | 1683 | 1682 | EALQTAH |
| 663 | 675 | 663 | RQYYLTKENLLHP |
| 1178 | 1188 | 1181 | LLNIGGYLQMA |
| 35 | 40 | 38 | PDVKAI |
| 1466 | 1471 | 1468 | DLIHPG |
| 1999 | 2004 | 2000 | ASFLAN |
| 583 | 588 | 588 | QKLIRM |

5. CAP1
MSTEESQFNVQGYNIITILKRLEAATSRLEDITIFQEEANKNHYGVDSLTEKGTPKSRTVESSEATSDGKSL
ESTSFATFSEAPVEKSKLIVEFENFVESYVHPLVETSKKIDSLVGESAQYFYEAFVEQGKFLELVLQSQQPD
MTDPALAKALEPMNAKCTKINELKDSNRKSPFFNHLSTFSESNAVFYWIGIPTPVSYITDTKDTVKFWSDRV
LKEYKTKDQVHVEWVKQTLSVFDELKNYVKEYHTTGVAWNPKGKPFAEVVSQQTESAAKNSSSASGSAGGAA
PPPPPPPPPATFFDDTEKDSENPSPASGGINAVFAELNQGANITSGLKKVDKSEMTHKNPELRKQPPVAPKK
PAPPKKPSSLSGGVSSAPVKKPAKKELIDGTKWIIQNFTKADISDLSPITIEVEMHQSVFIGNCSDVTIQLK
GKANAVSVSETKNVALVIDSLISGVDVIKSYKFGIQVLGLVPMLSIDKSDEGTIYLSQESIDNDSQVFTSST
TALNINAPKENDDYEELAVPEQFVSKVVNGKLVTQIVEHAG*

| Rank | Sequence | Start position | Score |
|---|---|---|---|
| 1 | PALAKALEPMNAKCTK | 148 | 0.93 |
| 2 | KGTPKSRTVESSEATS | 52 | 0.91 |
| 2 | VFYWIGIPTPVSYITD | 189 | 0.91 |
| 3 | TVESSEATSDGKSLES | 59 | 0.90 |
| 3 | DVTIQLKGKANAVSVS | 426 | 0.90 |
| 3 | DKSEMTHKNPELRKQP | 339 | 0.90 |
| 4 | EGTIYLSQESIDNDSQ | 483 | 0.89 |
| 5 | ELRKQPPVAPKKPAPP | 349 | 0.88 |
| 5 | HPLVETSKKIDSLVGE | 103 | 0.88 |
| 6 | GGAAPPPPPPPPPATF | 285 | 0.86 |
| 6 | KEYHTTGVAWNPKGKP | 246 | 0.86 |
| 6 | EWVKQTLSVFDELKNY | 229 | 0.86 |
| 7 | MLSIDKSDEGTIYLSQ | 475 | 0.85 |
| 7 | TIEVEMHQSVFIGNCS | 410 | 0.85 |
| 7 | TIFQEEANKNHYGVDS | 33 | 0.85 |
| 8 | VDSLTEKGTPKSRTVE | 46 | 0.84 |
| 9 | KELIDGTKWIIQNFTK | 385 | 0.83 |
| 9 | GESAQYFYEAFVEQGK | 117 | 0.83 |
| 10 | SGLKKVDKSEMTHKNP | 333 | 0.82 |
| 10 | PATFFDDTEKDSENPS | 297 | 0.82 |
| 11 | PEQFVSKVVNGKLVTQ | 524 | 0.81 |
| 11 | HQSVFIGNCSDVTIQL | 416 | 0.81 |
| 11 | KWIIQNFTKADISDLS | 392 | 0.81 |
| 12 | SQVFTSSTTALNINAP | 497 | 0.80 |
| 13 | YEELAVPEQFVSKVVN | 518 | 0.79 |
| 13 | TEESQFNVQGYNIITI | 3 | 0.79 |
| 13 | TSRLEDITIFQEEANK | 26 | 0.79 |
| 13 | PVSYITDTKDTVKFWS | 198 | 0.79 |
| 13 | SQQPDMTDPALAKALE | 140 | 0.79 |
| 14 | GGVSSAPVKKPAKKEL | 372 | 0.78 |
| 14 | PVAPKKPAPPKKPSSL | 355 | 0.78 |
| 15 | FGIQVLGLVPMLSIDK | 465 | 0.77 |
| 16 | ALNINAPKENDDYEEL | 506 | 0.76 |
| 16 | LKEYKTKDQVHVEWVK | 217 | 0.76 |
| 17 | FAEVVSQQTESAAKNS | 262 | 0.75 |
| 17 | GVAWNPKGKPFAEVVS | 252 | 0.75 |

| Start | End | Max_score_pos | Sequence |
|---|---|---|---|
| 97 | 109 | 103 | FVESYVHPLVETS |
| 445 | 479 | 470 | NVALVIDSLISGVDVIKSYKFGIQVLGLVPMLSID |
| 520 | 542 | 529 | ELAVPEQFVSKVVNGKLVTQIVE |
| 133 | 141 | 136 | FLELVLQSQ |
| 223 | 240 | 228 | KDQVHVEWVKQTLSVFDE |
| 351 | 382 | 377 | RKQPPVAPKKPAPPKKPSSLSGGVSSAPVKKP |
| 262 | 269 | 264 | FAEVVSQQ |
| 112 | 131 | 125 | IDSLVGESAQYFYEAFVEQG |
| 403 | 432 | 427 | ISDLSPITIEVEMHQSVFIGNCSDVTIQLK |
| 186 | 202 | 199 | SNAVFYWIGIPTPVSYI |
| 435 | 441 | 438 | ANAVSVS |
| 148 | 154 | 151 | PALAKAL |
| 77 | 95 | 91 | FATFSEAPVEKSKLIVEFE |
| 43 | 49 | 46 | HYGVDSL |
| 8 | 22 | 16 | FNVQGYNIITILKRL |
| 242 | 248 | 247 | KNYVKEY |
| 318 | 324 | 322 | INAVFAE |
| 485 | 491 | 489 | TIYLSQE |
| 286 | 299 | 290 | GAAPPPPPPPPPAT |
| 496 | 502 | 500 | DSQVFTS |
| 208 | 219 | 219 | TVKFWSDRVLKE |
| 176 | 183 | 177 | FFNHLSTF |

6. CDC24
MEHPPAALRTFSTQSTSSLNSVSTVSSSRIVSSGPVNINNFNKPSTPKDHLFYRCESLKRKLQKIPGMEPFL
NQAFNQAEQLSEQQALALAQERSNGNGHSNGKRHQSLDGAMNRLSVGSDSSSIQGSLTRMATNASTSSLISG
MPNNNTLFTFTAGVLPANISVDPATHLWKLFQQGAPFCVLINHILPDSQIPVVSSDDLRICKKSVYDFLIAV
KTQLNFDDENMFTISNVFSDNAQDLIKIIDVINKLLAEYSDASDSGGGDEDVNMDVQITDERSKVFREIIET
ERKYVQDLELMCKYRQDLIEAENLSSEQIHLLFPNLNEIIDFQRRFLNGLECNINVPIRYQRIGSVFIHASL
GPFNAYEPWTIGQLTAIDLINKEAANLKKSSSLLDPGFELQSYILKPIQRLCKYPLLLKELIKTSPEYSKQD
PHGSSSSTSFNELLVAKTAMKELANQVNEAQRRAENIEHLEKLKERVGNWRGFNLDAQGELLFHGQVGVKDA
ENEKEYVAYLFEKIVFFFTEIDDNKKSDKQEKKSKFSTRKRSTSSNLSSSTTNLLESINNSRKDNTLPLELK
GRVYISEIYNISAPNTPGSTLIISWSGRKESGSFTLRYRSEEARNQWEKCLRDLKTNEMNKQIHKKLRDSDS
SFNTDDSAIYDYTGISTSPVNQSTQQQYYDHRGSHSSRHHSSSSTLSMMKNNRVKSGDLSRISSTSTTLDSF
SNNLNGSPNTTNPSLTSSDATKTIPTFDVAIKLLYKSTELSEPLIVNAQIEYNDLLQKIISQIITSNLVADD
VNISRLRYKDDEGDFVNLNSDDDWGLVLDMLTSEDFYQTSSNEKRSVTVWVS*

| Rank | Sequence | Start position | Score |
|---|---|---|---|
| 1 | SSSIQGSLTRMATNAS | 122 | 0.96 |
| 2 | SGGGDEDVNMDVQITD | 261 | 0.92 |
| 3 | RVYISEIYNISAPNTP | 578 | 0.91 |
| 3 | KELIKTSPEYSKQDPH | 419 | 0.91 |
| 3 | YSDASDSGGGDEDVNM | 255 | 0.91 |
| 4 | GLVLDMLTSEDFYQTS | 817 | 0.90 |
| 5 | TKTIPTFDVAIKLLYK | 741 | 0.89 |
| 5 | QSTQQQYYDHRGSHSS | 670 | 0.89 |
| 6 | SSTSTTLDSFSNNLNG | 711 | 0.88 |
| 6 | ARNQWEKCLRDLKTNE | 619 | 0.88 |
| 7 | IYNISAPNTPGSTLII | 584 | 0.87 |
| 8 | SRLRYKDDEGDFVNLN | 796 | 0.86 |
| 8 | PSTPKDHLFYRCESLK | 44 | 0.86 |
| 8 | PWTIGQLTAIDLINKE | 368 | 0.86 |
| 9 | SRIVSSGPVNINNFNK | 28 | 0.85 |
| 10 | NTTNPSLTSSDATKTI | 729 | 0.84 |
| 10 | HHSSSSTLSMMKNNRV | 687 | 0.84 |
| 11 | DFYQTSSNEKRSVTVW | 827 | 0.83 |
| 12 | NNLNGSPNTTNPSLTS | 722 | 0.82 |
| 12 | SVFIHASLGPFNAYEP | 353 | 0.82 |
| 12 | KVFREIIETERKYVQD | 280 | 0.82 |
| 13 | QKIISQIITSNLVADD | 777 | 0.81 |
| 13 | HKKLRDSDSSFNTDDS | 640 | 0.81 |
| 13 | DLRICKKSVYDFLIAV | 201 | 0.81 |
| 13 | TSSLISGMPNNNTLFT | 138 | 0.81 |
| 14 | QERSNGNGHSNGKRHQ | 92 | 0.80 |
| 14 | YTGISTSPVNQSTQQQ | 660 | 0.80 |
| 14 | KESGSFTLRYRSEEAR | 605 | 0.80 |
| 14 | GELLFHGQVGVKDAEN | 491 | 0.80 |
| 15 | KIPGMEPFLNQAFNQA | 64 | 0.79 |
| 15 | TLIISWSGRKESGSFT | 596 | 0.79 |
| 15 | AQRRAENIEHLEKLKE | 462 | 0.79 |
| 16 | VGVKDAENEKEYVAYL | 499 | 0.78 |
| 16 | LVAKTAMKELANQVNE | 446 | 0.78 |
| 16 | YSKQDPHGSSSSTSFN | 428 | 0.78 |
| 16 | LKPIQRLCKYPLLLKE | 405 | 0.78 |
| 16 | FELQSYILKPIQRLCK | 398 | 0.78 |
| 16 | LKKSSSLLDPGFELQS | 387 | 0.78 |
| 16 | NEIIDFQRRFLNGLEC | 325 | 0.78 |
| 16 | RKYVQDLELMCKYRQD | 290 | 0.78 |
| 16 | SLTRMATNASTSSLIS | 128 | 0.78 |
| 17 | RKRSTSSNLSSSTTNL | 543 | 0.77 |
| 17 | KLKERVGNWRGFNLDA | 474 | 0.77 |
| 17 | MPNNNTLFTFTAGVLP | 145 | 0.77 |
| 18 | NEKEYVAYLFEKIVFF | 506 | 0.76 |
| 18 | CKYRQDLIEAENLSSE | 300 | 0.76 |
| 19 | NNRVKSGDLSRISSTS | 699 | 0.75 |
| 19 | TNLLESINNSRKDNTL | 556 | 0.75 |
| 19 | FTEIDDNKKSDKQEKK | 522 | 0.75 |

| Start | End | Max_score_pos | Sequence |
|---|---|---|---|
| 153 | 219 | 182 | TFTAGVLPANISVDPATHLWKLFQQGAPFCVLINHILPDSQI |
| | | | PVVSSDDLRICKKSVYDFLIAVKTQ |
| 390 | 425 | 414 | SSSLLDPGFELQSYILKPIQRLCKYPLLLKELIKTS |
| 746 | 758 | 752 | TFDVAIKLLYKST |
| 351 | 363 | 357 | IGSVFIHASLGPF |
| 490 | 502 | 496 | QGELLFHGQVGVK |
| 761 | 769 | 763 | SEPLIVNAQ |
| 509 | 523 | 512 | EYVAYLFEKIVFFFT |
| 314 | 323 | 319 | SEQIHLLFPN |
| 240 | 256 | 244 | DLIKIIDVINKLLAEYS |
| 19 | 36 | 33 | LNSVSTVSSSRIVSSGPV |
| 572 | 588 | 582 | PLELKGRVYISEIYNIS |
| 443 | 450 | 448 | NELLVAKT |
| 291 | 307 | 295 | KYVQDLELMCKYRQDLI |
| 772 | 793 | 778 | YNDLLQKIISQIITSNLVADDV |
| 48 | 65 | 52 | KDHLFYRCESLKRKLQKI |
| 817 | 822 | 821 | GLVLDM |
| 81 | 92 | 88 | QLSEQQALALAQ |
| 337 | 349 | 343 | GLECNINVPIRYQ |
| 371 | 379 | 377 | IGQLTAIDL |
| 624 | 629 | 628 | EKCLRD |
| 4 | 11 | 6 | PPAALRTF |
| 279 | 284 | 283 | SKVFRE |
| 595 | 600 | 596 | STLIIS |
| 138 | 143 | 139 | TSSLIS |
| 665 | 671 | 665 | TSPVNQS |
| 673 | 679 | 676 | QQQYYDH |
| 685 | 693 | 691 | SRHHSSSST |

7. NOT5
MSARKLQQEFDKLNKKISEGLQAFDEIKDKINATESASQREKLENDLKKELKKLQRSRDQLKQWLGDSSIKL
DKNVLQENRTKIEHAMDQFKELEKSSKIKQFSNEGLELQSQQKRSRFGDDAKYQEACTYINEVIEQLNGQNE
ELEQELDSLSGQSKRKGGSSIQSSIDDVKYKIERNNSHISKLEEVLENLDNDKLDPARIDDIKDDLDYYVEN
NQDEDYVEYDEFYDQLEVDEEDDVEVQGSLAQMAAETEDERRRDEERKREEKEKEKQQQNPPRTSSSVSSSS
SSNQNNIGNNTPPATQIKPSVVTVAAIGDQNNNSASSASSTSTPVKKLKPTLAPAPAPPPITSGTSYSNAIK
AAQTASTSSTSNSSIAHTANDNNNTNKGNRSVSPLASTDNHTHAPAAVSTPVKVLPPGLNHDTSMNSTLRSE
SSSPLVGHAKVNNNHELQISRSQSPMVSNENKVFSDTISRIVNVAHSRLNDPLPLQSITGLLETSLLNCPDS
YDAEKPRQYNPVNVHPSSIDYPQEPMYELNSSHYMKKFDNDTLFFCFYYGDGIDSISKYNAAKELSRRGWVF
NTEFSQWFSKDSKNGGKNRSMSVIQREEENGNIIGDNSNGNEELREKIDDNGGIPSNYKYFDYEKTWLTRRR
ENYQFATENRQIFQ*

| Rank | Sequence | Start position | Score |
|---|---|---|---|
| 1 | GGIPSNYKYFDYEKTW | 628 | 0.96 |
| 1 | PSSIDYPQEPMYELNS | 520 | 0.96 |
| 1 | RSESSSPLVGHAKVNN | 430 | 0.96 |
| 2 | ASSASSTSTPVKKLKP | 323 | 0.94 |
| 3 | IDDIKDDLDYYVENNQ | 203 | 0.92 |
| 4 | QWLGDSSIKLDKNVLQ | 63 | 0.91 |
| 4 | CFYYGDGIDSISKYNA | 550 | 0.91 |
| 4 | TSLLNCPDSYDAEKPR | 496 | 0.91 |
| 4 | SSTSNSSIAHTANDNN | 368 | 0.91 |
| 4 | GSSIQSSIDDVKYKIE | 162 | 0.91 |
| 5 | SLAQMAAETEDERRRD | 245 | 0.90 |
| 6 | THAPAAVSTPVKVLPP | 402 | 0.88 |
| 6 | CTYINEVIEQLNGQNE | 129 | 0.88 |
| 7 | ASTDNHTHAPAAVSTP | 396 | 0.86 |
| 7 | VSSSSSSNQNNIGNNT | 284 | 0.86 |
| 7 | YDEFYDQLEVDEEDDV | 225 | 0.86 |
| 8 | PMYELNSSHYMKKFDN | 529 | 0.85 |
| 8 | LQISRSQSPMVSNENK | 449 | 0.85 |
| 8 | PVKKLKPTLAPAPAPP | 332 | 0.85 |
| 9 | ERKREEKEKEKQQQNP | 262 | 0.84 |
| 10 | HDTSMNSTLRSESSSP | 421 | 0.83 |
| 10 | SNAIKAAQTASTSSTS | 356 | 0.83 |
| 10 | DVEVQGSLAQMAAETE | 239 | 0.83 |
| 10 | LELQSQQKRSRFGDDA | 108 | 0.83 |
| 11 | PSVVTVAAIGDQNNNS | 307 | 0.82 |
| 11 | MSARKLQQEFDKLNKK | 1 | 0.82 |
| 12 | NGNIIGDNSNGNEELR | 606 | 0.81 |
| 12 | FSQWFSKDSKNGGKNR | 580 | 0.81 |
| 12 | SYDAEKPRQYNPVNVH | 504 | 0.81 |
| 12 | AETEDERRRDEERKRE | 251 | 0.81 |
| 12 | DAKYQEACTYINEVIE | 122 | 0.81 |
| 13 | LEEVLENLDNDKLDPA | 186 | 0.80 |
| 13 | SEGLQAFDEIKDKINA | 18 | 0.80 |
| 14 | DYEKTWLTRRRENYQF | 638 | 0.79 |
| 14 | NPVNVHPSSIDYPQEP | 514 | 0.79 |
| 14 | DEDYVEYDEFYDQLEV | 219 | 0.79 |
| 15 | KELSRRGWVFNTEFSQ | 567 | 0.78 |
| 16 | NEELREKIDDNGGIPS | 617 | 0.77 |
| 16 | MSVIQREEENGNIIGD | 597 | 0.77 |
| 16 | DTISRIVNVAHSRLND | 468 | 0.77 |
| 16 | SASQREKLENDLKKEL | 36 | 0.77 |
| 16 | APPPITSGTSYSNAIK | 345 | 0.77 |
| 17 | TNKGNRSVSPLASTDN | 385 | 0.75 |

| Start | End | Max_score_pos | Sequence |
|---|---|---|---|
| 302 | 315 | 312 | ATQIKPSVVTVAAI |
| 547 | 554 | 550 | LFFCFYYG |
| 403 | 419 | 414 | HAPAAVSTPVKVLPPGL |
| 434 | 441 | 440 | SSPLVGHA |
| 470 | 479 | 476 | ISRIVNVAHS |
| 482 | 504 | 487 | NDPLPLQSITGLLETSLLNCPDS |
| 391 | 396 | 394 | SVSPLA |
| 513 | 526 | 518 | YNPVNVHPSSIDYP |
| 239 | 249 | 243 | DVEVQGSLAQM |
| 126 | 138 | 132 | QEACTYINEVIEQ |
| 329 | 350 | 343 | TSTPVKKLKPTLAPAPAPPPIT |
| 228 | 234 | 231 | FYDQLEV |
| 209 | 215 | 211 | DLDYYVE |
| 183 | 192 | 187 | ISKLEEVLEN |
| 281 | 287 | 285 | SSSVSSS |
| 165 | 175 | 171 | IQSSIDDVKYK |
| 221 | 226 | 225 | DYVEYD |
| 449 | 454 | 452 | LQISRS |
| 18 | 25 | 24 | SEGLQAFD |
| 149 | 156 | 153 | ELDSLSGQ |
| 597 | 602 | 598 | MSVIQR |
| 49 | 54 | 54 | KELKKL |
| 632 | 639 | 633 | SNYKYFDY |
| 356 | 365 | 361 | SNAIKAAQTA |
| 4 | 10 | 5 | RKLQQEF |

8. IPF11281(GPR1)
MPDLISIATSPTKAFTPAATPTTIPTTIATIAASVSAATATVTTIIKDSTDDSTTTASILSALFNDIILPTI
FKRDYSTRDHKANQLGQFTDHQARVQRIVAISSSCGSIAAVLIAMYFLFAIDPKRIVFRHQLIFFLLFFDLL
KACILLLYPTRILTHSSAYYNHNFCQVVGFFTATAIEGADIAIFAFAFHTYLLIFKPSFNTKVKNSNRVEGG
LYKFRYYVYSLSFFVPLIVASLAFIHSDGYDSLVCWCYLPMRPVWLRLVLSWVPRYCIVVGIFVIYGLIYIR
VISEFKTLGGVFKNAAGNGGAGNLHLASNSNPTFFSSLKYFFVSMKDQWFPNMSDDTIAPITSRHSHSHNAS
GTIASPHRNVIGEIDNDDGDDDSEELAEALEDESVDYQDIELNKQSSRNSYRHHNSDIQQANLENFRRRQRI
IQKQMKSIFIYPFAYCLVWLFPFILQATQFNYEEDHHAVYWLNVLGALSQPLNGFVDTLVFFYRERPWRNTA
MKNFEKENRQRVDNIIVNNLEQRKYSEGAESAQTVATASKRIAKNSLSASSGLVNINAYKPWRQFMNKYRFP
FYQLPTDKNIAKFQDRYIRRKLRDSRKLDKLVQEVTRDRQDLTFPTNIAEKYGDGSGNGSGSGSGGHHGGST
ISNTNDSSPMSMGAGINWTEPTNAHDFSNILNTGGNSNVSSWGTKDVPGFKPNFGKFTFGNRSSNLLSRKSS
TVIGLHGTGRNVRQPSNDSFNDPVRSLGGRRNSSLVIGNNTTLNKPYEIVSSPTSSTFTPIDRVKSNEDIDE
LHTVDNDTDKADDNDDGELDLMEFLKKGPPM*

| Rank | Sequence | Start position | Score |
|---|---|---|---|
| 1 | STVIGLHGTGRNVRQP | 720 | 0.95 |
| 2 | YLLIFKPSFNTKVKNS | 195 | 0.93 |
| 3 | VSSWGTKDVPGFKPNF | 687 | 0.92 |
| 3 | VTTIIKDSTDDSTTTA | 42 | 0.92 |
| 4 | TFTPIDRVKSNEDIDE | 777 | 0.91 |
| 4 | NGSGSGSGGHHGGSTI | 634 | 0.91 |
| 4 | SRHSHSHNASGTIASP | 351 | 0.91 |
| 5 | NEDIDELHTVDNDTDK | 787 | 0.90 |
| 5 | TIFKRDYSTRDHKANQ | 71 | 0.90 |
| 6 | TGRNVRQPSNDSFNDP | 728 | 0.88 |
| 7 | GHHGGSTISNTNDSSP | 642 | 0.87 |
| 7 | HHAVYWLNVLGALSQP | 468 | 0.87 |
| 8 | DRYIRRKLRDSRKLDK | 591 | 0.86 |
| 8 | TIAPITSRHSHSHNAS | 345 | 0.86 |
| 9 | GAGINWTEPTNAHDFS | 661 | 0.85 |
| 9 | FFVSMKDQWFPNMSDD | 329 | 0.85 |
| 9 | GGAGNLHLASNSNPTF | 307 | 0.85 |
| 10 | TVDNDTDKADDNDDGE | 795 | 0.84 |
| 10 | GGRRNSSLVIGNNTTL | 748 | 0.84 |
| 10 | LNTGGNSNVSSWGTKD | 679 | 0.84 |
| 10 | SDIQQANLENFRRRQR | 416 | 0.84 |
| 10 | NDDGDDDSEELAEALE | 376 | 0.84 |
| 11 | TASILSALFNDIILPT | 56 | 0.83 |
| 11 | TIATIAASVSAATATV | 27 | 0.83 |
| 11 | SLAFIHSDGYDSLVCW | 237 | 0.83 |
| 12 | NTNDSSPMSMGAGINW | 651 | 0.82 |
| 12 | HRNVIGEIDNDDGDDD | 367 | 0.82 |
| 12 | DSLVCWCYLPMRPVWL | 247 | 0.82 |
| 12 | PDLISIATSPTKAFTP | 2 | 0.82 |
| 13 | QSSRNSYRHHNSDIQQ | 405 | 0.81 |
| 13 | CYLPMRPVWLRLVLSW | 253 | 0.81 |
| 13 | PTRILTHSSAYYNHNF | 153 | 0.81 |
| 14 | FFYRERPWRNTAMKNF | 493 | 0.80 |
| 14 | SGTIASPHRNVIGEID | 360 | 0.80 |
| 14 | YGLIYIRVISEFKTLG | 282 | 0.80 |
| 14 | NRVEGGLYKFRYYVYS | 211 | 0.80 |
| 14 | IVAISSSCGSIAAVLI | 100 | 0.80 |
| 15 | PYEIVSSPTSSTFTPI | 766 | 0.79 |
| 15 | NIAEKYGDGSGNGSGS | 623 | 0.79 |
| 15 | NFEKENRQRVDNIIVN | 507 | 0.79 |
| 15 | MKSIFIYPFAYCLVWL | 437 | 0.79 |
| 15 | GVFKNAAGNGGAGNLH | 298 | 0.79 |
| 16 | ATSPTKAFTPAATPTT | 8 | 0.78 |
| 16 | LVNINAYKPWRQFMNK | 557 | 0.78 |
| 17 | FKPNFGKFTFGNRSSN | 698 | 0.77 |
| 17 | NLEQRKYSEGAESAQT | 523 | 0.77 |
| 17 | PWRNTAMKNFEKENRQ | 499 | 0.77 |
| 17 | VPLIVASLAFIHSDGY | 231 | 0.77 |
| 17 | HQLIFFLLFFDLLKAC | 132 | 0.77 |
| 18 | SLSASSGLVNINAYKP | 550 | 0.76 |
| 18 | ALEDESVDYQDIELNK | 389 | 0.76 |
| 18 | QVVGFFTATAIEGADI | 170 | 0.76 |
| 19 | TRDRQDLTFPTNIAEK | 612 | 0.75 |
| 19 | LQATQFNYEEDHHAVY | 457 | 0.75 |

| Start | End | Max_score_pos | Sequence |
|---|---|---|---|
| 246 | 293 | 252 | YDSLVCWCYLPMRPVWLRLVLSWVPRYCIVVGIFVIY |
| | | | GLIYIRVISEF |
| 94 | 178 | 150 | QARVQRIVAISSSCGSIAAVLIAMYFLFAIDPKRIVF |
| | | | RHQLIFFLLFFDLLKACILLLYPTRILTHSSAYYNHN |
| | | | FCQVVGFFTAT |
| 439 | 461 | 450 | SIFIYPFAYCLVWLFPFILQATQ |
| 215 | 244 | 232 | GGLYKFRYYVYSLSFFVPLIVASLAFIHSD |
| 467 | 496 | 474 | DHHAVYWLNVLGALSQPLNGFVDTLVFFYR |
| 323 | 333 | 328 | FSSLKYFFVSM |
| 182 | 201 | 195 | GADIAIFAFAFHTYLLIFKP |
| 57 | 72 | 61 | ASILSALFNDIILPTI |
| 25 | 45 | 34 | PTTIATIAASVSAATATVTTI |
| 575 | 581 | 578 | FPFYQLP |
| 604 | 611 | 609 | LDKLVQEV |
| 719 | 726 | 723 | SSTVIGLH |
| 766 | 773 | 770 | PYEIVSSP |
| 548 | 561 | 555 | KNSLSASSGLVNIN |
| 4 | 10 | 5 | LISIATS |
| 753 | 758 | 755 | SSLVIG |
| 395 | 400 | 397 | VDYQDI |
| 536 | 545 | 539 | AQTVATASKR |
| 362 | 373 | 368 | TIASPHRNVIGE |
| 741 | 747 | 746 | NDPVRSL |
| 346 | 354 | 348 | IAPITSRHS |

9. PPR1
MSESPSQPPQKKHKPTTTGPSSSSSSKLTRAISACKRCRTKKIKCDQKFPQCGKCEVNGVECIGVDSVTGRE
IPRSYIVHLEERVKFLEEKLRLQDRSGFVDEGVSSTPPGSSTIKKKSNEISINEPLIKKESPLINTKLDSIA
FSKIMSTAVRVQNRLSDPNIKPGNGNVNLGHNSTTTNENGIDINNDISRAVLPPKSTAMQFLRVFFHQCNSQ
LPLFHREEFLRDYFIPIYGEFDESISLASNNTKINKSFFNSSSSDGDKTPCWFDVYKSKIQQLLTENTQNDI
QTIANNIIPPLKYRKPLYFLNLVFAVATSANHLRYPIHISESFRLAAMRFSQDVDNSIDPLEHLQGILLYAG
YSIMRPTNPGVWYIMGEALRICVDLDLQNELKTKSKQNFNIDNFTRDKRRRIFWCCYSIDRQICFYLDRPFG
IPDESINTPYPSSLDDSKIIPNDNSTDYYYYKNHHHHHHHDDDNDDNEDDINFSYKNVSLLFFKIRKIQSQV
TKILYTNAEIPREYKDLNHWKQSILIQLTQWKQELQSKLISKQLNCDFNEIFFQLNYHHTLLYIHGLSPKNY
KLSLIDYENLTNSSIQVINCYTELLKTKSINYTWAGVHNLFMAGTSYLYALYNSKEIRLINSIDQVQKITND
CLLVLQSLIGRCDAANYCCEIFHNLTMIIINLKYAPKDKKHLTELKPANSIPSSSFSSSSSFSSSSSTSSSK
ISRESLYRINNGNVHSNLFHLVTELDHLNPLTKTKTINNNNSDHFDITNDEKQDHDSTIITDDLSSPPIDSS
LLPLNQQTILQWNDEELQNFLNELNQSNQSNQSSPNNNSSIRDEKKTFELIHDMPNEIIWDEFFANKQ*

| Rank | Sequence | Start position | Score |
|---|---|---|---|
| 1 | ANNIIPPLKYRKPLYF | 292 | 0.92 |
| 2 | DSTIITDDLSSPPIDS | 776 | 0.91 |
| 2 | SDGDKTPCWFDVYKSK | 260 | 0.91 |
| 3 | SPPIDSSLLPLNQQTI | 786 | 0.90 |
| 3 | CCEIFHNLTMIIINLK | 666 | 0.90 |
| 3 | DESISLASNNTKINKS | 238 | 0.90 |
| 4 | DESINTPYPSSLDDSK | 435 | 0.89 |
| 5 | HFDITNDEKQDHDSTI | 764 | 0.88 |
| 5 | TKSINYTWAGVHNLFM | 603 | 0.88 |
| 5 | KSKIQQLLTENTQNDI | 273 | 0.88 |
| 6 | SSIRDEKKTFELIHDM | 831 | 0.86 |
| 6 | QSLIGRCDAANYCCEI | 654 | 0.86 |
| 6 | GILLYAGYSIMRPTNP | 354 | 0.86 |
| 6 | SSTPPGSSTIKKKSNE | 106 | 0.86 |
| 7 | TKTINNNNSDHFDITN | 754 | 0.85 |
| 7 | TDYYYYKNHHHHHHHD | 458 | 0.85 |
| 8 | YPSSLDDSKIIPNDNS | 442 | 0.84 |
| 8 | CYSIDRQICFYLDRPF | 416 | 0.84 |
| 8 | NKSFFNSSSSDGDKTP | 251 | 0.84 |
| 8 | SSSSSSKLTRAISACK | 21 | 0.84 |
| 8 | SPLINTKLDSIAFSKI | 133 | 0.84 |
| 9 | NSSIQVINCYTELLKT | 588 | 0.83 |
| 9 | HHHDDDNDDNEDDINF | 470 | 0.83 |
| 9 | GVWYIMGEALRICVDL | 370 | 0.83 |
| 9 | AISACKRCRTKKIKCD | 31 | 0.83 |
| 9 | DYFIPIYGEFDESISL | 228 | 0.83 |
| 9 | GHNSTTTNENGIDINN | 174 | 0.83 |
| 10 | AEIPREYKDLNHWKQS | 512 | 0.82 |
| 10 | PLKYRKPLYFLNLVFA | 298 | 0.82 |
| 10 | NGIDINNDISRAVLPP | 183 | 0.82 |
| 10 | NRLSDPNIKPGNGNVN | 157 | 0.82 |
| 10 | FSKIMSTAVRVQNRLS | 145 | 0.82 |
| 11 | IRKIQSQVTKILYTNA | 497 | 0.81 |
| 12 | IRLINSIDQVQKITND | 633 | 0.80 |
| 12 | YLDRPFGIPDESINTP | 426 | 0.80 |
| 13 | LRLQDRSGFVDEGVSS | 92 | 0.79 |
| 13 | AGTSYLYALYNSKEIR | 619 | 0.79 |
| 13 | QLNYHHTLLYIHGLSP | 558 | 0.79 |
| 13 | GYSIMRPTNPGVWYIM | 360 | 0.79 |
| 13 | ESPSQPPQKKHKPTTT | 3 | 0.79 |
| 13 | SSTIKKKSNEISINEP | 112 | 0.79 |
| 14 | PLIKKESPLINTKLDS | 127 | 0.78 |
| 15 | HREEFLRDYFIPIYGE | 221 | 0.77 |
| 15 | HKPTTTGPSSSSSSKL | 13 | 0.77 |
| 16 | HDMPNEIIWDEFFANK | 844 | 0.76 |
| 16 | LTMIIINLKYAPKDKK | 673 | 0.76 |
| 17 | REIPRSYIVHLEERVK | 71 | 0.75 |
| 17 | SILIQLTQWKQELQSK | 527 | 0.75 |
| 17 | KFPQCGKCEVNGVECI | 48 | 0.75 |
| 17 | ESFRLAAMRFSQDVDN | 329 | 0.75 |
| 17 | KSTAMQFLRVFFHQCN | 199 | 0.75 |

| Start | End | Max_score_pos | Sequence |
|---|---|---|---|
| 635 | 660 | 652 | LINSIDQVQKITNDCLLVLQSLIGRC |
| 294 | 318 | 311 | NIIPPLKYRKPLYFLNLVFAVATSA |
| 379 | 386 | 382 | LRICVDLD |
| 662 | 683 | 668 | AANYCCEIFHNLTMIIINLKYA |
| 412 | 430 | 415 | IFWCCYSIDRQICFYLDRP |
| 40 | 68 | 65 | TKKIKCDQKFPQCGKCEVNGVECIGVDSV |
| 621 | 630 | 625 | TSYLYALYNS |
| 589 | 603 | 594 | SSIQVINCYTELLKT |
| 555 | 583 | 568 | IFFQLNYHHTLLYIHGLSPKNYKLSLIDY |
| 487 | 511 | 493 | YKNVSLLFFKIRKIQSQVTKILYTN |
| 203 | 223 | 210 | MQFLRVFFHQCNSQLPLFHRE |
| 348 | 361 | 355 | PLEHLQGILLYAGY |
| 75 | 95 | 78 | RSYIVHLEERVKFLEEKLRLQ |
| 266 | 280 | 269 | PCWFDVYKSKIQQLL |
| 735 | 751 | 739 | HSNLFHLVTELDHLNPL |
| 28 | 38 | 35 | LTRAISACKRC |
| 526 | 534 | 529 | QSILIQLTQ |
| 192 | 199 | 194 | SRAVLPPK |
| 320 | 336 | 324 | HLRYPIHISESFRLAAM |
| 459 | 472 | 469 | DYYYYKNHHHHHHH |
| 785 | 802 | 793 | SSPPIDSSLLPLNQQTIL |
| 227 | 235 | 232 | RDYFIPIYG |
| 150 | 158 | 153 | STAVRVQNR |
| 539 | 551 | 542 | LQSKLISKQLNCD |
| 610 | 616 | 614 | WAGVHNL |
| 141 | 147 | 143 | DSIAFSK |
| 696 | 713 | 701 | ANSIPSSSFSSSSSFSSS |
| 129 | 138 | 137 | IKKESPLINT |
| 723 | 728 | 724 | RESLYR |
| 840 | 845 | 841 | FELIHD |
| 5 | 12 | 7 | PSQPPQKK |

10. IPF3598(SIP3)
MVKSPKSDKSKPLPSQPHQETLLHHFKLISVNFKEAALDSPSFRASMNHLDLQINTIEQWLTALASSFKKIP
KYLKEVQSYSNSFLEHLVPTFIQDGIIDQEYTVTGLNTTLDGLKTVWGLSIQALSVDAKNLKSIELFKRHHV
IKYKETRKRYEDYQAKYDKYLSIYLSSSKSKDPLMVIEDAKQLYQVRKEYIHASLDLVIEIQNLSKNLNKLL
VGVNTDLWRNKWNIFGSRGVGDAIKEEWDKIQRIQSWNDSYTLAIEKLNSDMLAARNQVEEGCHIQFQPSTN
VNDYKSTIINNRTLRDIDEPGVEKHGYLFMKTWTEKSSKPIWVHRWAFIKNGVFGLLVVSPSQTFVQETDKI
GILLCNVRYAPNEDRRFCFEIRTNDFTAVFQAESLVELKSWLKVFENEKFRISGPEAISNGLFNIASGRFPP
IISEFSSTVDTVIDQQLTNAKVTLAGGQIVAASSLSNHLERFEDFFKKYMYFEIPKICPPFMTDTTKSSIMA
YCLTSPTQIPNALTANIWGSVNWGLYYLHDTARDSSTYLTGKDAEMIKFQEEHFENDKFYPDFYPKEYVNLD
IQMRALFETAVEPGEYCVLSYSCIWSPNSKQELSGRCFVTNYHMYFYMQALGFVALFKGFLGHLVSVEFVSQ
KNYDLMKVYNIDGVIKMKVFLDDAICIKKKLVYLINNIVSDKPKSLEGVLADFSDIEKEIAVEKSDQKNLRE
ISQLSKGLSSKSLASEKLLLSGETSSILPGKSGRMIKHRVNFTPDYNLISDRTYPAPPKAIFHALLGDNSVV
FRSQLSFASTKYFLQKPWATSSKGTLYRDFNVPAMYDGKDCFVQVRQEIDNMEDNTYYTFTHEMSKFELLLG
SPYKTVFKIVIVEHISKRSKVFVYSKTYFDRLSVWNPLVIRLNNQVDVNKVRKLEKSISEAVKEIGTHGMIV
RAIYLYGKLSHTSKPEAVTSTSVIKFGIVSLFKLGLGKAFSKAYSFAVKSFIKPFQLVVLLLKSLRMNVFLV
FIIVLLSFLNLFLAGKTATSYWNTRSASKLAQEYVTKEPRMLQRSVYLKDLESILNENISIAESRPFSLFKQ
NSFIFNLDADSDWSNYFGSNARDVARSLKSSFQDIGIKRHELLVKLKILKSMEEEIIQAEWQNWLMSEAQKC
DYVMDNVVGQIDEVDNYQEGVDNIIEYCHECKKILANLV*

| Rank | Sequence | Start position | Score |
|---|---|---|---|
| 1 | PGVEKHGYLFMKTWTE | 308 | 0.93 |
| 2 | AEMIKFQEEHFENDKF | 548 | 0.92 |
| 2 | KSSIMAYCLTSPTQIP | 499 | 0.92 |
| 3 | PAMYDGKDCFVQVRQE | 825 | 0.91 |
| 3 | KEAALDSPSFRASMNH | 34 | 0.91 |
| 4 | SEAQKCDYVMDNVVGQ | 1147 | 0.90 |
| 5 | QDGIIDQEYTVTGLNT | 95 | 0.89 |
| 5 | PKAIFHALLGDNSVVF | 778 | 0.89 |
| 5 | AVEPGEYCVLSYSCIW | 586 | 0.89 |
| 6 | ATSSKGTLYRDFNVPA | 811 | 0.88 |
| 6 | ALLGDNSVVFRSQLSF | 784 | 0.88 |
| 6 | KEVQSYSNSFLEHLVP | 76 | 0.88 |
| 6 | GRMIKHRVNFTPDYNL | 753 | 0.88 |
| 6 | HDTARDSSTYLTGKDA | 533 | 0.88 |
| 6 | KSTIINNRTLRDIDEP | 293 | 0.88 |
| 7 | DGVIKMKVFLDDAICI | 660 | 0.87 |
| 7 | KFRISGPEAISNGLFN | 409 | 0.87 |
| 8 | CNVRYAPNEDRRFCFE | 365 | 0.86 |
| 8 | KETRKRYEDYQAKYDK | 148 | 0.86 |
| 9 | GCHIQFQPSTNVNDYK | 278 | 0.85 |
| 10 | YGKLSHTSKPEAVTST | 942 | 0.84 |
| 10 | NMEDNTYYTFTHEMSK | 843 | 0.84 |
| 10 | CVLSYSCIWSPNSKQE | 593 | 0.84 |
| 10 | YFEIPKICPPFMTDTT | 483 | 0.84 |
| 10 | KPIWVHRWAFIKNGVF | 327 | 0.84 |
| 10 | GLSIQALSVDAKNLKS | 120 | 0.84 |
| 10 | QEGVDNIIEYCHECKK | 1170 | 0.84 |
| 10 | VGQIDEVDNYQEGVDN | 1160 | 0.84 |
| 10 | QAEWQNWLMSEAQKCD | 1138 | 0.84 |
| 10 | DSDWSNYFGSNARDVA | 1090 | 0.84 |
| 11 | DRTYPAPPKAIFHALL | 771 | 0.83 |
| 11 | IQRIQSWNDSYTLAIE | 247 | 0.83 |
| 11 | IKEEWDKIQRIQSWND | 240 | 0.83 |
| 11 | ISIAESRPFSLFKQNS | 1067 | 0.83 |
| 12 | QVRQEIDNMEDNTYYT | 836 | 0.82 |
| 12 | RALFETAVEPGEYCVL | 580 | 0.82 |
| 12 | LLVGVNTDLWRNKWNI | 215 | 0.82 |
| 13 | TSVIKFGIVSLFKLGL | 957 | 0.81 |
| 13 | FVSQKNYDLMKVYNID | 645 | 0.81 |
| 13 | FMTDTTKSSIMAYCLT | 493 | 0.81 |
| 13 | SQPHQETLLHHFKLIS | 15 | 0.81 |
| 13 | HHVIKYKETRKRYEDY | 142 | 0.81 |
| 14 | FVYSKTYFDRLSVWNP | 886 | 0.80 |
| 14 | GVLADFSDIEKEIAVE | 696 | 0.80 |
| 14 | SVNWGLYYLHDTARDS | 524 | 0.80 |
| 14 | LRDIDEPGVEKHGYLF | 302 | 0.80 |
| 14 | DLVIEIQNLSKNLNKL | 200 | 0.80 |
| 14 | SKLAQEYVTKEPRMLQ | 1036 | 0.80 |
| 15 | QEEHFENDKFYPDFYP | 554 | 0.79 |
| 15 | TFVQETDKIGILLCNV | 352 | 0.79 |
| 15 | TNVNDYKSTIINNRTL | 287 | 0.79 |
| 16 | KSDKSKPLPSQPHQET | 6 | 0.78 |
| 16 | KFYPDFYPKEYVNLDI | 562 | 0.78 |
| 16 | DKYLSIYLSSSKSKDP | 162 | 0.78 |
| 17 | VDTVIDQQLTNAKVTL | 441 | 0.76 |
| 18 | HGMIVRAIYLYGKLSH | 932 | 0.75 |
| 18 | KIVIVEHISKRSKVFV | 872 | 0.75 |
| 18 | GLFNIASGRFPPIISE | 421 | 0.75 |
| 18 | RRFCFEIRTNDFTAVF | 375 | 0.75 |
| 18 | SSFQDIGIKRHELLVK | 1110 | 0.75 |

| Start | End | Max_score_pos | Sequence |
|---|---|---|---|
| 977 | 1000 | 995 | SKAYSFAVKSFIKPFQLVVLLLKS |
| 1002 | 1024 | 1010 | RMNVFLVFIIVLLSFLNLFLAGK |
| 589 | 602 | 596 | PGEYCVLSYSCIWS |
| 338 | 357 | 344 | KNGVFGLLVVSPSQTFVQET |
| 859 | 879 | 873 | FELLLGSPYKTVFKIVIVEHI |
| 610 | 650 | 643 | SGRCFVTNYHMYFYMQALGFVALFKGFLGHLVSVEFVSQKN |
| 831 | 839 | 836 | KDCFVQVRQ |
| 1119 | 1129 | 1125 | RHELLVKLKIL |
| 360 | 370 | 366 | IGILLCNVRYA |
| 951 | 975 | 967 | PEAVTSTSVIKFGIVSLFKLGLGKA |
| 664 | 686 | 680 | KMKVFLDDAICIKKKLVYLINNI |
| 933 | 948 | 939 | GMIVRAIYLYGKLSHT |
| 774 | 809 | 794 | YPAPPKAIFHALLGDNSVVFRSQLSFASTKYFLQKP |
| 157 | 172 | 167 | YQAKYDKYLSIYLSSS |
| 60 | 93 | 88 | WLTALASSFKKIPKYLKEVQSYSNSFLEHLVPTF |
| 19 | 41 | 25 | QETLLHHFKLISVNFKEAALDSP |
| 177 | 206 | 200 | PLMVIEDAKQLYQVRKEYIHASLDLVIEIQ |
| 881 | 891 | 886 | KRSKVFVYSKT |
| 1051 | 1062 | 1053 | QRSVYLKDLESI |
| 1174 | 1188 | 1180 | DNIIEYCHECKKILA |
| 451 | 470 | 464 | NAKVTLAGGQIVAASSLSNH |
| 893 | 907 | 901 | FDRLSVWNPLVIRLN |
| 115 | 131 | 125 | LKTVWGLSIQALSVDAK |
| 502 | 511 | 504 | IMAYCLTSPT |
| 276 | 284 | 282 | EEGCHIQFQ |
| 213 | 220 | 216 | NKLLVGVN |
| 483 | 493 | 489 | YFEIPKICPPF |
| 1149 | 1164 | 1152 | AQKCDYVMDNVVGQID |
| 135 | 147 | 145 | SIELFKRHHVIKY |
| 526 | 534 | 532 | NWGLYYLHD |
| 387 | 405 | 397 | TAVFQAESLVELKSWLKVF |
| 694 | 701 | 696 | LEGVLADF |
| 1035 | 1045 | 1040 | ASKLAQEYVTK |
| 564 | 576 | 570 | YPDFYPKEYVNLD |
| 428 | 447 | 442 | GRFPPIISEFSSTVDTVIDQ |
| 721 | 742 | 737 | ISQLSKGLSSKSLASEKLLLSG |
| 653 | 662 | 659 | LMKVYNIDGV |
| 10 | 17 | 15 | SKPLPSQP |
| 328 | 335 | 334 | PIWVHRWA |
| 310 | 318 | 313 | VEKHGYLFM |
| 1104 | 1115 | 1107 | VARSLKSSFQDI |
| 1074 | 1082 | 1077 | PFSLFKQNS |
| 49 | 55 | 51 | HLDLQIN |
| 257 | 263 | 258 | YTLAIEK |
| 923 | 929 | 923 | SEAVKEI |
| 821 | 827 | 821 | DFNVPAM |
| 707 | 712 | 707 | EIAVEK |
| 376 | 381 | 380 | RFCFEI |
| 100 | 108 | 102 | DQEYTVTGL |
| 745 | 750 | 749 | SSILPG |

11. IPF9385
MSPDSISSSVNQDLSTPTPPTSLSSTSTNSNTNASSGQKRIRATGEALEFLISEFETNPNPSPERRKFISDK
AQMNEKAVRIWFQNRRAKQRKFERQMLRKETDSPGNYAGIYNTYTPNPPTVTTDMTNFNVNGSAGGATADFN
DKLKNISSIPVEVNEKYCFIDCRSLSVGSWQRIKTGFHQSNLLTNNLINLAPVTLNQVMSNADLLIILSKKN
LELNYFFSAISNNSRILFRIFYPLNAVVKCSLFDNNYYQNNNTNGTNSSDDNNISEIRLNLCQKPKFSVYFF
NGSNTNNQWSICDDFSEGQQVSCAFAANDNSLPSNGKNQSFSNTIPHVLVGSTLSLQYLSQFILQHQQRQRQ
QQRQAQPQPQPQPHNQFDFNSQPFETIPTSAINNNFNTTKTVNSSSMQGFVPGDFQDLPAIYESISNSNHTT
TTDLKDTNATTTTTNKHTPSSTTFTPQNLNGSNQSQTNVTYTNNYNESPFSIASTTNNNNTNSYRSNSQSHN
PIFSDQLFYESSESASTNSPQFAMKKMNSETKLYINGNVSSNSSTNGPPLDDDNNLFDGVTRFTTTETSPED
DIIGMFTSQAHEPSAFELANGVTSSGSSYTHINNGSLTKSDKTFTGLSETSNNNNNTNGINFVDDFHVGNEF
GDIDFEHHYSQDHHQQQQKNDNNNGNTNLDSFIDFEN*

| Rank | Sequence | Start position | Score |
|---|---|---|---|
| 1 | SPFSIASTTNNNNTNS | 480 | 0.94 |
| 2 | HHQQQQKNDNNNGNTN | 661 | 0.93 |
| 2 | SSTNGPPLDDDNNLFD | 547 | 0.93 |
| 2 | YAGIYNTYTPNPPTVT | 109 | 0.93 |
| 3 | QPQPQPHNQFDFNSQP | 368 | 0.92 |
| 4 | CQKPKFSVYFFNGSNT | 278 | 0.91 |
| 5 | SEGQQVSCAFAANDNS | 304 | 0.89 |
| 6 | TSSGSSYTHINNGSLT | 599 | 0.88 |
| 7 | RKFISDKAQMNEKAVR | 66 | 0.87 |
| 7 | MFTSQAHEPSAFELAN | 581 | 0.87 |
| 7 | SNHTTTTDLKDTNATT | 428 | 0.87 |
| 7 | GSAGGATADFNDKLKN | 134 | 0.87 |
| 8 | QMLRKETDSPGNYAGI | 97 | 0.86 |
| 8 | TFTGLSETSNNNNNTN | 619 | 0.86 |
| 8 | KCSLFDNNYYQNNNTN | 245 | 0.86 |
| 9 | TGEALEFLISEFETNP | 44 | 0.85 |
| 9 | KNISSIPVEVNEKYCF | 148 | 0.85 |
| 10 | SASTNSPQFAMKKMNS | 518 | 0.84 |
| 10 | TFTPQNLNGSNQSQTN | 455 | 0.84 |
| 10 | AFAANDNSLPSNGKNQ | 312 | 0.84 |
| 10 | VGSWQRIKTGFHQSNL | 171 | 0.84 |
| 11 | GVTRFTTTETSPEDDI | 563 | 0.83 |
| 11 | SEFETNPNPSPERRKF | 53 | 0.83 |
| 11 | LFRIFYPLNAVVKCSL | 233 | 0.83 |
| 11 | PNPPTVTTDMTNFNVN | 118 | 0.83 |
| 12 | LKDTNATTTTTNKHTP | 436 | 0.82 |
| 12 | YESISNSNHTTTTDLK | 422 | 0.82 |
| 12 | QKRIRATGEALEFLIS | 38 | 0.82 |
| 13 | HNPIFSDQLFYESSES | 503 | 0.81 |
| 13 | PFETIPTSAINNNFNT | 383 | 0.81 |
| 13 | TPPTSLSSTSTNSNTN | 18 | 0.81 |
| 14 | GNVSSNSSTNGPPLDD | 541 | 0.80 |
| 14 | TKLYINGNVSSNSSTN | 535 | 0.80 |
| 14 | SSSMQGFVPGDFQDLP | 404 | 0.80 |
| 15 | QWSICDDFSEGQQVSC | 296 | 0.79 |
| 15 | PVEVNEKYCFIDCRSL | 154 | 0.79 |
| 16 | TNSSDDNNISEIRLNL | 262 | 0.77 |
| 17 | AVRIWFQNRRAKQRKF | 79 | 0.76 |
| 17 | AFELANGVTSSGSSYT | 591 | 0.76 |
| 17 | TYTNNYNESPFSIAST | 472 | 0.76 |
| 17 | SNQSQTNVTYTNNYNE | 464 | 0.76 |
| 18 | YCFIDCRSLSVGSWQR | 161 | 0.75 |

| Start | End | Max_score_pos | Sequence |
|---|---|---|---|
| 232 | 249 | 246 | ILFRIFYPLNAVVKCSLF |
| 330 | 355 | 335 | SNTIPHVLVGSTLSLQYLSQFILQHQ |
| 306 | 314 | 312 | GQQVSCAFA |
| 149 | 174 | 163 | NISSIPVEVNEKYCFIDCRSLSVGSW |
| 204 | 216 | 211 | SNADLLIILSKKN |
| 192 | 202 | 194 | INLAPVTLNQV |
| 638 | 646 | 641 | FVDDFHVGN |
| 272 | 288 | 285 | EIRLNLCQKPKFSVYFF |
| 48 | 54 | 50 | LEFLISE |
| 218 | 225 | 224 | ELNYFFSA |
| 408 | 425 | 419 | QGFVPGDFQDLPAIYESI |
| 503 | 516 | 510 | HNPIFSDQLFYESS |
| 4 | 13 | 7 | DSISSSVNQD |
| 297 | 303 | 301 | WSICDDF |
| 480 | 485 | 483 | SPFSIA |
| 584 | 598 | 592 | SQAHEPSAFELANGV |
| 653 | 664 | 658 | FEHHYSQDHHQQ |
| 363 | 374 | 371 | RQAQPQPQPQPH |
| 20 | 25 | 22 | PTSLSS |
| 384 | 390 | 389 | FETIPTS |
| 600 | 606 | 605 | SSGSSYT |
| 118 | 123 | 120 | PNPPTV |
| 109 | 115 | 115 | YAGIYNT |

12. IPF9413
MDKEYQEYQISDMPSFIGWACHGILKQNRTTSEFLLNSTQSLLFSTRLSIPTILAGLEYINQRFSNKEIYHL
QDQEIFQILVVSFLLSNKMNDDATFTNKSWEQASGIPLSVLNREEREWLNEVKFNLAVTKYEANISVLDQCW
KTWVNKYGSCHSEPPSSPTYYTPEVDAYYYSSHHHSYSSPISYSQHSYSNPYPQHQQCNYQYQYQPQYQPTY
QSHTQYLVPQPPPPPPPSYYDPAIVSVNYGGFIYT*

| Rank | Sequence | Start position | Score |
|---|---|---|---|
| 1 | LVPQPPPPPPPSYYDP | 223 | 0.93 |
| 1 | QPTYQSHTQYLVPQPP | 213 | 0.93 |
| 2 | GSCHSEPPSSPTYYTP | 152 | 0.91 |
| 3 | SQHSYSNPYPQHQQCN | 188 | 0.90 |
| 3 | HHSYSSPISYSQHSYS | 178 | 0.90 |
| 4 | SSPTYYTPEVDAYYYS | 160 | 0.86 |
| 5 | IPTILAGLEYINQRFS | 50 | 0.84 |
| 5 | TPEVDAYYYSSHHHSY | 166 | 0.84 |
| 5 | KSWEQASGIPLSVLNR | 100 | 0.84 |
| 6 | QILVVSFLLSNKMNDD | 79 | 0.83 |
| 6 | GILKQNRTTSEFLLNS | 23 | 0.83 |
| 6 | PSFIGWACHGILKQNR | 14 | 0.83 |
| 6 | EREWLNEVKFNLAVTK | 117 | 0.83 |
| 7 | EYQISDMPSFIGWACH | 7 | 0.82 |
| 8 | HQQCNYQYQYQPQYQP | 199 | 0.81 |
| 9 | PPPPSYYDPAIVSVNY | 230 | 0.78 |
| 10 | SVLNREEREWLNEVKF | 111 | 0.77 |
| 11 | WKTWVNKYGSCHSEPP | 144 | 0.76 |
| 12 | FNLAVTKYEANISVLD | 126 | 0.75 |

| Start | End | Max_score_pos | Sequence |
|---|---|---|---|
| 69 | 88 | 84 | IYHLQDQEIFQILVVSFLLS |
| 136 | 143 | 140 | NISVLDQC |
| 161 | 248 | 225 | SPTYYTPEVDAYYYSSHHHSYSSPISYSQHSYSNPYP |
| | | | QHQQCNYQYQYQPQYQPTYQSHTQYLVPQPPPPPPPS |
| | | | YYDPAIVSVNYGGF |
| 106 | 113 | 110 | SGIPLSVL |
| 123 | 133 | 127 | EVKFNLAVTKY |
| 33 | 60 | 51 | EFLLNSTQSLLFSTRLSIPTILAGLEYI |
| 17 | 25 | 23 | IGWACHGIL |
| 150 | 159 | 151 | KYGSCHSEPP |

13. SPT6
MMKKKISAKKKNQQQQQLHQPTMSEVTEEERTRYEEEEDVRDSPSDSSEESEDDEEEIQKVREGFIVDDEED
EVQTKKRKSHKRKRDKERPHYDDALDDDDLELLLENSGLKRGSSSSGKFKRLKRKQIEDDEDEIESQDHQGE
QQLRDIFSDDEEVEEEAAPRIMDEFDGFIEEDDFSDEDEQTRLERREQRKKKKQGPRIDTSNLSNVDRQSLS
ELFEVFGDGNEYDWALEAQELEDAGAIDKEEPASLDEVFEHSELKERMLTEEDNLIRIIDVPERYQMYRSAL
TYIDLDDEELELEKTWVANTLLKEKKAFLRDDWVEPFKQCVGQVVQFVSKENLEVPFIWNHRRDYLEYVDPD
APIPGSVRELMISEDDVWRIVKLDIEYHSLYEKRLNTEKIIDSLEIDDELVKDIKTLDSMVAIQDMHDYIQF
TYSKEIRQREETQNRKHSKFALYERIRENVLYDAVKAYGITAKEFGENVQDQSSKGFEVPYRIHATDDPWES
PDDMIERLIQDDEVIFRDEKTARDAVRRTFADEIFYNPKIRHEVRSTYKLYASISVAVTEKGRASIDAHSPF
ADIKYAINRSPADLIAKPDVLLRMLEAERLGLVVIKVETKDFANWFDCLFNCLKSDGFSDISEKWNQERQAV
LRTAISRLCAVVALNTKEDLRRECERLIASKVRHGLLAKIEQAPFTPYGFDIGSKANVLALTFGKGDYDSAV
VGVYIKHDGKVSRFFKSTENPSRNRETEDAFKGQLKQFFDEDETPDVVVVSGYNANTKRLHDVVYNFVSEYG
ISVKSEFDDGSSQLVKVIWGQDETARLYQNSERAKKEFPDKPTLVKYAISLGRYLQDPLLEYITLGDDILSL
TFHEHQKLISNDLVKEVVESAFVDLVNAVGVDINESVRDSRLAQTLKYVGGLGPRKASGMLRNIAQKLGSVL
TTRSQLIEYELTTRTIFINCSAALKISLNKSINVKDFEIEILDTTRIHPEDYQLAMKMAADALDMDEESELH
EKGGVIKELLENDPSKLNLLNLNDFANQIYKLTHKLKFRSLQAIRLELIQGFAEIRSPFRILTNEDAFFILT
GEKPQMLKNTVIPATITKVTKNHHDPYARIRGLKVVTPSLIQGTIDENAIPRDAEYVQGQVVQAVVLELYTD
TFAAVLSLRREDISRAMKGGVVREYGKWDYKAEDEDIKREKAKENAKLAKTRNIQHPFYRNFNYKQAEEYLA
PQNVGDYVIRPSSKGASYLTITWKVGNNLFQHLLVEERSRGRFKEYIVDGKTYEDLDQLAFQHIQVIAKNVT
DMVRHPKFREGTLSVVHEWLESYTRANPKSSAYVFCYDHKSPGNFLLLFKVNVSAKVVTWHVKTEVGGYELR
SSVYPNMLSLCNGFKQAVKMSSQQTKSYNTGYY*

| Rank | Sequence | Start position | Score |
|---|---|---|---|
| 1 | DDPWESPDDMIERLIQ | 499 | 0.95 |
| 1 | KLDIEYHSLYEKRLNT | 382 | 0.95 |
| 1 | HHDPYARIRGLKVVTP | 1103 | 0.95 |
| 2 | PFIWNHRRDYLEYVDP | 344 | 0.94 |
| 2 | DGFIEEDDFSDEDEQT | 170 | 0.94 |
| 3 | EQAPFTPYGFDIGSKA | 689 | 0.91 |
| 3 | PSLIQGTIDENAIPRD | 1118 | 0.91 |
| 4 | EERTRYEEEEDVRDSP | 29 | 0.90 |
| 4 | LSVVHEWLESYTRANP | 1309 | 0.90 |
| 5 | AFKGQLKQFFDEDETP | 750 | 0.89 |
| 5 | TEKIIDSLEIDDELVK | 397 | 0.89 |
| 5 | AFLRDDWVEPFKQCVG | 315 | 0.89 |
| 5 | YQMYRSALTYIDLDDE | 281 | 0.89 |
| 5 | DWALEAQELEDAGAID | 229 | 0.89 |
| 5 | GGVVREYGKWDYKAED | 1171 | 0.89 |
| 5 | TITKVTKNHHDPYARI | 1095 | 0.89 |
| 5 | AFFILTGEKPQMLKNT | 1075 | 0.89 |
| 6 | YERIRENVLYDAVKAY | 455 | 0.88 |
| 6 | KGASYLTITWKVGNNL | 1238 | 0.88 |
| 6 | GGVIKELLENDPSKLN | 1011 | 0.88 |
| 7 | RIHPEDYQLAMKMAAD | 982 | 0.87 |
| 7 | LVKVIWGQDETARLYQ | 806 | 0.87 |
| 7 | ERLIASKVRHGLLAKI | 673 | 0.87 |
| 7 | GPRIDTSNLSNVDRQS | 199 | 0.87 |
| 7 | DEDEQTRLERREQRKK | 180 | 0.87 |
| 7 | TRNIQHPFYRNFNYKQ | 1203 | 0.87 |
| 8 | SQLIEYELTTRTIFIN | 940 | 0.86 |
| 8 | DETPDVVVVSGYNANT | 762 | 0.86 |
| 8 | AHSPFADIKYAINRSP | 572 | 0.86 |
| 8 | KAYGITAKEFGENVQD | 468 | 0.86 |
| 8 | ELMISEDDVWRIVKLD | 369 | 0.86 |
| 8 | HLLVEERSRGRFKEYI | 1256 | 0.86 |
| 9 | TLVKYAISLGRYLQDP | 835 | 0.85 |
| 9 | KKRKSHKRKRDKERPH | 77 | 0.85 |
| 9 | KGDYDSAVVGVYIKHD | 713 | 0.85 |
| 9 | SKANVLALTFGKGDYD | 702 | 0.85 |
| 9 | LRTAISRLCAVVALNT | 649 | 0.85 |
| 9 | ADEIFYNPKIRHEVRS | 535 | 0.85 |
| 9 | DELVKDIKTLDSMVAI | 408 | 0.85 |
| 9 | PRIMDEFDGFIEEDDF | 163 | 0.85 |
| 10 | RPHYDDALDDDDLELL | 90 | 0.84 |
| 10 | VETKDFANWFDCLFNC | 613 | 0.84 |
| 10 | DEEEIQKVREGFIVDD | 54 | 0.84 |
| 10 | SEESEDDEEEIQKVRE | 48 | 0.84 |
| 10 | VEEEAAPRIMDEFDGF | 157 | 0.84 |
| 11 | EYGISVKSEFDDGSSQ | 790 | 0.83 |
| 11 | EVPYRIHATDDPWESP | 490 | 0.83 |
| 11 | RRDYLEYVDPDAPIPG | 350 | 0.83 |
| 12 | ARLYQNSERAKKEFPD | 817 | 0.82 |
| 12 | KVSRFFKSTENPSRNR | 730 | 0.82 |
| 12 | EGFIVDDEEDEVQTKK | 63 | 0.82 |
| 12 | DDEVIFRDEKTARDAV | 515 | 0.82 |
| 12 | TWHVKTEVGGYELRSS | 1355 | 0.82 |
| 12 | KAEDEDIKREKAKENA | 1183 | 0.82 |
| 13 | MKMAADALDMDEESEL | 992 | 0.81 |
| 13 | YASISVAVTEKGRASI | 555 | 0.81 |
| 13 | NPKIRHEVRSTYKLYA | 541 | 0.81 |
| 13 | QSSKGFEVPYRIHATD | 484 | 0.81 |
| 13 | FEVFGDGNEYDWALEA | 219 | 0.81 |
| 13 | YVFCYDHKSPGNFLLL | 1329 | 0.81 |
| 13 | KEYIVDGKTYEDLDQL | 1268 | 0.81 |
| 14 | GVDINESVRDSRLAQT | 894 | 0.80 |
| 14 | KQFFDEDETPDVVVVS | 756 | 0.80 |
| 14 | QGFAEIRSPFRILTNE | 1058 | 0.80 |
| 15 | RREQRKKKKQGPRIDT | 189 | 0.79 |
| 15 | ESYTRANPKSSAYVFC | 1317 | 0.79 |
| 15 | HIQVIAKNVTDMVRHP | 1287 | 0.79 |
| 15 | YGKWDYKAEDEDIKRE | 1177 | 0.79 |
| 15 | AIPRDAEYVQGQVVQA | 1129 | 0.79 |
| 16 | EIEILDTTRIHPEDYQ | 974 | 0.78 |
| 16 | VVSGYNANTKRLHDVV | 769 | 0.78 |
| 16 | IRIIDVPERYQMYRSA | 272 | 0.78 |
| 16 | RDIFSDDEEVEEEAAP | 148 | 0.78 |
| 17 | RKRDKERPHYDDALDD | 84 | 0.77 |
| 17 | EEDVRDSPSDSSEESE | 37 | 0.77 |
| 17 | DYVIRPSSKGASYLTI | 1230 | 0.77 |
| 18 | CLKSDGFSDISEKWNQ | 628 | 0.76 |
| 18 | KRKQIEDDEDEIESQD | 125 | 0.76 |
| 19 | DISEKWNQERQAVLRT | 636 | 0.75 |
| 19 | RQREETQNRKHSKFAL | 439 | 0.75 |
| 19 | QFTYSKEIRQREETQN | 431 | 0.75 |
| 19 | KQCVGQVVQFVSKENL | 326 | 0.75 |
| 19 | IESQDHQGEQQLRDIF | 136 | 0.75 |
| 19 | LRREDISRAMKGGVVR | 1160 | 0.75 |

| Start | End | Max_score_pos | Sequence |
|---|---|---|---|
| 1133 | 1160 | 1144 | DAEYVQGQVVQAVVLELYTDTFAAVLSL |
| 646 | 663 | 659 | QAVLRTAISRLCAVVALN |
| 765 | 773 | 770 | PDVVVVSGY |
| 322 | 346 | 330 | VEPFKQCVGQVVQFVSKENLEVPFI |
| 717 | 734 | 722 | DSAVVGVYIKHDGKVSRF |
| 604 | 614 | 610 | ERLGLVVIKVE |
| 1254 | 1261 | 1256 | FQHLLVEE |
| 1326 | 1336 | 1330 | SSAYVFCYDHK |
| 1340 | 1357 | 1344 | NFLLLFKVNVSAKVVTWH |
| 780 | 797 | 786 | LHDVVYNFVSEYGISVKS |
| 833 | 896 | 884 | KPTLVKYAISLGRYLQDPLLEYITLGDDILSLTFHEH |
| | | | QKLISNDLVKEVVESAFVDLVNAVGVD |
| 1104 | 1123 | 1118 | HDPYARIRGLKVVTPSLIQG |
| 804 | 812 | 807 | SQLVKVIWG |
| 621 | 632 | 627 | WFDCLFNCLKSD |
| 545 | 563 | 559 | RHEVRSTYKLYASISVAVT |
| 460 | 472 | 465 | ENVLYDAVKAYGI |
| 1308 | 1316 | 1310 | TLSVVHEWL |
| 1280 | 1294 | 1288 | LDQLAFQHIQVIAKN |
| 953 | 965 | 959 | FINCSAALKISLN |
| 378 | 393 | 380 | WRIVKLDIEYHSLYEK |
| 272 | 280 | 274 | IRIIDVPER |
| 703 | 710 | 709 | KANVLALT |
| 588 | 600 | 595 | ADLIAKPDVLLRM |
| 488 | 496 | 494 | GFEVPYRIH |
| 673 | 699 | 683 | ERLIASKVRHGLLAKIEQAPFTPYGFD |
| 1367 | 1389 | 1370 | LRSSVYPNMLSLCNGFKQAVKMS |
| 353 | 370 | 356 | YLEYVDPDAPIPGSVREL |
| 907 | 916 | 913 | AQTLKYVGGL |
| 928 | 946 | 933 | IAQKLGSVLTTRSQLIEYE |
| 1219 | 1235 | 1225 | AEEYLAPQNVGDYVIRP |
| 215 | 222 | 218 | LSELFEVF |
| 284 | 293 | 290 | YRSALTYIDL |
| 1088 | 1098 | 1093 | KNTVIPATITK |
| 1035 | 1060 | 1051 | NQIYKLTHKLKFRSLQAIRLELIQGF |
| 450 | 457 | 452 | SKFALYER |
| 58 | 68 | 64 | IQKVREGFIVD |
| 102 | 108 | 103 | LELLLEN |
| 418 | 424 | 420 | DSMVAIQ |
| 251 | 258 | 257 | LDEVFEHS |
| 15 | 21 | 18 | QQQLHQP |
| 1239 | 1247 | 1243 | GASYLTITW |
| 1171 | 1176 | 1176 | GGVVRE |
| 1269 | 1274 | 1269 | EYIVDG |
| 1076 | 1081 | 1078 | FFILTG |
| 407 | 414 | 413 | DDELVKDI |
| 570 | 582 | 580 | IDAHSPFADIKYA |
| 515 | 521 | 515 | DDEVIFR |
| 1296 | 1302 | 1298 | TDMVRHP |
| 985 | 991 | 987 | PEDYQLA |
| 752 | 758 | 756 | KGQLKQF |

14. SET1
MSYNNRSGGGASGGYSRRGYHGSHRGGYRTGRSKYPEDRYLVGGMLSLNKGSHYESSDNRYIPNEIGSKSPE
NRSHRSSTKDGRTPSGLSTPLSSSDKVSTPISIESINGSDRNTGVNNKDSEFPKLSHHSDFTSTIPFSRSIN
PQKNFMVINDSHTPKTDKGIQSKKIRYNGEGVNHVSDPRIAQSNSNLQKPTKKTKKTPYKQLPQPKFVYNSD
SLGPAPMSTIIIWDLPISTSEPFLRNFVSRYGNPLEEMTFITDPTTAVPLGIVTFKFQGNPQKASELAKNFI
KTVRQDELKIDGATLKIALNDNENQLLNRKLESAKKKMLQQRLQREQEEEKRRQKLVEEQKKQELLKKKEKE
HQESVKKEKSVEHESTIVSTRDKNLVYKPNSTVLSMRHNHKIISSVILPKDLEKYIKSRPYILIRDKYVPTK
KISSHDIKRALKKYDWTRVLSDKSGFFIVFNSLNECERCFLNEDNKKFFEYKLVMEMAIPEGFTNNIRENES
KSTNDVLDEATNILIKEFQTFLAKDIRERIIAPNILDLLAHDKYPELVEELKSREQAAKPKVLVTNNQLKEN
ALSILEKQRQLFQQRLPSFRMSHDRTQQHKPKRRNSIIPMQHALNFDDDEDSESHSQSESEDEDEDETTASR
PLTPVVSTMKRERSSTITSIEDDIELEEREIKKQKVKVPAIEAEIAPESSPEEGEEEEKEEVEIKQEAEEVD
IKFQPTEESPRTVYPEIPFSGDFDLNALQHTIKDSEDLLLAQEVLSETTPSGLSNIEYWSWKSKNRKDVQEI
SQEEEYIEELPESLQSTTGSFKSEGVRKIPEIEKIGYLPHRKRTNKPIKTIQYEDEDEEKPNENTNAVQSSR
VNRANNRRFAADITAQIGSESDVLSLNALTKRKKPVTFARSAIHNWGLYAMEPIAAKEMIIEYVGERIRQQV
AEHREKSYLKTGIGSSYLFRIDDNTVIDATKKGGIARFINHCCSPSCTAKIIKVEGKKRIVIYALRDIEANE
ELTYDYKFERETNDEERIRCLCGAPGCKGYLN*

| Rank | Sequence | Start position | Score |
|---|---|---|---|
| 1 | KEMIIEYVGERIRQQV | 921 | 0.94 |
| 1 | EIEKIGYLPHRKRTNK | 823 | 0.94 |
| 1 | DRTQQHKPKRRNSIIP | 600 | 0.94 |
| 2 | YPEIPFSGDFDLNALQ | 734 | 0.93 |
| 2 | VEIKQEAEEVDIKFQP | 710 | 0.93 |
| 2 | SKKIRYNGEGVNHVSD | 166 | 0.93 |
| 2 | TSTIPFSRSINPQKNF | 134 | 0.93 |
| 3 | KTGIGSSYLFRIDDNT | 946 | 0.92 |
| 3 | SGLSTPLSSSDKVSTP | 87 | 0.92 |
| 3 | YILIRDKYVPTKKISS | 421 | 0.92 |
| 3 | GSHRGGYRTGRSKYPE | 22 | 0.92 |
| 3 | SIESINGSDRNTGVNN | 104 | 0.92 |
| 4 | KKRIVIYALRDIEANE | 993 | 0.91 |
| 4 | DEDEEKPNENTNAVQS | 847 | 0.91 |
| 4 | IKTIQYEDEDEEKPNE | 840 | 0.91 |
| 5 | YWSWKSKNRKDVQEIS | 778 | 0.90 |
| 6 | ESTIVSTRDKNLVYKP | 374 | 0.88 |
| 7 | RERIIAPNILDLLAHD | 531 | 0.87 |
| 8 | NEIGSKSPENRSHRSS | 64 | 0.86 |
| 8 | SHSQSESEDEDEDETT | 630 | 0.86 |
| 8 | NILIKEFQTFLAKDIR | 516 | 0.86 |
| 9 | RSSTKDGRTPSGLSTP | 77 | 0.85 |
| 9 | DEDETTASRPLTPVVS | 640 | 0.85 |
| 9 | KGSHYESSDNRYIPNE | 50 | 0.85 |
| 9 | EMAIPEGFTNNIRENE | 488 | 0.85 |
| 9 | TGRSKYPEDRYLVGGM | 30 | 0.85 |
| 10 | LFRIDDNTVIDATKKG | 954 | 0.84 |
| 10 | VQEISQEEEYIEELPE | 789 | 0.84 |
| 10 | AQEVLSETTPSGLSNI | 761 | 0.84 |
| 10 | KKKEKEHQESVKKEKS | 355 | 0.84 |
| 10 | TAVPLGIVTFKFQGNP | 262 | 0.84 |
| 11 | TKKGGIARFINHCCSP | 966 | 0.83 |
| 11 | MTFITDPTTAVPLGIV | 254 | 0.83 |
| 11 | MSTIIIWDLPISTSEP | 223 | 0.83 |
| 11 | FMVINDSHTPKTDKGI | 149 | 0.83 |
| 12 | GGGASGGYSRRGYHGS | 8 | 0.82 |
| 12 | EELTYDYKFERETNDE | 1008 | 0.82 |
| 13 | NTVIDATKKGGIARFI | 960 | 0.81 |
| 13 | ESLQSTTGSFKSEGVR | 804 | 0.81 |
| 13 | ESSPEEGEEEEKEEVE | 696 | 0.81 |
| 13 | SRSINPQKNFMVINDS | 140 | 0.81 |
| 14 | KRERSSTITSIEDDIE | 658 | 0.80 |
| 14 | PVVSTMKRERSSTITS | 652 | 0.80 |
| 15 | DITAQIGSESDVLSLN | 876 | 0.79 |
| 15 | TPSGLSNIEYWSWKSK | 769 | 0.79 |
| 15 | KSREQAAKPKVLVTNN | 556 | 0.79 |
| 15 | KPTKKTKKTPYKQLPQ | 193 | 0.79 |
| 16 | TNAVQSSRVNRANNRR | 857 | 0.78 |
| 16 | EVDIKFQPTEESPRTV | 718 | 0.78 |
| 16 | AIEAEIAPESSPEEGE | 688 | 0.78 |
| 16 | PKRRNSIIPMQHALNF | 607 | 0.78 |
| 17 | QPTEESPRTVYPEIPF | 724 | 0.77 |
| 17 | EEEEKEEVEIKQEAEE | 703 | 0.77 |
| 17 | NSLNECERCFLNEDNK | 463 | 0.77 |
| 17 | TVRQDELKIDGATLKI | 290 | 0.77 |
| 17 | VSRYGNPLEEMTFITD | 244 | 0.77 |
| 17 | NGEGVNHVSDPRIAQS | 172 | 0.77 |
| 18 | AIHNWGLYAMEPIAAK | 906 | 0.76 |
| 18 | DLPISTSEPFLRNFVS | 230 | 0.76 |
| 18 | PYKQLPQPKFVYNSDS | 202 | 0.76 |
| 19 | PELVEELKSREQAAKP | 549 | 0.75 |
| 19 | TRVLSDKSGFFIVFNS | 449 | 0.75 |
| 19 | LQREQEEEKRRQKLVE | 331 | 0.75 |
| 19 | FERETNDEERIRCLCG | 1016 | 0.75 |

| Start | End | Max_score_pos | Sequence |
|---|---|---|---|
| 974 | 991 | 980 | FINHCCSPSCTAKIIKVE |
| 261 | 272 | 266 | TTAVPLGIVTFK |
| 400 | 415 | 405 | HKIISSVILPKDLEKY |
| 647 | 656 | 652 | SRPLTPVVST |
| 756 | 768 | 761 | EDLLLAQEVLSET |
| 1026 | 1037 | 1029 | IRCLCGAPGCKG |
| 995 | 1003 | 998 | RIVIYALRD |
| 563 | 571 | 565 | KPKVLVTNN |
| 884 | 893 | 890 | ESDVLSLNAL |
| 536 | 555 | 542 | APNILDLLAHDKYPELVEEL |
| 681 | 692 | 686 | KQKVKVPAIEAE |
| 203 | 215 | 209 | YKQLPQPKFVYNS |
| 731 | 738 | 736 | RTVYPEIP |
| 482 | 489 | 483 | EYKLVMEM |
| 457 | 465 | 462 | GFFIVFNSL |
| 467 | 474 | 470 | ECERCFLN |
| 88 | 106 | 102 | GLSTPLSSSDKVSTPISIE |
| 417 | 437 | 421 | KSRPYILIRDKYVPTKKISSH |
| 299 | 306 | 304 | DGATLKIA |
| 38 | 45 | 43 | DRYLVGGM |
| 176 | 184 | 179 | VNHVSDPRI |
| 384 | 396 | 388 | NLVYKPNSTVLSM |
| 575 | 593 | 590 | ENALSILEKQRQLFQQRLP |
| 124 | 132 | 127 | FPKLSHHSD |
| 951 | 956 | 952 | SSYLFR |
| 827 | 833 | 829 | IGYLPHR |
| 223 | 238 | 229 | MSTIIIWDLPISTSEP |
| 241 | 248 | 241 | RNFVSRYG |
| 718 | 724 | 722 | EVDIKFQ |
| 615 | 621 | 617 | PMQHALN |
| 800 | 806 | 805 | EELPESL |
| 516 | 528 | 520 | NILIKEFQTFLAK |
| 745 | 751 | 750 | LNALQHT |
| 925 | 931 | 927 | IEYVGER |
| 372 | 380 | 376 | EHESTIVST |
| 897 | 906 | 902 | KKPVTFARSA |
| 933 | 939 | 934 | RQQVAEH |
| 135 | 140 | 140 | STIPES |
| 448 | 455 | 449 | WTRVLSDK |
| 342 | 347 | 345 | QKLVEE |
| 942 | 948 | 946 | KSYLKTG |
| 439 | 445 | 440 | IKRALKK |
| 875 | 880 | 878 | ADITAQ |

15. SAS3
MVSHLLNQLKITNNHIYSNIVPQDLDKRTIRAKPTNDYSLKSMIKNNTHQKSTIPITNTTKIKSVHDDSNSN
IKRVKRSFKHNNIFVKLTKKKCIVVINYDKTKLSTITRSKLDTVPLLPNSTSFTSENQNQNQVDTSILSEIQ
LPYKGILKYPDCIINDTDPTKLDTERFNKYLDEGIKLRNKTTCHLETETETETETETKTEIETELQSQVFSN
LLHPILNESNQSTESTPVPNYSNLNKSKINRIVLRDFEINTWYIAPYPEEYSQCEVLYICEYCLKYMNSPMS
YRRHQLKNCNFSNNHPPGLEIYRDQKSKISIWEVDGRKNINYCQNLCLLAKLFLNSKTLYYDVEPFIFYVLT
EIDEKNPSNYHFVGYFSKEKLNSSDYNVSCILTLPIYQRKGYGNLLIDFSYLLSRNEFKYGTPEKPLSDLGL
LSYRNYWKVTIAYKLKQIYDKYFSCNANGDGDSVSDNARLSLSIDTLCKLTGMIPSDVIVGLEQLDSLARNP
ITHNYAIVINLDKINTEIAKWEKKSYTKLVYEKLLWKPMLFGPSGGINSAPSIQPPQPQSTSVTTTTMSARE
GTTNSHPKPVIPQNSISLITNFLKDDINNPYTFEEEGFKEIEAHRETENEEIKNASLVEYVTCYPGIVVNNH
FVNGGGGSGESNGSNDQIKGHKKMLKKRKRIIDDDDDDDDDDDDDDDEIEKIFEIDEIPSNDENEPDFEDDS
DDVDDFMDDDEEEVVEIKDNDSDEDVSEDIIEILDDDDQEEEDWRRTWKRRVPSPPKRKTVNVLTGNNNKPR
GRGRPRGTFKLKA*

| Rank | Sequence | Start position | Score |
|---|---|---|---|
| 1 | KRTIRAKPTNDYSLKS | 27 | 0.95 |
| 1 | DPTKLDTERFNKYLDE | 162 | 0.95 |
| 2 | KRIIDDDDDDDDDDDD | 677 | 0.94 |
| 2 | QSTSVTTTTMSAREGT | 563 | 0.94 |
| 2 | PDCIINDTDPTKLDTE | 154 | 0.94 |
| 3 | QSTESTPVPNYSNLNK | 227 | 0.93 |
| 4 | QEEEDWRRTWKRRVPS | 759 | 0.92 |
| 4 | APSIQPPQPQSTSVTT | 554 | 0.92 |
| 4 | LSEIQLPYKGILKYPD | 140 | 0.92 |
| 5 | EGTTNSHPKPVIPQNS | 576 | 0.91 |
| 6 | DFEDDSDDVDDFMDDD | 715 | 0.90 |
| 7 | IFEIDEIPSNDENEPD | 700 | 0.89 |
| 7 | EPFIFYVLTEIDEKNP | 352 | 0.89 |
| 7 | KGILKYPDCIINDTDP | 148 | 0.89 |
| 8 | NTWYIAPYPEEYSQCE | 256 | 0.88 |
| 9 | SLVEYVTCYPGIVVNN | 632 | 0.87 |
| 9 | KSVHDDSNSNIKRVKR | 63 | 0.87 |
| 9 | MNSPMSYRRHQLKNCN | 283 | 0.87 |
| 9 | KSKINRIVLRDFEINT | 242 | 0.87 |
| 10 | EKKSYTKLVYEKLLWK | 526 | 0.86 |
| 10 | KLKQIYDKYFSCNANG | 446 | 0.86 |
| 11 | LETETETETETETKTE | 189 | 0.85 |
| 12 | KPRGRGRPRGTFKLKA | 790 | 0.84 |
| 12 | FKEIEAHRETENEEIK | 614 | 0.84 |
| 12 | HQKSTIPITNTTKIKS | 49 | 0.84 |
| 12 | KPTNDYSLKSMIKNNT | 33 | 0.84 |
| 13 | SGESNGSNDQIKGHKK | 656 | 0.83 |
| 13 | HLLNQLKITNNHIYSN | 4 | 0.83 |
| 13 | PYPEEYSQCEVLYICE | 262 | 0.83 |
| 14 | EVVEIKDNDSDEDVSE | 733 | 0.82 |
| 14 | EEIKNASLVEYVTCYP | 626 | 0.82 |
| 14 | QKSKISIWEVDGRKNI | 313 | 0.82 |
| 15 | CEVLYICEYCLKYMNS | 270 | 0.81 |
| 16 | EDIIEILDDDDQEEED | 748 | 0.80 |
| 16 | YFSCNANGDGDSVSDN | 454 | 0.80 |
| 17 | RRTWKRRVPSPPKRKT | 765 | 0.79 |
| 17 | FGPSGGINSAPSIQPP | 545 | 0.79 |
| 17 | KTEIETELQSQVFSNL | 202 | 0.79 |
| 18 | GLEQLDSLARNPITHN | 493 | 0.78 |
| 18 | SLSIDTLCKLTGMIPS | 473 | 0.78 |
| 18 | SCILTLPIYQRKGYGN | 389 | 0.78 |
| 18 | NIVPQDLDKRTIRAKP | 19 | 0.78 |
| 18 | DKTKLSTITRSKLDTV | 101 | 0.78 |
| 19 | EKLLWKPMLFGPSGGI | 536 | 0.77 |
| 19 | RNPITHNYAIVINLDK | 502 | 0.77 |
| 19 | KVTIAYKLKQIYDKYF | 440 | 0.77 |
| 19 | EFKYGTPEKPLSDLGL | 417 | 0.77 |
| 19 | PGLEIYRDQKSKISIW | 305 | 0.77 |
| 20 | DDDDDDDDDDDEIEKI | 685 | 0.76 |
| 20 | SISLITNFLKDDINNP | 591 | 0.76 |
| 20 | DSVSDNARLSLSIDTL | 464 | 0.76 |
| 20 | KSMIKNNTHQKSTIPI | 41 | 0.76 |
| 20 | YFSKEKLNSSDYNVSC | 375 | 0.76 |
| 20 | ERFNKYLDEGIKLRNK | 169 | 0.76 |
| 21 | LQSQVFSNLLHPILNE | 209 | 0.75 |

| Start | End | Max_score_pos | Sequence |
|---|---|---|---|
| 258 | 283 | 273 | WYIAPYPEEYSQCEVLYICEYCLKYM |
| 630 | 648 | 642 | NASLVEYVTCYPGIVVNNH |
| 84 | 100 | 97 | NIFVKLTKKKCIVVINY |
| 328 | 361 | 337 | INYCQNLCLLAKLFLNSKTLYYDVEPFIFYVLTE |
| 386 | 399 | 391 | YNVSCILTLPIYQR |
| 530 | 542 | 536 | YTKLVYEKLLWKP |
| 487 | 500 | 489 | PSDVIVGLEQLDSL |
| 113 | 122 | 116 | LDTVPLLPNS |
| 507 | 515 | 512 | HNYAIVINL |
| 210 | 222 | 216 | QSQVFSNLLHPIL |
| 370 | 376 | 373 | YHFVGYF |
| 471 | 485 | 477 | RLSLSIDTLCKLTGM |
| 440 | 458 | 444 | KVTIAYKLKQIYDKYFSCN |
| 136 | 159 | 154 | DTSILSEIQLPYKGILKYPDCIIN |
| 4 | 9 | 5 | HLLNQL |
| 403 | 415 | 408 | GNLLIDFSYLLSR |
| 583 | 597 | 585 | PKPVIPQNSISLITN |
| 425 | 436 | 430 | KPLSDLGLLSYR |
| 17 | 24 | 18 | YSNIVPQD |
| 246 | 252 | 247 | NRIVLRD |
| 554 | 567 | 557 | APSIQPPQPQSTSV |
| 231 | 237 | 237 | STPVPNY |
| 62 | 68 | 63 | IKSVHDD |
| 732 | 737 | 736 | EEVVEI |
| 316 | 321 | 319 | KISIWE |
| 748 | 754 | 752 | EDIIEIL |

16. RPC53
MSNRLESLNPRKPVSSSSSSGSKSAAKFKPKVVQRKSKEERAKVAPTIKQEPQPRQPLPNSRGRGGARGRGG
RNNYAGTHMVSNGFLSAGAVSIGNSSGSKLGLTSDMIYNSNGDLSSSSTPDFIANFKSKQKGSTPGGQSDEE
DEDDDPTKINMTQKYRFNEEDTVLFPVRPFRDDGITRAENEIAMPDVEIKQEPNDSTAGSTPMPISLTQSRE
TTVKSELIEEKIEQIKETKSKLEKKIAQGGDSFVSEETDKVISDHQQILDILTGKFDKLSTKTEDSHQKQQT
QKDDVDDIDVELENDKTEINFDDQYVLFQLPKHLPTYTQPPSAVKLEPGVQSVEVDEPATEEKEISKLATNN
SKLRGKIGKINIHQSGKITIDLGNDIRLNVTKGAPTDFLQELALIEINPPPKPEDNEEEDVQMVDDDGRSIT
GKVVRLGTVNDKIIATPCIQ*

| Rank | Sequence | Start position | Score |
|---|---|---|---|
| 1 | VEIKQEPNDSTAGSTP | 191 | 0.96 |
| 2 | KVVQRKSKEERAKVAP | 31 | 0.93 |
| 3 | SGKITIDLGNDIRLNV | 375 | 0.92 |
| 4 | SSGSKLGLTSDMIYNS | 97 | 0.91 |
| 4 | NGDLSSSSTPDFIANF | 113 | 0.91 |
| 5 | GRSITGKVVRLGTVND | 428 | 0.89 |
| 5 | KKIAQGGDSFVSEETD | 240 | 0.89 |
| 6 | APTIKQEPQPRQPLPN | 45 | 0.88 |
| 6 | SVEVDEPATEEKEISK | 340 | 0.88 |
| 7 | NSRGRGGARGRGGRNN | 60 | 0.87 |
| 7 | LALIEINPPPKPEDNE | 402 | 0.87 |
| 7 | NVTKGAPTDFLQELAL | 389 | 0.87 |
| 7 | SELIEEKIEQIKETKS | 221 | 0.87 |
| 8 | AVSIGNSSGSKLGLTS | 91 | 0.86 |
| 8 | STPGGQSDEEDEDDDP | 135 | 0.86 |
| 9 | EEDEDDDPTKINMTQK | 143 | 0.85 |
| 10 | GGRNNYAGTHMVSNGF | 71 | 0.84 |
| 10 | VQMVDDDGRSITGKVV | 421 | 0.84 |
| 11 | LEPGVQSVEVDEPATE | 334 | 0.82 |
| 11 | HQQILDILTGKFDKLS | 261 | 0.82 |
| 12 | RGKIGKINIHQSGKIT | 364 | 0.81 |
| 13 | PKPEDNEEEDVQMVDD | 411 | 0.80 |
| 13 | SKEERAKVAPTIKQEP | 37 | 0.80 |
| 13 | HLPTYTQPPSAVKLEP | 321 | 0.80 |
| 13 | DKLSTKTEDSHQKQQT | 273 | 0.80 |
| 14 | DVELENDKTEINFDDQ | 297 | 0.79 |
| 14 | ENEIAMPDVEIKQEPN | 183 | 0.79 |
| 15 | DSTAGSTPMPISLTQS | 199 | 0.76 |
| 15 | TKINMTQKYRFNEEDT | 151 | 0.76 |
| 15 | SSSSSSGSKSAAKFKP | 15 | 0.76 |
| 15 | SDMIYNSNGDLSSSST | 106 | 0.76 |
| 16 | KTEINFDDQYVLFQLP | 304 | 0.75 |
| 16 | KSAAKFKPKVVQRKSK | 23 | 0.75 |

| Start | End | Max_score_pos | Sequence |
|---|---|---|---|
| 311 | 346 | 316 | DQYVLFQLPKHLPTYTQPPSAVKLEPGVQSVEVDEP |
| 166 | 174 | 170 | TVLFPVRPF |
| 432 | 439 | 438 | TGKVVRLG |
| 396 | 408 | 402 | TDFLQELALIEIN |
| 25 | 36 | 31 | AAKFKPKVVQRK |
| 88 | 95 | 89 | SAGAVSIG |
| 256 | 270 | 265 | KVISDHQQILDILTG |
| 42 | 49 | 45 | AKVAPTIK |
| 11 | 17 | 16 | RKPVSSS |
| 207 | 212 | 211 | MPISLT |
| 387 | 393 | 389 | RLNVTKG |
| 188 | 194 | 192 | MPDVEIK |

17. IPF2140(CAF40)
MSSVGNPVHSLASTDSTASSSSSNKALNDEQIYQWISELVTGNNRERALLELGKKREQYDDLALVLWNSFGV
ITVLLEEIISVYPYLNPPNLSASISNRVCNALALLQCVASNVQTRTLFLNANLPLYLYPFLSTNARQRSFEY
LRLTSLGVIGALVKNDTPEVINFLLTTEIVPLCLNIMEISSELSKTVAIFILQKILLDDQGLAYVCTTFERF
HTVASVLSKMIDQLSIAVNSTNSQQQQQQQGQQAQQQQQQQQQTQSVPSSNSSGRLLKHVVRCYMRLSDNLE
ARKALANILPEPLRDGTFSTILQDDLATKRCLSQLLSNINEPQ*

| Rank | Sequence | Start position | Score |
|---|---|---|---|
| 1 | YQWISELVTGNNRERA | 33 | 0.86 |
| 2 | FGVITVLLEEIISVYP | 70 | 0.85 |
| 2 | LQKILLDDQGLAYVCT | 196 | 0.85 |
| 2 | MSSVGNPVHSLASTDS | 1 | 0.85 |
| 3 | TDSTASSSSSNKALND | 14 | 0.84 |
| 4 | MRLSDNLEARKALANI | 281 | 0.82 |
| 5 | QGLAYVCTTFERFHTV | 204 | 0.81 |
| 5 | LVKNDTPEVINFLLTT | 156 | 0.81 |
| 6 | FSTILQDDLATKRCLS | 306 | 0.79 |
| 7 | ANILPEPLRDGTFSTI | 294 | 0.78 |
| 7 | ASNVQTRTLFLNANLP | 111 | 0.78 |
| 8 | KKREQYDDLALVLWNS | 54 | 0.77 |
| 8 | LGVIGALVKNDTPEVI | 150 | 0.77 |

| Start | End | Max_score_pos | Sequence |
|---|---|---|---|
| 97 | 114 | 107 | SNRVCNALALLQCVASNV |
| 162 | 181 | 175 | PEVINFLLTTEIVPLCLNIM |
| 270 | 282 | 276 | GRLLKHVVRCYMR |
| 116 | 134 | 130 | TRTLFLNANLPLYLYPFLS |
| 60 | 95 | 74 | DDLALVLWNSFGVITVLLEEIISVYPYLNPPNLSAS |
| 316 | 326 | 322 | TKRCLSQLLSN |
| 188 | 213 | 207 | SKTVAIFILQKILLDDQGLAYVCTTF |
| 216 | 235 | 220 | FHTVASVLSKMIDQLSIAVN |
| 142 | 159 | 155 | FEYLRLTSLGVIGALVKN |
| 5 | 14 | 10 | GNPVHSLAST |
| 290 | 300 | 295 | RKALANILPEP |
| 33 | 40 | 37 | YQWISELV |
| 307 | 313 | 312 | STILQDD |
| 260 | 266 | 261 | TQSVPSS |
| 247 | 256 | 251 | GQQAQQQQQQ |

18. IPF19724(TBF1)
MSDQLEKDIEESIANLDYQQNQEHHETEQDKDKEHQDVEKQSSEEETKGIEHVTDSNIDVIEVTKSRDTEEV
IENSPVDPQLKEQQESTTKMSSSERDLVDEIDELFTNSTKIVTENNQPSETNKRAYESVETPQELTPNDKRQ
KLDANTETSVPTELESVNNHNEQSQPIEPTQERQPSTTETTYSISVPVSTTNEVERASSSINEQEDLEMIAK
QYQQATNLEIERAMEGHGDGGQHFSTQENGQPSGSSLISSIVPSDSELLNTNQAYAAYTSLSSQLEQHTSAS
AMLSSATLSALPLSIIAPVYLPPRIQLLINTLPTLDNLATQLLRTVATSPYQKIIDLASNPDTSAGATYRDL
TSLFEFTKRLYSEDDPFLTVEHIAPGMWKEGEETPSIFKPKQQSIESTLRKVNLATFLAATLGTMEIGFFYL
NESFLDVFCPSNNLDPSNALSNLGGYQNGLQSTDSPVGARVGKLLKPQATLYLDLKTQAYISAIEAGERSKE
EILEDILPDDLHVYLMSRRNAKLLSPTETDFVWRCKQRKESLLNYTEETPLSEQYDWFTFLRDLFDYVSKNI
AYLIWGKMGKTMKNRREDTPHTQELLDNTTGSTQMPNQLSSSSGQASSTPSVVDPNKMLVSEMREANIAVPK
PSQRRAWSREEEKALRHALELKGPHWATILELFGQGGKISEALKNRTQVQLKDKARNWKKFFLRSGLEIPSY
LRGVTGGVDDGKRKKDNVTKKTAAAPVPNMSEQLQQQQQRQQEKQEKQQQEEQQAQQSEPQQEPQQEPQQEQ
QQEQQQEQQQEQQQEQQQEQQQEQQQEQQQEQREETQQTEQEQPDQPQEEQQQEKEQPDQQQQEKEQPDQQQ
PDQQHPDRQQQEQIQQPENSDK*

| Rank | Sequence | Start position | Score |
|---|---|---|---|
| 1 | QQSIESTLRKVNLATF | 402 | 0.92 |
| 1 | HNEQSQPIEPTQERQP | 164 | 0.92 |
| 2 | EEQQQEKEQPDQQQQE | 841 | 0.90 |
| 2 | GVTGGVDDGKRKKDNV | 723 | 0.90 |
| 3 | GGKISEALKNRTQVQL | 684 | 0.89 |
| 3 | SEEETKGIEHVTDSNI | 43 | 0.89 |
| 3 | QLLINTLPTLDNLATQ | 314 | 0.89 |
| 3 | DQLEKDIEESIANLDY | 3 | 0.89 |
| 4 | NGLQSTDSPVGARVGK | 460 | 0.88 |
| 5 | DQQQQEKEQPDQQQPD | 851 | 0.87 |
| 5 | SSSGQASSTPSVVDPN | 617 | 0.87 |
| 5 | YRDLTSLFEFTKRLYS | 357 | 0.87 |
| 5 | TETTYSISVPVSTTNE | 182 | 0.87 |
| 5 | ETSVPTELESVNNHNE | 151 | 0.87 |
| 5 | TPQELTPNDKRQKLDA | 133 | 0.87 |
| 6 | ISAIEAGERSKEEILE | 493 | 0.86 |
| 7 | NRREDTPHTQELLDNT | 590 | 0.85 |
| 7 | SSLISSIVPSDSELLN | 251 | 0.85 |
| 7 | SINEQEDLEMIAKQYQ | 204 | 0.85 |
| 8 | QPDQQQPDQQHPDRQQ | 859 | 0.84 |
| 8 | QTEQEQPDQPQEEQQQ | 830 | 0.84 |
| 8 | TEEVIENSPVDPQLKE | 69 | 0.84 |
| 8 | TGSTQMPNQLSSSSGQ | 606 | 0.84 |
| 8 | NYTEETPLSEQYDWFT | 548 | 0.84 |
| 8 | EFTKRLYSEDDPFLTV | 365 | 0.84 |
| 8 | EGHGDGGQHFSTQENG | 231 | 0.84 |
| 9 | GLEIPSYLRGVTGGVD | 714 | 0.83 |
| 9 | KEEILEDILPDDLHVY | 503 | 0.83 |
| 9 | PGMWKEGEETPSIFKP | 385 | 0.83 |
| 9 | PLSIIAPVYLPPRIQL | 300 | 0.83 |
| 10 | LIWGKMGKTMKNRRED | 579 | 0.82 |
| 10 | TMEIGFFYLNESFLDV | 424 | 0.82 |
| 11 | GKRKKDNVTKKTAAAP | 731 | 0.81 |
| 11 | QRRAWSREEEKALRHA | 651 | 0.81 |
| 11 | DLHVYLMSRRNAKLLS | 514 | 0.81 |
| 11 | TVEHIAPGMWKEGEET | 379 | 0.81 |
| 11 | QEHHETEQDKDKEHQD | 22 | 0.81 |
| 12 | GARVGKLLKPQATLYL | 470 | 0.80 |
| 12 | DVEKQSSEEETKGIEH | 37 | 0.80 |
| 13 | STTKMSSSERDLVDEI | 88 | 0.79 |
| 13 | KKTAAAPVPNMSEQLQ | 740 | 0.79 |
| 13 | EVTKSRDTEEVIENSP | 62 | 0.79 |
| 13 | DFVWRCKQRKESLLNY | 534 | 0.79 |
| 13 | IEHVTDSNIDVIEVTK | 50 | 0.79 |
| 13 | QKIIDLASNPDTSAGA | 340 | 0.79 |
| 13 | KQYQQATNLEIERAME | 216 | 0.79 |
| 13 | TQERQPSTTETTYSIS | 174 | 0.79 |
| 14 | SEPQQEPQQEPQQEQQ | 778 | 0.78 |
| 14 | KMLVSEMREANIAVPK | 633 | 0.78 |
| 14 | VSKNIAYLIWGKMGKT | 572 | 0.78 |
| 15 | NSPVDPQLKEQQESTT | 75 | 0.77 |
| 15 | FSTQENGQPSGSSLIS | 240 | 0.77 |
| 16 | KLLSPTETDFVWRCKQ | 526 | 0.76 |
| 16 | TNEVERASSSINEQED | 195 | 0.76 |
| 17 | EQQAQQSEPQQEPQQE | 772 | 0.75 |
| 17 | DWFTFLRDLFDYVSKN | 560 | 0.75 |
| 17 | RTVATSPYQKIIDLAS | 332 | 0.75 |

| Start | End | Max_score_pos | Sequence |
|---|---|---|---|
| 288 | 346 | 306 | SAMLSSATLSALPLSIIAPVYLPPRIQLLINTLPT |
| | | | LDNLATQLLRTVATSPYQKIIDLA |
| 186 | 193 | 189 | YSISVPVS |
| 428 | 444 | 439 | GFFYLNESFLDVFCPSN |
| 250 | 263 | 256 | GSSLISSIVPSDSE |
| 512 | 520 | 518 | PDDLHVYLM |
| 624 | 638 | 626 | STPSVVDPNKMLVSE |
| 465 | 496 | 484 | TDSPVGARVGKLLKPQATLYLDLKTQAYISAI |
| 377 | 383 | 380 | FLTVEHI |
| 709 | 727 | 718 | FFLRSGLEIPSYLRGVTGG |
| 409 | 421 | 415 | LRKVNLATFLAAT |
| 58 | 64 | 61 | IDVIEVT |
| 565 | 581 | 571 | LRDLFDYVSKNIAYLIW |
| 643 | 649 | 647 | NIAVPKP |
| 74 | 81 | 79 | ENSPVDPQ |
| 663 | 670 | 666 | LRHALELK |
| 270 | 284 | 274 | AYAAYTSLSSQLEQH |
| 743 | 748 | 745 | AAAPVP |
| 674 | 681 | 678 | WATILELF |
| 525 | 530 | 528 | AKLLSP |
| 360 | 365 | 363 | LTSLFE |
| 128 | 134 | 134 | YESVETP |
| 396 | 403 | 398 | SIFKPKQQ |
| 615 | 621 | 617 | LSSSSGQ |
| 35 | 41 | 38 | HQDVEKQ |

19. IPF11711
MIVGKRDHHQRMEMAKPLRSLIDKLTTCDINELPQYLQENFKWQRPRGDLIHWIPLLNRFDEIFEQKIEKYG
LDKDNVKLSLVSPEDERLIVSCLQFTYILLDHCFDKQVYSSSERIYALINSSSLEIRLRALEVGIVLAEKFV
QTTSSRFSAPKPVRNKVLEIAKSFPPLVPIDSTLKQLAENKKNNNHRDTDEKPSIIGDHYNFVYTLDPEKKY
PSKWKSINYQYYKSVPNTPTLNKNASKSKSNDKKKEDTVTEGLHIFHLPEESVRKLTVQQLFEKGMEVLPPE
SWFCFGIHAQMTKSFNSTSSDAMQLREKLIQIKCLAVGFTCCMLSSQVTSTKLFETEPYIFSFLVEAISPEN
SSLVSRDVYFAAIRALECISFKKVWGAELIRTMGGNVSHGILFQCLRHIWKMVKDQKEDYFEEGYIHFFNLI
GNLISNKSLVPKLTAGGILDDLMPFLNLPTKYRWSCSAAVHLITMYLASAKDSLDEFVTNDGFTLLIGNIRR
EVDFALENPEFGGGAPKDAIVYYSITLRQANYIRNLMKLVADLIQSDSGDRLRNLFDSPLLESFNKVLTHPH
VFGPSILAATIDSVFFIIHNEPTAFSILNEAKVIDTILDNYESLFLPSGPLLQILPEVLGAICLNNEGLNKV
KDKKLIQVFFKSFYNLNNAKELVRTESSTNLGCSLDELGRHYTSLKPIILQQLSELIENMSEYVNQRLPGIE
FYTSDSGSLFSGKGDDSPVKVENGKEITSWENEDSAYLLDNVFNFLGGLLQDSGQWGTDVIQKISFKSWIKL
LTLRNAPWDYSMSNGIVSFMGILKYFDDEKREYGLPVIIEELDKTLKLDSVLSYIKYKGEVSYFETIEPQQA
TILLQDLNIINVLLYSLTEIYINLGSLFNERLGQIVSLFSNSNLLMNLVQLLERSIIEEAIIVSNTPDEVLK
MTHNFPNDSPPLQINVCDPSEIKADTKGTSAKFKNTLQLRTGSYYFRGYIPLILASVVRSCIPKRQDHAEGQ
ARQDAVEILLSLGKEFTDSIGRKFNNSYYEESFILNIAYVALYILNQKERSKDQVYTPFAISLFQNGFFKVA
EATCISLWNKLLIMDPELASATLDLKYISTQESSIIKNALGQILMIFAKTVNHENIPNVPFAKFYFYQGFES
NIEQSLTSALLLQIRSVALSLVEHTVGSKSQLTGTNKHPDNVPTPLIEQIAVIIKDIFVGKKELSDSEFIPF
DTRNISPPSDQFAYLISLGMSEDQAAHFFEHGCNLSDIASGKFLRCVEIDLKEEQWQSIAESIRDEKIDFSI
DFEKFKSTKDILKERKAVDFENIWMNIAQSFPKSISFISDFIMLVSYKEFYDEMLDYTPKIKWPIEDKELFG
INLYIIALLLQTGKPHIHRRDLMLNAQTLINPDIISTDTVNEKFFPSLLLVLERMMILLEEPEHEMAPELPF
TIQKKKEFDVATKEFKAKTFDLLVKLEVKDNLESAYGLARVLVLYARDKSYADRLAGSQILKDLLKLVRTNV
KRKTVIEALRTSVILIFRYCFETSRMAENIMNIEVTALFNNPIRQIKDLHACLRESAPLVFRDIDMAVGVIC
NNILLEGYTGEESYVSKIAIRKRKRDESMQDVEMSEPEASKPLLEFLTFSKKTQEEVKPRATALNFFIHQLI
PTHSLESSTGAEFERRCAISSIAKMCLLSLVSSTISGDNDSSKAKKEDADLALIRRFVVDLMMKILKDVSQS
NTLGSIKYGKLLDLFELSGSLISTKFRDSVGPLLNKQATQYDQFHISKIYIEKQVPNLLTNLIAEFDLNFPQ
IDKVVKAALKPMTFLGKNKVEYEELFAGDANQGDHNDDDDNLPEDVDYHEETPDLFRNSTLGMYDVESEGDD
EFYDAEDPVDAMMTGEDLSGASDDGDDDDDEDDDGDDDIDDMSSELSAIDSDLDGGDNADDIEMEIEVDPYD
DERGSEDIDEDASDMEDIEIIDDLDLVSQTDGDDDGDDDDGRDDEEDSSSWDDDEMSEYDEDELDGWIEQLD
DSEDSNDEVQSRRRPRDPFTSLNNDAESGLRQRLFLDGEADFDDDNAIESDGELSEMDSRSDFEVRMVTPSR
GRRRRILDRADFNELERASPALSVLLDGLFRDRNFGSIEISRYXHLXHDTSTIGRLXEXMMHXGRVSKHXNQ
DNKLHIKXTXERWSDVLKMYYPRDGGDLVYPLTTTIIGRIKDESQAIANRKKEEQEKAKKEREEKRRKQLEE
EEKRRQEESRQRQESTANVPEREPVMVRIGDRDVDISGTEIDRDYIEALPEDMREEVFASYVRERRANASST
DTDVREIDPDFLDALPDNIRTEILQQETMARRFANFESSSAQDEFEVDDAGEEDVFEDADDARPSGSGRSSS
AATTRAQKKPAGKVYFSPLVDKQGIAALIRLLFSPLTISQREHIYHALQYMCYNKQSRIEIMSLMIAILNDC
FTNNRPAQKVYTQVCNKAGGNKDSKQQYKLPVGATQISVGIQIVEAVDYLLERNNHLKYFLLTEHENTFILR
KDKKSASKESKFPINYMLKILDNKLVTDDQTLLDILARVFQVASRALHALKNSANADDKDDDKENEKEKEKD
KPHAPPPPVIPDSNYRLIIKILTGNDCSNTTFRRTISAMQNFSVLPNAQKLFSLELSDKASELGQTIITDLN
NLTKELVAGGGSDSKSFSKFSAHWSDQAKLLRILTALDYMFENKEKNKEKGKEDEIEELTDLYKKLALGSLW
DALSETLRVLEEKPQLHNIANALLPLIEALMVVCKHSKVRELPIKDILKYEAKKIDFTKEPIESLFFSFTDE
HKKILNQMVRSNPNLMSGPFGMLVRNPRVLEFDNKKNYFDRKLHQDKKENRKMLVSVRRDQVFLDSYRSLFF
KPKDEFRNSKLEINFKGEQGIDAGGVTREWYQVLSRQMFNPDYALFTPVVSDETTFHPNRTSYINPEHLSFF
KFIGRIIGKAIYDNCFLDCHFSRAVYKRILGKPQSLKDMETLDLEYFKSLMWMLENDITDVITEDFSVETDD
YGEHKIIDLIPNGRNIPVTEENKNEYVKKVVEYRLQTSVEEQMENFLIGFHEIIPKDLVAIFDEKELELLIS
GLPDIDVSDWQNHTSYNNYSPSSLQIQWFWRAVKSFDNEERARLLQFATGTSKVPLNGFKELSGASGTCKFS
IHRDYGSTDRLPSSHTCFNQIDLPAYDCYETLRGSLLMAITEGHEGFGLA*

| Rank | Sequence | Start position | Score |
|---|---|---|---|
| 1 | PFTIQKKKEFDVATKE | 1439 | 0.97 |
| 2 | LSAIDSDLDGGDNADD | 1918 | 0.96 |
| 3 | PSIIGDHYNFVYTLDP | 197 | 0.95 |
| 4 | SGASDDGDDDDDEDDD | 1891 | 0.94 |
| 5 | VSDWQNHTSYNNYSPS | 3103 | 0.93 |
| 5 | ADDIEMEIEVDPYDDE | 1931 | 0.93 |
| 5 | MSEDQAAHFFEHGCNL | 1244 | 0.93 |
| 6 | TPVVSDETTFHPNRTS | 2927 | 0.92 |
| 6 | VESEGDDEFYDAEDPV | 1866 | 0.92 |
| 6 | SQLTGTNKHPDNVPTP | 1182 | 0.92 |
| 7 | EIKADTKGTSAKFKNT | 957 | 0.91 |
| 7 | EEAIIVSNTPDEVLKM | 922 | 0.91 |
| 7 | DRLPSSHTCFNQIDLP | 3177 | 0.91 |
| 7 | QSRIEIMSLMIAILND | 2432 | 0.91 |
| 7 | DVREIDPDFLDALPDN | 2307 | 0.91 |
| 7 | EVFASYVRERRANASS | 2288 | 0.91 |
| 7 | KWPIEDKELFGINLYI | 1358 | 0.91 |
| 8 | PEVLGAICLNNEGLNK | 632 | 0.90 |
| 8 | PVTEENKNEYVKKVVE | 3041 | 0.90 |
| 8 | REHIYHALQYMCYNKQ | 2417 | 0.90 |
| 8 | NSTLGMYDVESEGDDE | 1858 | 0.90 |
| 8 | SSTISGDNDSSKAKKE | 1688 | 0.90 |
| 8 | DEKIDFSIDFEKFKST | 1289 | 0.90 |
| 8 | AVIIKDIFVGKKELSD | 1203 | 0.90 |
| 9 | SGKGDDSPVKVENGKE | 731 | 0.89 |
| 9 | SGTCKFSIHRDYGSTD | 3162 | 0.89 |
| 9 | HKIIDLIPNGRNIPVT | 3028 | 0.89 |
| 9 | VTREWYQVLSRQMFNP | 2906 | 0.89 |
| 9 | SGRSSSAATTRAQKKP | 2371 | 0.89 |
| 9 | DPEKKYPSKWKSINYQ | 211 | 0.89 |
| 9 | NDEVQSRRRPRDPFTS | 2022 | 0.89 |
| 9 | DSSSWDDDEMSEYDED | 1991 | 0.89 |
| 9 | TDGDDDGDDDDGRDDE | 1974 | 0.89 |
| 9 | PVDAMMTGEDLSGASD | 1880 | 0.89 |
| 9 | KVEYEELFAGDANQGD | 1819 | 0.89 |
| 9 | ETSRMAENIMNIEVTA | 1534 | 0.89 |
| 9 | KLLIMDPELASATLDL | 1090 | 0.89 |
| 10 | YRLIIKILTGNDCSNT | 2607 | 0.88 |
| 10 | MVRIGDRDVDISGTEI | 2258 | 0.88 |
| 10 | QEESRQRQESTANVPE | 2238 | 0.88 |
| 10 | LXEXMMHXGRVSKHXN | 2144 | 0.88 |
| 10 | FGSIEISRYXHLXHDT | 2123 | 0.88 |
| 11 | EIYINLGSLFNERLGQ | 883 | 0.87 |
| 11 | VHLITMYLASAKDSLD | 472 | 0.87 |
| 11 | AGGILDDLMPFLNLPT | 447 | 0.87 |
| 11 | GRIIGKAIYDNCFLDC | 2956 | 0.87 |
| 11 | SELGQTIITDLNNLTK | 2653 | 0.87 |
| 11 | AQKVYTQVCNKAGGNK | 2455 | 0.87 |
| 11 | RSLIDKLTTCDINELP | 19 | 0.87 |
| 12 | IPKRQDHAEGQARQDA | 998 | 0.86 |
| 12 | GGLLQDSGQWGTDVIQ | 767 | 0.86 |
| 12 | RLPGIEFYTSDSGSLF | 715 | 0.86 |
| 12 | SELIENMSEYVNQRLP | 702 | 0.86 |
| 12 | RKLHQDKKENRKMLVS | 2849 | 0.86 |
| 12 | TIIGRIKDESQAIANR | 2195 | 0.86 |
| 12 | DERGSEDIDEDASDME | 1945 | 0.86 |
| 12 | DANQGDHNDDDDNLPE | 1829 | 0.86 |
| 12 | PEHEMAPELPFTIQKK | 1430 | 0.86 |
| 12 | HAEGQARQDAVEILLS | 1004 | 0.86 |
| 13 | ENDITDVITEDFSVET | 3007 | 0.85 |
| 13 | LLRILTALDYMFENKE | 2694 | 0.85 |
| 13 | HALKNSANADDKDDDK | 2568 | 0.85 |
| 13 | YKSVPNTPTLNKNASK | 228 | 0.85 |
| 13 | WKSINYQYYKSVPNTP | 220 | 0.85 |
| 13 | VLKMYYPRDGGDLVYP | 2176 | 0.85 |
| 13 | SRGRRRRILDRADFNE | 2087 | 0.85 |
| 13 | PKPVRNKVLEIAKSFP | 154 | 0.85 |
| 14 | NFPNDSPPLQINVCDP | 940 | 0.84 |
| 14 | LPVIIEELDKTLKLDS | 827 | 0.84 |
| 14 | AKVIDTILDNYESLFL | 607 | 0.84 |
| 14 | VGKRDHHQRMEMAKPL | 3 | 0.84 |
| 14 | GIHAQMTKSFNSTSSD | 294 | 0.84 |
| 14 | VLSRQMFNPDYALFTP | 2913 | 0.84 |
| 14 | EFEVDDAGEEDVFEDA | 2348 | 0.84 |
| 14 | LSEMDSRSDFEVRMVT | 2070 | 0.84 |
| 14 | IESDGELSEMDSRSDF | 2064 | 0.84 |
| 14 | DEFYDAEDPVDAMMTG | 1872 | 0.84 |
| 14 | YHEETPDLFRNSTLGM | 1848 | 0.84 |
| 14 | TFSKKTQEEVKPRATA | 1632 | 0.84 |
| 14 | NPDIISTDTVNEKFFP | 1399 | 0.84 |
| 14 | TRNISPPSDQFAYLIS | 1226 | 0.84 |
| 15 | LQLRTGSYYFRGYIPL | 973 | 0.83 |
| 15 | SNTPDEVLKMTHNFPN | 928 | 0.83 |
| 15 | ERLIVSCLQFTYILLD | 88 | 0.83 |
| 15 | FFIIHNEPTAFSILNE | 591 | 0.83 |
| 15 | SVETDDYGEHKIIDLI | 3019 | 0.83 |
| 15 | MSGPFGMLVRNPRVLE | 2824 | 0.83 |
| 15 | EKEKDKPHAPPPPVIP | 2588 | 0.83 |
| 15 | KLHIKXTXERWSDVLK | 2163 | 0.83 |
| 15 | TSTIGRLXEXMMHXGR | 2138 | 0.83 |
| 15 | LVPIDSTLKQLAENKK | 171 | 0.83 |
| 15 | SLESSTGAEFERRCAI | 1660 | 0.83 |
| 15 | HQLIPTHSLESSTGAE | 1653 | 0.83 |
| 15 | KFVQTTSSRFSAPKPV | 142 | 0.83 |
| 15 | LVSYKEFYDEMLDYTP | 1340 | 0.83 |
| 15 | LGKEFTDSIGRKFNNS | 1020 | 0.83 |
| 16 | KEITSWENEDSAYLLD | 745 | 0.82 |
| 16 | FYTSDSGSLFSGKGDD | 721 | 0.82 |
| 16 | GCSLDELGRHYTSLKP | 680 | 0.82 |
| 16 | HWSDQAKLLRILTALD | 2687 | 0.82 |
| 16 | EEEKRRQEESRQRQES | 2232 | 0.82 |
| 16 | KEEQEKAKKEREEKRR | 2212 | 0.82 |
| 16 | RRRPRDPFTSLNNDAE | 2028 | 0.82 |
| 16 | KQLAENKKNNNHRDTD | 179 | 0.82 |
| 16 | KPRATALNFFIHQLIP | 1642 | 0.82 |
| 17 | PLLQILPEVLGAICLN | 626 | 0.81 |
| 17 | GGAPKDAIVYYSITLR | 517 | 0.81 |
| 17 | MPFLNLPTKYRWSCSA | 455 | 0.81 |
| 17 | QRPRGDLIHWIPLLNR | 44 | 0.81 |
| 17 | CCMLSSQVTSTKLFET | 329 | 0.81 |
| 17 | NELPQYLQENFKWQRP | 31 | 0.81 |
| 17 | FKSLMWMLENDITDVI | 2999 | 0.81 |
| 17 | YKRILGKPQSLKDMET | 2978 | 0.81 |
| 17 | LVRNPRVLEFDNKKNY | 2831 | 0.81 |
| 17 | ELPIKDILKYEAKKID | 2777 | 0.81 |
| 17 | RRTISAMQNFSVLPNA | 2625 | 0.81 |
| 17 | TTRAQKKPAGKVYFSP | 2379 | 0.81 |
| 17 | LQQETMARRFANFESS | 2328 | 0.81 |
| 17 | SKIAIRKRKRDESMQD | 1600 | 0.81 |
| 17 | ALLLQTGKPHIHRRDI | 1375 | 0.81 |
| 17 | RKFNNSYYEESFILNI | 1030 | 0.81 |
| 18 | IGNIRREVDFALENPE | 499 | 0.80 |
| 18 | ASAKDSLDEFVTNDGF | 480 | 0.80 |
| 18 | LRHIWKMVKDQKEDYF | 406 | 0.80 |
| 18 | LECISFKKVWGAELIR | 376 | 0.80 |
| 18 | DSKQQYKLPVGATQIS | 2471 | 0.80 |
| 18 | SPLTISQREHIYHALQ | 2410 | 0.80 |
| 18 | GWIEQLDDSEDSNDEV | 2010 | 0.80 |
| 18 | SKIYIEKQVPNLLTNL | 1775 | 0.80 |
| 18 | DESMQDVEMSEPEASK | 1610 | 0.80 |
| 18 | CLRESAPLVFRDIDMA | 1564 | 0.80 |
| 18 | MILLEEPEHEMAPELP | 1424 | 0.80 |
| 18 | GKKELSDSEFIPFDTR | 1212 | 0.80 |
| 19 | KKLIQVFFKSFYNLNN | 651 | 0.79 |
| 19 | HGILFQCLRHIWKMVK | 399 | 0.79 |
| 19 | RAVKSFDNEERARLLQ | 3127 | 0.79 |
| 19 | HEIIPKDLVAIFDEKE | 3075 | 0.79 |
| 19 | AGKVYFSPLVDKQGIA | 2387 | 0.79 |
| 19 | MSEYDEDELDGWIEQL | 2000 | 0.79 |
| 19 | EVDPYDDERGSEDIDE | 1939 | 0.79 |
| 19 | HNDDDDNLPEDVDYHE | 1835 | 0.79 |
| 19 | SGSLISTKFRDSVGPL | 1746 | 0.79 |
| 19 | KSTKDILKERKAVDFE | 1302 | 0.79 |
| 19 | ESNIEQSLTSALLLQI | 1151 | 0.79 |
| 19 | DLKYISTQESSIIKNA | 1104 | 0.79 |
| 19 | KQVYSSSERIYALINS | 108 | 0.79 |
| 20 | LGRHYTSLKPIILQQL | 686 | 0.78 |
| 20 | EQKIEKYGLDKDNVKL | 65 | 0.78 |
| 20 | YFEEGYIHFFNLIGNL | 420 | 0.78 |
| 20 | YRSLFFKPKDEFRNSK | 2875 | 0.78 |
| 20 | FEKGMEVLPPESWFCF | 278 | 0.78 |
| 20 | AGGGSDSKSFSKFSAH | 2672 | 0.78 |
| 20 | IVEAVDYLLERNNHLK | 2491 | 0.78 |
| 20 | DARPSGSGRSSSAATT | 2365 | 0.78 |
| 20 | SKPLLEFLTFSKKTQE | 1624 | 0.78 |
| 21 | GILKYFDDEKREYGLP | 813 | 0.77 |
| 21 | CFNQIDLPAYDCYETL | 3185 | 0.77 |
| 21 | TGTSKVPLNGFKELSG | 3145 | 0.77 |
| 21 | SGLPDIDVSDWQNHTS | 3096 | 0.77 |
| 21 | DMETLDLEYFKSLMWM | 2990 | 0.77 |
| 21 | LRVLEEKPQLHNIANA | 2743 | 0.77 |
| 21 | ANASSTDTDVREIDPD | 2299 | 0.77 |
| 21 | DKVVKAALKPMTFLGK | 1802 | 0.77 |
| 21 | KQATQYDQFHISKIYI | 1764 | 0.77 |
| 22 | FKSWIKLLTLRNAPWD | 786 | 0.76 |
| 22 | RDYGSTDRLPSSHTCF | 3171 | 0.76 |
| 22 | PINYMLKILDNKLVTD | 2533 | 0.76 |
| 22 | KPHIHRRDIMLNAQTL | 1382 | 0.76 |
| 22 | RKAVDFENIWMNIAQS | 1311 | 0.76 |
| 22 | PELASATLDLKYISTQ | 1096 | 0.76 |
| 22 | QRMEMAKPLRSLIDKL | 10 | 0.76 |
| 23 | PVKVENGKEITSWENE | 738 | 0.75 |
| 23 | VDFALENPEFGGGAPK | 506 | 0.75 |
| 23 | RLLQFATGTSKVPLNG | 3139 | 0.75 |
| 23 | YSPSSLQIQWFWRAVK | 3115 | 0.75 |
| 23 | PPESWFCFGIHAQMTK | 286 | 0.75 |
| 23 | CSNTTFRRTISAMQNF | 2619 | 0.75 |
| 23 | PHAPPPPVIPDSNYRL | 2594 | 0.75 |
| 23 | DYIEALPEDMREEVFA | 2276 | 0.75 |
| 23 | XGRVSKHXNQDNKLHI | 2151 | 0.75 |
| 23 | DFDDDNAIESDGELSE | 2057 | 0.75 |
| 23 | TNLIAEFDLNFPQIDK | 1788 | 0.75 |
| 23 | EFERRCAISSIAKMCL | 1668 | 0.75 |
| 23 | AGSQILKDLLKLVRTN | 1496 | 0.75 |
| 23 | VLYARDKSYADRLAGS | 1483 | 0.75 |
| 23 | KEFDVATKEFKAKTFD | 1446 | 0.75 |

| Start | End | Max_score_pos | Sequence |
|---|---|---|---|
| 1670 | 1690 | 1685 | ERRCAISSIAKMCLLSLVSST |
| 975 | 1001 | 991 | LRTGSYYFRGYIPLILASVVRSCIPKR |
| 89 | 147 | 93 | RLIVSCLQFTYILLDHCFDKQVYSSSERIYALINSSSLEIR |
| | | | LRALEVGIVLAEKFVQTT |
| 1413 | 1429 | 1417 | FPSLLLVLERMMILLEE |
| 1475 | 1488 | 1481 | AYGLARVLVLYARD |
| 1042 | 1054 | 1050 | ILNIAYVALYILN |
| 616 | 642 | 637 | NYESLFLPSGPLLQILPEVLGAICLNN |
| 1157 | 1183 | 1171 | SLTSALLLQIRSVALSLVEHTVGSKSQ |
| 2387 | 2399 | 2393 | AGKVYFSPLVDKQ |
| 1459 | 1467 | 1465 | TFDLLVKLE |
| 465 | 484 | 471 | RWSCSAAVHLITMYLASAKD |
| 2752 | 2778 | 2769 | LHNIANALLPLIEALMVVCKHSKVREL |
| 1368 | 1387 | 1374 | GINLYIIALLLQTGKPHIHR |
| 2106 | 2117 | 2111 | ASPALSVLLDGL |
| 2964 | 2987 | 2970 | YDNCFLDCHFSRAVYKRILGKPQS |
| 76 | 85 | 81 | DNVKLSLVSP |
| 873 | 891 | 879 | IINVLLYSLTEIYINLGSL |
| 315 | 339 | 322 | EKLIQIKCLAVGFTCCMLSSQVTST |
| 561 | 596 | 574 | DSPLLESFNKVLTHPHVFGPSILAATIDSVFFIIHN |
| 2455 | 2465 | 2460 | AQKVYTQVCNK |
| 1011 | 1021 | 1017 | QDAVEILLSLG |
| 2922 | 2933 | 2927 | DYALFTPVVSDE |
| 344 | 356 | 350 | TEPYIFSFLVEAI |
| 1577 | 1587 | 1581 | DMAVGVICNNI |
| 1514 | 1534 | 1529 | RKTVIEALRTSVILIFRYCFE |
| 521 | 534 | 526 | KDAIVYYSITLRQA |
| 151 | 181 | 171 | FSAPKPVRNKVLEIAKSFPPLVPIDSTLKQL |
| 2547 | 2571 | 2559 | TDDQTLLDILARVFQVASRALHALK |
| 945 | 956 | 950 | SPPLQINVCDPS |
| 837 | 858 | 846 | TLKLDSVLSYIKYKGEVSYFET |
| 398 | 411 | 403 | SHGILFQCLRHIWK |
| 2505 | 2511 | 2507 | LKYFLLT |
| 825 | 833 | 828 | YGLPVIIEE |
| 3051 | 3063 | 3052 | VKKVVEYRLQTSV |
| 256 | 267 | 262 | TEGLHIFHLPEE |
| 1265 | 1274 | 1270 | GKFLRCVEID |
| 1706 | 1720 | 1712 | DLALIRRFVVDLMMK |
| 1801 | 1812 | 1807 | IDKVVKAALKPM |
| 2475 | 2499 | 2492 | QYKLPVGATQISVGIQIVEAVDYLL |
| 540 | 550 | 546 | LMKLVADLIQS |
| 1232 | 1242 | 1239 | PSDQFAYLISL |
| 3180 | 3199 | 3194 | PSSHTCFNQIDLPAYDCYET |
| 2289 | 2296 | 2292 | VFASYVRE |
| 897 | 903 | 899 | GQIVSLF |
| 2401 | 2415 | 2406 | IAALIRLLFSPLTIS |
| 361 | 384 | 366 | SSLVSRDVYFAAIRALECISFKKV |
| 2187 | 2195 | 2191 | DLVYPLTTT |
| 269 | 278 | 275 | VRKLTVQQLF |
| 650 | 661 | 655 | DKKLIQVFFKSF |
| 2417 | 2431 | 2423 | REHIYHALQYMCYNK |
| 688 | 704 | 698 | RHYTSLKPIILQQLSEL |
| 1557 | 1575 | 1562 | QIKDLHACLRESAPLVFRD |
| 1325 | 1345 | 1340 | QSFPKSISFISDFIMLVSYKE |
| 909 | 919 | 915 | LMNLVQLLERS |
| 1192 | 1214 | 1201 | DNVPTPLIEQIAVIIKDIFVGKK |
| 49 | 58 | 53 | DLIHWIPLLN |
| 1497 | 1511 | 1505 | GSQILKDLLKLVRTN |
| 1642 | 1662 | 1653 | KPRATALNFFIHQLIPTHSLE |
| 2594 | 2604 | 2599 | PHAPPPPVIPD |
| 1596 | 1603 | 1601 | ESYVSKIA |
| 3091 | 3104 | 3096 | LELLISGLPDIDVS |
| 438 | 445 | 444 | NKSLVPKL |
| 2723 | 2738 | 2729 | LTDLYKKLALGSLWDA |
| 1076 | 1096 | 1082 | FFKVAEATCISLWNKLLIMDP |
| 1061 | 1074 | 1066 | DQVYTPFAISLFQN |
| 224 | 234 | 228 | NYQYYKSVPNT |
| 1736 | 1752 | 1742 | YGKLLDLFELSGSLIST |
| 3071 | 3086 | 3080 | LIGFHEIIPKDLVAIF |
| 2606 | 2614 | 2612 | NYRLIIKIL |
| 283 | 296 | 294 | EVLPPESWFCFGIH |
| 1136 | 1149 | 1142 | IPNVPFAKFYFYQG |
| 2861 | 2881 | 2872 | MLVSVRRDQVFLDSYRSLFFK |
| 756 | 772 | 760 | AYLLDNVFNFLGGLLQD |
| 2174 | 2180 | 2179 | SDVLKMY |
| 2691 | 2703 | 2695 | QAKLLRILTALDY |
| 778 | 796 | 783 | TDVIQKISFKSWIKLLTLR |
| 197 | 211 | 207 | PSIIGDHYNFVYTLD |
| 2633 | 2649 | 2646 | NFSVLPNAQKLFSLELS |
| 2909 | 2916 | 2914 | EWYQVLSR |
| 2437 | 2448 | 2442 | IMSLMIAILNDC |
| 1755 | 1764 | 1759 | RDSVGPLLNK |
| 1624 | 1634 | 1630 | SKPLLEFLTFS |
| 861 | 871 | 868 | PQQATILLQDL |
| 1965 | 1973 | 1971 | IDDLDLVSQ |
| 1768 | 1794 | 1784 | QYDQFHISKIYIEKQVPNLLTNLIAEF |
| 2780 | 2788 | 2781 | IKDILKYEA |
| 600 | 612 | 611 | AFSILNEAKVIDT |
| 2668 | 2673 | 2669 | KELVAG |
| 423 | 434 | 428 | EGYIHFFNLIGN |
| 1098 | 1110 | 1106 | LASATLDLKYIST |
| 2945 | 2958 | 2952 | NPEHLSFFKFIGRI |
| 3162 | 3172 | 3167 | SGTCKFSIHRD |
| 3138 | 3144 | 3141 | ARLLQFA |
| 2533 | 2544 | 2543 | PINYMLKILDNK |
| 33 | 39 | 35 | LPQYLQE |
| 922 | 929 | 924 | EEAIIVSN |
| 2829 | 2840 | 2834 | GMLVRNPRVLEF |
| 2047 | 2053 | 2049 | RQRLFLD |
| 806 | 818 | 812 | NGIVSFMGILKYF |
| 1116 | 1131 | 1125 | IKNALGQILMIFAKTV |
| 2995 | 3001 | 2999 | DLEYFKS |
| 1249 | 1262 | 1260 | AAHFFEHGCNLSDI |
| 17 | 28 | 19 | PLRSLIDKLTTC |
| 2740 | 2750 | 2742 | SETLRVLEEKP |
| 3117 | 3123 | 3118 | PSSLQIQ |
| 711 | 722 | 714 | YVNQRLPGIEFY |
| 678 | 684 | 682 | NLGCSLD |
| 2798 | 2804 | 2800 | IESLFFS |
| 451 | 461 | 457 | LDDLMPFLNLP |
| 3201 | 3207 | 3206 | RGSLLMA |
| 2313 | 2321 | 2316 | PDFLDALPD |
| 1843 | 1849 | 1845 | PEDVDYH |
| 3147 | 3153 | 3149 | TSKVPLN |
| 1437 | 1442 | 1441 | ELPFTI |
| 495 | 500 | 497 | FTLLIG |
| 1917 | 1923 | 1918 | ELSAIDS |
| 2681 | 2687 | 2684 | FSKFSAH |
| 668 | 673 | 673 | KELVRT |
| 932 | 937 | 936 | DEVLKM |
| 3126 | 3131 | 3128 | WRAVKS |
| 1219 | 1224 | 1219 | SEFIPF |
| 1448 | 1453 | 1453 | FDVATK |

20. IPF1009
MSNNPHRTHKRQKSSVSNPGYYFTPETKSEIQQQQPQQQESQQQQQQQQQQHSQTHNIYDNDNYMNYNFPPT
SNRPRASTTTGTTSTTHPGSELSHESHSVHTSPLKRTASSELDQPIPAMAPSSPLVSSAPYYYQQPSQQQNL
SYHDHHHQQQQQQSTPQGQQLSQQTQSNSQSGVPPPLYGTSSSIPPGSTMQPSTSFAFHTSHSTYNPSFDSS
NLYNSAFRLPEYPTTSSSSLLSTTGGKQFQQSSALLPSGTLPPSILGTSSSSHVSALRQHQKNNLSISSHLT
LFSLSGNNSSQLQSQGSSFQQSETGVDDTKRSSKESTTIFNDLLFHLTSVDGSNINTFLLSILRKINSPFTL
DDFYNLLYNDRQRTLLDNSNYQNRIDKTIVSPSDTDMTVSIINQLLNFFKTPSMLVDYFPNMEDKDNKLANI
NYHELLRTFLAIKILHDILIQLPISEDDDPQNYTIPRLSIYKTYYIICQKLIASYPSASNTRNEQQKLILGQ
SKLGKLIKLVYPNLLIKRLGSRGESKYNYLGVMWNANIVQEEIKQLCDEHELNDLNEIFNSDNNNPFASIAP
SGSATTGSTPRRGLSHKRTSSKQKIKTEPVAGNPFLQPLQTSHHHHHSHHHHHEEQSQEESLSQQMGEHITA
PRLSFLRANSKYPTDVNLSVLDDDNWFVRLSYECYARQPALNRDLIQQIFLKNEFLLNNSSLLRNLMDSIIK
PLVMQETYSNVDLVLYLAILLEILPYLLLVKSSTNINLLKNLRLNLLHLINNFNNELKKLDSPKFPIERSTI
FLVLVKKLINLNDLLITFIKLINRDNCKTTMSSDIENFLKINSQTVKLDDDDNSFFFNLNTTSMGEVNFNFK
NEILSNDLIYTLIGYNFDPTTNSELKSSISMNFINEEINVIDEFFKNDLLNFLSTDFHAGLDDDNGEEDDDE
EAGPGSTHPMGATGSQGSLSPEPVSTGNPSVPPTRNTSISEVNAENKRGNEAVLTPKETAKLNSLISLIDKR
LLSSQFKSKYPILMYNNCISYILNDILKHIFLKQQQQQLQSSSSQLHDTQPLTQQDTAQGIGSSSSNANNTN
SSFGNWWVFNSFIQEYMSLIGELVGLHDNLV*

| Rank | Sequence | Start position | Score |
|---|---|---|---|
| 1 | GEHITAPRLSFLRANS | 643 | 0.94 |
| 2 | QQQQQHSQTHNIYDND | 47 | 0.93 |
| 3 | VKLDDDDNSFFFNLNT | 838 | 0.92 |
| 3 | SGSATTGSTPRRGLSH | 577 | 0.92 |
| 3 | THNIYDNDNYMNYNFP | 55 | 0.92 |
| 3 | PGSTMQPSTSFAFHTS | 190 | 0.92 |
| 4 | MDSIIKPLVMQETYSN | 715 | 0.91 |
| 4 | YNFPPTSNRPRASTTT | 67 | 0.91 |
| 4 | QLPISEDDDPQNYTIP | 453 | 0.91 |
| 4 | PGYYFTPETKSEIQQQ | 19 | 0.91 |
| 5 | PVSTGNPSVPPTRNTS | 959 | 0.90 |
| 6 | QKLIASYPSASNTRNE | 481 | 0.89 |
| 6 | LRKINSPFTLDDFYNL | 351 | 0.89 |
| 7 | NGEEDDDEEAGPGSTH | 929 | 0.88 |
| 7 | PSTSFAFHTSHSTYNP | 196 | 0.88 |
| 8 | GNEAVLTPKETAKLNS | 985 | 0.87 |
| 8 | NTSISEVNAENKRGNE | 972 | 0.87 |
| 8 | GSQGSLSPEPVSTGNP | 950 | 0.87 |
| 8 | DLLNFLSTDFHAGLDD | 912 | 0.87 |
| 8 | DKTIVSPSDTDMTVSI | 386 | 0.87 |
| 8 | FRLPEYPTTSSSSLLS | 223 | 0.87 |
| 8 | TAQGIGSSSSNANNTN | 1065 | 0.87 |
| 9 | PSVPPTRNTSISEVNA | 965 | 0.86 |
| 9 | DFHAGLDDDNGEEDDD | 920 | 0.86 |
| 9 | VRLSYECYARQPALNR | 676 | 0.86 |
| 9 | SMLVDYFPNMEDKDNK | 413 | 0.86 |
| 9 | SETGVDDTKRSSKEST | 310 | 0.86 |
| 9 | SSELDQPIPAMAPSSP | 111 | 0.86 |
| 9 | QFKSKYPILMYNNCIS | 1013 | 0.86 |
| 10 | TSHSTYNPSFDSSNLY | 204 | 0.85 |
| 10 | PIPAMAPSSPLVSSAP | 117 | 0.85 |
| 11 | PGSELSHESHSVHTSP | 90 | 0.84 |
| 11 | LSVLDDDNWFVRLSYE | 666 | 0.84 |
| 11 | GESKYNYLGVMWNANI | 527 | 0.84 |
| 11 | QSNSQSGVPPPLYGTS | 170 | 0.84 |
| 11 | PLVSSAPYYYQQPSQQ | 126 | 0.84 |
| 12 | NNSSLLRNLMDSIIKP | 706 | 0.83 |
| 12 | QKIKTEPVAGNPFLQP | 599 | 0.83 |
| 13 | LVMQETYSNVDLVLYL | 722 | 0.82 |
| 13 | STTIFNDLLFHLTSVD | 324 | 0.82 |
| 13 | DSSNLYNSAFRLPEYP | 214 | 0.82 |
| 14 | YGTSSSIPPGSTMQPS | 182 | 0.81 |
| 15 | NEEINVIDEFFKNDLL | 899 | 0.80 |
| 15 | HELNDLNEIFNSDNNN | 554 | 0.80 |
| 16 | TTGTTSTTHPGSELSH | 81 | 0.79 |
| 16 | CYARQPALNRDLIQQI | 682 | 0.79 |
| 16 | SLSQQMGEHITAPRLS | 637 | 0.79 |
| 16 | NPHRTHKRQKSSVSNP | 4 | 0.79 |
| 16 | SSSSQLHDTQPLTQQD | 1049 | 0.79 |
| 17 | FASIAPSGSATTGSTP | 571 | 0.78 |
| 17 | SSSSNANNTNSSFGNW | 1071 | 0.78 |
| 18 | KFPIERSTIFLVLVKK | 784 | 0.77 |
| 18 | IFNSDNNNPFASIAPS | 562 | 0.77 |
| 18 | GVMWNANIVQEEIKQL | 535 | 0.77 |
| 19 | IGYNFDPTTNSELKSS | 877 | 0.76 |
| 19 | NDLIYTLIGYNFDPTT | 870 | 0.76 |
| 19 | ELKKLDSPKFPIERST | 776 | 0.76 |
| 19 | QLSQQTQSNSQSGVPP | 164 | 0.76 |
| 20 | HESHSVHTSPLKRTAS | 96 | 0.75 |
| 20 | NSELKSSISMNFINEE | 886 | 0.75 |
| 20 | HHHEEQSQEESLSQQM | 627 | 0.75 |
| 20 | HHHHHSHHHHHEEQSQ | 619 | 0.75 |
| 20 | SEIQQQQPQQQESQQQ | 29 | 0.75 |

| Start | End | Max_score_pos | Sequence |
|---|---|---|---|
| 730 | 753 | 747 | NVDLVLYLAILLEILPYLLLVKSS |
| 789 | 813 | 795 | RSTIFLVLVKKLINLNDLLITFIKL |
| 507 | 522 | 513 | LGKLIKLVYPNLLIKR |
| 467 | 489 | 480 | IPRLSIYKTYYIICQKLIASYPS |
| 434 | 457 | 453 | YHELLRTFLAIKILHDILIQLPIS |
| 759 | 770 | 767 | LKNLRLNLLHLI |
| 329 | 338 | 335 | NDLLFHLTSV |
| 113 | 139 | 130 | ELDQPIPAMAPSSPLVSSAPYYYQQPS |
| 716 | 726 | 720 | DSIIKPLVMQE |
| 675 | 687 | 679 | FVRLSYECYARQP |
| 998 | 1063 | 1037 | LNSLISLIDKRLLSSQFKSKYPILMYNNCISYILNDILKHI |
| | | | FLKQQQQQLQSSSSQLHDTQPLTQQ |
| 694 | 702 | 696 | IQQIFLKNE |
| 661 | 670 | 666 | PTDVNLSVLD |
| 345 | 354 | 348 | TFLLSILRKI |
| 283 | 292 | 289 | ISSHLTLFSL |
| 174 | 190 | 179 | QSGVPPPLYGTSSSIPP |
| 267 | 275 | 270 | SSHVSALRQ |
| 1093 | 1108 | 1101 | IQEYMSLIGELVGLHD |
| 607 | 630 | 613 | AGNPFLQPLQTSHHHHHSHHHHHE |
| 869 | 879 | 875 | SNDLIYTLIGY |
| 398 | 409 | 402 | TVSIINQLLNFF |
| 95 | 105 | 103 | SHESHSVHTSP |
| 246 | 265 | 259 | QQSSALLPSGTLPPSILGTS |
| 412 | 419 | 417 | PSMLVDYF |
| 953 | 960 | 957 | GSLSPEPV |
| 646 | 655 | 651 | ITAPRLSFLR |
| 141 | 155 | 147 | QQNLSYHDHHHQQQQ |
| 541 | 554 | 553 | NIVQEEIKQLCDEH |
| 233 | 239 | 235 | SSSLLST |
| 531 | 537 | 533 | YNYLGVM |
| 572 | 577 | 573 | ASIAPS |
| 499 | 505 | 500 | KLILGQS |
| 387 | 393 | 390 | KTIVSPS |
| 987 | 992 | 990 | EAVLTP |
| 13 | 24 | 19 | KSSVSNPGYYFT |
| 198 | 206 | 203 | TSFAFHTSH |
| 962 | 969 | 968 | TGNPSVPP |
| 833 | 841 | 838 | INSQTVKLD |
| 219 | 228 | 225 | YNSAFRLPEY |
| 299 | 308 | 300 | QLQSQGSSFQ |
| 162 | 168 | 163 | GQQLSQQ |
| 44 | 53 | 49 | QQQQQQQQHS |

21. IPF2971
MTSTITKTNNSITRSFEDDKFLLPQLKSLNKTWIFSEDAVINNSPTRHQKLTISQELKNKESMHDFLIRLGQ
KLKVDGRTILAATIYLHRFYMRVPISQSKYYVVSAALTISCKLNDNYRTPDKVALLSCNVKLPPNAKPIDEQ
SEMYWRWKDQLLFREELMLRKLNFDLNLTLPYEIRDHIFKNFMLLDQEDESVKLFSTHKLDILKMTTSLIES
LSSLPVILCYEMNIMFGTCLIITILEGKKIIDEKLNIPTAFLYRFLDTDSETCLKCFHFIKNLLKFSQDDPH
IISNKASAKRLLDIRSRTFHEIAKQGDLKQPQPQPQPQPQQHENETTTKEKKPEDNQGEGNNATEKIENGHT
TNSTNTDPKSQDNKVDVIEKKEAKEINKSETQDDHTQERSTIAAENKVPDTESNVTKSEITKSNNEIMAEKN
PDVKNSNSNSDDTGYTSNQLEQGKDTKNEELEKILDSEKISTPNNGTTTDKPAADVHDSTNGTNENSIGEKR
VLEQDSNDTNVDSPSSKIAKVE*

| Rank | Sequence | Start position | Score |
|---|---|---|---|
| 1 | DAVINNSPTRHQKLTI | 38 | 0.95 |
| 2 | NSTNTDPKSQDNKVDV | 362 | 0.94 |
| 2 | TCLIITILEGKKIIDE | 234 | 0.94 |
| 2 | AKPIDEQSEMYWRWKD | 138 | 0.94 |
| 3 | CKLNDNYRTPDKVALL | 113 | 0.93 |
| 4 | SEKISTPNNGTTTDKP | 469 | 0.92 |
| 5 | NGTTTDKPAADVHDST | 477 | 0.89 |
| 5 | NNEIMAEKNPDVKNSN | 424 | 0.89 |
| 5 | HENETTTKEKKPEDNQ | 330 | 0.89 |
| 5 | AALTISCKLNDNYRTP | 107 | 0.89 |
| 6 | SETQDDHTQERSTIAA | 389 | 0.88 |
| 6 | YWRWKDQLLFREELML | 148 | 0.88 |
| 7 | DVKNSNSNSDDTGYTS | 434 | 0.87 |
| 7 | PYEIRDHIFKNFMLLD | 175 | 0.87 |
| 8 | NNSITRSFEDDKFLLP | 9 | 0.86 |
| 8 | SEITKSNNEIMAEKNP | 418 | 0.86 |
| 9 | TESNVTKSEITKSNNE | 411 | 0.85 |
| 10 | NEELEKILDSEKISTP | 460 | 0.84 |
| 10 | DDTGYTSNQLEQGKDT | 443 | 0.84 |
| 10 | PQPQPQPQQHENETTT | 321 | 0.84 |
| 10 | NKTWIFSEDAVINNSP | 30 | 0.84 |
| 10 | SLPVILCYEMNIMFGT | 219 | 0.84 |
| 11 | AKEINKSETQDDHTQE | 383 | 0.83 |
| 12 | EKKPEDNQGEGNNATE | 338 | 0.81 |
| 13 | PISQSKYYVVSAALTI | 96 | 0.80 |
| 13 | RVLEQDSNDTNVDSPS | 504 | 0.80 |
| 14 | GRTILAATIYLHRFYM | 78 | 0.79 |
| 14 | TSTITKTNNSITRSFE | 2 | 0.79 |
| 15 | KVDVIEKKEAKEINKS | 374 | 0.78 |
| 15 | TEKIENGHTTNSTNTD | 352 | 0.78 |
| 15 | DPHIISNKASAKRLLD | 286 | 0.78 |
| 15 | EELMLRKLNFDLNLTL | 159 | 0.78 |
| 16 | NKESMHDFLIRLGQKL | 59 | 0.77 |
| 16 | NQLEQGKDTKNEELEK | 450 | 0.77 |
| 16 | NLLKFSQDDPHIISNK | 278 | 0.77 |
| 16 | TSLIESLSSLPVILCY | 211 | 0.77 |
| 17 | HEIAKQGDLKQPQPQP | 308 | 0.75 |

| Start | End | Max_score_pos | Sequence |
|---|---|---|---|
| 213 | 228 | 223 | LIESLSSLPVILCYEM |
| 81 | 116 | 106 | ILAATIYLHRFYMRVPISQSKYYVVSAALTISCKLN |
| 122 | 138 | 127 | PDKVALLSCNVKLPPNA |
| 267 | 283 | 272 | ETCLKCFHFIKNLLKFS |
| 232 | 241 | 238 | FGTCLIITIL |
| 20 | 29 | 23 | KFLLPQLKSL |
| 253 | 262 | 259 | IPTAFLYRFL |
| 195 | 207 | 199 | SVKLFSTHKLDIL |
| 171 | 181 | 173 | NLTLPYEIRDH |
| 64 | 77 | 73 | HDFLIRLGQKLKVD |
| 516 | 523 | 522 | DSPSSKIA |
| 296 | 302 | 297 | AKRLLDI |
| 50 | 55 | 54 | KLTISQ |
| 153 | 158 | 158 | DQLLFR |
| 316 | 328 | 319 | LKQPQPQPQPQPQ |
| 502 | 508 | 503 | EKRVLEQ |
| 285 | 293 | 287 | DDPHIISNK |

22. IPF1798
MTPSSTKKIKQRRSTSCTVCRTIKRKCDGNTPCSNCLKRNQECIYPDVDKRKKRYSIEYITNLENTNQQLHD
QLQSLIDLKDNPYQLHLKITEILESSSSFLDNSETKSDSSLGSPELSKSEASLANSFTLGGELVVSSREQGA
NFHVHLNQQQQQQQPSPQSLSQSSASEVSTRSSPASPNSTISLAPQILRIPSRPFQQQTRQNLLRQSDLPLH
YPISGKTSGPNASNITGSIASTISGSRKSSISVDISPPPSLPVFPTSGPTLPTLLPEPLPRNDFDFAPKFFP
APGGKSNMAFGATTVYDADESMVMNVNQIEERWGTGIKLAKLRNVPNIQNRSSSSSSTLIKVNKRTIEEVIK
MITNSKAKKYFALAFKYFDRPILCYLIPRGKVIKLYEEICAHKNDLATVEDILGLYPTNQFISIELIAALIA
SGALYDDNIDCVREYLTLSKTEMFINNSGCLVFNESSYPKLQAMLVCALLELGLGELTTAWELSGIALRMGI
DLGFDSFIYDDSDKEIDNLRNLVFWGSYIIDKYAGLIFGRITMLYVDNSVPLIFLPNRQGKLPCLAQLIIDT
QPMISSIYETIPETKNDPEMSKKIFLERYNLLQGYNKSLGAWKRGLSREYFWNKSILINTITDESVDHSLKI
AYYLIFLIMNKPFLKLPIGSDIDTFIEIVDEMEIIMRYIPDDKHLLNLVVYYALVLMIQSLVAQVSYTNANN
YTQNSKFMNQLLFFIDRMGEVLRVDIWLICKKVHSNFQQKVEYLEKLMLDLTEKMEQRRRDEENLMMQQEEF
YAQQQQQQQQQQQQPKHEYHDHQQEQEQQEQLQEEHSEKDIKIEIKDEPQPQEEHIHQDYPMKEEEENLNQL
SEPQTNEEDNPAEDMLQNEQFMRMVDILFIRGIENDQEEGEEQQQQQQQQEQVQQEQVQQEQVQQDQMELEE
DELPQQMPTPPEQPDEPEIPQLPEILDPTFFNSIVDNNGSTFNNIFSFDTEGFRL*

| Rank | Sequence | Start position | Score |
|---|---|---|---|
| 1 | LPEILDFTFFNSIVDN | 958 | 0.97 |
| 2 | AGLIFGRITMLYVDNS | 538 | 0.96 |
| 3 | GKVIKLYEEICAHKND | 390 | 0.94 |
| 4 | QECIYPDVDKRKKPRY | 41 | 0.93 |
| 5 | MEIIMRYIPDDKHLLN | 680 | 0.92 |
| 5 | TTVYDADESMVMNVNQ | 301 | 0.92 |
| 5 | ASTISGSRKSSISVDI | 236 | 0.92 |
| 5 | CRTIKRKCDGNTPCSN | 20 | 0.92 |
| 6 | IWLICKKVHSNFQQKV | 746 | 0.91 |
| 7 | IRGIENDQEEGEEQQQ | 894 | 0.89 |
| 7 | HIHQDYPMKEEEENLN | 847 | 0.89 |
| 7 | YEEICAHKNDLATVED | 396 | 0.89 |
| 8 | LPIGSDIDTFIEIVDE | 664 | 0.88 |
| 8 | AALIASGALYDDNIDC | 428 | 0.88 |
| 9 | HYPISGKTSGPNASNI | 216 | 0.87 |
| 10 | KCDGNTPCSNCLKRNQ | 26 | 0.86 |
| 11 | PTPPEQPDEPEIPQLP | 944 | 0.85 |
| 11 | ELPQQMPTPPEQPDEP | 938 | 0.85 |
| 11 | EEVIKMITNSKAKKYF | 356 | 0.85 |
| 12 | EERWGTGIKLAKLRNV | 318 | 0.84 |
| 13 | KMEQRRRDEENLMMQQ | 774 | 0.83 |
| 13 | VLMIQSLVAQVSYTNA | 703 | 0.83 |
| 13 | SSREQGANFHVHLNQQ | 138 | 0.83 |
| 14 | EQLQEEHSEKDIKIEI | 822 | 0.82 |
| 14 | VAQVSYTNANNYTQNS | 710 | 0.82 |
| 14 | AQLIIDTQPMISSIYE | 570 | 0.82 |
| 15 | QMELEEDELPQQMPTP | 931 | 0.81 |
| 15 | LGAWKRGLSREYFWNK | 615 | 0.81 |
| 15 | ISSIYETIPETKNDPE | 580 | 0.81 |
| 15 | VPLIFLPNRQGKLPCL | 554 | 0.81 |
| 15 | DVDKRKKRYSIEYITN | 47 | 0.81 |
| 15 | DRPILCYLIPRGKVIK | 379 | 0.81 |
| 15 | FFAPGGKSNMAFGATT | 287 | 0.81 |
| 15 | QSSASEVSTRSSPASP | 166 | 0.81 |
| 16 | PQPQEEHIHQDYPMKE | 841 | 0.80 |
| 16 | SSSSSSTLIKVNKRTI | 340 | 0.80 |
| 17 | WELSGIALRMGIDLGF | 493 | 0.79 |
| 17 | SISVDISPPPSLPVFP | 246 | 0.79 |
| 17 | PQILRIPSRPFQQQTR | 189 | 0.79 |
| 18 | SIVDNNGSTFNNIFSF | 969 | 0.78 |
| 18 | GFDSFIYDDSDKEIDN | 507 | 0.78 |
| 19 | KRYSIEYITNLENTNQ | 53 | 0.77 |
| 19 | ESMVMNVNQIEERWGT | 308 | 0.77 |
| 19 | EPLPRNDFDFAPKFFP | 273 | 0.77 |
| 19 | LRQSDLPLHYPISGKT | 208 | 0.77 |
| 20 | PQTNEEDNPAEDMLQN | 867 | 0.76 |
| 20 | KIEIKDEPQPQEEHIH | 834 | 0.76 |
| 20 | LRMGIDLGFDSFIYDD | 500 | 0.76 |
| 20 | SPPPSLPVFPTSGPTL | 252 | 0.76 |
| 21 | QQQQPKHEYHDHQQEQ | 803 | 0.75 |
| 21 | IPETKNDPEMSKKIFL | 587 | 0.75 |
| 21 | REYLTLSKTEMFINNS | 445 | 0.75 |
| 21 | HKNDLATVEDILGLYP | 402 | 0.75 |
| 21 | DSSLGSPELSKSEASL | 110 | 0.75 |

| Start | End | Max_score_pos | Sequence |
|---|---|---|---|
| 689 | 716 | 700 | DDKHLLNLVVYYALIVLMIQSLVAQVSYT |
| 470 | 487 | 480 | YPKLQAMLVCALLELGLG |
| 368 | 404 | 384 | KKYFALAFKYFDRPILCYLIPRGKVIKLYEEICAHKN |
| 565 | 575 | 569 | KLPCLAQLIID |
| 546 | 560 | 557 | TMLYVDNSVPLIFLP |
| 642 | 659 | 653 | VDHSLKIAYYLIFLIMNK |
| 741 | 756 | 751 | VLRVDIWLICKKVHSN |
| 16 | 26 | 20 | SCTVCRTIKRK |
| 207 | 220 | 216 | LLRQSDLPLHYPIS |
| 406 | 436 | 429 | LATVEDILGLYPTNQFISIELIAALIASGAL |
| 245 | 262 | 257 | SSISVDISPPPSLPVFPT |
| 33 | 48 | 46 | CSNCLKRNQECIYPDV |
| 132 | 140 | 134 | GGELVVSSR |
| 83 | 102 | 88 | NPYQLHLKITEILESSSSFL |
| 440 | 451 | 446 | NIDCVREYLTLS |
| 184 | 199 | 189 | TISLAPQILRIPSRPF |
| 661 | 667 | 663 | FLKLPIG |
| 459 | 467 | 462 | NSGCLVFNE |
| 70 | 80 | 77 | LHDQLQSLIDL |
| 264 | 274 | 273 | GPTLPTLLPEP |
| 145 | 153 | 147 | NFHVHLNQQ |
| 888 | 895 | 892 | MVDILFIR |
| 729 | 735 | 733 | NQLLFFI |
| 914 | 929 | 919 | QEQVQQEQVQQEQVQQ |
| 758 | 770 | 764 | QQKVEYLEKLMLD |
| 524 | 543 | 529 | RNLVFWGSYIIDKYAGLIFG |
| 344 | 350 | 347 | SSTLIKV |
| 955 | 965 | 959 | IPQLPEILDPT |
| 325 | 334 | 330 | IKLAKLRNVP |
| 599 | 612 | 608 | KIFLERYNLLQGYN |
| 155 | 167 | 161 | QQQQPSPQSLSQS |
| 629 | 636 | 634 | NKSILINT |
| 283 | 289 | 285 | APKFFPA |
| 300 | 306 | 301 | ATTVYDA |
| 580 | 586 | 581 | ISSIYET |
| 791 | 806 | 795 | EFYAQQQQQQQQQQQQ |
| 121 | 128 | 128 | SEASLANS |
| 169 | 175 | 169 | ASEVSTR |
| 507 | 514 | 509 | GFDSFIYD |
| 808 | 814 | 809 | KHEYHDH |
| 356 | 361 | 357 | EEVIKM |

23. IPF4805
MSTVPQVTQDDYTETLKVWRSFKVAQLKDVCRSLELNVGGRKQDLVDRGEAFLSSKFNNNDQIGFHAAKSLI
FMRLQGDPLPSYRDMHYAIRTGRFKLTAPTLIGTSSSTNQLSNIHSGDSKPYKGHTLYFKATPLYRFLRLIH
STPMLLIPNGRNSVQTCHFIFTEEEYKFLQDKPPHIKLYILCGIPDMQRSNATNNVSIEYPVETVIYFNKHE
FKDTFRGISGETNTAVPVDITKYINSPPQRNEIVFCHSANNAGYMMYLYLVEVIPAERLIEQVQNRPAIPKS
ETIRNIKDMSRYDGIQTTKLPLRDPLSYTKLANPTKSVHCDHYMCFNGMLFIEQQRLVDEWKCPVCSREIKF
EDLRISEYFEEIIKNVGPDVDEIIIMQDGSWKPVVGDDTNTTKKRTESASPEAIILLSDDEDDVSADEVDAN
VHLENKEDESVNINRVDNTPEAEVDITESNNNQETQIDNESIQSDDLTEYLIDHEHQQHEEASDKVEDNNPT
LPSQQSPQAETNNDETSNMKTTLQEDTSAPLPKNGVQENDAPLGDAIDTEQNLREDQTAEAVDPADSPISVI
NVTLDPPNEANKENSTESSISLSNLSPKNRESSLSSSSPQSVPPSMITPISPMSAQASSDKAQVLHGDSNTS
NQPRYTSSPDSAASPLSLTGPNDINEENRTPQSHNLNNTDALHGQDSSNSQAVSNSRSIQSVPTAAVSSKNQ
GNQHSDDQIRSLNEEIKKLRQALYYRDLYIKRLPLNQQHALLQQQQQQQLQLQQLSQQRPNDHQIHHFQQQQ
LLQQQQQRQLRQLEQQQRLQRQQWQQQQQLLQQQQQQLPRQLPLQSPQQLPQQQHLSPEDQSQFLQLTQLPQ
FRRLQQLQSQQRQSQQRQANPHQQHQQNYLQQPQHNNAQLHLNRNSHQLPQQQQQQQHHHYQQQQQQQQQQQ
RLQLPQQLRSSPQNRSFQTQPNEQQRVNSLSRSTDATPLSRQIVLDFGTSVPLPTQSSRPTLEEQGSFNLNL
LRHTPNLQTVASHSPWSPVATEPKNRSFSDSNIAVVNGLNESDPIEDRPLASLSGRSKSTNNMSNHNTSNVS
SSNYQVSEKQQQVHRLQSINANSNKKEDTEVETTTDTNLQQGSSAENTTDEQNSFRCNVQKPMLAIKNSDPS
QNHGQVEDTQTKNSINGTSTGVGGTAVFEGNSKLQTNSVQSHSNSPRKEQNSTSVVPNGNPQQIVIGNPSSM
VEKKDNGIDYNRNTFQIEDSVVNFVGRDTVNRIIGLTNLKDIQKVVENINNRKIVIDSNVEADRNRKRMILQ
KFDFDTKSLISDLMKNGGSNYEKSKLINTRRGEKSRLATHWDDKLEKNLSKYNAELQKLDKTLMLLRKQAES
IEARDAQIGGQSLNSTPVDNANSRGTPSSETDTAAGQSPKKNHLVNIPPQTNQSASQNETLQLSSIVTPSNR
DSSNKIRSGPPLQTSQVPTSKRQRVIGMLGIELNEDALKISDRKHGLQNSVTPINNKIKNISLGASNTNNCK
DRLGDLQTQFSLNQNITSGTMHDPIVLDMSDEE*

| Rank | Sequence | Start position | Score |
|---|---|---|---|
| 1 | HGDSNTSNQPRYTSSP | 642 | 0.94 |
| 1 | KLPLRDPLSYTKLANP | 307 | 0.94 |
| 1 | NSVQSHSNSPRKEQNS | 1189 | 0.94 |
| 2 | NEENRTPQSHNLNNTD | 673 | 0.93 |
| 3 | SNQPRYTSSPDSAASP | 648 | 0.91 |
| 3 | ISVINVTLDPPNEANK | 573 | 0.91 |
| 3 | TKYINSPPQRNEIVFC | 237 | 0.91 |
| 3 | VETVIYFNKHEFKDTF | 206 | 0.91 |
| 3 | LCGIPDMQRSNATNNV | 185 | 0.91 |
| 3 | RGTPSSETDTAAGQSP | 1392 | 0.91 |
| 3 | PRKEQNSTSVVPNGNP | 1198 | 0.91 |
| 4 | PSMITPISPMSAQASS | 620 | 0.90 |
| 4 | HEEASDKVEDNNPTLP | 491 | 0.90 |
| 4 | ATHWDDKLEKNLSKYN | 1334 | 0.90 |
| 5 | SSPQSVPPSMITPISP | 613 | 0.89 |
| 5 | GVQENDAPLGDAIDTE | 539 | 0.89 |
| 6 | PQVTQDDYTETLKVWR | 5 | 0.88 |
| 6 | VDEWKCPVCSREIKFE | 346 | 0.88 |
| 7 | QIVIGNPSSMVEKKDN | 1215 | 0.87 |
| 7 | SGRSKSTNNMSNHNTS | 1062 | 0.87 |
| 8 | SRSTDATPLSRQIVLD | 967 | 0.86 |
| 8 | PSYRDMHYAIRTGRFK | 82 | 0.86 |
| 8 | LQEDTSAPLPKNGVQE | 527 | 0.86 |
| 8 | PEAEVDITESNNNQET | 452 | 0.86 |
| 8 | LRISEYFEEIIKNVGP | 363 | 0.86 |
| 8 | IRNIKDMSRYDGIQTT | 291 | 0.86 |
| 8 | CHFIFTEEEYKFLQDK | 161 | 0.86 |
| 8 | SKLINTRRGEKSRLAT | 1320 | 0.86 |
| 8 | IHSGDSKPYKGHTLYF | 116 | 0.86 |
| 8 | ENTTDEQNSFRCNVQK | 1126 | 0.86 |
| 9 | VDRGEAFLSSKFNNND | 46 | 0.85 |
| 9 | QDGSWKPVVGDDTNTT | 387 | 0.85 |
| 9 | RPAIPKSETIRNIKDM | 282 | 0.85 |
| 9 | NVSIEYPVETVIYFNK | 199 | 0.85 |
| 9 | KSLISDLMKNGGSNYE | 1303 | 0.85 |
| 9 | KIVIDSNVEADRNRKR | 1277 | 0.85 |
| 9 | SMVEKKDNGIDYNRNT | 1223 | 0.85 |
| 9 | LQTVASHSPWSPVATE | 1015 | 0.85 |
| 10 | KRTESASPEAIILLSD | 404 | 0.84 |
| 10 | RGISGETNTAVPVDIT | 222 | 0.84 |
| 10 | DRLGDLQTQFSLNQNI | 1513 | 0.84 |
| 10 | TFQIEDSVVNFVGRDT | 1238 | 0.84 |
| 10 | TGVGGTAVFEGNSKLQ | 1172 | 0.84 |
| 10 | HGQVEDTQTKNSINGT | 1155 | 0.84 |
| 10 | SFRCNVQKPMLAIKNS | 1134 | 0.84 |
| 11 | QSVPTAAVSSKNQGNQ | 708 | 0.83 |
| 11 | LGDAIDTEQNLREDQT | 547 | 0.83 |
| 11 | PVVGDDTNTTKKRTES | 393 | 0.83 |
| 11 | SVHCDHYMCFNGMLFI | 325 | 0.83 |
| 11 | TPMLLIPNGRNSVQTC | 146 | 0.83 |
| 11 | LSSIVTPSNRDSSNKI | 1431 | 0.83 |
| 11 | DTAAGQSPKKNHLVNI | 1400 | 0.83 |
| 11 | EDRPLASLSGRSKSTN | 1054 | 0.83 |
| 12 | VNINRVDNTPEAEVDI | 443 | 0.82 |
| 12 | QSLNSTPVDNANSRGT | 1379 | 0.82 |
| 12 | AESIEARDAQIGGQSL | 1366 | 0.82 |
| 12 | NGLNESDPIEDRPLAS | 1045 | 0.82 |
| 12 | RSFSDSNIAVVNGLNE | 1034 | 0.82 |
| 12 | PTLIGTSSSTNQLSNI | 101 | 0.82 |
| 13 | NRSFQTQPNEQQRVNS | 950 | 0.81 |
| 13 | AEAVDPADSPISVINV | 563 | 0.81 |
| 13 | FEEIIKNVGPDVDEII | 369 | 0.81 |
| 13 | MLFIEQQRLVDEWKCP | 337 | 0.81 |
| 13 | SASQNETLQLSSIVTP | 1422 | 0.81 |
| 13 | HSPWSPVATEPKNRSF | 1021 | 0.81 |
| 14 | HNNAQLHLNRNSHQLP | 899 | 0.80 |
| 14 | LPSQQSPQAETNNDET | 505 | 0.80 |
| 14 | PVCSREIKFEDLRISE | 352 | 0.80 |
| 14 | DGIQTTKLPLRDPLSY | 301 | 0.80 |
| 14 | SGTMHDPIVLDMSDEE | 1530 | 0.80 |
| 14 | KHGLQNSVTPINNKIK | 1484 | 0.80 |
| 14 | TLYFKATPLYRFLRLI | 128 | 0.80 |
| 15 | TQSSRPTLEEQGSFNL | 991 | 0.79 |
| 15 | LREDQTAEAVDPADSP | 557 | 0.79 |
| 15 | AIILLSDDEDDVSADE | 413 | 0.79 |
| 15 | CHSANNAGYMMYLYLV | 252 | 0.79 |
| 15 | NRKRMILQKFDFDTKS | 1289 | 0.79 |
| 15 | KNSINGTSTGVGGTAV | 1164 | 0.79 |
| 16 | KSLIFMRLQGDPLPSY | 69 | 0.78 |
| 16 | SAASPLSLTGPNDINE | 659 | 0.78 |
| 16 | PQAETNNDETSNMKTT | 511 | 0.78 |
| 16 | ENKEDESVNINRVDNT | 436 | 0.78 |
| 16 | ADEVDANVHLENKEDE | 426 | 0.78 |
| 16 | GPPLQTSQVPTSKRQR | 1449 | 0.78 |
| 16 | DYTETLKVWRSFKVAQ | 11 | 0.78 |
| 17 | EQQRVNSLSRSTDATP | 959 | 0.77 |
| 17 | QQLRSSPQNRSFQTQP | 942 | 0.77 |
| 17 | KKLRQALYYRDLYIKR | 737 | 0.77 |
| 17 | ALHGQDSSNSQAVSNS | 689 | 0.77 |
| 18 | DDQIRSLNEEIKKLRQ | 726 | 0.76 |
| 18 | GGRKQDLVDRGEAFLS | 39 | 0.76 |
| 18 | FNKHEFKDTFRGISGE | 212 | 0.76 |
| 18 | HLVNIPPQTNQSASQN | 1411 | 0.76 |
| 18 | NVSSSNYQVSEKQQQV | 1078 | 0.76 |
| 19 | LKDIQKVVENINNRKI | 1263 | 0.75 |
| 19 | NFVGRDTVNRIIGLTN | 1247 | 0.75 |
| 19 | NGIDYNRNTFQIEDSV | 1230 | 0.75 |
| 19 | TEVETTTDTNLQQGSS | 1109 | 0.75 |

| Start | End | Max_score_pos | Sequence |
|---|---|---|---|
| 261 | 279 | 266 | MMYLYLVEVIPAERLIEQV |
| 171 | 189 | 183 | KFLQDKPPHIKLYILCGIP |
| 247 | 255 | 252 | NEIVFCHSA |
| 339 | 357 | 352 | FIEQQRLVDEWKCPVCSRE |
| 1428 | 1437 | 1432 | TLQLSSIVTP |
| 158 | 165 | 161 | VQTCHFIF |
| 322 | 333 | 328 | PTKSVHCDHYMC |
| 15 | 37 | 33 | TLKVWRSFKVAQLKDVCRSLELN |
| 201 | 214 | 203 | SIEYPVETVIYFNK |
| 972 | 992 | 978 | ATPLSRQIVLDFGTSVPLPTQ |
| 563 | 581 | 575 | AEAVDPADSPISVINVTLD |
| 1242 | 1249 | 1247 | EDSVVNFV |
| 1040 | 1046 | 1044 | NIAVVNG |
| 707 | 718 | 712 | IQSVPTAAVSSK |
| 935 | 947 | 941 | QQRLQLPQQLRSS |
| 430 | 436 | 432 | DANVHLE |
| 231 | 237 | 233 | AVPVDIT |
| 738 | 778 | 772 | KLRQALYYRDLYIKRLPLNQQHALLQQQQQQQLQLQQLSQQ |
| 1409 | 1416 | 1414 | KNHLVNIP |
| 1081 | 1099 | 1096 | SSNYQVSEKQQQVHRLQSI |
| 411 | 418 | 414 | PEAIILLS |
| 854 | 874 | 859 | SQFLQLTQLPQFRRLQQLQSQ |
| 819 | 851 | 833 | QQQLLQQQQQQLPRQLPLQSPQQLPQQQHLSPE |
| 126 | 152 | 139 | GHTLYFKATPLYRFLRLIHSTPMLLIP |
| 1014 | 1029 | 1018 | NLQTVASHSPWSPVAT |
| 1276 | 1283 | 1281 | RKIVIDSN |
| 658 | 667 | 663 | DSAASPLSLT |
| 1535 | 1541 | 1537 | DPIVLDM |
| 4 | 10 | 6 | VPQVTQD |
| 1266 | 1272 | 1267 | IQKVVEN |
| 1214 | 1220 | 1216 | QQIVIGN |
| 783 | 797 | 791 | HQIHHFQQQQLLQQQ |
| 1449 | 1460 | 1454 | GPPLQTSQVPTS |
| 885 | 897 | 896 | PHQQHQQNYLQQP |
| 637 | 643 | 640 | KAQVLHG |
| 305 | 319 | 311 | TTKLPLRDPLSYTKL |
| 609 | 633 | 617 | SLSSSSPQSVPPSMITPISPMSAQA |
| 98 | 106 | 101 | LTAPTLIGT |
| 64 | 75 | 69 | GFHAAKSLIFMR |
| 1136 | 1147 | 1137 | RCNVQKPMLAIK |
| 1517 | 1523 | 1521 | DLQTQFS |
| 1205 | 1210 | 1206 | TSVVPN |
| 391 | 397 | 393 | WKPVVGD |
| 911 | 933 | 922 | HQLPQQQQQQQHHHYQQQQQQQQ |
| 480 | 489 | 485 | TEYLIDHEHQ |
| 1303 | 1308 | 1306 | KSLISD |
| 595 | 601 | 598 | SISLSNL |
| 505 | 511 | 508 | LPSQQSP |
| 373 | 386 | 382 | IKNVGPDVDEIIIM |
| 1488 | 1493 | 1488 | QNSVTP |
| 1005 | 1012 | 1009 | NLNLLRHT |
| 1358 | 1364 | 1362 | TLMLLRK |
| 78 | 84 | 79 | GDPLPSY |
| 1188 | 1194 | 1189 | TNSVQSH |
| 532 | 538 | 537 | SAPLPKN |
| 901 | 907 | 905 | NAQLHLN |
| 799 | 812 | 802 | QRQLRQLEQQQRLQ |
| 698 | 704 | 698 | SQAVSNS |
| 1348 | 1356 | 1353 | YNAELQKLD |
| 86 | 91 | 91 | DMHYAI |

24. RBT4
MKFSQVATTAAIFAGLTTAEIAYVTQTRGVTVGETATVATTVTVGATVTGGDQGQDQVQQSAAPEAGDIQQS
AVPEADDIQQSAVPEAEPTADADGGNGIAITEVFTTTIMGQEIVYSGVYYSYGEEHTYGDVQVQTLTIGGGG
FPSDDQYPTTEVSAEASPSAVTTSSAVATPDAKVPDSTKDASQPAATTASGSSSGSNDFSGVKDTQFAQQIL
DAHNKKRARHGVPDLTWDATGYEYAQKFRDQSSCRGNSHTSSGTYGETXAVGYADGAAALQAWYEEAGKDGL
SYSYGSSSVYNHFTQVVWKSTTKLGCAYKDCRAQNWGLYVVCSYDPAGNVMGTDPKTGKSYMAENVLRPQ*

| Rank | Sequence | Start position | Score |
|---|---|---|---|
| 1 | NVMGTDPKTGKSYMAE | 337 | 0.91 |
| 1 | TLTIGGGGFPSDDQYP | 137 | 0.91 |
| 2 | DLTWDATGYEYAQKFR | 230 | 0.90 |
| 3 | TVTVGATVTGGDQGQD | 41 | 0.89 |
| 3 | TATVATTVTVGATVTG | 35 | 0.89 |
| 3 | CRAQNWGLYVVCSYDP | 319 | 0.89 |
| 4 | SAVPEAEPTADADGGN | 83 | 0.88 |
| 5 | AGDIQQSAVPEADDIQ | 66 | 0.87 |
| 5 | QFAQQILDAHNKKRAR | 210 | 0.87 |
| 5 | AVATPDAKVPDSTKDA | 170 | 0.87 |
| 6 | TKLGCAYKDCRAQNWG | 310 | 0.86 |
| 6 | TGYEYAQKFRDQSSCR | 236 | 0.86 |
| 6 | YVTQTRGVTVGETATV | 23 | 0.86 |
| 6 | SGVYYSYGEEHTYGDV | 118 | 0.86 |
| 7 | YVVCSYDPAGNVMGTD | 327 | 0.85 |
| 7 | TSSGTYGETXAVGYAD | 256 | 0.85 |
| 7 | EASPSAVTTSSAVATP | 159 | 0.85 |
| 7 | GQEIVYSGVYYSYGEE | 112 | 0.85 |
| 8 | QVQQSAAPEAGDIQQS | 57 | 0.84 |
| 9 | YGSSSVYNHFTQVVWK | 292 | 0.83 |
| 9 | GEEHTYGDVQVQTLTI | 125 | 0.83 |
| 10 | ARHGVPDLTWDATGYE | 224 | 0.82 |
| 10 | TTAEIAYVTQTRGVTV | 17 | 0.82 |
| 10 | GGFPSDDQYPTTEVSA | 143 | 0.82 |
| 11 | SGSSSGSNDFSGVKDT | 194 | 0.81 |
| 12 | GETXAVGYADGAAALQ | 262 | 0.80 |
| 13 | ADDIQQSAVPEAEPTA | 77 | 0.79 |
| 13 | TVTGGDQGQDQVQQSA | 47 | 0.79 |
| 13 | EEAGKDGLSYSYGSSS | 281 | 0.79 |
| 14 | EPTADADGGNGIAITE | 89 | 0.78 |
| 14 | KFRDQSSCRGNSHTSS | 243 | 0.78 |
| 15 | QPAATTASGSSSGSND | 187 | 0.75 |
| 15 | AKVPDSTKDASQPAAT | 176 | 0.75 |

| Start | End | Max_score_pos | Sequence |
|---|---|---|---|
| 324 | 333 | 329 | WGLYVVCSYD |
| 113 | 125 | 119 | QEIVYSGVYYSYG |
| 35 | 47 | 41 | TATVATTVTVGAT |
| 130 | 138 | 136 | YGDVQVQTL |
| 288 | 307 | 303 | LSYSYGSSSVYNHFTQVVWK |
| 311 | 319 | 316 | KLGCAYKDC |
| 71 | 77 | 72 | QSAVPEA |
| 82 | 88 | 83 | QSAVPEA |
| 157 | 181 | 168 | SAEASPSAVTTSSAVATPDAKVPDS |
| 55 | 63 | 61 | QDQVQQSAA |
| 212 | 218 | 213 | AQQILDA |
| 18 | 27 | 24 | TAEIAYVTQT |
| 4 | 16 | 13 | SQVATTAAIFAGL |
| 272 | 279 | 277 | GAAALQAW |
| 225 | 231 | 229 | RHGVPDL |
| 186 | 191 | 188 | SQPAAT |

25. IPF5761
MTKEQIDEPRYKRIAVIGGGPTGLAAVKALSLEPVNFSCIDLFERRDRLGGLWYHHGDKSLVKPEIPSLSPS
QEEIVSDNATPADEYFSAIYEYMETNIVHQIMEYSGVAFPANSKKYPTRSQVLEYIDDYIKSIPKDTVNISI
NSNVVSLEKVNEIWHIEIEDVIKKTRAKLRYDAVIIANGHFSNPYIPDVPGLSSWNKNYPGTITHSKYYESP
AKFRDKRVLVVGNSASGVDISIQLSVCAKDVFVSIRDQESPHFEDGFCKHIGLIEEYNYETRSVRTTDREVV
SDIDYVIFCTGYLYALPFLKQERNITDGFQVYDLYKQIFNIYDPSLTFLALLRDVIPMPISESQAALIARVY
SGRYKLPPTEEMERYYQLELKEKGRGGKFHNYKYPRDVAYCQMLQTLIDEQGLHTPGLVAPIWDESLIKKRS
ETRAEKNARLKNVVEHVKRLRAEGKDFSLLE*

| Rank | Sequence | Start position | Score |
|---|---|---|---|
| 1 | LEYIDDYIKSIPKDTV | 125 | 0.94 |
| 2 | DAVIIANGHFSNPYIP | 176 | 0.92 |
| 3 | ESLIKKRSETRAEKNA | 425 | 0.91 |
| 3 | LKEKGRGGKFHNYKYP | 380 | 0.91 |
| 4 | KFHNYKYPRDVAYCQM | 388 | 0.88 |
| 4 | AALIARVYSGRYKLPP | 353 | 0.88 |
| 4 | PGTITHSKYYESPAKF | 204 | 0.88 |
| S | QTLIDEQGLHTPGLVA | 405 | 0.87 |
| 5 | DREVVSDIDYVIFCTG | 284 | 0.87 |
| 6 | QLSVCAKDVFVSIRDQ | 239 | 0.86 |
| 7 | PPTEEMERYYQLELKE | 367 | 0.85 |
| 7 | YETRSVRTTDREVVSD | 275 | 0.85 |
| 7 | NISINSNVVSLEKVNE | 141 | 0.85 |
| 8 | GGLWYHHGDKSLVKPE | 50 | 0.84 |
| 8 | IAVIGGGPTGLAAVKA | 14 | 0.84 |
| 9 | GLIEEYNYETRSVRTT | 268 | 0.83 |
| 9 | VNEIWHIEIEDVIKKT | 154 | 0.83 |
| 10 | FSAIYEYMETNIVHQI | 88 | 0.82 |
| 10 | TKEQIDEPRYKRIAVI | 2 | 0.82 |
| 11 | FSCIDLFERRDRLGGL | 37 | 0.81 |
| 11 | FVSIRDQESPHFEDGF | 248 | 0.81 |
| 11 | VHQIMEYSGVAFPANS | 100 | 0.81 |
| 12 | FNIYDPSLTFLALLRD | 327 | 0.79 |
| 12 | GLSSWNKNYPGTITHS | 195 | 0.79 |
| 13 | SETRAEKNARLKNVVE | 432 | 0.77 |
| 14 | DLYKQIFNIYDPSLTF | 321 | 0.76 |
| 15 | KALSLEPVNFSCIDLF | 28 | 0.7S |

| Start | End | Max_score_pos | Sequence |
|---|---|---|---|
| 233 | 252 | 241 | GVDISIQLSVCAKDVFVSIR |
| 285 | 308 | 294 | REVVSDIDYVIFCTGYLYALPFLK |
| 145 | 154 | 151 | NSNVVSLEKV |
| 395 | 408 | 401 | PRDVAYCQMLQTLI |
| 222 | 229 | 225 | KRVLVVGN |
| 316 | 347 | 337 | GFQVYDLYKQIFNIYDPSLTFLALLRDVIPMP |
| 23 | 42 | 27 | GLAAVKALSLEPVNFSCIDL |
| 350 | 367 | 357 | ESQAALIARVYSGRYKLP |
| 442 | 452 | 448 | LKNVVEHVKRL |
| 261 | 270 | 267 | DGFCKHIGLI |
| 413 | 422 | 419 | LHTPGLVAPI |
| 172 | 183 | 178 | KLRYDAVIIANG |
| 121 | 135 | 125 | RSQVLEYIDDYIKSI |
| 186 | 196 | 191 | SNPYIPDVPGL |
| 12 | 19 | 14 | KRIAVIGG |
| 57 | 72 | 64 | GDKSLVKPEIPSLSPS |
| 106 | 113 | 109 | YSGVAFPA |
| 375 | 380 | 378 | YYQLEL |
| 97 | 104 | 103 | TNIVHQIM |
| 85 | 93 | 89 | DEYFSAIYE |
| 209 | 216 | 210 | HSKYYESP |
| 162 | 168 | 163 | IEDVIKK |
| 137 | 143 | 137 | KDTVNIS |

26. IPF1428
MSPDNEPQPPNEDELLNNILPSYHMFQSTVSKNLTPTNENYSIDPPTYEMTPITSETPSLLTFSRMQSPVDE
RLETDNYFPQSDNDSVTYNQESEDMWKNSILANADKLPNLTHKKNSMSECLQIDIQVTEKVCQSGIKPIFMD
PSNREFKQGDYLHGYVTIRNTSDQPIPFDMVYVVFEGTFTTLDTSSGTISTEMPALRFKFLTMLDLFASWSY
ANIDRLITDNGDPHDWCNGETDPYDNTLLSIDVKRLFQPNVTYKRFFTFKIPDKLLDSTCDQYNLPTHTEIP
PTLGIDRNSFPPSFLLANQHLIVKDLSFSDSCLAYRIDARVIGKASDYKYKVDKDQYVVSKEASCPIRVVPT
PNLEMEYNFQQLKQEAELYYRAFVDSVMVKIEYGNELLNNKPGYSNTSRPNLSPMMSNDSVKLRQLYDVADD
TFKTNLRSGKSMRDEDYYQCLIPFKKKSITGSSKYLGIISLSTIKEHYKIRYTPPTRFGKAPPPNDTELLIP
LELNYFTESSTPLKNLPEIKAIDVEVVALSIRSKKHPIPIEFTTDMLFAEKEIDIKKSQPANFNSLVVARFS
NYLNEFHKLIKGVGNEALRLETKLYQDVKCLASLKTKYINLPISNLVFETTSQNGIGTTTEVKSLQWQEEQS
EKGKLFTKKFAVRMNLNNCSSKSNDNSSKGLDRITLVPSFQTCFASRLYYIKMTVRLNHGDSLLVNVPLNIH
RY*

| Rank | Sequence | Start position | Score |
|---|---|---|---|
| 1 | YKIRYTPPTRFGKAPP | 480 | 0.96 |
| 2 | ARVIGKASDYKYKVDK | 327 | 0.94 |
| 2 | CQSGIKPIFMDPSNRE | 134 | 0.94 |
| 3 | TRFGKAPPPNDTELLI | 488 | 0.93 |
| 4 | LRSGKSMRDEDYYQCL | 438 | 0.91 |
| 4 | DWCNGETDPYDNTLLS | 231 | 0.91 |
| 4 | DRLITDNGDPHDWCNG | 220 | 0.91 |
| 5 | LFTKKFAVRMNLNNCS | 653 | 0.90 |
| 5 | QLKQEAELYYRAFVDS | 371 | 0.90 |
| 5 | HGYVTIRNTSDQPIPF | 157 | 0.90 |
| 6 | VVSKEASCPIRVVPTP | 346 | 0.89 |
| 6 | QSTVSKNLTPTNENYS | 27 | 0.89 |
| 7 | TNENYSIDPPTYEMTP | 37 | 0.88 |
| 7 | ASDYKYKVDKDQYVVS | 333 | 0.88 |
| 8 | FQTCFASRLYYIKMTV | 688 | 0.87 |
| 8 | TSSGTISTEMPALRFK | 188 | 0.87 |
| 9 | FSRMQSPVDERLETDN | 63 | 0.86 |
| 9 | SQNGIGTTTEVKSLQW | 628 | 0.86 |
| 10 | EDMWKNSILANADKLP | 95 | 0.85 |
| 10 | DERLETDNYFPQSDND | 71 | 0.85 |
| 10 | TSETPSLLTFSRMQSP | 54 | 0.85 |
| 10 | ALSIRSKKHPIPIEFT | 532 | 0.85 |
| 10 | THTEIPPTLGIDRNSF | 283 | 0.85 |
| 11 | NNKPGYSNTSRPNLSP | 399 | 0.84 |
| 12 | DNEPQPPNEDELLNNI | 4 | 0.83 |
| 13 | VDSVMVKIEYGNELLN | 384 | 0.82 |
| 13 | DSTCDQYNLPTHTEIP | 273 | 0.82 |
| 14 | PQSDNDSVTYNQESED | 81 | 0.79 |
| 15 | SNDNSSKGLDRITLVP | 671 | 0.77 |
| 15 | TKYINLPISNLVFETT | 612 | 0.77 |
| 15 | EKEIDIKKSQPANFNS | 554 | 0.77 |
| 15 | PTYEMTPITSETPSLL | 46 | 0.77 |
| 15 | CLQIDIQVTEKVCQSG | 122 | 0.77 |
| 16 | KGVGNEALRLETKLYQ | 587 | 0.76 |
| 16 | TDMLFAEKEIDIKKSQ | 548 | 0.76 |
| 16 | PTLGIDRNSFPPSFLL | 289 | 0.76 |
| 17 | SCPIRVVPTPNLEMEY | 352 | 0.75 |
| 17 | MVYVVFEGTFTTLDTS | 174 | 0.75 |
| 17 | KNSMSECLQIDIQVTE | 116 | 0.75 |

| Start | End | Max_score_pos | Sequence |
|---|---|---|---|
| 708 | 719 | 714 | GDSLLVNVPLNI |
| 335 | 361 | 356 | DYKYKVDKDQYVVSKEASCPIRVVPTP |
| 522 | 545 | 531 | EIKAIDVEVVALSIRSKKHPIPIE |
| 601 | 626 | 607 | YQDVKCLASLKTKYINLPISNLVFET |
| 449 | 458 | 452 | YYQCLIPFKK |
| 168 | 181 | 178 | QPIPFDMVYVVFEG |
| 500 | 508 | 504 | ELLIPLELN |
| 567 | 576 | 572 | FNSLVVARFS |
| 375 | 392 | 381 | EAELYYRAFVDSVMVKIE |
| 299 | 333 | 311 | PPSFLLANQHLIVKDLSFSDSCLAYRIDARVIGKA |
| 681 | 702 | 688 | RITLVPSFQTCFASRLYYIKMT |
| 120 | 141 | 132 | SECLQIDIQVTEKVCQSGIKPI |
| 420 | 432 | 424 | SVKLRQLYDVADD |
| 464 | 485 | 470 | SSKYLGIISLSTIKEHYKIRYT |
| 242 | 252 | 247 | NTLLSIDVKRL |
| 153 | 163 | 158 | GDYLHGYVTIR |
| 268 | 282 | 273 | PDKLLDSTCDQYNLP |
| 580 | 588 | 586 | NEFHKLIKG |
| 57 | 63 | 61 | TPSLLTF |
| 17 | 33 | 22 | NNILPSYHMFQSTVSKN |
| 254 | 260 | 255 | QPNVTYK |
| 205 | 214 | 213 | LTMLDLFASW |
| 284 | 291 | 290 | HTEIPPTL |
| 67 | 72 | 71 | QSPVDE |

27. MEP2
MSGNFTGTGTGGDVFKVDLNEQFDRADMVWIGTASVLVWIMIPGVGLLYSGISRKKHALSLMWAALMAACVA
AFQWFWWGYSLVFAHNGSVFLGTLQNFCLKDVLGAPSIVKTVPDILFCLYQGMFAAVTAILMAGAGCERARL
GPMMVFLFIWLTVVYCPIAYWTWGGNGWLVSLGALDFAGGGPVHENSGFAALAYSLWLGKRHDPVAKGKVPK
YKPHSVSSIVMGTIFLWFGWYGFNGGSTGNSSMRSWYACVNTNLAAATGGLTWMLVDWFRTGGKWSTVGLCM
GAIAGLVGITPAAGYVPVYTSVIFGIVPAIICNFAVDLKDLLQIDDGMDVWALHGVGGFVGNFMTGLFAADY
VAMIDGTEIDGGWMNHHWKQLGYQLAGSCAVAAWSFTVTSIILLAMDRIPFLRIRLHEDEEMLGTDLAQIGE
YAYYADDDPETNPYVLEPIRSTTISQPLPHIDGVADGSSNNDSGEAKN*

| Rank | Sequence | Start position | Score |
|---|---|---|---|
| 1 | TGTGTGGDVFKVDLNE | 6 | 0.98 |
| 2 | IGEYAYYADDDPETNP | 430 | 0.92 |
| 3 | DGTEIDGGWMNHHWKQ | 365 | 0.91 |
| 4 | STTISQPLPHIDGVAD | 453 | 0.89 |
| 4 | GLTWMLVDWFRTGGKW | 266 | 0.89 |
| 5 | AIAGLVGITPAAGYVP | 290 | 0.88 |
| 5 | VGLCMGAIAGLVGITP | 284 | 0.88 |
| 6 | HIDGVADGSSNNDSGE | 462 | 0.87 |
| 6 | DWFRTGGKWSTVGLCM | 273 | 0.87 |
| 6 | DFAGGGPVHENSGFAA | 180 | 0.87 |
| 6 | AILMAGAGCERARLGP | 131 | 0.87 |
| 7 | LWFGWYGFNGGSTGNS | 232 | 0.86 |
| 8 | IVMGTIFLWFGWYGFN | 225 | 0.84 |
| 8 | YCPIAYWTWGGNGWLV | 159 | 0.84 |
| 8 | FLFIWLTVVYCPIAYW | 150 | 0.84 |
| 9 | GGSTGNSSMRSWYACV | 241 | 0.83 |
| 9 | LWLGKRHDPVAKGKVP | 200 | 0.83 |
| 10 | WFWWGYSLVFAHNGSV | 76 | 0.82 |
| 10 | LVWIMIPGVGLLYSGI | 37 | 0.82 |
| 11 | YTSVIFGIVPAIICNF | 307 | 0.81 |
| 11 | GITPAAGYVPVYTSVI | 296 | 0.81 |
| 12 | NPYVLEPIRSTTISQP | 444 | 0.80 |
| 12 | FAALAYSLWLGKRHDP | 193 | 0.80 |
| 13 | CVAAFQWFWWGYSLVF | 70 | 0.79 |
| 14 | GWLVSLGALDFAGGGP | 171 | 0.78 |
| 14 | AGCERARLGPMMVFLF | 137 | 0.78 |
| 15 | FVGNFMTGLFAADYVA | 347 | 0.77 |
| 15 | MVWIGTASVLVWIMIP | 28 | 0.77 |
| 15 | GKVPKYKPHSVSSIVM | 212 | 0.77 |
| 16 | HWKQLGYQLAGSCAVA | 377 | 0.76 |
| 16 | GAPSIVKTVPDILFCL | 106 | 0.76 |
| 17 | LGTLQNFCLKDVLGAP | 93 | 0.75 |
| 17 | MDVWALHGVGGFVGNF | 336 | 0.75 |

| Start | End | Max_score_pos | Sequence |
|---|---|---|---|
| 146 | 164 | 160 | PMMVFLFIWLTVVYCPIAY |
| 283 | 332 | 317 | TVGLCMGAIAGLVGITPAAGYVPVYTSVIFGIVPAIICN |
| | | | FAVDLKDLLQI |
| 80 | 136 | 120 | GYSLVFAHNGSVFLGTLQNFCLKDVLGAPSIVKTVPDIL |
| | | | FCLYQGMFAAVTAILMAG |
| 66 | 75 | 71 | LMAACVAAFQ |
| 31 | 52 | 37 | IGTASVLVWIMIPGVGLLYSGI |
| 378 | 407 | 401 | WKQLGYQLAGSCAVAAWSFTVTSIILLAMD |
| 172 | 181 | 176 | WLVSLGALDF |
| 206 | 229 | 223 | HDPVAKGKVPKYKPHSVSSIVMGT |
| 338 | 347 | 341 | VWALHGVGGF |
| 444 | 451 | 448 | NPYVLEPI |
| 193 | 203 | 199 | FAALAYSLWLG |
| 354 | 364 | 358 | GLFAADYVAMI |
| 252 | 259 | 253 | WYACVNTN |
| 14 | 20 | 16 | VFKVDLN |
| 456 | 466 | 463 | ISQPLPHIDGV |
| 409 | 415 | 414 | IPFLRIR |
| 55 | 64 | 58 | KKHALSLMWA |
| 425 | 438 | 433 | TDLAQIGEYAYYAD |
| 269 | 274 | 269 | WMLVDW |

28. PTH1
MITKCYVQVFRYRRVCHNTKYSFNSVVSIKWLHSAGSSLSQANSSKTSQSSLTSSGFLYYEDPHRYNGSDVS
ANASETTSTSTAKPVIHSATPYSDKYYQPIISQGAKGFDKLIIPVGFCADNQTVSLEEDKESPIQQDQLQEI
VNSFDAPIDVSIGYGSGILPQDGYDKDKSTSNNTANDSKQLDFMFLVKDCGKFHQENLKQNRDHYSIKSLRL
IKKVQGTNGMYFNPFIKINEKLVKYGVISSKSALMDLSEWHSLYFAGRLQKPVNFITTNDPRVKFLNQYNLK
NAMTIAIFLIDGEGNSRQATFNERQLYEQITKLSYLGDFRMYIGGENPNKSKNIVAKQFHHFKKLYEPILQY
FIHKNFLIIVDNDPVNRTFKPNLNVNNRIKLITGLPLKFRQQLYGRYYEKSIKEIVIDDHLSQNLTKIISRT
IIISSITQAIRGLLSAGLFNSIKYAVAKQIKFWTSKK*

| Rank | Sequence | Start position | Score |
|---|---|---|---|
| 1 | DGYDKDKSTSNNTAND | 166 | 0.95 |
| 2 | PVIHSATPYSDKYYQP | 86 | 0.92 |
| 3 | GFLYYEDPHRYNGSDV | 56 | 0.91 |
| 4 | FRMYIGGENPNKSKNI | 327 | 0.90 |
| 4 | AGRLQKPVNFITTNDP | 262 | 0.90 |
| 4 | YGSGILPQDGYDKDKS | 158 | 0.90 |
| 5 | NFLIIVDNDPVNRTFK | 365 | 0.85 |
| 6 | YQPIISQGAKGFDKLI | 99 | 0.84 |
| 6 | NDPVNRTFKPNLNVNN | 372 | 0.84 |
| 6 | VNFITTNDPRVKFLNQ | 269 | 0.84 |
| 6 | RVCHNTKYSFNSVVSI | 14 | 0.84 |
| 7 | KCYVQVFRYRRVCHNT | 4 | 0.82 |
| 7 | QGTNGMYFNPFIKINE | 221 | 0.82 |
| 7 | DAPIDVSIGYGSGILP | 149 | 0.82 |
| 7 | DQLQEIVNSFDAPIDV | 139 | 0.82 |
| 8 | TTSTSTAKPVIHSATP | 78 | 0.81 |
| 9 | HYSIKSLRLIKKVQGT | 208 | 0.80 |
| 10 | GFCADNQTVSLEEDKE | 118 | 0.79 |
| 11 | NGSDVSANASETTSTS | 67 | 0.78 |
| 11 | SQSSLTSSGFLYYEDP | 48 | 0.78 |
| 11 | TQAIRGLLSAGLFNSI | 439 | 0.78 |
| 11 | EIVIDDHLSQNLTKII | 414 | 0.78 |
| 11 | VVSIKWLHSAGSSLSQ | 26 | 0.78 |
| 12 | LSEWHSLYFAGRLQKP | 253 | 0.77 |
| 12 | FDKLIIPVGFCADNQT | 110 | 0.77 |
| 13 | KFRQQLYGRYYEKSIK | 398 | 0.76 |
| 14 | TIAIFLIDGEGNSRQA | 292 | 0.75 |

| Start | End | Max_score_pos | Sequence |
|---|---|---|---|
| 4 | 19 | 7 | KCYVQVFRYRRVCHNT |
| 235 | 248 | 241 | NEKLVKYGVISSKS |
| 110 | 129 | 117 | FDKLIIPVGFCADNQTVSLE |
| 23 | 42 | 29 | FNSVVSIKWLHSAGSSLSQA |
| 341 | 373 | 360 | NIVAKQFHHFKKLYEPILQYFIHKNFLIIVDND |
| 186 | 197 | 192 | DFMFLVKDCGKF |
| 430 | 462 | 448 | SRTIIISSITQAIRGLLSAGLFNSIKYAVAKQI |
| 413 | 420 | 418 | KEIVIDDH |
| 293 | 298 | 295 | IAIFLI |
| 209 | 220 | 217 | YSIKSLRLIKKV |
| 390 | 407 | 394 | KLITGLPLKFRQQLYGRY |
| 84 | 105 | 101 | AKPVIHSATPYSDKYYQPIISQ |
| 137 | 157 | 153 | QQDQLQEIVNSFDAPIDVSIG |
| 314 | 325 | 321 | LYEQITKLSYLG |
| 256 | 271 | 259 | WHSLYFAGRLQKPVNF |
| 49 | 63 | 57 | QSSLTSSGFLYYEDP |
| 159 | 166 | 161 | GSGILPQD |
| 422 | 428 | 423 | SQNLTKI |

29. ENA22
MSSTKENSNYASGDTKERANSDLSESDRSTPPRDPNSTFQAYRLTIDEVAQEFNTSIVDGLGAHDAENRIQA
YGPNNLGEGDKISYPKILAHQVFNAMILVLIISMIIALAIKDWISGGVIAFVVFLNISVGFVQEVKAEKTMG
SLKNLSSPTARVTRNGDDFTIPAEEVVPGDIVHIKVGDTVPADLRLFDCMNLETDEALLTGESLPVAKNFEV
VYTDYSVPVPVGDRLNLVYSSSIVSKGRGSGIVFATGLNTEIGAIAQSLKGNSGLIRRVDKSNDRKPQKREY
GQAAAGTIYDVVGNILGVTVGTPLQRKLSWLAIFLFCVAVVFAIIVMGSQKFHVNKEVAIYAICVALSMIPS
ALILVLTITMAVGAQVMVTKNVIVRKFDSLEALGGINDICSDKTGTLTQGKMIAKKVWLPNIGTLDVQNSNE
PYNPTVGDVRFAPYSPKFVKETDEEIDFNKPYPDPMPESMHKWLMTATLANIATVNQTKDEDTGELLWKAHG
DATEIAIQVFTTRLNYGRESIAQEYEHLAEFPFDSSIKRMSAIYKKDGETRVYTKGAVERLLGLCDYWYGER
TEDDYDSQTLVKLTEDDAKLIEENMAALSSQGLRVLAFATKELGDADMNDREQVESHLIFQGLIGIYDPPRE
ESAQSVKSCHKAGINVHMLTGDHPGTAKAIAQEVGILPHNLYHYSEDVVKVMVMSANDFDALTDDEIDNLPV
LPLVIARCAPKTKVRMIDALHRRKKFAAMTGDGVNDSPSLKKADVGIAMGLNGSDVAKDASDIVLTDDNFAS
ILNAIEEGRRMSANIQKFVLQLLAENVAQAFYLMIGLAFLDETGYSVFPLSPVEVLWILVVTSTFPAIGLAQ
NAASDDILEKPPNNTIFTWEVIIDMFAYGVIMAATCLLSFVIVVYGAGNGDLGIDCNATNADKDLCSLVFEG
RSTAFASMTWQALILAWECLDPKKSLLLIPFSELWANQFLFWSIVGGFVTVFPVIYIPVINTKVFLHKSITW
EWGVAVGTTALFLLGAEAWKWGKRVFARSSKAKNPEYELERNDPFQRYASFSRANTMVV*

| Rank | Sequence | Start position | Score |
|---|---|---|---|
| 1 | KVMVMSANDFDALTDD | 698 | 0.96 |
| 2 | QGLIGIYDPPREESAQ | 637 | 0.94 |
| 2 | SESDRSTPPRDPNSTF | 24 | 0.94 |
| 3 | MLTGDHPGTAKAIAQE | 666 | 0.93 |
| 4 | AGTIYDVVGNILGVTV | 293 | 0.92 |
| 5 | LNAIEEGRRMSANIQK | 794 | 0.91 |
| 5 | VRMIDALHRRKKFAAM | 734 | 0.91 |
| 6 | SMTWQALILAWECLDP | 943 | 0.90 |
| 6 | KWLMTATLANIATVNQ | 474 | 0.90 |
| 7 | SSKAKNPEYELERNDP | 1037 | 0.89 |
| 8 | EVIIDMFAYGVIMAAT | 884 | 0.88 |
| 8 | DATEIAIQVFTTRLNY | 505 | 0.88 |
| 8 | SKGRGSGIVFATGLNT | 241 | 0.88 |
| 8 | YASGDTKERANSDLSE | 10 | 0.88 |
| 9 | YGAGNGDLGIDCNATN | 909 | 0.87 |
| 9 | DYWYGERTEDDYDSQT | 570 | 0.87 |
| 9 | MSAIYKKDGETRVYTK | 544 | 0.87 |
| 9 | HIKVGDTVPADLRLFD | 177 | 0.87 |
| 9 | SITWEWGVAVGTTALF | 1005 | 0.87 |
| 10 | YPKILAHQVFNAMILV | 86 | 0.86 |
| 10 | VKETDEEIDFNKPYPD | 451 | 0.86 |
| 11 | NGSDVAKDASDIVLTD | 772 | 0.85 |
| 11 | AGINVHMLTGDHPGTA | 660 | 0.85 |
| 11 | SNEPYNPTVGDVRFAP | 430 | 0.85 |
| 11 | SGLIRRVDKSNDRKPQ | 269 | 0.85 |
| 11 | VQEVKAEKTMGSLKNL | 134 | 0.85 |
| 12 | RRKKFAAMTGDGVNDS | 742 | 0.84 |
| 12 | GDVRFAPYSPKFVKET | 439 | 0.84 |
| 12 | VLTITMAVGAQVMVTK | 365 | 0.84 |
| 12 | LSSPTARVTRNGDDFT | 149 | 0.84 |
| 13 | MPESMHKWLMTATLAN | 468 | 0.83 |
| 13 | IGAIAQSLKGNSGLIR | 258 | 0.83 |
| 13 | EVVYTDYSVPVPVGDR | 215 | 0.83 |
| 13 | AEAWKWGKRVFARSSK | 1024 | 0.83 |
| 14 | LILAWECLDPKKSLLL | 949 | 0.82 |
| 14 | TPPRDPNSTFQAYRLT | 30 | 0.82 |
| 15 | VGIAMGLNGSDVAKDA | 765 | 0.81 |
| 15 | TGDGVNDSPSLKKADV | 750 | 0.81 |
| 15 | ENRIQAYGPNNLGEGD | 67 | 0.81 |
| 15 | YGRESIAQEYEHLAEF | 520 | 0.81 |
| 15 | ELLWKAHGDATEIAIQ | 497 | 0.81 |
| 15 | MGSLKNLSSPTARVTR | 143 | 0.81 |
| 16 | DGLGAHDAENRIQAYG | 59 | 0.80 |
| 16 | ALGGINDICSDKTGTL | 392 | 0.80 |
| 16 | IVRKFDSLEALGGIND | 383 | 0.80 |
| 17 | YGVIMAATCLLSFVIV | 892 | 0.79 |
| 17 | AIIVMGSQKFHVNKEV | 331 | 0.79 |
| 17 | DWISGGVIAFVVFLNI | 114 | 0.79 |
| 18 | EVAIYAICVALSMIPS | 345 | 0.78 |
| 18 | ERANSDLSESDRSTPP | 17 | 0.78 |
| 19 | ASDIVLTDDNFASILN | 780 | 0.77 |
| 19 | GVTVGTPLQRKLSWLA | 305 | 0.77 |
| 20 | GFVTVFPVIYIPVINT | 983 | 0.76 |
| 20 | IGLAQNAASDDILEKP | 860 | 0.76 |
| 20 | TGYSVFPLSPVEVLWI | 835 | 0.76 |
| 20 | KELGDADMNDREQVES | 617 | 0.76 |
| 20 | SQTLVKLTEDDAKLIE | 583 | 0.76 |
| 20 | VWLPNIGTLDVQNSNE | 417 | 0.76 |
| 20 | CSDKTGTLTQGKMIAK | 400 | 0.76 |
| 20 | STFQAYRLTIDEVAQE | 37 | 0.76 |
| 21 | TAKAIAQEVGILPHNL | 674 | 0.75 |
| 21 | ESAQSVKSCHKAGINV | 649 | 0.75 |
| 21 | GETRVYTKGAVERLLG | 552 | 0.75 |
| 21 | TRNGDDFTIPAEEVVP | 157 | 0.75 |

| Start | End | Max_score_pos | Sequence |
|---|---|---|---|
| 313 | 337 | 326 | QRKLSWLAIFLFCVAVVFAIIVMGS |
| 836 | 865 | 850 | GYSVFPLSPVEVLWILVVTSTFPAIGLAQN |
| 882 | 911 | 905 | TWEVIIDMFAYGVIMAATCLLSFVIVVYGA |
| 717 | 732 | 721 | NLPVLPLVIARCAPKT |
| 117 | 138 | 123 | SGGVIAFVVFLNISVGFVQEVK |
| 339 | 387 | 352 | KFHVNKEVAIYAICVALSMIPSALILVLTITMAVGAQVMVT |
| | | | KNVIVRKF |
| 199 | 242 | 224 | DEALLTGESLPVAKNFEVVYTDYSVPVPVGDRLNLVYSSSI |
| | | | VSK |
| 973 | 1005 | 993 | NQFLFWSIVGGFVTVFPVIYIPVINTKVFLHKS |
| 84 | 114 | 102 | ISYPKILAHQVFNAMILVLIISMIIALAIKD |
| 807 | 832 | 813 | IQKFVLQLLAENVAQAFYLMIGLAFL |
| 927 | 935 | 931 | KDLCSLVFE |
| 674 | 702 | 698 | TAKAIAQEVGILPHNLYHYSEDVVKVMVM |
| 167 | 194 | 173 | AEEVVPGDIVHIKVGDTVPADLRLFDCM |
| 947 | 969 | 964 | QALILAWECLDPKKSLLLIPFSE |
| 562 | 572 | 568 | VERLLGLCDYW |
| 603 | 616 | 612 | ALSSQGLRVLAFAT |
| 651 | 667 | 655 | AQSVKSCHKAGINVHML |
| 630 | 645 | 633 | VESHLIFQGLIGIYDP |
| 1011 | 1024 | 1021 | GVAVGTTALFLLGA |
| 293 | 311 | 306 | AGTIYDVVGNILGVTVGTP |
| 508 | 516 | 512 | EIAIQVFTT |
| 583 | 590 | 586 | SQTLVKLT |
| 246 | 252 | 251 | SGIVFAT |
| 435 | 451 | 448 | NPTVGDVRFAPYSPKFV |
| 781 | 787 | 783 | SDIVLTD |
| 414 | 420 | 416 | AKKVWLP |
| 481 | 487 | 484 | LANIATV |
| 526 | 536 | 534 | AQEYEHLAEFP |
| 259 | 264 | 263 | GAIAQS |
| 39 | 52 | 47 | FQAYRLTIDEVAQE |
| 791 | 796 | 794 | ASILNA |
| 759 | 768 | 762 | SLKKADVGIA |
| 1032 | 1037 | 1036 | RVFARS |
| 1054 | 1059 | 1056 | QRYASF |
| 397 | 402 | 398 | NDICSD |

30. MDL1
MIGMNRLIFSKAFTSSCKSMGKVPFTKSITRANTRYFKPTSILQQIRFNSKSSTTPNTEANSNGSTNSQSDT
KKPRPKLTSEIFKLLRLAKPESKLIFFALICLVTTSATTMTLPLMIGKIIDTTKKDDDDDKGKDNDDKDDTQ
PSDKLIFGLPQPQFYSALGVLFIVSASTNFGRIYLLRSVGERLVARLRSRLFSKILAQDAYFFDLGPSKTGM
KTGDLISRIASDTQIISKSLSMNISDGIRAIISGCVGLSMMCYVSWKLSLCMSLIFPPLITMSWFYGRKIKA
LSKLIQENIGDMTKVTEEKLNGVKVIQTFSQQQSVVHSYNQEIKNIFNSSMREAKLAGFFYSTNGFIGNVTM
IGLLIMGTKLIGAGELTVGDLSSFMMYAVYTGTSVFGLGNFYTELMKGIGAAERVFELVEYQPRISNHLGKK
VDELNGDIEFKGIDFTYPSRPESGIFKDLNLHIKQGENVCLVGPSGSGKSTVSQLLLRFYDPEKGTIQIGDD
VITDLNLNHYRSKLGYVQQEPLLFSGTIKENILFGKEDATDEEINNALNLSYASNFVRHLPDGLDTKIGASN
STQLSGGQKQRVSLARTLIRDPKILILDEATSALDSVSEEIVMSNLIQLNKNRGVTLISIAHRLSTIKNSDR
IIVFNQDGQIVEDGKFNELHNDPNSQFNKLLKSHSLE*

| Rank | Sequence | Start position | Score |
|---|---|---|---|
| 1 | TNSQSDTKKPRPKLTS | 66 | 0.94 |
| 1 | SGTIKENILFGKEDAT | 529 | 0.94 |
| 2 | KGTIQIGDDVITDLNL | 496 | 0.92 |
| 2 | TTKKDDDDDKGKDNDD | 124 | 0.92 |
| 3 | TKSITRANTRYFKPTS | 26 | 0.91 |
| 4 | DGQIVEDGKFNELHND | 655 | 0.90 |
| 4 | SKLIQENIGDMTKVTE | 290 | 0.90 |
| 5 | SKSSTTPNTEANSNGS | 50 | 0.89 |
| 5 | IGKIIDTTKKDDDDDK | 118 | 0.89 |
| 6 | DFTYPSRPESGIFKDL | 446 | 0.88 |
| 6 | SSMREAKLAGFFYSTN | 337 | 0.88 |
| 7 | AGFFYSTNGFIGNVTM | 345 | 0.87 |
| 7 | TQIISKSLSMNISDGI | 229 | 0.87 |
| 8 | FGKEDATDEEINNALN | 538 | 0.86 |
| 8 | YVSWKLSLCMSLIFPP | 259 | 0.86 |
| 9 | TKLIGAGELTVGDLSS | 368 | 0.85 |
| 9 | TMSWFYGRKIKALSKL | 277 | 0.85 |
| 10 | MMYAVYTGTSVFGLGN | 385 | 0.84 |
| 10 | FSKILAQDAYFFDLGP | 196 | 0.84 |
| 10 | SCKSMGKVPFTKSITR | 16 | 0.84 |
| 11 | HRLSTIKNSDRIIVFN | 638 | 0.83 |
| 12 | TLISIAHRLSTIKNSD | 632 | 0.82 |
| 12 | AERVFELVEYQPRISN | 412 | 0.82 |
| 13 | SDGIRAIISGCVGLSM | 241 | 0.81 |
| 14 | GLLIMGTKLIGAGELT | 362 | 0.80 |
| 15 | DSVSEEIVMSNLIQLN | 611 | 0.79 |
| 15 | PTSILQQIRFNSKSST | 39 | 0.79 |
| 15 | TKVTEEKLNGVKVIQT | 301 | 0.79 |
| 16 | GYVQQEPLLFSGTIKE | 519 | 0.77 |
| 17 | TEANSNGSTNSQSDTK | 58 | 0.76 |
| 17 | LMKGIGAAERVFELVE | 405 | 0.76 |
| 17 | TGMKTGDLISRIASDT | 214 | 0.76 |
| 18 | QSVVHSYNQEIKNIFN | 321 | 0.75 |
| 18 | IISGCVGLSMMCYVSW | 247 | 0.75 |
| 18 | FGRIYLLRSVGERLVA | 174 | 0.75 |
| 18 | DDKGKDNDDKDDTQPS | 131 | 0.75 |

| Start | End | Max_score_pos | Sequence |
|---|---|---|---|
| 80 | 108 | 102 | TSEIFKLLRLAKPESKLIFFALICLVTTS |
| 146 | 171 | 165 | SDKLIFGLPQPQFYSALGVLFIVSAS |
| 469 | 477 | 474 | ENVCLVGPS |
| 176 | 210 | 180 | RIYLLRSVGERLVARLRSRLFSKILAQDAYFFDLG |
| 310 | 328 | 324 | GVKVIQTFSQQQSVVHSYN |
| 482 | 492 | 486 | STVSQLLLRFY |
| 245 | 279 | 249 | RAIISGCVGLSMMCYVSWKLSLCMSLIFPPLITMS |
| 412 | 424 | 418 | AERVFELVEYQPR |
| 631 | 641 | 634 | VTLISIAHRLS |
| 514 | 530 | 524 | YRSKLGYVQQEPLLFSG |
| 676 | 682 | 681 | NKLLKSH |
| 586 | 593 | 591 | QRVSLART |
| 286 | 292 | 291 | IKALSKL |
| 596 | 617 | 600 | RDPKILILDEATSALDSVSEEI |
| 360 | 368 | 361 | MIGLLIMGT |
| 553 | 567 | 563 | NLSYASNFVRHLPDG |
| 386 | 399 | 392 | MYAVYTGTSVFGLG |
| 8 | 18 | 10 | IFSKAFTSSCK |
| 619 | 625 | 620 | MSNLIQL |
| 38 | 46 | 45 | KPTSILQQI |
| 375 | 381 | 379 | ELTVGDL |
| 20 | 27 | 26 | MGKVPFTK |
| 648 | 654 | 649 | RIIVFNQ |
| 228 | 235 | 233 | DTQIISKS |
| 460 | 466 | 464 | DLNLHIK |
| 113 | 119 | 117 | TLPLMIG |
| 656 | 661 | 657 | GQIVED |

31. ALP1
MSVEYPNSVTSLDKKPQVDLENVIEDASTSSEHRVAQTENLNRSLGARTINLICLGGVIGTGIFLGMGKMLS
NAGPLGLLLNYLIMGSMIYFMMLSLGEMSVQYPISGSFAVYTKRFGSDSLAFATLFNYWLNDCVSVAADLVA
LQLVMQYWTNFHWYVISIIFWVFLLLLNVLHVRLYAEAEYSLALLKVVTIIIFFIVSIICNAGKNPQHEYIG
FKYWSYGDAPFVDGIRGFSKVFASAAYSFGGLESVSLTAGETKNPTRVIPKTVQMTFFRVLIFYILTAFFIG
MNIPYDYPNLLTKKVATSPFTIVFQMVGAKGAGSFMNAVIMTSIVSAGNHALYAGSRLAYNLSLHGYIPKIF
LPMNRFRVPYVAVIITWLIGGLCFASAFVGSGELWSWLQAIVGLSNLISWWVIGVVSIRFRRGLEKQGRTHE
LLFKNWSYPYGPLYVVILGGFIILVQGWTTFSPFSVNDFFQSYLELGVFPLCFVFWWLVVRKGKDKFVKFED
MDFDTDRYYETPEEIEKNRYANSLKGWAKFKYNFADNFL*

| Rank | Sequence | Start position | Score |
|---|---|---|---|
| 1 | GGLESVSLTAGETKNP | 246 | 0.90 |
| 1 | HEYIGFKYWSYGDAPF | 212 | 0.90 |
| 2 | IEDASTSSEHRVAQTE | 24 | 0.87 |
| 2 | YWSYGDAPFVDGIRGF | 219 | 0.87 |
| 3 | TGIFLGMGKMLSNAGP | 61 | 0.86 |
| 3 | TWLIGGLCFASAFVGS | 376 | 0.86 |
| 3 | NIPYDYPNLLTKKVAT | 290 | 0.86 |
| 4 | YYETPEEIEKNRYANS | 512 | 0.85 |
| 5 | EMSVQYPISGSFAVYT | 99 | 0.83 |
| 5 | KFEDMDFDTDRYYETP | 501 | 0.83 |
| 5 | FQMVGAKGAGSFMNAV | 312 | 0.83 |
| 6 | VQGWTTFSPFSVNDFF | 457 | 0.82 |
| 6 | AGSRLAYNLSLHGYIP | 342 | 0.82 |
| 6 | MTSIVSAGNHALYAGS | 329 | 0.82 |
| 6 | SVEYPNSVTSLDKKPQ | 2 | 0.82 |
| 7 | SGELWSWLQAIVGLSN | 391 | 0.80 |
| 8 | HRVAQTENLNRSLGAR | 33 | 0.79 |
| 9 | AGETKNPTRVIPKTVQ | 255 | 0.78 |
| 10 | LCFVFWWLVVRKGKDK | 483 | 0.75 |
| 10 | KQGRTHELLFKNWSYP | 426 | 0.75 |
| 10 | SDSLAFATLFNYWLND | 119 | 0.75 |
| 10 | ISGSFAVYTKRFGSDS | 106 | 0.75 |

| Start | End | Max_score_pos | Sequence |
|---|---|---|---|
| 464 | 494 | 483 | SPFSVNDFFQSYLELGVFPLCFVFWWLVVRK |
| 441 | 460 | 447 | PYGPLYVVILGGFIILVQGW |
| 132 | 150 | 145 | LNDCVSVAADLVALQLVMQ |
| 367 | 392 | 371 | RVPYVAVIITWLIGGLCFASAFVGSG |
| 156 | 205 | 189 | HWYVISIIFWVFLLLLNVLHVRLYAEAEYSLALLKVVT |
| | | | IIIFFIVSIICN |
| 262 | 286 | 279 | TRVIPKTVQMTFFRVLIFYILTAFF |
| 409 | 419 | 415 | SWWVIGVVSIR |
| 50 | 58 | 55 | INLICLGGV |
| 76 | 88 | 82 | PLGLLLNYLIMGS |
| 395 | 407 | 401 | WSWLQAIVGLSNL |
| 234 | 246 | 240 | FSKVFASAAYSFG |
| 328 | 362 | 330 | IMTSIVSAGNHALYAGSRLAYNLSLHGYIPKIFLP |
| 98 | 115 | 104 | GEMSVQYPISGSFAVYTK |
| 292 | 315 | 312 | PYDYPNLLTKKVATSPFTIVFQMV |
| 120 | 130 | 125 | DSLAFATLFNY |
| 248 | 254 | 251 | LESVSLT |
| 14 | 23 | 21 | KKPQVDLENV |
| 4 | 12 | 6 | EYPNSVTSL |
| 224 | 231 | 225 | DAPFVDGI |
| 431 | 437 | 433 | HELLFKN |
| 212 | 220 | 217 | HEYIGFKYW |
| 497 | 502 | 499 | DKFVKF |

32. RAD23
MQIIFKDFKKQTVSLDVELTDTVLSTKEKLAQEKSCESSQIKLVYSGKVLQDDKDLQSYKLKEGASIIFMIN
KTKKTPTPVPETKSTTGTSNVENKSTTESSTQNKAQGSTNESTTTTSSSSAPAPAPAGATTTTSEQQQPQPA
ASNESTFAVGSEREASIQNIMEMGYERPQVEAALRAAFNNPHRAVEYLLTGIPESLQHPVAPAQPPATGTAP
AQQTEGNTSESGQQGEDEEHEGDESTQHENLFEAAAAAAAGAGAGGAGSGAGAGAGSAEGDIGGLGDDQQMQ
LLRAALQSNPELIQPLLEQLAASNPQIANLIQQDPEAFIRMFLSGAPGSGNDLGFEFEDESGETGAGGAAAA
ATGEDEQGTIRIQLSEQDNNAINRLCELGFERDIVIQVYLACDKNEEVAADILFRDM*

| Rank | Sequence | Start position | Score |
|---|---|---|---|
| 1 | PELIQPLLEQLAASNP | 298 | 0.93 |
| 1 | TGIPESLQHPVAPAQP | 194 | 0.93 |
| 2 | PETKSTTGTSNVENKS | 82 | 0.91 |
| 2 | PATGTAPAQQTEGNTS | 210 | 0.91 |
| 2 | TSSSSAPAPAPAGATT | 118 | 0.91 |
| 3 | AVEYLLTGIPESLQHP | 188 | 0.90 |
| 4 | AGAGGAGSGAGAGAGS | 258 | 0.89 |
| 5 | ERPQVEAALRAAFNNP | 170 | 0.88 |
| 6 | ESSQIKLVYSGKVLQD | 37 | 0.85 |
| 6 | EASIQNIMEMGYERPQ | 158 | 0.85 |
| 6 | PAPAGATTTTSEQQQP | 126 | 0.85 |
| 7 | ANLIQQDPEAFIRMFL | 316 | 0.84 |
| 7 | MQLLRAALQSNPELIQ | 287 | 0.84 |
| 7 | GSTNESTTTTSSSSAP | 109 | 0.84 |
| 8 | KTKKTPTPVPETKSTT | 73 | 0.83 |
| 8 | DESGETGAGGAAAAAT | 347 | 0.83 |
| 8 | SESGQQGEDEEHEGDE | 225 | 0.83 |
| 8 | VAPAQPPATGTAPAQQ | 204 | 0.83 |
| 9 | ASIIFMINKTKKTPTP | 65 | 0.80 |
| 10 | SAEGDIGGLGDDQQMQ | 273 | 0.79 |
| 10 | EDEEHEGDESTQHENL | 232 | 0.79 |
| 11 | DIVIQVYLACDKNEEV | 393 | 0.78 |
| 11 | TIRIQLSEQDNNAINR | 369 | 0.78 |
| 12 | PAQQTEGNTSESGQQG | 216 | 0.77 |
| 13 | SSTQNKAQGSTNESTT | 101 | 0.75 |

| Start | End | Max_score_pos | Sequence |
|---|---|---|---|
| 392 | 403 | 397 | RDIVIQVYLACD |
| 10 | 27 | 16 | KQTVSLDVELTDTVLSTK |
| 32 | 53 | 47 | QEKSCESSQIKLVYSGKVLQDD |
| 186 | 210 | 203 | HRAVEYLLTGIPESLQHPVAPAQPP |
| 287 | 313 | 302 | MQLLRAALQSNPELIQPLLEQLAASNP |
| 171 | 180 | 175 | RPQVEAALRA |
| 383 | 389 | 387 | NRLCELG |
| 56 | 62 | 59 | LQSYKLK |
| 315 | 322 | 316 | IANLIQQD |
| 64 | 70 | 68 | GASIIFM |
| 121 | 129 | 126 | SSAPAPAPA |
| 249 | 257 | 250 | EAAAAAAAG |
| 78 | 84 | 79 | PTPVPET |

33. RFA1
MSSLQLSKGALKQVFSKEGHDSVQIPMILQITNIKAFDVSPSDSKKFRILVNDGVYSTHGLIDESCSEYIKN
NNCQRYAIVQVNAFSIFATSKHFFVIKNFEVLAPTSEKSPNNIIPIDTYFLEHPEENYLTVMKKSESRDRES
PVPGVTPPLAQSTNSFKSEVGGGVAAQSKPAGTHRKVSPIETISPYQNNWTIKARVSYKGDLRTWSNSKGEG
KVFGFNLLDESDEIKASAFNETAERAHKLLEEGKVYYISKARVAAARKKFNTLSHPYELTFDKDTEITECFD
ESDVPKLNFNFVKLDQVQNLEANAIIDVLGALKTVFPPFQITAKSTGKVFDRRNILVVDETGFGIELGLWNN
TATDFNIEEGTVVAVKGCKVSDYDGRTLSLTQAGSIIPNPGTPESFKLKGWYDNIGIHESFKSLKIDNAGSG
GDKISQRISINQALEEHSGSTEKPDYFSIKASVTFCKPENFAYPACPNLVQNADATRPAQVCNKKLVFQDND
GTWRCERCAKTYEEPTWRYVLSCSVTDSTGHMWVTLFNDQAEKLLGIDATELVKKKEQKSEVANQIMNNTLF
KEFSLRVKAKQETYNDELKTRYSAAGINELDYASESQFLIKKLDQLLK*

| Rank | Sequence | Start position | Score |
|---|---|---|---|
| 1 | KSESRDRESPVPGVTP | 136 | 0.96 |
| 2 | YFSIKASVTFCKPENF | 458 | 0.95 |
| 3 | AKTYEEPTWRYVLSCS | 513 | 0.94 |
| 3 | DVSPSDSKKFRILVND | 38 | 0.94 |
| 4 | HGLIDESCSEYIKNNN | 59 | 0.93 |
| 5 | GSIIPNPGTPESFKLK | 394 | 0.91 |
| 6 | NWTIKARVSYKGDLRT | 193 | 0.89 |
| 7 | SGSTEKPDYFSIKASV | 450 | 0.88 |
| 7 | AGSGGDKISQRISINQ | 429 | 0.88 |
| 7 | PGTPESFKLKGWYDNI | 400 | 0.88 |
| 7 | YLTVMKKSESRDRESP | 130 | 0.88 |
| 8 | SVTFCKPENFAYPACP | 464 | 0.87 |
| 8 | DKDTEITECFDESDVP | 278 | 0.87 |
| 9 | DGVYSTHGLIDESCSE | 53 | 0.86 |
| 9 | DNDGTWRCERCAKTYE | 502 | 0.86 |
| 9 | NFEVLAPTSEKSPNNI | 100 | 0.86 |
| 10 | TGFGIELGLWNNTATD | 349 | 0.85 |
| 11 | TECFDESDVPKLNFNF | 284 | 0.84 |
| 11 | THRKVSPIETISPYQN | 177 | 0.84 |
| 11 | EHPEENYLTVMKKSES | 124 | 0.84 |
| 12 | GCKVSDYDGRTLSLTQ | 377 | 0.83 |
| 12 | GDLRTWSNSKGEGKVF | 204 | 0.83 |
| 13 | RISINQALEEHSGSTE | 439 | 0.82 |
| 13 | IIPIDTYFLEHPEENY | 115 | 0.82 |
| 14 | SLKIDNAGSGGDKISQ | 423 | 0.81 |
| 14 | ANAIIDVLGALKTVFP | 310 | 0.81 |
| 15 | GVAAQSKPAGTHRKVS | 167 | 0.80 |
| 16 | YNDELKTRYSAAGINE | 590 | 0.79 |
| 16 | GRTLSLTQAGSIIPNP | 385 | 0.79 |
| 16 | FNIEEGTVVAVKGCKV | 365 | 0.79 |
| 16 | VYYISKARVAAARKKF | 251 | 0.79 |
| 16 | LKQVFSKEGHDSVQIP | 11 | 0.79 |
| 17 | PIETISPYQNNWTIKA | 183 | 0.78 |
| 18 | AFSIFATSKHFFVIKN | 85 | 0.77 |
| 18 | KKKEQKSEVANQIMNN | 558 | 0.77 |
| 18 | PVPGVTPPLAQSTNSF | 145 | 0.77 |
| 19 | CSVTDSTGHMWVTLFN | 527 | 0.76 |
| 19 | NLVQNADATRPAQVCN | 480 | 0.76 |
| 19 | AYPACPNLVQNADATR | 474 | 0.76 |
| 19 | NSKGEGKVFGFNLLDE | 211 | 0.76 |
| 20 | LLGIDATELVKKKEQK | 548 | 0.75 |
| 20 | PPLAQSTNSFKSEVGG | 151 | 0.75 |

| Start | End | Max_score_pos | Sequence |
|---|---|---|---|
| 521 | 530 | 526 | WRYVLSCSVT |
| 370 | 383 | 375 | GTVVAVKGCKVSDY |
| 490 | 501 | 496 | PAQVCNKKLVFQ |
| 76 | 107 | 78 | QRYAIVQVNAFSIFATSKHFFVIKNFEVLAPT |
| 473 | 485 | 479 | FAYPACPNLVQNA |
| 297 | 308 | 302 | FNFVKLDQVQNL |
| 248 | 263 | 254 | EGKVYYISKARVAAAR |
| 313 | 330 | 326 | IIDVLGALKTVFPPFQIT |
| 20 | 32 | 26 | HDSVQIPMILQIT |
| 458 | 471 | 466 | YFSIKASVTFCKPE |
| 342 | 348 | 346 | NILVVDE |
| 144 | 156 | 152 | SPVPGVTPPLAQS |
| 4 | 17 | 13 | LQLSKGALKQVFSK |
| 56 | 69 | 58 | YSTHGLIDESCSEY |
| 47 | 54 | 53 | FRILVNDG |
| 268 | 276 | 272 | TLSHPYELT |
| 611 | 621 | 616 | ESQFLIKKLDQ |
| 577 | 586 | 582 | KEFSLRVKAK |
| 197 | 203 | 199 | KARVSYK |
| 165 | 173 | 171 | GGGVAAQSK |
| 178 | 189 | 184 | HRKVSPIETISP |
| 36 | 42 | 37 | AFDVSPS |
| 117 | 125 | 120 | PIDTYFLEH |
| 554 | 559 | 554 | TELVKK |
| 509 | 515 | 512 | CERCAKT |
| 387 | 398 | 390 | TLSLTQAGSIIP |
| 546 | 551 | 550 | EKLLGI |
| 418 | 424 | 423 | HESFKSL |

34. IPF9141
MSLQKISAFPDGNSFVHFNHSIGKLVIANSEGLMKILNTNDPESQPISIDILDNLTSLSSHEDKSIVLTTTE
GKLELIDLSTNTSKGVLYRSELPLRDTVFINQGNRVLCGGDENKLVIVDLQTTGEEDSNNKVSTISLPDQVV
NISYNTSGELSAISLSNGNVQIYSVVNEQPNLIYTINSVIPTKIHTSMDKVDYNDEHHDELFSTKTQWSTNG
QLLLVPTIDNQIHVYDRQDWTKVVKEFNNDNVKIIDFNLSAQGNLAIMSLNSFKVYNFSSEKLINEDDFEFD
EDGYPLNIIWKDNNLFVGSTVGETLHLRNVVKGKTDDVLNSLFISDAEEEEEEEEREVGRKKLGVEEDEGND
TDALLRDSDIEQDINVEDRIGQRKRNRRPYKLHEEDDLVIDEDDNENLGFDDRVVSNGYSTNGHKRYKSRSP
SEAIPKISKIKPYSPGSTPFEHRGASADRRYLTMNNIGYTWVVQNKDTTDDATSGSGSGGNSITVSFFDRSL
NTEYHFTDYHNFDLASMNQRGVLLGTSSTGHIYYRSHNEATNDSWDRKLPLLVSEYITSICITNNASATNTI
IVGTNLGYLRFFNQFGVCINILKTLPVVTLIASATVNAKLFVINQVTTNVYSYSILDINQDYKYIPHNAPMP
LKDHTPLIKGIFFNEFNDPCIVGGEDDTLLILHSWRESNNAKWIPILNCHKVLTEYGTNSNKKNWKCWPLGF
IGDKLNCLILKNNSQYPGFPLPLPIELEVELPIKNKFEEDEAEENFLRSLTLGKLISDSLNDELPGEIEEDE
VMERLNQYSMLFDKSLLKLFGESCKESKLGKAFSIARLIKTDKALLAASRIAERMEFLNLASKIGQLRESLV
DIDGDSD*

| Rank | Sequence | Start position | Score |
|---|---|---|---|
| 1 | HKVLTEYGTNSNKKNW | 698 | 0.95 |
| 1 | TGHIYYRSHNEATNDS | 533 | 0.95 |
| 2 | GVEEDEGNDTDALLRD | 352 | 0.92 |
| 3 | DTTDDATSGSGSGGNS | 479 | 0.90 |
| 3 | KLHEEDDLVIDEDDNE | 391 | 0.90 |
| 3 | EEEEEEREVGRKKLGV | 338 | 0.90 |
| 4 | ASRIAERMEFLNLASK | 840 | 0.89 |
| 4 | ASADRRYLTMNNIGYT | 457 | 0.89 |
| 5 | PGEIEEDEVMERLNQY | 785 | 0.88 |
| 5 | HTPLIKGIFFNEFNDP | 652 | 0.88 |
| 5 | VGRKKLGVEEDEGNDT | 346 | 0.88 |
| 6 | PSEAIPKISKIKPYSP | 432 | 0.87 |
| 6 | EGLMKILNTNDPESQP | 31 | 0.87 |
| 6 | IHTSMDKVDYNDEHHD | 188 | 0.87 |
| 6 | QVVNISYNTSGELSAI | 142 | 0.87 |
| 6 | LVIVDLQTTGEEDSNN | 117 | 0.87 |
| 7 | PLLVSEYITSICITNN | 554 | 0.86 |
| 7 | NTEYHFTDYHNFDLAS | 505 | 0.86 |
| 7 | YSPGSTPFEHRGASAD | 445 | 0.86 |
| 7 | QIHVYDRQDWTKVVKE | 227 | 0.86 |
| 7 | VSTISLPDQVVNISYN | 134 | 0.86 |
| 8 | RVVSNGYSTNGHKRYK | 413 | 0.85 |
| 8 | LQKISAFPDGNSFVHF | 3 | 0.85 |
| 9 | DLVIDEDDNENLGFDD | 397 | 0.84 |
| 9 | ALLRDSDIEQDINVED | 363 | 0.84 |
| 10 | ERLNQYSMLFDKSLLK | 795 | 0.82 |
| 10 | MNNIGYTWVVQNKDTT | 466 | 0.82 |
| 10 | NGHKRYKSRSPSEAIP | 422 | 0.82 |
| 10 | VPTIDNQIHVYDRQDW | 221 | 0.82 |
| 10 | SGELSAISLSNGNVQI | 151 | 0.82 |
| 11 | GSTVGETLHLRNVVKG | 306 | 0.81 |
| 11 | KLINEDDFEFDEDGYP | 278 | 0.81 |
| 12 | NLASKIGQLRESLVDI | 851 | 0.80 |
| 12 | TNNASATNTIIVGTNL | 567 | 0.80 |
| 13 | DKSIVLTTTEGKLELI | 63 | 0.78 |
| 13 | LGYLRFFNQFGVCINI | 582 | 0.78 |
| 13 | GGNSITVSFFDRSLNT | 491 | 0.78 |
| 13 | KLVIANSEGLMKILNT | 24 | 0.78 |
| 14 | HDELFSTKTQWSTNGQ | 202 | 0.77 |
| 15 | KLFGESCKESKLGKAF | 810 | 0.76 |
| 15 | GFIGDKLNCLILKNNS | 719 | 0.76 |
| 15 | ITSICITNNASATNTI | 561 | 0.76 |
| 16 | ILDINQDYKYIPHNAP | 631 | 0.75 |
| 16 | SATVNAKLFVINQVTT | 609 | 0.75 |
| 16 | FDEDGYPLNIIWKDNN | 287 | 0.75 |

| Start | End | Max_score_pos | Sequence |
|---|---|---|---|
| 164 | 173 | 167 | VQIYSVVNEQ |
| 590 | 621 | 604 | QFGVCINILKTLPVVTLIASATVNAKLFVINQ |
| 116 | 123 | 120 | KLVIVDLQ |
| 216 | 224 | 221 | GQLLLVPTI |
| 552 | 568 | 555 | KLPLLVSEYITSICITN |
| 623 | 635 | 629 | TTNVYSYSILDIN |
| 691 | 703 | 698 | WIPILNCEKVLTE |
| 724 | 731 | 727 | KLNCLILK |
| 310 | 321 | 316 | GETLHLRNVVKG |
| 135 | 147 | 141 | STISLPDQVVNIS |
| 737 | 752 | 742 | PGFPLPLPIELEVELP |
| 676 | 682 | 679 | TLLILHS |
| 326 | 333 | 329 | VLNSLFIS |
| 302 | 308 | 306 | NLFVGST |
| 22 | 29 | 25 | IGKLVIAN |
| 86 | 103 | 91 | KGVLYRSELPLRDTVFIN |
| 471 | 477 | 472 | YTWVVQN |
| 154 | 159 | 157 | LSAISL |
| 666 | 671 | 667 | DPCIVG |
| 715 | 722 | 718 | CWPLGFIG |
| 805 | 815 | 809 | DKSLLKLFGES |
| 175 | 188 | 186 | NLIYTINSVIPTKI |
| 525 | 530 | 529 | GVLLGT |
| 833 | 842 | 840 | TDKALLAASR |
| 495 | 501 | 497 | ITVSFFD |
| 44 | 61 | 49 | SQPISIDILDNLTSLSSH |
| 767 | 778 | 769 | LRSLTLGKLISD |
| 859 | 867 | 861 | LRESLVDID |
| 75 | 80 | 78 | LELIDL |
| 13 | 20 | 19 | NSFVHFNH |
| 268 | 274 | 269 | SFKVYNF |
| 411 | 418 | 417 | DDRVVSNG |
| 822 | 831 | 828 | GKAFSIARLI |
| 575 | 588 | 585 | TIIVGTNLGYLRFF |
| 534 | 539 | 538 | GHIYYR |
| 227 | 233 | 230 | QIHVYDR |
| 64 | 70 | 67 | KSIVLTT |
| 651 | 659 | 658 | DHTPLIKGI |
| 4 | 9 | 6 | QKISAF |
| 237 | 242 | 240 | TKVVKE |
| 637 | 644 | 640 | DYKYIPHN |
| 850 | 857 | 853 | LNLASKIG |
| 433 | 449 | 442 | SEAIPKISKIKPYSPGS |
| 248 | 256 | 250 | VKIIDFNLS |
| 395 | 401 | 398 | EDDLVID |
| 348 | 353 | 350 | RKKLGV |

35. IPF19872
MENIENLKLYINSLSQSISAYESALSPLQNKQLSDMILNINTTSSTTSTTSTSEEQQIQILNNFAYLLISTL
FSYLKSLGIDTDSHPIKMELSRIKSSMNRLKNIKNEINGDTNKQEEEEEEKEKLKKSKEYLSRTLGVRDVGS
SVDVKSMGTSAISKQNFQGKHIKFDDHDNADEKKDDGNKNDLKKSKNKKPNSTGSKSNSKSKSKLNVKESKI
TKPKSNKKSSTKNKNK*

| Rank | Sequence | Start position | Score |
|---|---|---|---|
| 1 | SQSISAYESALSPLQN | 15 | 0.89 |
| 2 | GKHIKFDDHDNADEKK | 163 | 0.88 |
| 3 | KESKITKPKSNKKSST | 212 | 0.86 |
| 3 | DEKKDDGNKNDLKKSK | 175 | 0.86 |
| 4 | SLGIDTDSHPIKMELS | 78 | 0.85 |
| 5 | KKPNSTGSKSNSKSKS | 192 | 0.82 |
| 6 | TTSSTTSTTSTSEEQQ | 42 | 0.81 |
| 7 | TSAISKQNFQGKHIKF | 153 | 0.80 |
| 8 | MELSRIKSSMNRLKNI | 90 | 0.79 |
| 8 | VDVKSMGTSAISKQNF | 146 | 0.79 |
| 9 | LSPLQNKQLSDMILNI | 25 | 0.78 |
| 10 | TNKQEEEEEEKEKLKK | 113 | 0.77 |
| 11 | EYLSRTLGVRDVGSSV | 131 | 0.76 |
| 12 | GNKNDLKKSKNKKPNS | 181 | 0.75 |

| Start | End | Max_score_pos | Sequence |
|---|---|---|---|
| 56 | 82 | 69 | QQIQILNNFAYLLISTLFSYLKSLGID |
| 129 | 149 | 145 | SKEYLSRTLGVRDVGSSVDVK |
| 6 | 31 | 26 | NLKLYINSLSQSISAYESALSPLQNK |
| 208 | 214 | 208 | KLNVKES |
| 84 | 95 | 95 | DSHPIKMELSRI |
| 163 | 168 | 168 | GKHIKF |

36. LPD1
MLRSFKSIPANGKLAQFVRYASTKKYDVVVIGGGPGGYVAAIKAAQLGLNTACIEKRGALGGTCLNVGCIPS
KSLLNNSHLLHQIQHEAKERGISIQGEVGVDFPKLMAAKEKAVKQLTGGIEMLFKKNKVDYLKGAGSFVNEK
TVKVTPIDGSEAQEVEADHIIVATGSEPTPFPGIEIDEERIVTSTGILSLKEVPERLAIIGGGIIGLEMASV
YARLGSKVTVIEFQNAIGAGMDAEVAKQSQKLLAKQGLDFKLGTKVVKGERDGEVVKIEVEDVKSGKKSDLE
ADVLLVAIGRRPFTEGLNFEAIGLEKDNKGRLIIDDQFKTKHDHIRVIGDVTFGPMLAHKAEEEGIAAAEYI
KKGHGHVNYANIPSVMYTHPEVAWVGLNEEQLKEQGIKYKVGKFPFIANSRAKTNMDTDGFVKFIADAETQR
VLGVHIIGPNAGEMIAEAGLALEYGASTEDISRTCHAHPTLSEAFKEAALATFDKPINF*

| Rank | Sequence | Start position | Score |
|---|---|---|---|
| 1 | SVMYTHPEVAWVGLNE | 374 | 0.95 |
| 2 | GVHIIGPNAGEMIAEA | 435 | 0.94 |
| 3 | TEDISRTCHAHPTLSE | 460 | 0.90 |
| 4 | AKTNMDTDGFVKFIAD | 412 | 0.89 |
| 4 | AEYIKKGHGHVNYANI | 357 | 0.89 |
| 5 | HIRVIGDVTFGPMLAH | 332 | 0.86 |
| 5 | LLVAIGRRPFTEGLNF | 292 | 0.86 |
| 5 | EKTVKVTPIDGSEAQE | 143 | 0.86 |
| 6 | AFKEAALATFDKPINF | 476 | 0.85 |
| 6 | VTVIEFQNAIGAGMDA | 224 | 0.85 |
| 7 | VAAIKAAQLGLNTACI | 39 | 0.84 |
| 8 | EQLKEQGIKYKVGKFP | 390 | 0.83 |
| 9 | ALEYGASTEDISRTCH | 453 | 0.82 |
| 9 | VEDVKSGKKSDLEADV | 276 | 0.82 |
| 9 | DVVVIGGGPGGYVAAI | 27 | 0.82 |
| 10 | TCLNVGCIPSKSLLNN | 63 | 0.81 |
| 10 | KGAGSFVNEKTVKVTP | 135 | 0.81 |
| 11 | TACIEKRGALGGTCLN | 51 | 0.80 |
| 11 | HPTLSEAFKEAALATF | 470 | 0.80 |
| 11 | LGTKVVKGERDGEVVK | 258 | 0.80 |
| 11 | QNAIGAGMDAEVAKQS | 230 | 0.80 |
| 11 | LRSFKSIPANGKLAQF | 2 | 0.80 |
| 11 | TSTGILSLKEVPERLA | 187 | 0.80 |
| 12 | GISIQGEVGVDFPKLM | 93 | 0.79 |
| 12 | FPGIEIDEERIVTSTG | 175 | 0.79 |
| 12 | VKQLTGGIEMLFKKNK | 115 | 0.79 |
| 13 | HGHVNYANIPSVMYTH | 364 | 0.78 |
| 14 | FPFIANSRAKTNMDTD | 404 | 0.77 |
| 15 | EEEGIAAAEYIKKGHG | 350 | 0.76 |
| 15 | IGLEMASVYARLGSKV | 209 | 0.76 |
| 15 | ATGSEPTPFPGIEIDE | 167 | 0.76 |
| 16 | EVGVDFPKLMAAKEKA | 99 | 0.75 |
| 16 | ADHIIVATGSEPTPFP | 161 | 0.75 |

| Start | End | Max_score_pos | Sequence |
|---|---|---|---|
| 288 | 297 | 294 | EADVLLVAIG |
| 431 | 440 | 436 | QRVLGVHIIG |
| 61 | 87 | 67 | GGTCLNVGCIPSKSLLNNSHLLHQIQH |
| 25 | 33 | 28 | KYDVVVIGG |
| 269 | 279 | 273 | GEVVKIEVEDV |
| 464 | 476 | 470 | SRTCHAHPTLSEA |
| 14 | 21 | 17 | LAQFVRYA |
| 185 | 206 | 194 | IVTSTGILSLKEVPERLAIIGG |
| 36 | 56 | 41 | GGYVAAIKAAQLGLNTACIEK |
| 211 | 230 | 217 | LEMASVYARLGSKVTVIEFQ |
| 156 | 169 | 164 | AQEVEADHIIVATG |
| 364 | 386 | 382 | HGHVNYANIPSVMYTHPEVAWVG |
| 143 | 151 | 148 | EKTVKVTPI |
| 332 | 348 | 336 | HIRVIGDVTFGPMLAHK |
| 259 | 265 | 260 | GTKVVKG |
| 241 | 253 | 251 | VAKQSQKLLAKQG |
| 97 | 108 | 103 | QGEVGVDFPKLM |
| 421 | 426 | 423 | FVKFIA |
| 398 | 409 | 404 | KYKVGKFPFIAN |
| 131 | 140 | 134 | VDYLKGAGSF |
| 449 | 457 | 455 | EAGLALEYG |
| 112 | 119 | 116 | EKAVKQLT |
| 319 | 324 | 323 | RLIIDD |
| 479 | 486 | 479 | EAALATFD |

37. PDB1
MSSLSSVTRSAKLATQSLKYNTRPSLSKIGQFQTSKITYRANSTQSTPVKEITVRDALNQALSEELDRDEDV
FLMGEEVAQYNGAYKVSRGLLDKFGEKRVIDTPITEMGFTGLAVGAALHGLKPVLEFMTWNFAMQGIDHILN
SAAKTLYMSGGKQPCNITFRGPNGAAAGVAAQHSQCYAAWYGSIPGLKVLSPYSAEDYKGLLKAAIRDPNPV
VFLENEIAYGETFKVSEEFSSPDFILPIGKAKIEKEGTDLTIVGHSRALKFAVEAAEILEKDFGIKAEVLNL
RSIKPLDVPAIVDSVKKTNHLVTVENGFPGFGVGSEICAQIMESEAFDYLDAPVERVTGCEVPTPYAKELED
FAFPDTEVILRACKKVLSL*

| Rank | Sequence | Start position | Score |
|---|---|---|---|
| 1 | GQFQTSKITYRANSTQ | 30 | 0.92 |
| 2 | CAQIMESEAFDYLDAP | 326 | 0.91 |
| 3 | RGLLDKFGEKRVIDTP | 90 | 0.90 |
| 4 | KRVIDTPITEMGFTGL | 99 | 0.89 |
| 5 | AFDYLDAPVERVTGCE | 334 | 0.87 |
| 5 | KAAIRDPNPVVFLENE | 207 | 0.87 |
| 6 | YRANSTQSTPVKEITV | 39 | 0.86 |
| 7 | YAAWYGSIPGLKVLSP | 181 | 0.85 |
| 7 | AMQGIDHILNSAAKTL | 135 | 0.85 |
| 8 | STPVKEITVRDALNQA | 46 | 0.84 |
| 8 | VERVTGCEVPTPYAKE | 342 | 0.84 |
| 8 | SAEDYKGLLKAAIRDP | 198 | 0.84 |
| 9 | NLRSIKPLDVPAIVDS | 287 | 0.83 |
| 10 | GVAAQHSQCYAAWYGS | 172 | 0.82 |
| 11 | VSEEFSSPDFILPIGK | 231 | 0.81 |
| 11 | KVLSPYSAEDYKGLLK | 192 | 0.81 |
| 11 | YMSGGKQPCNITFRGP | 151 | 0.81 |
| 12 | AQYNGAYKVSRGLLDK | 80 | 0.80 |
| 12 | AVEAAEILEKDFGIKA | 268 | 0.80 |
| 13 | ENEIAYGETFKVSEEF | 220 | 0.79 |
| 14 | TPYAKELEDFAFPDTE | 352 | 0.78 |
| 15 | GFTGLAVGAALHGLKP | 110 | 0.77 |
| 16 | VPAIVDSVKKTNHLVT | 296 | 0.76 |

| Start | End | Max_score_pos | Sequence |
|---|---|---|---|
| 365 | 376 | 376 | DTEVILRACKKV |
| 282 | 303 | 297 | KAEVLNLRSIKPLDVPAIVDSV |
| 170 | 200 | 194 | AAGVAAQHSQCYAAWYGSIPGLKVLSPYSAE |
| 213 | 222 | 216 | PNPVVFLENE |
| 113 | 129 | 117 | GLAVGAALHGLKPVLEF |
| 335 | 356 | 348 | FDYLDAPVERVTGCEVPTPYAK |
| 322 | 329 | 324 | GSEICAQI |
| 305 | 313 | 309 | KTNHLVTVE |
| 256 | 274 | 259 | LTIVGHSRALKFAVEAAEI |
| 46 | 52 | 51 | STPVKEI |
| 238 | 246 | 241 | PDFILPIGK |
| 4 | 19 | 5 | LSSVTRSAKLATQSLK |
| 203 | 210 | 207 | KGLLKAAI |
| 85 | 95 | 90 | AYKVSRGLLDK |
| 140 | 150 | 144 | DHILNSAAKTL |
| 25 | 30 | 29 | SLSKIG |
| 228 | 234 | 234 | TFKVSEE |
| 71 | 77 | 71 | DVFLMGE |

38. MDH12
MVKVTVAGAAGGIGQPLSLLLKLNPNVDELALFDIVNAKGVAADLSHINTPAVVTGHQPANKEDKTAITEAL
QGTDLVIIPAGVPRKPGMTRADLFNINASIIRDLVANIARVAPTAAILIISNPVNATVPIAAEVLKKLGVFN
PRKLFGVTTLDSVRAETFLGELTNTDPTKLKGKISVIGGHSGDTIVPLINYDAGVGVLSDSDYKNFVHRVQF
GGDEVVKAKNGAGSATLSMAYAGYRFADYVISSLTGGATPAGRIPDSSYIYLPGVSGGKEFSAKYVDGVDFF
SVPVVLSQGEIRSFVNPFEELTVTKEEKKLVEVALKGLKGSITQGTEFVNASKL*

| Rank | Sequence | Start position | Score |
|---|---|---|---|
| 1 | AGGIGQPLSLLLKLNP | 10 | 0.94 |
| 2 | KGSITQGTEFVNASKL | 327 | 0.92 |
| 3 | ASIIRDLVANIARVAP | 100 | 0.91 |
| 4 | LVIIPAGVPRKPGMTR | 77 | 0.90 |
| 5 | GDTIVPLINYDAGVGV | 186 | 0.89 |
| 6 | SLTGGATPAGRIPDSS | 249 | 0.88 |
| 7 | PGMTRADLFNINASII | 88 | 0.87 |
| 8 | SSYIYLPGVSGGKEFS | 263 | 0.86 |
| 9 | QGEIRSFVNPFEELTV | 296 | 0.85 |
| 9 | HRVQFGGDEVVKAKNG | 212 | 0.85 |
| 10 | PAVVTGHQPANKEDKT | 51 | 0.83 |
| 11 | TVPIAAEVLKKLGVFN | 129 | 0.82 |
| 12 | LSMAYAGYRFADYVIS | 233 | 0.80 |
| 13 | TFLGELTNTDPTKLKG | 161 | 0.79 |
| 13 | LVANIARVAPTAAILI | 106 | 0.79 |
| 14 | KKLGVFNPRKLFGVTT | 138 | 0.77 |
| 15 | AAILIISNPVNATVPI | 117 | 0.76 |
| 16 | TPAGRIPDSSYIYLPG | 255 | 0.75 |

| Start | End | Max_score_pos | Sequence |
|---|---|---|---|
| 278 | 297 | 291 | SAKYVDGVDFFSVPVVLSQG |
| 14 | 25 | 20 | GQPLSLLLKLNP |
| 316 | 329 | 320 | KKLVEVALKGLKGS |
| 74 | 87 | 81 | GTDLVIIPAGVPRK |
| 28 | 57 | 33 | DELALFDIVNAKGVAADLSHINTPAVVTGH |
| 262 | 272 | 268 | DSSYIYLPGVS |
| 127 | 143 | 139 | NATVPIAAEVLKKLGVF |
| 208 | 215 | 213 | KNFVHRVQ |
| 187 | 196 | 192 | DTIVPLINYD |
| 4 | 9 | 5 | VTVAGA |
| 242 | 250 | 248 | FADYVISSL |
| 103 | 125 | 120 | IRDLVANIARVAPTAAILIISNP |
| 198 | 205 | 200 | GVGVLSDS |
| 148 | 157 | 154 | LFGVTTLDSV |
| 176 | 183 | 181 | GKISVIGG |
| 219 | 225 | 222 | DEVVKAK |
| 300 | 306 | 302 | RSFVNPF |
| 233 | 240 | 239 | LSMAYAGY |

39. CAR1
MSSIQYKYHPDKKASIITAPFSGGQPKGGVELGPDYILKAGFQKQIESLGWTTDLKEPLEGTDYEKMKTNDK
DDFGVKNSKIVSESCQKIHDAVKGSLAEGKLPITIGGDHSIGTATVSASLVHDPSTCVVWVDAHADINTPKT
TDSGNLHGCPLSFIMGIDRDSYPPEFSWVPQVLKSNKLVYIGLRDVDDGEKEILRKHNIAAFSMYHVDKYGI
GKVVEMALDKVNPNRDCPVHLSYDVDAIDPSFVPATGTRVEGGLSLREGLFIAEEIAQSGLLQSLDIVETNP
MLAETEEHVLDTVSAACAIGRCALGQTLL*

| Rank | Sequence | Start position | Score |
|---|---|---|---|
| 1 | MSSIQYKYHPDKKASI | 1 | 0.96 |
| 2 | PLEGTDYEKMKTNDKD | 58 | 0.92 |
| 3 | ASLVHDPSTCVVWVDA | 120 | 0.91 |
| 4 | DHSIGTATVSASLVHD | 110 | 0.90 |
| 5 | KMKTNDKDDFGVKNSK | 66 | 0.86 |
| 5 | KVVEMALDKVNPNRDC | 218 | 0.86 |
| 6 | INTPKTTDSGNLHGCP | 139 | 0.84 |
| 7 | APFSGGQPKGGVELGP | 19 | 0.83 |
| 7 | FIMGIDRDSYPPEFSW | 157 | 0.83 |
| 8 | DCPVHLSYDVDAIDPS | 232 | 0.82 |
| 9 | KLPITIGGDHSIGTAT | 102 | 0.81 |
| 10 | KGGVELGPDYILKAGF | 27 | 0.80 |
| 11 | SLGWTTDLKEPLEGTD | 48 | 0.78 |
| 11 | TVSAACAIGRCALGQT | 300 | 0.78 |
| 11 | LVYIGLRDVDDGEKEI | 182 | 0.78 |
| 12 | DKYGIGKVVEMALDKV | 212 | 0.77 |
| 13 | PSFVPATGTRVEGGLS | 246 | 0.76 |
| 13 | STCVVWVDAHADINTP | 127 | 0.76 |
| 14 | SLDIVETNPMLAETEE | 280 | 0.75 |

| Start | End | Max_score_pos | Sequence |
|---|---|---|---|
| 232 | 253 | 236 | DCPVHLSYDVDAIDPSFVPATG |
| 114 | 136 | 132 | GTATVSASLVHDPSTCVVWVDAH |
| 294 | 314 | 304 | EEHVLDTVSAACAIGRCALGQ |
| 167 | 188 | 176 | PPEFSWVPQVLKSNKLVYIGLR |
| 150 | 158 | 153 | LHGCPLSFI |
| 274 | 285 | 281 | QSGLLQSLDIVE |
| 79 | 97 | 85 | NSKIVSESCQKIHDAVKGS |
| 216 | 227 | 217 | IGKVVEMALDKV |
| 206 | 214 | 208 | FSMYHVDKY |
| 29 | 41 | 35 | GVELGPDYILKAG |
| 4 | 9 | 7 | IQYKYE |
| 13 | 20 | 18 | KASIITAP |
| 263 | 272 | 269 | REGLFIAEEI |
| 100 | 106 | 104 | EGKLPIT |

40. IPF6881
MTKEQLNNDSKNSVVEEEDGGAQFQSYLNNDGIDELTPSVRKHRVSSLSLSDLNQWQNGLTKLSSSTSLSKK
NSSSANLKKVDSLAKLSRNASIIKRKKKEIIDHERVASYAFCFDIDGVILRGPDTIPQAVEAMKLLNGENKY
HIKVPSIFVTNGGGKPEQQRADDLSKRLNCTITKEQIIQGHTPMKDLVDVYKNVLVVGGVGNVCRNVAESYG
FKNVYTPLDIMKWNPAVSPYHDLTEEERVCTKDVDFHKIPIDAIMVFADSRNWAADQQIILELLLSVNGVMG
TQSKTFDEGPQIYFAHSDFIWATNYKLSRYGMGALQVSIAALYREHTGKELKVNRFGKPQKGTFKFANKVLS
HWRQGVLDEHLKKLSVNDPNANDADILINEDGEEIINQAKLENYNWSDSEDDEDDEDAVNGGSSTAKKALKD
VGKIADVGKPDKITLELPPASTVYFVGDTPESDIRFANSHDASWHSILVKTGVYQAGTEPKYKPKHLCNDVL
EAVKYAIEREHAMELAEWNETAQDVNEDDKGSRLNFADLVMTPSDKKENDTTEKKPSGVSSTSKSSIAEAEE
VEVPDILAAQIEKLKDVSVSK*

| Rank | Sequence | Start position | Score |
|---|---|---|---|
| 1 | VGKIADVGKPDKITLE | 433 | 0.92 |
| 1 | EQIIQGHTPMKDLVDV | 179 | 0.92 |
| 2 | ESDIRFANSHDASWHS | 463 | 0.91 |
| 2 | LELPPASTVYFVGDTP | 447 | 0.91 |
| 2 | PDTIPQAVEAMKLLNG | 125 | 0.91 |
| 3 | KKEIIDHERVASYAFC | 99 | 0.89 |
| 3 | ADLVMTPSDKKENDTT | 541 | 0.89 |
| 3 | REHAMELAEWNETAQD | 513 | 0.89 |
| 3 | AVSPYHDLTEEERVCT | 232 | 0.89 |
| 3 | KPEQQRADDLSKRLNC | 159 | 0.89 |
| 3 | VEEEDGGAQFQSYLNN | 15 | 0.89 |
| 4 | GVMGTQSKTFDEGPQI | 285 | 0.88 |
| 5 | NETAQDVNEDDKGSRL | 523 | 0.87 |
| 5 | YQAGTEPKYKPKHLCN | 486 | 0.87 |
| 5 | DEHLKKLSVNDPNAND | 368 | 0.87 |
| 5 | GIDELTPSVRKHRVSS | 32 | 0.87 |
| 6 | QAKLENYNWSDSEDDE | 398 | 0.86 |
| 7 | HSILVKTGVYQAGTEP | 477 | 0.85 |
| 7 | YFVGDTPESDIRFANS | 456 | 0.85 |
| 7 | DILINEDGEEIINQAK | 385 | 0.85 |
| 7 | GKPQKGTFKFANKVLS | 345 | 0.85 |
| 8 | CRNVAESYGFKNVYTP | 208 | 0.84 |
| 9 | AALYREHTGKELKVNR | 328 | 0.83 |
| 9 | EGPQIYFAHSDFIWAT | 296 | 0.83 |
| 10 | SGVSSTSKSSIAEAEE | 561 | 0.82 |
| 10 | NWSDSEDDEDDEDAVN | 405 | 0.82 |
| 10 | TNYKLSRYGMGALQVS | 311 | 0.82 |
| 10 | CFDIDGVILRGPDTIP | 114 | 0.82 |
| 11 | SDFIWATNYKLSRYGM | 305 | 0.81 |
| 12 | KIPIDAIMVFADSRNW | 254 | 0.79 |
| 12 | IMKWNPAVSPYHDLTE | 226 | 0.79 |
| 13 | GFKNVYTPLDIMKWNP | 216 | 0.78 |
| 14 | LVVGGVGNVCRNVAES | 199 | 0.77 |
| 15 | KKENDTTEKKPSGVSS | 550 | 0.76 |
| 16 | KVDSLAKLSRNASIIK | 81 | 0.75 |

| Start | End | Max_score_pos | Sequence |
|---|---|---|---|
| 189 | 214 | 201 | KDLVDVYKNVLVVGGVGNVCRNVAES |
| 272 | 286 | 280 | DQQIILELLLSVNGV |
| 105 | 123 | 111 | HERVASYAFCFDIDGVILR |
| 321 | 333 | 328 | GALQVSIAALYRE |
| 495 | 510 | 501 | KPKHLCNDVLEAVKYA |
| 145 | 154 | 150 | HIKVPSIFVT |
| 444 | 460 | 455 | KITLELPPASTVYFVGD |
| 576 | 588 | 580 | EVEVPDILAAQIE |
| 36 | 53 | 48 | LTPSVRKHRVSSLSLSDL |
| 474 | 488 | 483 | ASWHSILVKTGVYQA |
| 355 | 377 | 373 | ANKVLSHWRQGVLDEHLKKLSVN |
| 231 | 238 | 234 | PAVSPYHD |
| 217 | 224 | 223 | FKNVYTPL |
| 243 | 265 | 247 | ERVCTKDVDFHKIPIDAIMVFAD |
| 79 | 88 | 85 | LKKVDSLAKL |
| 540 | 546 | 541 | FADLVMT |
| 427 | 440 | 436 | KKALKDVGKIADVG |
| 299 | 307 | 301 | QIYFAHSDF |
| 128 | 135 | 131 | IPQAVEAM |
| 60 | 69 | 66 | LTKLSSSTSL |
| 12 | 17 | 12 | NSVVEE |
| 560 | 566 | 562 | PSGVSST |

41. IPF4258
MSDSVDRVFVKAIATIRALSSRSNYGSLPRPPAENRIKLYGLYKQATEGDVDGVMPRPVGFTAEDEGAKKKW
DAWKREQGLSKTEAKKRYVSYLIETMRVYASGTSEARELLNELEYLWEQIKDLPSSDEETDHHHIPLPSRSP
TFSQTDRFSNRTPSITGARTTGTSNLNNIYSHSRRNTTLSLNEYVQQQRMQHQNQQQLHDTTSQPGAPVGGG
GGGGGSIYSLPGRMGANNVIEDFKNWQSEVNMVINKLTREFVNSRREVQGNENGDPSTGDRDEELDDVEIIK
RRIIHILKFVGWNALKFLKNFAVSLITFMFIVWCIKKNVHVERTYVKQPTNNANKSKKELIINMVLNTDENK
WFIRLLGFINRFIGFV*

| Rank | Sequence | Start position | Score |
|---|---|---|---|
| 1 | GGGSIYSLPGRMGANN | 219 | 0.95 |
| 2 | YGLYKQATEGDVDGVM | 40 | 0.90 |
| 2 | HVERTYVKQPTNNANK | 328 | 0.90 |
| 3 | SYLIETMRVYASGTSE | 92 | 0.88 |
| 3 | RPVGFTAEDEGAKKKW | 57 | 0.88 |
| 3 | PVGGGGGGGGSIYSLP | 212 | 0.88 |
| 3 | PSITGARTTGTSNLNN | 157 | 0.88 |
| 4 | AKKKWDAWKREQGLSK | 68 | 0.87 |
| 5 | RVYASGTSEARELLNE | 99 | 0.85 |
| 5 | HHHIPLPSRSPTFSQT | 134 | 0.85 |
| 6 | QQLHDTTSQPGAPVGG | 200 | 0.83 |
| 6 | RSPTFSQTDRFSNRTP | 142 | 0.83 |
| 7 | RREVQGNENGDPSTGD | 261 | 0.82 |
| 7 | LPSSDEETDHHHIPLP | 125 | 0.82 |
| 8 | WEQIKDLPSSDEETDH | 119 | 0.81 |
| 9 | PAENRIKLYGLYKQAT | 32 | 0.80 |
| 10 | KRRIIHILKFVGWNAL | 288 | 0.79 |
| 10 | HSRRNTTLSLNEYVQQ | 176 | 0.79 |
| 10 | LNNIYSHSRRNTTLSL | 170 | 0.79 |
| 11 | PGRMGANNVIEDFKNW | 227 | 0.78 |
| 12 | LSKTEAKKRYVSYLIE | 81 | 0.77 |
| 13 | NNVIEDFKNWQSEVNM | 233 | 0.76 |

| Start | End | Max_score_pos | Sequence |
|---|---|---|---|
| 5 | 20 | 11 | VDRVFVKAIATIRALS |
| 289 | 301 | 295 | RRIIHILKFVGWN |
| 303 | 336 | 321 | LKFLKNFAVSLITFMFIVWCIKKNVHVERTYVKQ |
| 88 | 103 | 92 | KRYVSYLIETMRVYAS |
| 134 | 143 | 137 | HHHIPLPSRS |
| 38 | 45 | 40 | KLYGLYKQ |
| 185 | 192 | 186 | LNEYVQQQ |
| 363 | 373 | 366 | IRLLGFINRFI |
| 209 | 214 | 210 | PGAPVG |
| 56 | 62 | 57 | PRPVGFT |
| 26 | 31 | 30 | GSLPRP |
| 112 | 120 | 114 | LNELEYLWE |
| 246 | 252 | 247 | VNMVINK |
| 199 | 205 | 199 | QQQLHDT |

42. IPF7489
MTRYRLTYQLKNIALEFGENDGKYFIQLGHSSTGKILNLSTLPSYLTERKVIIIDSVKSGTGRTPGKDIYSD
ILDPLFKELSIEHEYHATKSATSISELASSLKDHKVTIIFISGDTSINEFINSLNDSEKGEIAIFPIPGGTG
NSLSLSLNITNPLDAIIRLFSAGTTSPLNLYEVDFPQGSHYLIANELGSPVPSHLKFLVVLSWGFHASLVAD
SDTPELRKHGIKRFQLAAHQNLSRDQKYEGDFYINDVELNGPFAYWLVTASQRFEPTFEISPKGDILKDELY
VVTFNTQNTQYYIMDIMKEVYDKGSHIKNPNVVYKKLDKNDKIQLKTKNSKPLIQRRFCVDGSIIALPETES
HEIYIHVKDNSQHSWKLYIIH*

| Rank | Sequence | Start position | Score |
|---|---|---|---|
| 1 | GSHIKNPNVVYKKLDK | 312 | 0.95 |
| 2 | KVIIIDSVKSGTGRTP | 50 | 0.92 |
| 3 | SVKSGTGRTPGKDIYS | 56 | 0.90 |
| 3 | ISGDTSINEFINSLND | 113 | 0.90 |
| 4 | YIMDIMKEVYDKGSHI | 300 | 0.89 |
| 4 | GGTGNSLSLSLNITNP | 141 | 0.89 |
| 5 | ELSIEHEYHATKSATS | 80 | 0.86 |
| 5 | SHEIYIHVKDNSQHSW | 360 | 0.86 |
| 5 | NLSRDQKYEGDFYIND | 237 | 0.86 |
| 6 | YQLKNIALEFGENDGK | 8 | 0.84 |
| 6 | LVTASQRFEPTFEISP | 263 | 0.84 |
| 7 | YFIQLGHSSTGKILNL | 24 | 0.83 |
| 8 | SISELASSLKDHKVTI | 95 | 0.82 |
| 9 | KGDILKDELYVVTFNT | 279 | 0.80 |
| 9 | EPTFEISPKGDILKDE | 271 | 0.80 |
| 9 | FSAGTTSPLNLYEVDF | 164 | 0.80 |
| 10 | TGKILNLSTLPSYLTE | 33 | 0.79 |
| 10 | KGEIAIFPIPGGTGNS | 131 | 0.79 |
| 11 | GRTPGKDIYSDILDPL | 62 | 0.78 |
| 11 | SLNITNPLDAIIRLFS | 150 | 0.78 |
| 12 | DFYINDVELNGPFAYW | 247 | 0.77 |
| 12 | DFPQGSHYLIANELGS | 178 | 0.77 |
| 13 | RKHGIKRFQLAAHQNL | 223 | 0.75 |
| 13 | LVADSDTPELRKHGIK | 213 | 0.75 |

| Start | End | Max_score_pos | Sequence |
|---|---|---|---|
| 192 | 217 | 202 | GSPVPSHLKFLVVLSWGFHASLVADS |
| 48 | 57 | 54 | ERKVIIIDSV |
| 284 | 292 | 289 | KDELYVVTF |
| 361 | 370 | 364 | HEIYIHVKDN |
| 260 | 267 | 261 | AYWLVTAS |
| 317 | 324 | 323 | NPNVVYKK |
| 339 | 358 | 350 | KPLIQRRFCVDGSIIALPET |
| 94 | 114 | 111 | TSISELASSLKDHKVTIIFIS |
| 134 | 140 | 137 | IAIFPIP |
| 171 | 189 | 174 | PLNLYEVDFPQGSHYLIAN |
| 146 | 152 | 150 | SLSLSLN |
| 36 | 46 | 42 | ILNLSTLPSYL |
| 24 | 31 | 27 | YFIQLGHS |
| 5 | 16 | 7 | RLTYQLKNIALE |
| 156 | 166 | 160 | PLDAIIRLFSA |
| 228 | 237 | 233 | KRFQLAAHQN |
| 70 | 90 | 76 | YSDILDPLFKELSIEHEYHAT |

43. MYO5
MAIVKRGGRTKTKQQQVPAKSSGGGSSGGIKKAEFDITKKKEVGVSDLTLLSKITDEAINENLHKRFMNDTI
YTYIGHVLISVNPFRDLGIYTLENLNKYKGRNRLEVPPHVFAIAESMYYNLKSYGENQCVIISGESGAGKTE
AAKQIMQYIANVSVNQDNVEISKIKDMVLATNPLLESFGCAKTLRNNNSSRHGKYLEIKFSEGNYQPIAAHI
TNYLLEKQRVVSQITNERNFHIFYQFTKHCPPQYQQMFGIQGPETYVYTSAAKCINVDGVDDAKDFQDTLNA
MKIIGLTQQEQDNIFRMLASILWIGNISFVEDENGNAAIRDDSVTNFAAYLLDVNPEILKKAIIEKTIETSH
GMRRGSTYHSPLNIVQATAVRDALAKGIYNNLFEWIVERVNISLAGSQQQSSKSIGILDIYGFEIFERNSFE
QICINYVNEKLQQIFIQLTLKAEQDEYVQEQIKWTPIDYFNNKVVCDLIEATRPQPGLFAALNDSIKTAHAD
SEAADQVFAQRLSMVGASNRHFEDRRGKFIIKHYAGDVTYDVAGMTDKNKDAMLRDLLELVSTSQNSFINQV
LFPPDLLTQLTDSRKRPETASDKIKKSANILVDTLSQCTPSYIRTIKPNQTKKPRDYDNQQVLHQIKYLGLK
ENVRIRRAGFAYRSTFERFVQRFYLLSPATGYAGDYIWRGDDISAVKEILKSCHIPPSEYQLGTTKVFIKTP
ETLFALEDMRDKYWHNMAARIQRAWRRYVKRKEDAAKTIQNAWRIKKHGNQFEQFRDYGNGLLQGRKERRRM
SMLGSRAFMGDYLGCNYKSGYGRFIINQVGINESVILSSKGEILLSKFGRSSKRLPRIFIVTKTSIYIIAEV
LVEKRLQLQKEFTIPISGINYLGLSTFQDNWVAISLHSPTPTTPDVFINLDFKTELVAQLKKLNPGITIKIG
PTIEYQKKPGKFHTVKFIIGAGPEIPNNGDHYKSGTVSVKQGLPASSKNPKRPRGVSSKVDYSKYYNRGAAR
KTAAAAQATPRYNQPTPVANSGYSAQPAYPIPQQPQQYQPQQSQQQTPYPTQSSIPSVNQNQSRQPQRKVPP
PAPSLQVSAAQAALGKSPTQQRQTPAHNPVASPNRPASTTIATTTSHTSRPVKKTAPAPPVKKTAPPPPPPT
LVKPKFPTYKAMFDYDGSVAGSIPLVKDTVYYVTQVNGKWGLVKTMDETKEGWSPIDYLKECSPNETQKSAP
PPPPPPPAATASAGANGASNPISTTTSTNTTTSSHTTNATSNGSLGNGLADALKAKKQEETTLAGSLADALK
KRQGATRDSDDEEEEDDDDW*

| Rank | Sequence | Start position | Score |
|---|---|---|---|
| 1 | TKEGWSPIDYLKECSP | 1201 | 0.97 |
| 2 | PSEYQLGTTKVFIKTP | 705 | 0.95 |
| 2 | EKTIETSHGMRRGSTY | 353 | 0.95 |
| 3 | SPTPTTPDVFINLDFK | 902 | 0.93 |
| 4 | GKFIIKHYAGDVTYDV | 531 | 0.92 |
| 4 | MFGIQGPETYVYTSAA | 253 | 0.92 |
| 4 | NQCVIISGESGAGKTE | 129 | 0.92 |
| 5 | ARIQRAWRRYVKRKED | 739 | 0.91 |
| 5 | AGDVTYDVAGMTDKNK | 539 | 0.91 |
| 5 | DENGNAAIRDDSVTNF | 320 | 0.91 |
| 6 | PRGVSSKVDYSKYYNR | 989 | 0.90 |
| 6 | KFIIGAGPEIPNNGDH | 952 | 0.90 |
| 6 | GPTIEYQKKPGKFHTV | 936 | 0.90 |
| 6 | GITIKIGPTIEYQKKP | 930 | 0.90 |
| 6 | AAHITNYLLEKQRVVS | 213 | 0.90 |
| 7 | EATRPQPGLFAALNDS | 482 | 0.89 |
| 7 | GFEIFERNSFEQICIN | 422 | 0.89 |
| 8 | AKTIQNAWRIKKHGNQ | 756 | 0.88 |
| 8 | PATGYAGDYIWRGDDI | 676 | 0.88 |
| 8 | TPSYIRTIKPNQTKKP | 615 | 0.88 |
| 8 | AKCINVDGVDDAKDFQ | 268 | 0.88 |
| 8 | SGESGAGKTEAAKQIM | 135 | 0.88 |
| 8 | PVKKTAPAPPVKKTAP | 1131 | 0.88 |
| 9 | KRPETASDKIKKSANI | 591 | 0.87 |
| 9 | VQEQIKWTPIDYFNNK | 460 | 0.87 |
| 9 | QGATRDSDDEEEEDDD | 1299 | 0.87 |
| 9 | TASAGANGASNPISTT | 1234 | 0.87 |
| 9 | YLKECSPNETQKSAPP | 1210 | 0.87 |
| 10 | FGRSSKRLPRIFIVTK | 840 | 0.86 |
| 10 | HVLISVNPFRDLGIYT | 78 | 0.86 |
| 10 | QIMQYIANVSVNQDNV | 148 | 0.86 |
| 10 | YKAMFDYDGSVAGSIP | 1161 | 0.86 |
| 10 | QQSQQQTPYPTQSSIP | 1049 | 0.86 |
| 11 | NDTIYTYIGHVLISVN | 69 | 0.85 |
| 11 | KAEQDEYVQEQIKWTP | 453 | 0.85 |
| 11 | TSTNTTTSSHTTNATS | 1250 | 0.85 |
| 11 | AQAALGKSPTQQRQTP | 1090 | 0.85 |
| 11 | QQPQQYQPQQSQQQTP | 1041 | 0.85 |
| 11 | NSGYSAQPAYPIPQQP | 1028 | 0.85 |
| 12 | KSCHIPPSEYQLGTTK | 699 | 0.84 |
| 12 | LQQIFIQLTLKAEQDE | 443 | 0.84 |
| 12 | AGSQQQSSKSIGILDI | 405 | 0.84 |
| 12 | AKKQEETTLAGSLADA | 1279 | 0.84 |
| 12 | SAPPPPPPPPAATASA | 1222 | 0.84 |
| 12 | VKKTAPPPPPPTLVKP | 1141 | 0.84 |
| 12 | ATTTSHTSRPVKKTAP | 1122 | 0.84 |
| 12 | AAAAQATPRYNQPTPV | 1011 | 0.84 |
| 13 | VDYSKYYNRGAARKTA | 996 | 0.83 |
| 13 | GDHYKSGTVSVKQGLP | 965 | 0.83 |
| 13 | GPEIPNNGDHYKSGTV | 958 | 0.83 |
| 13 | KPGKFHTVKFIIGAGP | 944 | 0.83 |
| 13 | LGCNYKSGYGRFIINQ | 805 | 0.83 |
| 13 | PNQTKKPRDYDNQQVL | 624 | 0.83 |
| 13 | SFEQICINYVNEKLQQ | 430 | 0.83 |
| 13 | KHCPPQYQQMFGIQGP | 244 | 0.83 |
| 13 | AKSSGGGSSGGIKKAE | 19 | 0.83 |
| 13 | WGLVKTMDETKEGWSP | 1192 | 0.83 |
| 14 | GLPASSKNPKRPRGVS | 978 | 0.82 |
| 14 | DEAINENLHKRFMNDT | 56 | 0.82 |
| 14 | SHGMRRGSTYHSPLNI | 359 | 0.82 |
| 14 | AGSLADALKKRQGATR | 1288 | 0.82 |
| 14 | PTQQRQTPAHNPVASP | 1098 | 0.82 |
| 15 | DVAGMTDKNKDAMLRD | 545 | 0.81 |
| 15 | MVLATNPLLESFGCAK | 171 | 0.81 |
| 15 | ESMYYNLKSYGENQCV | 117 | 0.81 |
| 16 | ENLNKYKGRNRLEVPP | 95 | 0.80 |
| 16 | LNIVQATAVRDALAKG | 372 | 0.80 |
| 16 | SSGGIKKAEFDITKKK | 26 | 0.80 |
| 17 | PETLFALEDMRDKYWH | 720 | 0.79 |
| 17 | KVFIKTPETLFALEDM | 714 | 0.79 |
| 17 | AIVKRGGRTKTKQQQV | 2 | 0.79 |
| 17 | NVEISKIKDMVLATNP | 162 | 0.79 |
| 17 | ASNPISTTTSTNTTTS | 1242 | 0.79 |
| 17 | IPLVKDTVYYVTQVNG | 1175 | 0.79 |
| 17 | YPTQSSIPSVNQNQSR | 1057 | 0.79 |
| 18 | EFDITKKKEVGVSDLT | 34 | 0.78 |
| 18 | LWIGNISFVEDENGNA | 310 | 0.78 |
| 18 | TLVKPKFPTYKAMFDY | 1152 | 0.78 |
| 18 | PVASPNRPASTTIATT | 1109 | 0.78 |
| 19 | MSMLGSRAFMGDYLGC | 792 | 0.77 |
| 19 | HGKYLEIKFSEGNYQP | 196 | 0.77 |
| 20 | GTVSVKQGLPASSKNP | 971 | 0.76 |
| 20 | AKTLRNNNSSRHGKYL | 185 | 0.76 |
| 20 | TVYYVTQVNGKWGLVK | 1181 | 0.76 |
| 20 | PSVNQNQSRQPQRKVP | 1064 | 0.76 |
| 20 | PRYNQPTPVANSGYSA | 1018 | 0.76 |
| 21 | GDYIWRGDDISAVKEI | 682 | 0.75 |
| 21 | SKSIGILDIYGFEIFE | 412 | 0.75 |
| 21 | NRGAARKTAAAAQATP | 1003 | 0.75 |

| Start | End | Max_score_pos | Sequence |
|---|---|---|---|
| 1168 | 1188 | 1185 | DGSVAGSIPLVKDTVYYVTQV |
| 474 | 481 | 478 | NKVVCDLI |
| 845 | 890 | 863 | KRLPRIFIVTKTSIYIIAEVLVEKRLQLQKEFTIPISGINY |
| | | | LGLST |
| 73 | 86 | 80 | YTYIGHVLISVNPF |
| 106 | 118 | 111 | LEVPPHVFAIAES |
| 432 | 442 | 436 | EQICINYVNEK |
| 333 | 345 | 339 | TNFAAYLLDVNPE |
| 665 | 679 | 671 | ERFVQRFYLLSPATG |
| 691 | 709 | 700 | ISAVKEILKSCHIPPSEYQ |
| 128 | 135 | 133 | ENQCVIIS |
| 635 | 648 | 641 | NQQVLHQIKYLGLK |
| 894 | 903 | 899 | NWVAISLHSP |
| 368 | 383 | 377 | YHSPLNIVQATAVRDA |
| 573 | 586 | 579 | INQVLFPPDLLTQL |
| 148 | 161 | 155 | QIMQYIANVSVNQD |
| 42 | 53 | 48 | EVGVSDLTLLSK |
| 559 | 568 | 564 | RDLLELVSTS |
| 1075 | 1097 | 1084 | QRKVPPPAPSLQVSAAQAALGKS |
| 603 | 621 | 611 | SANILVDTLSQCTPSYIRT |
| 970 | 982 | 976 | SGTVSVKQGLPAS |
| 259 | 276 | 273 | PETYVYTSAAKCINVDGV |
| 237 | 251 | 248 | HIFYQFTKHCPPQYQ |
| 210 | 230 | 227 | QPIAAHITNYLLEKQRVVSQI |
| 917 | 927 | 923 | KTELVAQLKKL |
| 444 | 453 | 449 | QQIFIQLTLK |
| 1129 | 1162 | 1151 | SRPVKKTAPAPPVKKTAPPPPPPTLVKPKFPTYK |
| 304 | 313 | 309 | RMLASILWIG |
| 823 | 840 | 829 | INESVILSSKGEILLSKF |
| 989 | 1001 | 995 | PRGVSSKVDYSKY |
| 169 | 187 | 181 | KDMVLATNPLLESFGCAKT |
| 948 | 958 | 952 | FHTVKFIIGAG |
| 508 | 519 | 513 | ADQVFAQRLSMV |
| 1207 | 1214 | 1213 | PIDYLKEC |
| 487 | 496 | 491 | QPGLFAALND |
| 533 | 547 | 536 | FIIKHYAGDVTYDVA |
| 1053 | 1067 | 1063 | QQTPYPTQSSIPSVN |
| 907 | 914 | 913 | TPDVFINL |
| 410 | 424 | 418 | QSSKSIGILDIYGFE |
| 1032 | 1051 | 1036 | SAQPAYPIPQQPQQYQPQQS |
| 394 | 408 | 399 | EWIVERVNISLAGSQ |
| 804 | 810 | 807 | YLGCNYK |
| 1107 | 1113 | 1108 | HNPVASP |
| 457 | 463 | 462 | DEYVQEQ |
| 1274 | 1279 | 1276 | ADALKA |
| 14 | 20 | 16 | QQQVPAK |
| 720 | 726 | 723 | PETLFAL |
| 711 | 718 | 717 | GTTKVFIK |
| 1222 | 1236 | 1226 | SAPPPPPPPPAATAS |
| 197 | 203 | 202 | GKYLEIK |
| 1288 | 1294 | 1293 | AGSLADA |
| 1023 | 1029 | 1023 | PTPVANS |
| 1012 | 1017 | 1013 | AAAQAT |

44. PRA1
MNYLLFCLFFAFSVAAPVTVTRFVDASPTGYDWRADWVKGFPIDSSCNATQYNQLSTGLQEAQLLAEHARDH
TLRFGSKSPFFRKYFGNDTASAEVVGHFENVVGADKSSILFLCDDLDDKCKNDGWAGYWRGSNHSDQTIICD
LSFVTRRYLSQLCSGGYTVSKSKTNIFWAGDLLHRFWHLKSIGQLVIEHYADTYEEVLELAQENSTYAVRNS
NSLIYYALDVYAYDVTIPGEGCNGDGTSYKKSDFSSFEDSDSGSDSGASSTASSSHQHTDSNPSATTDANSH
CHTHADGEVHC*

| Rank | Sequence | Start position | Score |
|---|---|---|---|
| 1 | AYDVTIPGEGCNGDGT | 228 | 0.90 |
| 2 | DWVKGFPIDSSCNATQ | 36 | 0.89 |
| 2 | SQLCSGGYTVSKSKTN | 154 | 0.89 |
| 2 | DQTIICDLSFVTRRYL | 138 | 0.89 |
| 2 | KNDGWAGYWRGSNHSD | 123 | 0.89 |
| 3 | TRFVDASPTGYDWRAD | 21 | 0.88 |
| 4 | NSLIYYALDVYAYDVT | 217 | 0.87 |
| 5 | GEGCNGDGTSYKKSDF | 235 | 0.86 |
| 6 | HQHTDSNPSATTDANS | 272 | 0.85 |
| 7 | SPTGYDWRADWVKGFP | 27 | 0.84 |
| 7 | LELAQENSTYAVRNSN | 202 | 0.84 |
| 8 | EHARDHTLRFGSKSPF | 67 | 0.83 |
| 9 | SSFEDSDSGSDSGASS | 251 | 0.82 |
| 10 | DGTSYKKSDFSSFEDS | 241 | 0.81 |
| 10 | GQLVIEHYADTYEEVL | 187 | 0.81 |
| 11 | YWRGSNHSDQTIICDL | 130 | 0.80 |
| 12 | SSTASSSHQHTDSNPS | 265 | 0.79 |
| 13 | AFSVAAPVTVTRFVDA | 11 | 0.77 |
| 14 | LRFGSKSPFFRKYFGN | 74 | 0.75 |

| Start | End | Max_score_pos | Sequence |
|---|---|---|---|
| 4 | 27 | 6 | LLFCLFFAFSVAAPVTVTRFVDAS |
| 109 | 121 | 112 | SSILFLCDDLDDK |
| 139 | 165 | 145 | QTIICDLSFVTRRYLSQLCSGGYTVSK |
| 218 | 236 | 221 | SLIYYALDVYAYDVTIPGE |
| 173 | 196 | 192 | AGDLLHRFWHLKSIGQLVIEHYAD |
| 93 | 107 | 97 | SAEVVGHFENVVGAD |
| 286 | 292 | 290 | NSHCHTH |
| 61 | 68 | 62 | EAQLLAEH |
| 198 | 205 | 203 | YEEVLELA |
| 43 | 50 | 44 | IDSSCNAT |
| 267 | 274 | 271 | TASSSHQH |

45. AMYG2
MKLFLTIIFIIASVNAVKEYLFKSCSQSGFCNRNRHYATEVSNCENFQSPYSIDSIKVDNDTITGVVFKHLP
QLDHPIQFPFEISILEGNFRFKLTEKENLVAKNVNPVRYNETEKWAFKQGVTKSSDFDVSLRDNEARVIYGD
HEVLIQYHPIMFVFKYAGKEQLRINDKQFLNIEHRRTRDENDNNMLPQESDFNMFSDSFQDSKFDTLPLGPE
SIGLDFTLLGFSNLYGIPEHADSLLLRDTSSGEPYRLYNVDIFEYEPNSRLPMYGSIPLVVAAKPDVSIGIF
WLNSADTYVDIHKSKSSTVHWMSENGILDFIVIIEKSPAMVNSQYGKVTGNTQLPPLFSLGYHQCRWNYNDE
KDVLDVHAKFDEYEIPYDTIWLDIEYTDEKKYFTWHKENFATPEKMLRELDRTGRNLVAIIDPHIKTGYDVS
DEIIKKGLTMKDSNNNTYYGHCWPGESVWIDTLNPNSQSFWDKKHKQFMTPAPNIHLWNDMNEPSVFNGPET
SAPKDNLHFGQWEHRSIHNVFGLSYHETTFNSLLNRSPEKRPFILTRSYFAGSQRTAAMWTGDNMSKWEYLK
ISIPMVLTSNVVGMPFAGADVGGFFGNPSSELLTRWYQAGIWYPFFRAHAHIDSRRREPWLAGEPYTQYIRD
AIRLRYALLPLFYTSFYEASKTGTPVIKPVFYENTHNADSYAIDDEFFIGNSGLLVKPVTDEGAKEIEFYLP
DDKVYYDFTNGVLQGVYKGGKKPVQLSDIPMLLKGGSIIPMKTRYRRSSKLMKSDPYTLVIALDEEGSASGK
LYVDDGETFAQGTEVAFTVDNNIINAKKIGPTASIPIEKIIIASKDQTTTLNNPKLDINSDWSLPFSFDSHR
KIEHDEL*

| Rank | Sequence | Start position | Score |
|---|---|---|---|
| 1 | FGLSYHETTFNSLLNR | 525 | 0.93 |
| 2 | YQAGIWYPFFRAHAHI | 613 | 0.92 |
| 2 | NNTYYGHCWPGESVWI | 447 | 0.92 |
| 2 | DEIIKKGLTMKDSNNN | 433 | 0.92 |
| 2 | YEIPYDTIWLDIEYTD | 373 | 0.92 |
| 3 | EKIIIASKDQTTTLNN | 830 | 0.91 |
| 3 | IEHRRTRDENDNNMLP | 176 | 0.91 |
| 4 | GGSIIPMKTRYRRSSK | 755 | 0.90 |
| 4 | AGEPYTQYIRDAIRLR | 638 | 0.90 |
| 5 | NSDWSLPFSFDSHRKI | 851 | 0.88 |
| 5 | KHKQFMTPAPNIHLWN | 476 | 0.88 |
| 5 | CSQSGFCNRNRHYATE | 25 | 0.88 |
| 5 | TSSGEPYRLYNVDIFE | 245 | 0.88 |
| 6 | AKEIEFYLPDDKVYYD | 712 | 0.87 |
| 7 | QGTEVAFTVDNNIINA | 803 | 0.86 |
| 7 | GTPVIKPVFYENTHNA | 671 | 0.86 |
| 7 | TSFYEASKTGTPVIKP | 662 | 0.86 |
| 7 | TGDNMSKWEYLKISIP | 565 | 0.86 |
| 7 | HQCRWNYNDEKDVLDV | 351 | 0.86 |
| 8 | TRSYFAGSQRTAAMWT | 550 | 0.85 |
| 8 | IVIIEKSPAMVNSQYG | 319 | 0.85 |
| 9 | EGSASGKLYVDDGETF | 786 | 0.84 |
| 9 | EVLIQYHPIMFVFKYA | 146 | 0.84 |
| 10 | PSVFNGPETSAPKDNL | 496 | 0.83 |
| 10 | VAIIDPHIKTGYDVSD | 418 | 0.83 |
| 10 | VNSQYGKVTGNTQLPP | 329 | 0.83 |
| 11 | HAHIDSRRREPWLAGE | 625 | 0.82 |
| 11 | TPEKMLRELDRTGRNL | 402 | 0.82 |
| 12 | GPTASIPIEKIIIASK | 822 | 0.81 |
| 12 | CENFQSPYSIDSIKVD | 44 | 0.81 |
| 12 | FWLNSADTYVDIHKSK | 288 | 0.81 |
| 12 | NETEKWAFKQGVTKSS | 112 | 0.81 |
| 13 | QGVYKGGKKPVQLSDI | 734 | 0.80 |
| 13 | IHLWNDMNEPSVFNGP | 487 | 0.80 |
| 13 | PQESDFNMFSDSFQDS | 191 | 0.80 |
| 14 | KLMKSDPYTLVIALDE | 770 | 0.79 |
| 14 | GMPFAGADVGGFFGNP | 589 | 0.79 |
| 14 | PGESVWIDTLNPNSQS | 456 | 0.79 |
| 15 | FLTIIFIIASVNAVKE | 4 | 0.78 |
| 15 | TGNTQLPPLFSLGYHQ | 337 | 0.78 |
| 16 | DVHAKFDEYEIPYDTI | 365 | 0.77 |
| 16 | YGIPEHADSLLLRDTS | 231 | 0.77 |
| 17 | FKLTEKENLVAKNVNP | 93 | 0.76 |
| 17 | EFFIGNSGLLVKPVTD | 694 | 0.76 |
| 17 | DVGGFFGNPSSELLTR | 596 | 0.76 |
| 17 | PMVLTSNVVGMPFAGA | 580 | 0.76 |
| 17 | TWHKENFATPEKMLRE | 394 | 0.76 |
| 17 | DEKKYFTWHKENFATP | 388 | 0.76 |
| 17 | IWLDIEYTDEKKYFTW | 380 | 0.76 |
| 17 | RHYATEVSNCENFQSP | 35 | 0.76 |
| 17 | KQGVTKSSDFDVSLRD | 120 | 0.76 |
| 18 | PVFYENTHNADSYAID | 677 | 0.75 |
| 18 | SQRTAAMWTGDNMSKW | 557 | 0.75 |
| 18 | PMYGSIPLVVAAKPDV | 268 | 0.75 |
| 18 | FEYEPNSRLPMYGSIP | 259 | 0.75 |

| Start | End | Max_score_pos | Sequence |
|---|---|---|---|
| 268 | 290 | 276 | PMYGSIPLVVAAKPDVSIGIFWL |
| 64 | 88 | 69 | TGVVFKHLPQLDHPIQFPFEISILE |
| 776 | 783 | 780 | PYTLVIAL |
| 699 | 707 | 705 | NSGLLVKPV |
| 671 | 681 | 677 | GTPVIKPVFYE |
| 651 | 667 | 657 | RLRYALLPLFYTSFYEA |
| 139 | 160 | 150 | RVIYGDHEVLIQYHPIMFVFKY |
| 573 | 591 | 585 | EYLKISIPMVLTSNVVGMP |
| 316 | 324 | 318 | LDFIVIIEK |
| 4 | 30 | 11 | FLTIIFIIASVNAVKEYLFKSCSQSGF |
| 729 | 739 | 735 | TNGVLQGVYKG |
| 342 | 354 | 345 | LPPLFSLGYHQCR |
| 362 | 370 | 366 | DVLDVHAKF |
| 416 | 424 | 420 | NLVAIIDPH |
| 521 | 531 | 527 | IHNVFGLSYHE |
| 236 | 244 | 239 | HADSLLLRD |
| 742 | 757 | 746 | KPVQLSDIPMLLKGGS |
| 250 | 260 | 256 | PYRLYNVDIFE |
| 790 | 796 | 792 | SGKLYVD |
| 451 | 457 | 453 | YGHCWPG |
| 293 | 302 | 299 | ADTYVDIHKS |
| 804 | 810 | 808 | GTEVAFT |
| 100 | 109 | 103 | NLVAKNVNPV |
| 715 | 727 | 721 | IEFYLPDDKVYYD |
| 49 | 56 | 50 | SPYSIDSI |
| 209 | 234 | 227 | DTLPLGPESIGLDFTLLGFSNLYGIP |
| 38 | 44 | 39 | ATEVSNC |
| 128 | 134 | 130 | DFDVSLR |
| 614 | 628 | 625 | QAGIWYPFFRAHAHI |
| 823 | 836 | 831 | PTASIPIEKIIIAS |
| 545 | 552 | 550 | RPFILTRS |
| 855 | 861 | 857 | SLPFSFD |
| 606 | 612 | 611 | SELLTRW |
| 428 | 434 | 434 | GYDVSDE |
| 642 | 649 | 644 | YTQYIRDA |
| 373 | 385 | 383 | YEIPYDTIWLDIE |
| 326 | 335 | 334 | PAMVNSQYGK |

46. LP19
MTSTDEDSGKNQFSTPKAQQLSTNGSFPLIDQKTKPQLDMEKMRDILVEETCLYTKGVQDTDVDWFITDSSI
DPNADVQPSVNSPRETNLNSQATIPTSHLTSAISNSNENYTNKTKPSIAPIQEENIASETSPRHHRHEIEQQ
QPRRRSSVSVSPSGGFLSKLKSKFHKESPTPPGNHQDGLFKSGYSVNPDKKSNDSSNSSIASLSSSPRLVSG
SNLQRTMSTPAYTHDTCDPRLEEYIKFYRKSDRRASVASSSHSAKDECLPSVLVNASEPTNYNKXKDZVNAS
RVSGFFXRKXSVAMKNXETSSXRSXVSVSVTPQSQVKNGLENNPSFQGLKPLKRVAFHSSTXLIDPPQQIPS
RTPRKGNVEVLPNGTVNIRPLTDEERLEIEKSQKGLGGGIVVGGTGALGYIKKDSDPPKPGENNLNAQDDDN
NDDGSNQSSESSQLESEPSVDKHAKSFTIDTPMARHQAVNYSVPIKKMALDTMYARCCHLREILPIPAILKQ
IPKGSMAPIPVLQLRNPTPTMIEIQSFADFLRIAPVICVSLDGVNFSVEQFKILLSAMSAKKQLEKLSLRNT
PIDQKGWSLLCWFLSRNTVLNRLDITQCPSLSVNTLKKKKKKTDSKFDETLVRMVSNKDNRSDVDWELFVAT
LVARGGIEELILTGCCITDIEIFEKLISLAVSKKTSRLGLAFNQLTSRHMKIIVDEWLFKDFARGIDFGYND
FSSVHMLKILVDYSKRPDFDQILSKSTLSFVSLNSTNVSLGDIFKESFERVLMKLPNLKYVDFSNNQRLFGT
FGKSDNEEADANEVASVNYFISKLPLFPKLVRLHLENENFSKSSVLQIAEILPFCKSLGYFSILGSKLDFTC
GSALVNAVKNSQSLINVDADSDNFPDIFKERMGLYTMRNMERLLYSAKKADVKTPLLSEDSAGNVSMTEQLH
EILRLKSQQKLDLQSPEITKFIERAKSISHGLRQTINELLRMQLKNRLDLDGKETLIRLIFIDSSIEKGLML
IDPSLVDDNNKNAGYLTSMIGTREGNEETQQFEQSDHLDPQPAASVLANKSPSVMSRSDSRTSLNNLNKEEG
SVLKLAKLRDFHSPNSPYPESTGEELRNKLMSVELADLDKVIDFLSDLKKKGVSLEKVFQCHENQGANDHEE
GLLDIEHIKSRLQKLSVEQMDGVSKDVDTDADEINTGTDKTHTLNNTYDEVLKNLFK

| Rank | Sequence | Start position | Score |
|---|---|---|---|
| 1 | SFTIDTPMARHQAVNY | 457 | 0.94 |
| 1 | LMLIDPSLVDDNNKNA | 1005 | 0.94 |
| 2 | PIKKMALDTMYARCCH | 475 | 0.93 |
| 2 | QIPSRTPRKGNVEVLP | 356 | 0.93 |
| 2 | STXLIDPPQQIPSRTP | 347 | 0.93 |
| 2 | ADEINTGTDKTHTLNN | 1182 | 0.93 |
| 3 | SVTPQSQVKNGLENNP | 316 | 0.92 |
| 4 | TKPSIAPIQEENIASE | 116 | 0.91 |
| 4 | CHENQGANDHEEGLLD | 1140 | 0.91 |
| 4 | SPSVMSRSDSRTSLNN | 1058 | 0.91 |
| 5 | WFITDSSIDPNADVQP | 65 | 0.90 |
| 5 | PKGSMAPIPVLQLRNP | 505 | 0.90 |
| 5 | EEYIKFYRKSDRRASV | 238 | 0.90 |
| 6 | LVDYSKRPDFDQILSK | 729 | 0.89 |
| 6 | GYIKKDSDPPKPGENN | 408 | 0.89 |
| 6 | LFKSGYSVNPDKKSND | 183 | 0.89 |
| 6 | QFEQSDHLDPQPAASV | 1038 | 0.89 |
| 7 | TFGKSDNEEADANEVA | 791 | 0.88 |
| 7 | STPAYTHDTCDPRLEE | 224 | 0.88 |
| 7 | IASETSPRHHRHEIEQ | 128 | 0.88 |
| 7 | TSAISNSNENYTNKTK | 102 | 0.88 |
| 8 | DILVEETCLYTKGVQD | 45 | 0.87 |
| 8 | PRLVSGSNLQRTMSTP | 211 | 0.87 |
| 8 | KFHKESPTPPGNHQDG | 167 | 0.87 |
| 9 | SVSVSPSGGFLSKLKS | 151 | 0.86 |
| 9 | DKTHTLNNTYDEVLKN | 1190 | 0.86 |
| 10 | HGLRQTINELLRMQLK | 965 | 0.85 |
| 10 | AFNQLTSRHMKIIVDE | 688 | 0.85 |
| 10 | SVEQMDGVSKDVDTDA | 1167 | 0.85 |
| 10 | YLTSMIGTREGNEETQ | 1022 | 0.85 |
| 11 | TMRNMERLLYSAKKAD | 899 | 0.84 |
| 11 | SNNQRLFGTFGKSDNE | 783 | 0.84 |
| 12 | FKERMGLYTMRNMERL | 891 | 0.83 |
| 12 | PKLVRLHLENENFSKS | 819 | 0.83 |
| 12 | HQAVNYSVPIKKMALD | 467 | 0.83 |
| 13 | GCCITDIEIFEKLISL | 661 | 0.82 |
| 13 | ASEPTNYNKXKDVNAS | 272 | 0.82 |
| 13 | EIEQQQPRRRSSVSVS | 140 | 0.82 |
| 13 | FSTPKAQQLSTNGSFP | 13 | 0.82 |
| 14 | PSVLVNASEPTNYNKX | 266 | 0.81 |
| 14 | SHSAKDECLPSVLVNA | 257 | 0.81 |
| 14 | SNSSIASLSSSPRLVS | 200 | 0.81 |
| 15 | LIFIDSSIEKGLMLID | 994 | 0.80 |
| 15 | QATIPTSHLTSAISNS | 93 | 0.80 |
| 15 | FKESFERVLMKLPNLK | 763 | 0.80 |
| 15 | KKKKKTDSKFDETLVR | 613 | 0.80 |
| 15 | DFLRIAPVICVSLDGV | 532 | 0.80 |
| 15 | LQLRNPTPTMIEIQSF | 515 | 0.80 |
| 15 | HLREILPIPAILKQIP | 490 | 0.80 |
| 15 | QSSESSQLESEPSVDK | 438 | 0.80 |
| 15 | GVSKDVDTDADEINTG | 1173 | 0.80 |
| 16 | QKGWSLLCWFLSRNTV | 579 | 0.79 |
| 16 | TKGVQDTDVDWFITDS | 55 | 0.79 |
| 16 | MIEIQSFADFLRIAPV | 524 | 0.79 |
| 16 | EPSVDKHAKSFTIDTP | 448 | 0.79 |
| 16 | VGGTGALGYIKKDSDP | 401 | 0.79 |
| 16 | DQKTKPQLDMEKMRDI | 31 | 0.79 |
| 16 | FFXRKXSVAMKNXETS | 292 | 0.79 |
| 16 | GNHQDGLFKSGYSVNP | 177 | 0.79 |
| 16 | SVLANKSPSVMSRSDS | 1052 | 0.79 |
| 17 | AMSAKKQLEKLSLRNT | 560 | 0.78 |
| 17 | LSTNGSFPLIDQKTKP | 21 | 0.78 |
| 17 | PYPESTGEELRNKLMS | 1096 | 0.78 |
| 18 | TPLLSEDSAGNVSMTE | 917 | 0.77 |
| 18 | QSLINVDADSDNFPDI | 875 | 0.77 |
| 18 | RGGIEELILTGCCITD | 651 | 0.77 |
| 18 | DKVIDFLSDLKKKGVS | 1118 | 0.77 |
| 19 | RNTPIDQKGWSLLCWF | 573 | 0.76 |
| 19 | IPAILKQIPKGSMAPI | 497 | 0.76 |
| 19 | NLNAQDDDNNDDGSNQ | 423 | 0.76 |
| 20 | DFTCGSALVNAVKNSQ | 860 | 0.75 |
| 20 | ISLAVSKKTSRLGLAF | 674 | 0.75 |
| 20 | PSLSVNTLKKKKKKTD | 604 | 0.75 |
| 20 | XETSSXRSXVSVSVTP | 304 | 0.75 |
| 20 | MTSTDEDSGKNQFSTP | 1 | 0.75 |

| Start | End | Max_score_pos | Sequence |
|---|---|---|---|
| 261 | 273 | 267 | KDECLPSVLVNAS |
| 528 | 562 | 542 | IQSFADFLRIAPVICVSLDGVNFSEQFKILLSAM |
| 641 | 653 | 647 | DWELFVATLVARG |
| 486 | 505 | 489 | YARCCHLREILPIPAILKQI |
| 656 | 668 | 661 | EELILTGCCITDI |
| 511 | 519 | 515 | APIPVLQLR |
| 583 | 590 | 587 | WSLLCWFL |
| 601 | 612 | 606 | ITQCPSLSVNTL |
| 315 | 326 | 317 | SVSVTPQSQVKN |
| 671 | 682 | 677 | FEKLISLAVSKK |
| 1080 | 1091 | 1085 | GSVLKLAKLRDF |
| 804 | 826 | 824 | NEVASVNYFISKLPLFPKLVRLH |
| 834 | 859 | 847 | KSSVLQIAEILPFCKSLGYFSILGSK |
| 721 | 734 | 730 | FSSVHMLKILVDYS |
| 1005 | 1014 | 1011 | GLMLIDPSLV |
| 1130 | 1144 | 1138 | KKGVSLEKVFQCHEN |
| 861 | 872 | 869 | DFTCGSALVNAV |
| 467 | 480 | 474 | RHQAVNYSVPIKKM |
| 992 | 1000 | 995 | LIRLIFIDS |
| 45 | 57 | 55 | DILVEETCLYTKG |
| 740 | 763 | 750 | DQILSKSTLSFVSLNSTNVSLGDI |
| 1045 | 1063 | 1053 | HLDPQPAASVLANKSPSVM |
| 149 | 167 | 153 | RSSVSVSPSGGFLSKLKSK |
| 769 | 783 | 778 | ERVLMKLPNLKYVDF |
| 907 | 923 | 919 | LLYSAKKADVKTPLLSE |
| 398 | 405 | 399 | GGIVVGGT |
| 75 | 83 | 79 | NADVQPSVN |
| 935 | 952 | 938 | LHEILRLKSQQKLDLQSP |
| 1152 | 1170 | 1166 | EGLLDIEHIKSRLQKLSVE |
| 334 | 346 | 343 | FQGLKPLKRVAFH |
| 1110 | 1128 | 1123 | LMSVELADLDKVIDFLSDL |
| 367 | 374 | 373 | NVEVLPNG |
| 1201 | 1206 | 1206 | DEVLKN |
| 202 | 217 | 211 | SSIASLSSSPRLVSGS |
| 250 | 259 | 255 | RASVASSSHS |
| 698 | 705 | 703 | MKIIVDEW |
| 118 | 123 | 121 | PSIAPI |
| 95 | 104 | 102 | TIPTSHLTSA |
| 185 | 191 | 187 | KSGYSVN |
| 27 | 32 | 29 | FPLIDQ |
| 227 | 236 | 230 | AYTHDTCDPR |
| 354 | 360 | 354 | PPQQIPS |
| 443 | 455 | 453 | SSQLESEPSVDKH |
| 685 | 692 | 687 | RLGLAFNQ |
| 15 | 21 | 19 | TPKAQQL |
| 963 | 969 | 965 | SISHGLR |
| 238 | 244 | 243 | EEYIKFY |

47. ALG5
MIYYILAFFIFISLSIYATVIFFSHKPRKPFPSELTYKTNDSTDKSHPLPPRINTNSKFQDDGIDISLVIPC
YNETQRLGKMLDEAIEYLEKNHQSKYEIIVVDDGSSDGTDEYALQKANEFKLPSHIMRVVQLKQNRGKGGAV
THGLLHSRGKYALFADADGATSFPDVAKLVNYLANANGQPSIAIGSRAHMVNTDAVVKRSFIRNFLMYGLHA
LVFVFGIRDVRDTQCGFKMFNFEAVKNIFPHMHTERWIFDVEVLLLGEIQKFNMKELPVNWQEIDGSKVDLA
RDSIAMAIDLVVTRLAYLLGVYKLDECGRINKKEQ*

| Rank | Sequence | Start position | Score |
|---|---|---|---|
| 1 | STDKSHPLPPRINTNS | 42 | 0.87 |
| 2 | GSKVDLARDSIAMAID | 282 | 0.86 |
| 3 | IPCYNETQRLGKMLDE | 70 | 0.85 |
| 4 | AMAIDLVVTRLAYLLG | 293 | 0.84 |
| 5 | HALVFVFGIRDVRDTQ | 215 | 0.83 |
| 5 | DGSSDGTDEYALQKAN | 105 | 0.83 |
| 6 | VVTRLAYLLGVYKLDE | 299 | 0.82 |
| 7 | GKMLDEAIEYLEKNHQ | 80 | 0.81 |
| 7 | SELTYKTNDSTDKSHP | 33 | 0.81 |
| 7 | YLLGVYKLDECGRINK | 305 | 0.81 |
| 7 | SIAIGSRAHMVNTDAV | 185 | 0.81 |
| 7 | HSRGKYALFADADGAT | 150 | 0.81 |
| 7 | SLSIYATVIFFSHKPR | 13 | 0.81 |
| 8 | KYEIIVVDDGSSDGTD | 97 | 0.80 |
| 9 | PHMHTERWIFDVEVLL | 246 | 0.79 |
| 9 | TQCGFKMFNFEAVKNI | 229 | 0.79 |
| 9 | GGAVTHGLLHSRGKYA | 141 | 0.79 |
| 10 | LGEIQKFNMKELPVNW | 262 | 0.78 |
| 11 | HMVNTDAVVKRSFIRN | 193 | 0.77 |
| 12 | RVVQLKQNRGKGGAVT | 130 | 0.75 |

| Start | End | Max_score_pos | Sequence |
|---|---|---|---|
| 211 | 224 | 217 | MYGLHALVFVFGIR |
| 65 | 74 | 70 | DISLVIPCYN |
| 254 | 265 | 260 | IFDVEVLLLGEI |
| 293 | 315 | 306 | AMAIDLVVTRLAYLLGVYKLDEC |
| 4 | 26 | 5 | YILAFFIFISLSIYATVIFFSHK |
| 97 | 105 | 100 | KYEIIVVDD |
| 167 | 177 | 173 | FPDVAKLVNYL |
| 124 | 135 | 131 | LPSHIMRVVQLK |
| 143 | 160 | 147 | AVTHGLLHSRGKYALFAD |
| 197 | 204 | 203 | TDAVVKRS |
| 46 | 52 | 50 | SHPLPPR |
| 282 | 290 | 285 | GSKVDLARD |
| 185 | 190 | 185 | SIAIGS |
| 228 | 233 | 229 | DTQCGF |
| 272 | 277 | 275 | ELPVNW |

48. HWP1
MRLSTAQLIAIAYYMLSIGATVPQVDGQGETEEALIQKRSYDYYQEPCDDYPQQQQQQEPCDYPQQQQQEEP
CDYPQQQPQEPCDYPQQPQEPCDYPQQPQEPCDYPQQPQEPCDNPPQPDVPCDNPPQPDVPCDNPPQPDIPC
DNPPQPDIPCDNPPQPDQPDDNPPIPNIPTDWIPNIPTDWIPDIPEKPTTPATTPNIPATTTTSESSSSSSS
SSSSTTPKTSASTTPESSVPATTPNTSVPTTSSESTTPATSPESSVPVTSGSSILATTSESSSAPATTPNTS
VPTTTTEAKSSSTPLTTTTEHDTTVVTVTSCSNSVCTESEVTTGVIVITSKDTIYTTYCPLTETTPVSTAPA
TETPTGTVSTSTEQSTTVITVTSCSESSCTESEVTTGVVVVTSEETVYTTFCPLTENTPGTDSTPEASIPPM
ETIPAGSESSMPAGETSPAVPKSDVPATESAPVPEMTPAGSQPSIPAGETSPAVPKSDVPATESAPAPEMTP
AGTETKPAAPKSSAPATEPSPVAPGTESAPAGPGASSSPKSSVLASETSPIAPGAETAPAGSSGAITIPESS
AVVSTTEGAIPTTLESVPLMQPSANYSSVAPISTFEGAGNNMRLTFGAAIIGIAAFLI

| Rank | Sequence | Start position | Score |
|---|---|---|---|
| 1 | KDTIYTTYCPLTETTP | 339 | 0.97 |
| 1 | DVPCDNPPQPDVPCDN | 121 | 0.97 |
| 1 | QEPCDNPPQPDVPCDN | 111 | 0.97 |
| 2 | DVPCDNPPQPDIPCDN | 131 | 0.96 |
| 3 | PGTESAPAGPGASSSP | 528 | 0.95 |
| 3 | TETKPAAPKSSAPATE | 507 | 0.95 |
| 3 | DIPCDNPPQPDIPCDN | 141 | 0.95 |
| 4 | EPSPVAPGTESAPAGP | 522 | 0.93 |
| 4 | TFCPLTENTPGTDSTP | 410 | 0.93 |
| 5 | EGAIPTTLESVPLMQP | 583 | 0.92 |
| 5 | LASETSPIAPGAETAP | 548 | 0.92 |
| 5 | SAPATTPNTSVPTTTT | 279 | 0.92 |
| 5 | DIPCDNPPQPDQPDDN | 151 | 0.92 |
| 5 | QEPCDYPQQPQEPCDN | 101 | 0.92 |
| 6 | YDYYQEPCDDYPQQQQ | 41 | 0.91 |
| 6 | TSASTTPESSVPATTP | 225 | 0.91 |
| 6 | DWIPDIPEKPTTPATT | 183 | 0.91 |
| 7 | GETSPAVPKSDVPATE | 480 | 0.89 |
| 7 | GETSPAVPKSDVPATE | 446 | 0.89 |
| 7 | GSESSMPAGETSPAVP | 438 | 0.89 |
| 7 | ENTPGTDSTPEASIPP | 416 | 0.89 |
| 7 | SSSSSTTPKTSASTTP | 216 | 0.89 |
| 7 | TTPATTPNIPATTTTS | 193 | 0.89 |
| 8 | QQQQEEPCDYPQQQPQ | 66 | 0.88 |
| 8 | QQQQQEPCDYPQQQQQ | 54 | 0.88 |
| 8 | EETVYTTFCPLTENTP | 404 | 0.88 |
| 8 | SVPATTPNTSVPTTSS | 234 | 0.88 |
| 9 | QEPCDYPQQPQEPCDY | 91 | 0.87 |
| 9 | QEPCDYPQQPQEPCDY | 81 | 0.87 |
| 9 | SGAITIPESSAVVSTT | 567 | 0.87 |
| 9 | METIPAGSESSMPAGE | 432 | 0.87 |
| 10 | PATESAPVPEMTPAGS | 458 | 0.86 |
| 10 | CPLTETTPVSTAPATE | 347 | 0.86 |
| 10 | TSVPTTTTEAKSSSTP | 287 | 0.86 |
| 10 | SSESTTPATSPESSVP | 248 | 0.86 |
| 11 | TPVSTAPATETPTGTV | 353 | 0.85 |
| 11 | AKSSSTPLTTTTEHDT | 296 | 0.85 |
| 11 | MLSIGATVPQVDGQGE | 15 | 0.85 |
| 12 | APKSSAPATEPSPVAP | 513 | 0.84 |
| 12 | QPSIPAGETSPAVPKS | 474 | 0.84 |
| 12 | SCTESEVTTGVVVVTS | 388 | 0.84 |
| 12 | VIVITSKDTIYTTYCP | 333 | 0.84 |
| 12 | TSVPTTSSESTTPATS | 242 | 0.84 |
| 13 | APEMTPAGTETKPAAP | 499 | 0.83 |
| 13 | TGTVSTSTEQSTTVIT | 365 | 0.83 |
| 14 | YPQQQPQEPCDYPQQP | 75 | 0.82 |
| 14 | SDVPATESAPAPEMTP | 489 | 0.82 |
| 15 | TEHDTTVVTVTSCSNS | 307 | 0.81 |
| 15 | SILATTSESSSAPATT | 269 | 0.81 |
| 16 | SSVAPISTFEGAGNNM | 603 | 0.80 |
| 17 | PPIPNIPTDWIPNIPT | 167 | 0.79 |
| 18 | APGAETAPAGSSGAIT | 556 | 0.78 |
| 18 | DSTPEASIPPMETIPA | 422 | 0.78 |
| 18 | TSESSSSSSSSSSSTT | 207 | 0.78 |
| 19 | PEMTPAGSQPSIPAGE | 466 | 0.77 |
| 19 | TGVVVVTSEETVYTTF | 396 | 0.77 |
| 19 | TSCSESSCTESEVTTG | 382 | 0.77 |
| 20 | SVPLMQPSANYSSVAP | 592 | 0.75 |
| 20 | QPDQPDDNPPIPNIPT | 159 | 0.75 |

| Start | End | Max_score_pos | Sequence |
|---|---|---|---|
| 311 | 327 | 316 | TTVVTVTSCSNSVCTES |
| 395 | 406 | 400 | TTGVVVVTSEET |
| 376 | 392 | 381 | TTVITVTSCSESSCTES |
| 5 | 26 | 11 | TAQLIAIAYYMLSIGATVPQVD |
| 330 | 338 | 332 | TTGVIVITS |
| 408 | 415 | 410 | YTTFCPLT |
| 119 | 127 | 121 | QPDVPCDNP |
| 129 | 137 | 131 | QPDVPCDNP |
| 343 | 350 | 346 | YTTYCPLT |
| 600 | 609 | 605 | ANYSSVAPIS |
| 259 | 274 | 263 | ESSVPVTSGSSILATT |
| 569 | 582 | 578 | AITIPESSAVVSTT |
| 587 | 598 | 592 | PTTLESVPLMQP |
| 542 | 557 | 546 | SPKSSVLASETSPIAP |
| 483 | 497 | 488 | SPAVPKSDVPATESA |
| 449 | 468 | 454 | SPAVPKSDVPATESAPVPEM |
| 622 | 631 | 630 | FGAAIIGIAA |
| 139 | 147 | 141 | QPDIPCDNP |
| 149 | 157 | 151 | QPDIPCDNP |
| 70 | 117 | 75 | EEPCDYPQQQPQEPCDYPQQPQEPCDYPQQPQEPCDYPQQ |
| | | | PQEPCDNP |
| 58 | 66 | 63 | QEPCDYPQQ |
| 352 | 358 | 357 | TTPVSTA |
| 34 | 55 | 45 | ALIQKRSYDYYQEPCDDYPQQQ |
| 520 | 529 | 526 | ATEPSPVAPG |
| 232 | 238 | 234 | ESSVPAT |
| 472 | 478 | 476 | GSQPSIP |
| 512 | 518 | 516 | AAPKSSA |

49. ALS10
MLQQYTLLLIYLSVATAKTITGVFNSFNSLTWSNAATYNYKGPGTPTWNAVLGWSLDGTSASPGDTFTLNMP
CVFKFTTSQTSVDLTAHGVKYATCQFQAGEEFMTFSTLTCTVSNTLTPSIKALGTVTLPLAFNVGGTGSSVD
LEDSKCFTAGTNTVTFNDGGKKISINVDFERSNVDPKGYLTDSRVIPSLNKVSTLFVAPQCANGYTSGTMGF
ANTYGDVQIDCSNIHVGITKGLNDWNYPVSSESFSYTKTCSSNGIFITYKNVPAGYRPFVDAYISATDVNSY
TLSYANEYTCAGGYWQRAPFTLRWTGYRNSDAGSNGIVIVATTRTVTDSTTAVTTLPFDPNRDKTKTIEILK
PIPTTTITTSYVGVTTSYSTKTAPIGETATVIVDIPYHTTTTVTSKWTGTITSTTTHTNPTDSIDTVIVQVP
SPNPTVTTTEYWSQSFATTTTITGPPGNTDTVLIREPPNHTVTTTEYWSESYTTTSTFTAPPGGTDSVIIKE
PPNPTVTTTEYWSESYTTTTTVTAPPGGTDTVIIREPPNHTVTTTEYWSQSYTTTTTVIAPPGGTDSVIIRE
PPNPTVTTTEYWSQSYATTTTITAPPGETDTVLIREPPNHTVTTTEYWSQSYATTTTITAPPGETDTVLIRE
PPNHTVTTTEYWSQSYTTTTTVIAPPGGTDSVIIREPPNPTVTTTEYWSQSYATTTTITAPPGETDTXLIRE
PPNHTVTTTEYWSQSYATTTTITAPPGETDTVLIREPPNHTVTTTEYWSQSFATTTTVTAPPGGTDTVIIRE
PPNHTVTTTEYWSQSXATTTTXTAPPGXTDTVLIREPPNPTVTTTEYWSQSYTTATTVTAPPGGTDTVIIYD
TMSSSEISSFSRPHYTNHTTLWSTTWVIETKTITETSCEGDKGCSWVSVSTRIVTIPNNIETPMVTNTVDTT
TTESTLQSPSGIFSESGVSVETESSTFTTAQTNPSVPTTESEVVFTTKGNNGNGPYESPSTNVKSSMDENSE
FTTSTAASTSTDIENETIATTGSVEASSPIISSSADETTTVTTTAESTSVIEQQTNNNGGGNAPSATSTSSP
STTTTANSDSVITSTTSTNQSQSQSNSDTQQTTLSQQMTSSLVSLHMLTTFDGSGSVIQHSTWLCGLITLLS
LFI

| Rank | Sequence | Start position | Score |
|---|---|---|---|
| 1 | TGTITSTTTHTNPTDS | 408 | 0.95 |
| 1 | STTAVTTLPFDPNRDK | 337 | 0.95 |
| 2 | HGVKYATCQFQAGEEF | 89 | 0.94 |
| 2 | YWSESYTTTSTFTAPP | 479 | 0.94 |
| 2 | TLRWTGYRNSDAGSNG | 309 | 0.94 |
| 3 | TXLIREPPNHTVTTTE | 715 | 0.93 |
| 4 | ESEVVFTTKGNNGNGP | 976 | 0.92 |
| 4 | KTITETSCEGDKGCSW | 895 | 0.92 |
| 4 | SVDLEDSKCFTAGTNT | 142 | 0.92 |
| 5 | LGTVTLPLAFNVGGTG | 125 | 0.91 |
| 6 | YWSESYTTTTTVTAPP | 515 | 0.90 |
| 7 | TVLIREPPNHTVTTTE | 751 | 0.89 |
| 7 | TTTITAPPGETDTXLI | 703 | 0.89 |
| 7 | YWSQSYTTTTTVIAPP | 659 | 0.89 |
| 7 | TVLIREPPNHTVTTTE | 643 | 0.89 |
| 7 | TVLIREPPNHTVTTTE | 607 | 0.89 |
| 7 | YWSQSYTTTTTVIAPP | 551 | 0.89 |
| 7 | TVLIREPPNHTVTTTE | 463 | 0.89 |
| 7 | TTTITGPPGNTDTVLI | 451 | 0.89 |
| 7 | TSSPSTTTTANSDSVI | 1077 | 0.89 |
| 7 | MTFSTLTCTVSNTLTP | 105 | 0.89 |
| 8 | NGPYESPSTNVKSSMD | 989 | 0.88 |
| 8 | TVLIREPPNPTVTTTE | 823 | 0.88 |
| 8 | YWSQSXATTTTXTAPP | 803 | 0.88 |
| 8 | TVTSKWTGTITSTTTH | 402 | 0.88 |
| 8 | TKTCSSNGIFITYKNV | 253 | 0.88 |
| 8 | SGTMGFANTYGDVQID | 211 | 0.88 |
| 9 | SDSVITSTTSTNQSQS | 1088 | 0.87 |
| 9 | APSATSTSSPSTTTTA | 1071 | 0.87 |
| 9 | TSVIEQQTNNNGGGNA | 1056 | 0.87 |
| 10 | HTTLWSTTWVIETKTI | 882 | 0.86 |
| 10 | PPGXTDTVLIREPPNP | 817 | 0.86 |
| 10 | TVIIREPPNHTVTTTE | 787 | 0.86 |
| 10 | TTTITAPPGETDTVLI | 739 | 0.86 |
| 10 | SVIIREPPNPTVTTTE | 679 | 0.86 |
| 10 | TTTITAPPGETDTVLI | 631 | 0.86 |
| 10 | TTTITAPPGETDTVLI | 595 | 0.86 |
| 10 | SVIIREPPNPTVTTTE | 571 | 0.86 |
| 10 | TVIIREPPNHTVTTTE | 535 | 0.86 |
| 10 | TDSVIIKEPPNPTVTT | 497 | 0.86 |
| 10 | TSTNQSQSQSNSDTQQ | 1096 | 0.86 |
| 11 | SSEISSFSRPHYTNHT | 868 | 0.85 |
| 11 | TVIIYDTMSSSEISSF | 859 | 0.85 |
| 11 | SVDLTAHGVKYATCQF | 83 | 0.85 |
| 11 | TTTXTAPPGXTDTVLI | 811 | 0.85 |
| 11 | YWSQSYATTTTITAPP | 731 | 0.85 |
| 11 | YWSQSYATTTTITAPP | 695 | 0.85 |
| 11 | YWSQSYATTTTITAPP | 623 | 0.85 |
| 11 | YWSQSYATTTTITAPP | 587 | 0.85 |
| 11 | PIPTTTITTSYVGVTT | 361 | 0.85 |
| 12 | YWSQSYTTATTVTAPP | 839 | 0.84 |
| 12 | TDSIDTVIVQVPSPNP | 421 | 0.84 |
| 12 | TRTVTDSTTAVTTLPF | 331 | 0.84 |
| 12 | TWSNAATYNYKGPGTP | 31 | 0.84 |
| 12 | EYTCAGGYWQRAPFTL | 295 | 0.84 |
| 13 | SVETESSTFTTAQTNP | 955 | 0.83 |
| 13 | PNNIETPMVTNTVDTT | 921 | 0.83 |
| 13 | ASPGDTFTLNMPCVFK | 61 | 0.83 |
| 13 | GIFITYKNVPAGYRPF | 260 | 0.83 |
| 14 | TNTVDTTTTESTLQSP | 930 | 0.82 |
| 14 | TVIVDIPYHTTTTVTS | 390 | 0.82 |
| 14 | VGITKGLNDWNYPVSS | 232 | 0.82 |
| 14 | PKGYLTDSRVIPSLNK | 180 | 0.82 |
| 14 | SADETTTVTTTAESTS | 1042 | 0.82 |
| 14 | FTTSTAASTSTDIENE | 1009 | 0.82 |
| 15 | YWSQSFATTTTVTAPP | 767 | 0.81 |
| 15 | PTVTTTEYWSQSYATT | 688 | 0.81 |
| 15 | PTVTTTEYWSQSYATT | 580 | 0.81 |
| 15 | YSTKTAPIGETATVIV | 378 | 0.81 |
| 15 | TYNYKGPGTPTWNAVL | 37 | 0.81 |
| 15 | TIEILKPIPTTTITTS | 355 | 0.81 |
| 15 | SGSVIQHSTWLCGLIT | 1134 | 0.81 |
| 16 | TVTTTEYWSESYTTTT | 509 | 0.80 |
| 16 | DAYISATDVNSYTLSY | 277 | 0.80 |
| 16 | NVPAGYRPFVDAYISA | 267 | 0.80 |
| 16 | TCTVSNTLTPSIKALG | 111 | 0.80 |
| 17 | FQAGEEFMTFSTLTCT | 98 | 0.79 |
| 17 | SCEGDKGCSWVSVSTR | 901 | 0.79 |
| 17 | TVTAPPGGTDTVIIYD | 849 | 0.79 |
| 17 | TTTVTAPPGGTDTVII | 775 | 0.79 |
| 17 | HTVTTTEYWSQSYATT | 724 | 0.79 |
| 17 | HTVTTTEYWSQSYATT | 616 | 0.79 |
| 17 | TTTVTAPPGGTDTVII | 523 | 0.79 |
| 17 | TVTTTEYWSESYTTTS | 473 | 0,79 |
| 17 | PSLNKVSTLFVAPQCA | 191 | 0.79 |
| 17 | SPIISSSADETTTVTT | 1036 | 0.79 |
| 18 | PTVTTTEYWSQSYTTA | 832 | 0.78 |
| 18 | TTTVIAPPGGTDSVII | 667 | 0.78 |
| 18 | TTTVIAPPGGTDSVII | 559 | 0.78 |
| 18 | GIVIVATTRTVTDSTT | 324 | 0.78 |
| 18 | KKISINVDFERSNVDP | 165 | 0.78 |
| 18 | TNNNGGGNAPSATSTS | 1063 | 0.78 |
| 19 | HTVTTTEYWSQSXATT | 796 | 0.77 |
| 19 | TPSIKALGTVTLPLAF | 119 | 0.77 |
| 19 | TVTTTAESTSVIEQQT | 1048 | 0.77 |
| 19 | MDENSEFTTSTAASTS | 1003 | 0.77 |
| 20 | AQTNPSVPTTESEVVF | 966 | 0.76 |
| 20 | HTVTTTEYWSQSYTTT | 652 | 0.76 |
| 20 | HTVTTTEYWSQSYTTT | 544 | 0.76 |
| 20 | TPTWNAVLGWSLDGTS | 45 | 0.76 |
| 20 | TTTEYWSQSFATTTTI | 439 | 0.76 |
| 20 | PYHTTTTVTSKWTGTI | 396 | 0.76 |
| 20 | CANGYTSGTMGFANTY | 205 | 0.76 |
| 21 | TTWVIETKTITETSCE | 888 | 0.75 |
| 21 | HTVTTTEYWSQSFATT | 760 | 0.75 |
| 21 | TDVNSYTLSYANEYTC | 283 | 0.75 |
| 21 | LNDWNYPVSSESFSYT | 238 | 0.75 |
| 21 | YGDVQIDCSNIHVGIT | 220 | 0.75 |

| Start | End | Max_score_pos | Sequence |
|---|---|---|---|
| 4 | 17 | 11 | QYTLLLIYLSVATA |
| 424 | 437 | 430 | IDTVIVQVPSPNPT |
| 186 | 208 | 202 | DSRVIPSLNKVSTLFVAPQCANG |
| 388 | 398 | 394 | TATVIVDIPYH |
| 908 | 921 | 911 | CSWVSVSTRIVTIP |
| 1134 | 1152 | 1147 | SGSVIQHSTWLCGLITLLS |
| 1119 | 1128 | 1125 | TSSLVSLHML |
| 119 | 135 | 131 | TPSIKALGTVTLPLAFN |
| 323 | 331 | 328 | NGIVIVATT |
| 70 | 77 | 75 | NMPCVFKF |
| 81 | 99 | 94 | QTSVDLTAHGVKYATCQFQ |
| 109 | 117 | 111 | TLTCTVSNT |
| 262 | 293 | 277 | FITYKNVPAGYRPFVDAYISATDVNSYTLSYA |
| 49 | 55 | 53 | NAVLGWS |
| 222 | 235 | 230 | DVQIDCSNIHVGIT |
| 337 | 346 | 343 | STTAVTTLPF |
| 606 | 612 | 611 | DTVLIRE |
| 463 | 468 | 467 | TVLIRE |
| 750 | 756 | 755 | DTVLIRE |
| 642 | 648 | 647 | DTVLIRE |
| 369 | 377 | 371 | TSYVGVTTS |
| 498 | 503 | 503 | DSVIIK |
| 356 | 362 | 359 | IEILKPI |
| 559 | 566 | 563 | TTTVIAPP |
| 667 | 674 | 671 | TTTVIAPP |
| 570 | 575 | 575 | DSVIIR |
| 678 | 683 | 683 | DSVIIR |
| 1029 | 1041 | 1035 | TGSVEASSPIISS |
| 977 | 982 | 980 | SEVVFT |
| 859 | 864 | 860 | TVIIYD |
| 951 | 958 | 953 | ESGVSVET |
| 243 | 250 | 246 | YPVSSESF |
| 295 | 301 | 299 | EYTCAGG |
| 142 | 153 | 148 | SVDLEDSKCFTA |
| 1055 | 1061 | 1056 | STSVIEQ |
| 942 | 949 | 945 | LQSPSGIF |
| 1088 | 1094 | 1091 | SDSVITS |
| 870 | 877 | 876 | EISSFSRP |
| 888 | 894 | 894 | TTWVIET |
| 20 | 27 | 21 | ITGVFNSF |
| 969 | 974 | 969 | NPSVPT |

50. IPF5185
MKVSTIFAAASALFAATTTLAQDVACLVDNQQVAVVDLDTGVCPFTIPASLAAFFTFVSLEEYNVQFYYTIV
NNVRYNTDIRNAGKVINVPARNLYGAGAVPFFQVHLEKQLEANSTAAIRRRLMGETPIVKRDQIDDFIASIE
NTEGTALEGSTLEVVDYVPGSSSASPSGSASPSGSESGSGSDSATIRSTTVVSSSSCESSGDSAATATGANG
ESTVTEQNTVVVTITSCHNDACHATTVPATASIGVTTVHGTETIFTTYCPLSSYETVESTKVITITSCSENK
CQETTVEATPSTATTVSEGVVTEYVTYCPVSSVETVASTKVITVVACDEHKCHETTAVATPTEVTTVVEGST
THYVTYKPTGSGPTQGETYATNAITSEGTVYVPKTTAVTTHGSTFETVAYITVTKATPTKGGEQHQPGSPAG
AATSAPGAPAPGASGAHASTANKVTVEAQATPGTLTPENTVAGGVNGEQVAVSAKTTISQTTVAKASGSGKA
AISTFEGAAAASAGASVLALALIPLAYFI*

| Rank | Sequence | Start position | Score |
|---|---|---|---|
| 1 | TVTKATPTKGGEQHQP | 412 | 0.95 |
| 2 | KPTGSGPTQGETYATN | 367 | 0.93 |
| 3 | KGGEQHQPGSPAGAAT | 420 | 0.90 |
| 4 | PLSSYETVESTKVITI | 266 | 0.89 |
| 4 | TETIFTTYCPLSSYET | 257 | 0.89 |
| 4 | TLEVVDYVPGSSSASP | 155 | 0.89 |
| 5 | VVTEYVTYCPVSSVET | 308 | 0.88 |
| 6 | HASTANKVTVEAQATP | 449 | 0.86 |
| 6 | APGAPAPGASGAHAST | 437 | 0.86 |
| 6 | ATPSTATTVSEGVVTE | 296 | 0.86 |
| 7 | TNAITSEGTVYVPKTT | 381 | 0.85 |
| 7 | TKVITITSCSENKCQE | 276 | 0.85 |
| 8 | NVQFYYTIVNNVRYNT | 64 | 0.84 |
| 8 | VEAQATPGTLTPENTV | 458 | 0.84 |
| 8 | TTVVEGSTTHYVTYKP | 353 | 0.84 |
| 8 | SATIRSTTVVSSSSCE | 187 | 0.84 |
| 9 | SSSCESSGDSAATATG | 198 | 0.83 |
| 9 | RLMGETPIVKRDQIDD | 123 | 0.83 |
| 10 | TAVTTHGSTFETVAYI | 396 | 0.82 |
| 10 | VITVVACDEHKCHETT | 329 | 0.82 |
| 10 | SCSENKCQETTVEATP | 283 | 0.82 |
| 10 | GSASPSGSESGSGSDS | 172 | 0.82 |
| 11 | VINVPARNLYGAGAVP | 87 | 0.81 |
| 11 | QVAVVDLDTGVCPFTI | 32 | 0.81 |
| 11 | ESTVTEQNTVVVTITS | 217 | 0.81 |
| 12 | DEHKCHETTAVATPTE | 336 | 0.80 |
| 13 | GGVNGEQVAVSAKTTI | 475 | 0.79 |
| 13 | VVVTITSCHNDACHAT | 226 | 0.79 |
| 14 | ETTAVATPTEVTTVVE | 342 | 0.78 |
| 15 | PGTLTPENTVAGGVNG | 464 | 0.77 |
| 15 | PGASGAHASTANKVTV | 443 | 0.77 |
| 16 | TTVAKASGSGKAAIST | 493 | 0.76 |
| 17 | PVSSVETVASTKVITV | 317 | 0.75 |
| 17 | FIASIENTEGTALEGS | 139 | 0.75 |

| Start | End | Max_score_pos | Sequence |
|---|---|---|---|
| 301 | 350 | 332 | ATTVSEGVVTEYVTYCPVSSVETVASTKVITVVACDEHKCH |
| | | | ETTAVATPT |
| 514 | 530 | 524 | AASAGASVLALALIPLA |
| 21 | 75 | 25 | AQDVACLVDNQQVAVVDLDTGVCPFTIPASLAAFFTFVSLE |
| | | | EYNVQFYYTIVNNV |
| 155 | 174 | 159 | TLEVVDYVPGSSSASPSGSA |
| 192 | 204 | 198 | STTVVSSSSCESS |
| 223 | 254 | 230 | QNTVVVTITSCHNDACHATTVPATASIGVTTV |
| 97 | 111 | 104 | GAGAVPFFQVHLEKQ |
| 262 | 273 | 267 | TTYCPLSSYETV |
| 405 | 416 | 410 | FETVAYITVTKA |
| 275 | 284 | 281 | STKVITITSC |
| 479 | 487 | 484 | GEQVAVSAK |
| 87 | 95 | 89 | VINVPARNL |
| 387 | 401 | 392 | EGTVYVPKTTAVTTH |
| 352 | 359 | 353 | VTTVVEGS |
| 361 | 367 | 365 | THYVTYK |
| 4 | 19 | 6 | STIFAAASALFAATTT |
| 455 | 461 | 459 | KVTVEAQ |
| 492 | 498 | 493 | QTTVAKA |
| 128 | 134 | 133 | TPIVKRD |
| 430 | 450 | 440 | PAGAATSAPGAPAPGASGAHA |
| 137 | 142 | 138 | DDFIAS |

51. IPF15911
MSFWDNNKDSFKSAGKSTFKGITSGTKAVGQAGYRTYKKNEAKRKGVEYHDPIKNESKSGETNVPYNPSPLP
SKDKLSSYQPPPKRNVGTFGVPQRGEASHYSAPAPTSGQSTYPANQQQYQHPNEIQTSSGYQEPPPEYSVDS
NSDMQGFSAGSEYQRTAHPAPASTTFTPANQTSFIASPQPTSIATDNAIQNIQQQYNSVSKQPSPAPGLPPL
QHQPGNLVNPPLPPQVPQKSNVPPSLPSRTSVASVASSTSQQSVGQGSFVNAQEQPKPKPALPDPGSFAPPP
RRRDQQPIKPKILTNNSTMGNEKMSSPLVQGQSSSSNIGLHPSKSISEREHQSDYSDASSKPPSLPSRTSSS
HSNLPLKQKPPKPKKLQGDSSITTSHTPGYNSNYTHNVFSARSEEEYATPPKPPRPVEDEEYTNPPKPPRPV
EDEEYTNPPKPPRPVEDDEYTNPPKPPRPETQNSSVVTPRAIPDATELSNKKPPPPKPLKKPSTLDGSTSSP
PLYSELDNSFSKPKQIISESTNSQSAVSSELNSIFQKMNINKTESEAPASSPEVKPKPKPKPVPKPKPEMIT
KKQESPETSIRIATTTKPPPPVRRLSTPHKSPSPPPVPPARNYSRAPAPPPPKQSGPPNLDLELSSGWYAKT
NGPLQLPKVFHGINHKFSYTTSSGSYGKGTTTLTVRLKDLSIVTYKFEYSNNDISNVNVVIEKYVPSPIDTT
PSKQELIANHQRFGEYIASWCEHHRGKTVGRGECWDLAKEALQKGCGKHAFVSSYTIHGYPILQIGNVGNGV
YFINNSQQLDEVRRGDILQFNACTFYDASTGVTQSAGAPDHTSVVIGKSGDKLMVLEQNVGGKRYVVDGEIN
LKNLTKGEVYVYRAMPHEWAGEL*

| Rank | Sequence | Start position | Score |
|---|---|---|---|
| 1 | HKSPSPPPVPPARNYS | 605 | 0.96 |
| 1 | SGYQEPPPEYSVDSNS | 131 | 0.96 |
| 2 | TKKQESPETSIRIATT | 576 | 0.95 |
| 3 | DGSTSSPPLYSELDNS | 498 | 0.93 |
| 3 | DPGSFAPPPRRRDQQP | 280 | 0.93 |
| 4 | CEHHRGKTVGRGECWD | 741 | 0.92 |
| 4 | PPVPPARNYSRAPAPP | 611 | 0.92 |
| 5 | PRAIPDATELSNKKPP | 471 | 0.91 |
| 5 | PPKPPRPVEDDEYTNP | 440 | 0.91 |
| 5 | DSSITTSHTPGYNSNY | 379 | 0.91 |
| 6 | GEYIASWCEHHRGKTV | 734 | 0.90 |
| 6 | YSRAPAPPPPKQSGPP | 619 | 0.90 |
| 6 | LPLKQKPPKPKKLQGD | 364 | 0.90 |
| 6 | PKPKPALPDPGSFAPP | 272 | 0.90 |
| 7 | YGKGTTTLTVRLKDLS | 674 | 0.89 |
| 7 | YNSVSKQPSPAPGLPP | 200 | 0.89 |
| 8 | NVVIEKYVPSPIDTTP | 706 | 0.88 |
| 8 | FSYTTSSGSYGKGTTT | 665 | 0.88 |
| 8 | LSSGWYAKTNGPLQLP | 640 | 0.88 |
| 8 | PTSIATDNAIQNIQQQ | 184 | 0.88 |
| 9 | PPKPPRPVEDEEYTNP | 425 | 0.87 |
| 9 | PPKPPRPVEDEEYTNP | 410 | 0.87 |
| 9 | YKKNEAKRKGVEYHDP | 37 | 0.87 |
| 9 | PGLPPLQHQPGNLVNP | 211 | 0.87 |
| 10 | QELIANHQRFGEYIAS | 724 | 0.86 |
| 10 | IATTTKPPPPVRRLST | 588 | 0.86 |
| 11 | FYDASTGVTQSAGAPD | 817 | 0.85 |
| 11 | NKTESEAPASSPEVKP | 545 | 0.85 |
| 11 | EEEYATPPKPPRPVED | 404 | 0.85 |
| 11 | QTSFIASPQPTSIATD | 175 | 0.85 |
| 11 | AHPAPASTTFTPANQT | 161 | 0.85 |
| 12 | KQIISESTNSQSAVSS | 518 | 0.84 |
| 12 | GVEYHDPIKNESKSGE | 46 | 0.84 |
| 12 | PPKPPRPETQNSSVVT | 455 | 0.84 |
| 13 | PSPIDTTPSKQELIAN | 714 | 0.83 |
| 13 | PPKQSGPPNLDLELSS | 627 | 0.83 |
| 13 | SKSGETNVPYNPSPLP | 57 | 0.83 |
| 13 | TELSNKKPPPPKPLKK | 478 | 0.83 |
| 13 | HQPGNLVNPPLPPQVP | 218 | 0.83 |
| 13 | AGSEYQRTAHPAPAST | 153 | 0.83 |
| 14 | DKLSSYQPPPKRNVGT | 75 | 0.82 |
| 14 | KPVPKPKPEMITKKQE | 565 | 0.82 |
| 14 | DEEYTNPPKPPRPVED | 434 | 0.82 |
| 14 | DEEYTNPPKPPRPVED | 419 | 0.82 |
| 14 | SSTSQQSVGQGSFVNA | 253 | 0.82 |
| 14 | VNPPLPPQVPQKSNVP | 224 | 0.82 |
| 14 | PANQQQYQHPNEIQTS | 115 | 0.82 |
| 14 | PAPTSGQSTYPANQQQ | 105 | 0.82 |
| 15 | KEALQKGCGKHAFVSS | 759 | 0.81 |
| 15 | ASSPEVKPKPKPKPVP | 553 | 0.81 |
| 16 | EVRRGDILQFNACTFY | 803 | 0.80 |
| 16 | YNSNYTHNVFSARSEE | 390 | 0.80 |
| 17 | VGTFGVPQRGEASHYS | 88 | 0.79 |
| 17 | GECWDLAKEALQKGCG | 752 | 0.79 |
| 17 | LPKVFHGINHKFSYTT | 654 | 0.79 |
| 17 | RRRDQQPIKPKILTNN | 289 | 0.79 |
| 17 | TFKGITSGTKAVGQAG | 18 | 0.79 |
| 18 | PPPVRRLSTPHKSPSP | 595 | 0.78 |
| 18 | PKILTNNSTMGNEKMS | 298 | 0.78 |
| 18 | KAVGQAGYRTYKKNEA | 27 | 0.78 |
| 18 | TFTPANQTSFIASPQP | 169 | 0.78 |
| 19 | PSKSISEREHQSDYSD | 330 | 0.77 |
| 20 | IGNVGNGVYFINNSQQ | 785 | 0.76 |
| 20 | YSDASSKPPSLPSRTS | 343 | 0.76 |
| 21 | KDLSIVTYKFEYSNND | 686 | 0.75 |
| 21 | RTSSSHSNLPLKQKPP | 356 | 0.75 |
| 21 | SRTSVASVASSTSQQS | 244 | 0.75 |

| Start | End | Max_score_pos | Sequence |
|---|---|---|---|
| 704 | 716 | 710 | NVNVVIEKYVPSP |
| 651 | 662 | 655 | PLQLPKVFHGIN |
| 199 | 255 | 250 | QYNSVSKQPSPAPGLPPLQHQPGNLVNPPLPPQVPQKS |
| | | | NVPPS |
| | | | LPSRTSVASVASST |
| 871 | 878 | 875 | GEVYVYRA |
| 763 | 786 | 769 | QKGCGKHAFVSSYTIHGYPILQIG |
| 680 | 695 | 688 | TLTVRLKDLSIVTYKF |
| 466 | 472 | 471 | SSVVTPR |
| 833 | 840 | 836 | HTSVVIGK |
| 594 | 617 | 613 | PPPPVRRLSTPHKSPSPPPVPPAR |
| 809 | 819 | 812 | ILQFNACTFYD |
| 855 | 862 | 860 | KRYVVDGE |
| 503 | 509 | 507 | SPPLYSE |
| 257 | 269 | 263 | QQSVGQGSFVNAQ |
| 314 | 320 | 317 | SPLVQGQ |
| 134 | 143 | 139 | QEPPPEYSVD |
| 528 | 538 | 532 | QSAVSSELNSI |
| 46 | 52 | 50 | GVEYHDP |
| 65 | 83 | 80 | PYNPSPLPSKDKLSSYQPP |
| 734 | 744 | 738 | GEYIASWCEHH |
| 790 | 795 | 791 | NGVYFI |
| 295 | 300 | 298 | PIKPKI |
| 100 | 108 | 104 | SHYSAPAPT |
| 844 | 851 | 850 | KLMVLEQN |
| 325 | 333 | 331 | NIGLHPSKS |
| 361 | 369 | 367 | HSNLPLKQK |
| 486 | 494 | 488 | PPPKPLKKP |
| 563 | 570 | 565 | KPKPVPKP |
| 635 | 643 | 639 | NLDLELSSG |
| 176 | 186 | 181 | TSFIASPQPTS |
| 552 | 561 | 555 | PASSPEVKPK |
| 621 | 631 | 625 | RAPAPPPPKQS |
| 160 | 167 | 164 | TAHPAPAS |
| 395 | 401 | 397 | THNVFSA |
| 26 | 32 | 31 | TKAVGQA |
| 348 | 356 | 351 | SKPPSLPSR |
| 112 | 122 | 121 | STYPANQQQYQ |
| 274 | 287 | 275 | PKPALPDPGSFAPP |
| 821 | 828 | 827 | STGVTQSA |
| 723 | 730 | 724 | KQELIANH |
| 516 | 522 | 518 | KPKQIIS |

52. PLB4
MNLLISLLLLSISLVLGSSPSGGYAPGIVQCPINSNSNSSSSSSRNTTFSFIREADSISDLEKQWIKQRQLK
VNKSLIEFLKSANLSNFNPQNFIDAKDYQGINLGLAFSGGSYRAMLNGAGQLMALDSRSSSSPSESGSGSGS
LGGILQSANYIGGLSGSSWLLGSLAMQGWPTVEEVVFENPHDVWNLTSSRQLVNQTGLWTIVFPVMFDNMNK
ALSHMNFWDNNADGIKFDLEAKEKAGFETSLTDAWARGLAHQLFPKGKDNYGSSETWSDIRNIDAFANHDMP
FPFVTGLGRKPGTTVYNLNSTVIEMNPFEFGSFDPSLNTFTDIKYLGTNVSNGVPVDSCVNGFDNSGFIVGS
SSSLFNSCLNTLVCDNCNSLNSVIKWILKKFLTYKLMKMWLFTNPNPFFNSQYAKSDNITTSDTLYVIDGGI
GGEVIPLSTLMVKERALDIVFAFDSDTNTKTNWPDGSALISSYERQFSQQGSSSICPYVPDTKTFLEKGLTA
KPTFFGCDAKNLTALEKDGVIPPLVVYFANRPYEYYSNVSTFDLTFTDEQKKGLIKNGFDVATRLNGTIDPE
FKSCIACAVIRREEERRGIEQSDQCKKCFKNYCWDGTYASGPAENYVNFTDSSLTNGSTVFYGKADAKVSSS
KGGLFGFLKRDTQNNDEKEEFIGVVRESNSDSLKLSKYLTIASLFALYLVIM*

| Rank | Sequence | Start position | Score |
|---|---|---|---|
| 1 | TGLGRKPGTTVYNLNS | 293 | 0.94 |
| 1 | GSSPSGGYAPGIVQCP | 17 | 0.94 |
| 2 | LGLAFSGGSYRAMLNG | 105 | 0.92 |
| 3 | LWTIVFPVMFDNMNKA | 202 | 0.91 |
| 4 | FIVGSSSSLFNSCLNT | 356 | 0.90 |
| 5 | NGSTVFYGKADAKVSS | 632 | 0.89 |
| 5 | NYCWDGTYASGPAENY | 607 | 0.89 |
| 5 | SDTNTKTNWPDGSALI | 457 | 0.89 |
| 5 | SETWSDIRNIDAFANH | 270 | 0.89 |
| 5 | PGIVQCPINSNSNSSS | 26 | 0.89 |
| 5 | SRSSSSPSESGSGSGS | 129 | 0.89 |
| 6 | YGKADAKVSSSKGGLF | 638 | 0.87 |
| 7 | TVIEMNPFEFGSFDPS | 309 | 0.86 |
| 7 | DNMNKALSHMNFWDNN | 212 | 0.86 |
| 8 | KGLTAKPTFFGCDAKN | 500 | 0.85 |
| 9 | EQSDQCKKCFKNYCWD | 596 | 0.84 |
| 9 | KGLIKNGFDVATRLNG | 556 | 0.84 |
| 9 | VYFANRPYEYYSNVST | 530 | 0.84 |
| 9 | AMQGWPTVEEVVFENP | 169 | 0.84 |
| 10 | KGGLFGFLKRDTQNND | 649 | 0.83 |
| 10 | YEYYSNVSTFDLTFTD | 537 | 0.83 |
| 10 | SFIREADSISDLEKQW | 50 | 0.83 |
| 10 | GEVIPLSTLMVKERAL | 434 | 0.83 |
| 10 | SSSRNTTFSFIREADS | 42 | 0.83 |
| 11 | CAVIRREEERRGIEQS | 583 | 0.82 |
| 11 | QFSQQGSSSICPYVPD | 478 | 0.82 |
| 11 | FTDIKYLGTNVSNGVP | 328 | 0.82 |
| 12 | QNFIDAKDYQGINLGL | 92 | 0.81 |
| 13 | GPAENYVNFTDSSLTN | 617 | 0.80 |
| 13 | ATRLNGTIDPEFKSCI | 566 | 0.80 |
| 13 | EEVVFENPHDVWNLTS | 177 | 0.80 |
| 14 | MWLFTNPNPFFNSQYA | 399 | 0.79 |
| 14 | NGVPVDSCVNGFDNSG | 340 | 0.79 |
| 15 | VPDTKTFLEKGLTAKP | 491 | 0.78 |
| 15 | IVFAFDSDTNTKTNWP | 451 | 0.78 |
| 15 | SCLNTLVCDNCNSLNS | 367 | 0.78 |
| 15 | WARGLAHQLFPKGKDN | 251 | 0.78 |
| 15 | PHDVWNLTSSRQLVNQ | 184 | 0.78 |
| 16 | EKEEFIGVVRESNSDS | 665 | 0.77 |
| 16 | MPFPFVTGLGRKPGTT | 287 | 0.77 |
| 16 | DLEAKEKAGFETSLTD | 234 | 0.77 |
| 17 | LKVNKSLIEFLKSANL | 71 | 0.76 |
| 17 | RESNSDSLKLSKYLTI | 674 | 0.76 |
| 17 | TSLTDAWARGLAHQLF | 245 | 0.76 |
| 17 | GSSWLLGSLAMQGWPT | 160 | 0.76 |

| Start | End | Max_score_pos | Sequence |
|---|---|---|---|
| 521 | 534 | 527 | KDGVIPPLVVYFAN |
| 577 | 587 | 583 | FKSCIACAVIR |
| 339 | 350 | 345 | SNGVPVDSCVNG |
| 680 | 697 | 695 | SLKLSKYLTIASLFALYL |
| 4 | 19 | 6 | LISLLLLSISLVLGSS |
| 355 | 377 | 371 | GFIVGSSSSLFNSCLNTLVCDNC |
| 484 | 495 | 489 | SSSICPYVPDTK |
| 25 | 34 | 30 | APGIVQCPIN |
| 434 | 447 | 439 | GEVIPLSTLMVKER |
| 202 | 210 | 208 | LWTIVFPVM |
| 449 | 455 | 452 | LDIVFAF |
| 176 | 184 | 178 | VEEVVFENP |
| 253 | 262 | 258 | RGLAHQLFPK |
| 379 | 395 | 385 | SLNSVIKWILKKFLTYK |
| 422 | 430 | 425 | SDTLYVIDG |
| 598 | 608 | 603 | SDQCKKCFKNY |
| 468 | 478 | 472 | GSALISSYERQ |
| 288 | 295 | 292 | PFPFVTGL |
| 669 | 675 | 671 | FIGVVRE |
| 161 | 168 | 166 | SSWLLGSL |
| 68 | 81 | 78 | QRQLKVNKSLIEFL |
| 537 | 548 | 540 | YEYYSNVSTFDL |
| 102 | 111 | 106 | GINLGLAFSG |
| 193 | 200 | 199 | SRQLVNQT |
| 145 | 153 | 151 | LGGILQSAN |
| 635 | 640 | 638 | TVFYGK |
| 503 | 514 | 508 | TAKPTFFGCDAK |
| 330 | 336 | 336 | DIKYLGT |
| 642 | 648 | 646 | DAKVSSS |
| 301 | 309 | 309 | TTVYNLNST |

53. PCT1
MARLTRKRTIEKELNGSSRVTRTLSMESISSLFKRNKKRKHNDGNDSSVNSSDNENINITDDEEQDHIDTKP
NHKKRKIKTKAEEEFEANEKKLDEELPIDLRKYRPRGFRFNLPPEDRPIRIYADGVFDLFHLGHMKQLEQAK
KSFPNVELVCGIPSDIETHKRKGLTVLTDEQRCETLMHCKWVDEVIPNAPWCVTPEFLQEHKIDYVAHDDLP
YASSDSDDIYKPIKEQGKFLTTQRTEGISTSDIITKIIRDYDKYLMRNFSRGATRKELNVSWLKMNELEFKK
HINDFRTYWMKNKTNINNVSRDLYFEIREFMRGKKFDFQKFIEDGNSQNSSNHGSDEESTNSSKVSSPLSDF
ASKYIGNRNKDLNRKGILNNFKGWINRDDHSEQETEEEIKPIVIKPIRRSRRLSGGSSTSSVPSTPVKRTAS
SASTTPKRKSPLKKSSSVKNTPKTK

| Rank | Sequence | Start position | Score |
|---|---|---|---|
| 1 | QDHIDTKPNHKKRKIK | 65 | 0.91 |
| 1 | EFLQEHKIDYVAHDDL | 200 | 0.91 |
| 1 | DLRKYRPRGFRFNLPP | 101 | 0.91 |
| 2 | KRKIKTKAEEEFEANE | 76 | 0.90 |
| 2 | SWLKMNELEFKKHIND | 277 | 0.90 |
| 2 | TSDIITKIIRDYDKYL | 246 | 0.90 |
| 3 | PSTPVKRTASSASTTP | 423 | 0.89 |
| 3 | HCKWVDEVIPNAPWCV | 182 | 0.89 |
| 3 | TVLTDEQRCETLMHCK | 169 | 0.89 |
| 4 | SSTSSVPSTPVKRTAS | 417 | 0.88 |
| 5 | DHSEQETEEEIKPIVI | 389 | 0.87 |
| 6 | NINITDDEEQDHIDTK | 56 | 0.86 |
| 6 | SSDSDDIYKPIKEQGK | 219 | 0.86 |
| 7 | SSVNSSDNENINITDD | 47 | 0.85 |
| 7 | ASSASTTPKRKSPLKK | 431 | 0.85 |
| 7 | YFEIREFMRGKKFDFQ | 312 | 0.85 |
| 7 | PIRIYADGVFDLFKLG | 120 | 0.85 |
| 8 | KRTIEKELNGSSRVTR | 7 | 0.83 |
| 8 | DFASKYIGNRNKDLNR | 359 | 0.83 |
| 8 | PSDIETHKRKGLTVLT | 157 | 0.83 |
| 9 | NKKRKHNDGNDSSVNS | 36 | 0.82 |
| 9 | KKHINDFRTYWMKNKT | 287 | 0.82 |
| 9 | RTLSMESISSLFKRNK | 22 | 0.82 |
| 9 | DDLPYASSDSDDIYKP | 213 | 0.82 |
| 9 | RCETLMHCKWVDEVIP | 176 | 0.82 |
| 10 | IEDGNSQNSSNHGSDE | 330 | 0.80 |
| 10 | LMRNFSRGATRKELNV | 261 | 0.80 |
| 11 | RRLSGGSSTSSVPSTP | 411 | 0.79 |
| 12 | TEEEIKPIVIKPIRRS | 395 | 0.78 |
| 12 | FKGWINRDDHSEQETE | 381 | 0.78 |
| 12 | TQRTEGISTSDIITKI | 238 | 0.78 |
| 13 | TPKRKSPLKKSSSVKN | 437 | 0.76 |
| 13 | SSKVSSPLSDFASKYI | 350 | 0.76 |
| 13 | HGSDEESTNSSKVSSP | 341 | 0.76 |
| 13 | GSSRVTRTLSMESISS | 16 | 0.76 |
| 14 | KFDFQKFIEDGNSQNS | 323 | 0.75 |
| 14 | EVIPNAPWCVTPEFLQ | 188 | 0.75 |

| Start | End | Max_score_pos | Sequence |
|---|---|---|---|
| 147 | 160 | 153 | FPNVELVCGIPSDI |
| 121 | 137 | 131 | IRIYADGVFDLFHLGHM |
| 177 | 220 | 199 | CETLMHCKWVDEVIPNAPWCVTPEFLQEHKIDYVAHDDLPY |
| | | | ASS |
| 400 | 407 | 404 | KPIVIKPI |
| 419 | 430 | 424 | TSSVPSTPVKRT |
| 350 | 364 | 354 | SSKVSSPLSDFASKY |
| 308 | 315 | 310 | SRDLYFEI |
| 441 | 452 | 447 | KSPLKKSSSVKN |
| 167 | 174 | 169 | GLTVLTDE |
| 273 | 279 | 277 | ELNVSWL |
| 99 | 105 | 101 | PIDLRKY |
| 250 | 260 | 252 | ITKIIRDYDKY |

54. COX11
MNRLRIYTPIFRSTIVKPAVFRPYAFIVSRGIHTTGKLFQMQHQQQPSVPDQSSIEKQREWVDRLAREREER
QKYRNRTATYYTASLGIFFLALAFSAVPIYRAICQRTGWGGIPITDSTKFTPDKLIPVDTNKRIRIQFTCQS
SGILPWKFTPLQREVYVVPGETALAFYRAKNMSKEDIIGMATYSISPDNVAGYFNKIQCFCFEEQRLSAGEE
VDMPVFFFIDPDFAKDPAMRNIDDVVLHYSFFKAHYSDGELAAAPIGNMEMKASVVS

| Rank | Sequence | Start position | Score |
|---|---|---|---|
| 1 | RSTIVKPAVFRPYAFI | 12 | 0.96 |
| 2 | GIPITDSTKFTPDKLI | 113 | 0.94 |
| 3 | DRLAREREERQKYRNR | 63 | 0.93 |
| 3 | IGMATYSISPDNVAGY | 182 | 0.93 |
| 4 | AGEEVDMPVFFFIDPD | 213 | 0.89 |
| 5 | RLRIYTPIFRSTIVKP | 3 | 0.88 |
| 6 | MQHQQQPSVPDQSSIE | 41 | 0.87 |
| 7 | QSSIEKQREWVDRLAR | 52 | 0.84 |
| 8 | LPWKFTPLQREVYVVP | 148 | 0.83 |
| 9 | KYRNRTATYYTASLGI | 74 | 0.81 |
| 9 | ISPDNVAGYFNKIQCF | 189 | 0.81 |
| 10 | GETALAFYRAKNMSKE | 164 | 0.78 |
| 11 | GELAAAPIGNMEMKAS | 255 | 0.76 |
| 11 | RIRIQFTCQSSGILPW | 135 | 0.76 |

| Start | End | Max_score_pos | Sequence |
|---|---|---|---|
| 239 | 252 | 244 | DDVVLHYSFFKAHY |
| 200 | 208 | 203 | KIQCFCFEE |
| 137 | 17 | 1161 | RIQFTCQSSGILPWKFTPLQREVYVVPGETALAFY |
| 6 | 31 | 18 | IYTPIFRSTIVKPAVFRPYAFIVSRG |
| 82 | 108 | 92 | YYTASLGIFFLALAFSAVPIYRAICQR |
| 218 | 228 | 223 | DMPVFFFDPD |
| 124 | 131 | 127 | PDKLIPVD |
| 39 | 52 | 46 | FQMQHQQQPSVPDQ |
| 187 | 198 | 188 | YSISPDNVAGYF |

55. KEL1
MALFKLGGKLKKKDHQSDPDTTVSSSSSTNSANRKSTSRFSSILHSSAAPMPSISNQPAHTSRPPPHANLSV
TTPWNRFKLFDSPFPRYRHAAASIASEKNELFLMGGLKDGSVFGDTWKIVPQINHEGDIINYVAENIEVVNN
NNPPARVGHAAVLCGNAFIVYGGDTVDTDTNGFPDNNFYLFNINNHKYTIPNHILNKPNGRYGHTIGVISLN
NTSSRLYLFGGQLENDVFNDLYYFELNSFKSPKATWQLVEPLNDVKPPPLTNHSMSVYKNKVYVFGGVYNNE
KVSNDLWVFDAINDTWTQVTTTGDIPPPVNEHSSCVADDRMYVYGGNDFQGIIYSSLYVLDLQTLEWSSLQS
SAEKSGPGPRCGHSMTLLPKFNKILIMGGDKNDYVDSDPHNFETYESFNGEEVGTMVYELDLNIIDHFLAAS
APVNAPTIIPPVASYEELPKPKKPAASARNDLQGYDRHARSFSGGPEDFATPQASARGSPSPERTQGGGDNF
VEVDLPSTTISQVDDDPPYDTTSLNQPQEVTNGHVDDEPFRRRSLDPKFDDHSGAPEVAPVAVPVTEPVSAP
VTAPVAEPVVAPAVAPDASGKVKKIISELTNELVQLKATTKEQMQKATEKIEQLERQNSLLHQSQQRDAESY
TKQIEEKDVLINELKSSLDPSAWDPEQPQTATNISELNRYKLERLELNNKLLYLEQENVKLKDQFAEFEPFM
DHQIGELDKFQKVIKVQEEQIDKLSNQVKDQEALHKQIYDWKSKFESLSLEFENYRAIHNDDDISDGEVELQ
DDDRSILSSAKSRKDISSQLGNLVSLWNQKHASSSSRDLSAPPVINPESHPVVAKLQSQVDELLKIGKQNET
TFSQEIEALRKELQEKTTSLKTVEENYRESIQSVNNTSKALKLNQEELSNQRILMERLIKENNELKLYKKAS
SKKLGSRDGTPVVNEYQQGEDSPGVDELNNDDDDDEDVISTAHYNMKIKDLEADLYILKQERDQLKDNVTSL
QKQLYLAQNQ

| Rank | Sequence | Start position | Score |
|---|---|---|---|
| 1 | YQQGEDSPGVDELNND | 952 | 0.94 |
| 1 | AHTSRPPPHANLSVTT | 59 | 0.94 |
| 2 | NGHVDDEPFRRRSLDP | 536 | 0.93 |
| 2 | KILIMGGDKNDYVDSD | 383 | 0.93 |
| 3 | TGDIPPPVNEHSSCVA | 310 | 0.90 |
| 4 | LKSSLDPSAWDPEQPQ | 662 | 0.89 |
| 4 | MPSISNQPAHTSRPPP | 51 | 0.89 |
| 5 | SQEIEALRKELQEKTT | 867 | 0.88 |
| 5 | PSTTISQVDDDPPYDT | 510 | 0.88 |
| 5 | IVYGGDTVDTDTNGFP | 163 | 0.88 |
| 6 | DGEVELQDDDRSILSS | 786 | 0.86 |
| 6 | TPQASARGSPSPERTQ | 483 | 0.86 |
| 6 | APTIIPPVASYEELPK | 437 | 0.86 |
| 6 | NGEEVGTMVYELDLNI | 409 | 0.86 |
| 6 | FQGIIYSSLYVLDLQT | 337 | 0.86 |
| 6 | HSSCVADDRMYVYGGN | 320 | 0.86 |
| 6 | LKKKDHQSDPDTTVSS | 10 | 0.86 |
| 7 | RRRSLDPKFDDHSGAP | 545 | 0.85 |
| 7 | GPEDFATPQASARGSP | 477 | 0.85 |
| 7 | AEKSGPGPRCGHSMTL | 362 | 0.85 |
| 7 | PNHILNKPNGRYGHTI | 195 | 0.85 |
| 8 | EDVISTAHYNMKIKDL | 972 | 0.84 |
| 8 | LGSRDGTPVVNEYQQG | 940 | 0.84 |
| 8 | QEEQIDKLSNQVKDQE | 737 | 0.84 |
| 8 | MQKATEKIEQLERQNS | 620 | 0.84 |
| 8 | RHARSFSGGPEDFATP | 469 | 0.84 |
| 8 | TVSSSSSTNSANRKST | 22 | 0.84 |
| 8 | TVDTDTNGFPDNNFYL | 169 | 0.84 |
| 9 | SRDLSAPPVINPESHP | 828 | 0.83 |
| 9 | ANLSVTTPWNRFKLFD | 68 | 0.83 |
| 9 | LNIIDHFLAASAPVNA | 422 | 0.83 |
| 9 | TLEWSSLQSSAEKSGP | 352 | 0.83 |
| 10 | ESLSLEFENYRAIHND | 766 | 0.82 |
| 10 | TKQIEEKDVLINELKS | 649 | 0.82 |
| 10 | AVAPDASGKVKKIISE | 589 | 0.82 |
| 10 | EGDIINYVAENIEVVN | 128 | 0.82 |
| 11 | KTVEENYRESIQSVNN | 885 | 0.81 |
| 11 | RKELQEKTTSLKTVEE | 874 | 0.81 |
| 11 | DPEQPQTATNISELNR | 672 | 0.81 |
| 11 | ERTQGGGDNFVEVDLP | 495 | 0.81 |
| 11 | RGSPSPERTQGGGDNF | 489 | 0.81 |
| 11 | GVYNNEKVSNDLWVFD | 283 | 0.81 |
| 11 | NGRYGHTIGVISLNNT | 203 | 0.81 |
| 12 | QVDDDPPYDTTSLNQP | 516 | 0.80 |
| 12 | GHSMTLLPKFNKILIM | 372 | 0.80 |
| 13 | ADLYILKQERDQLKDN | 989 | 0.79 |
| 13 | DQEALHKQIYDWKSKF | 750 | 0.79 |
| 13 | SELNRYKLERLELNNK | 683 | 0.79 |
| 13 | FVEVDLPSTTISQVDD | 504 | 0.79 |
| 13 | RMYVYGGNDFQGIIYS | 328 | 0.79 |
| 14 | LFDSPFPRYRHAAASI | 81 | 0.78 |
| 14 | PKATWQLVEPLNDVKP | 248 | 0.78 |
| 15 | TAPVAEPVVAPAVAPD | 578 | 0.77 |
| 15 | TTSLNQPQEVTNGHVD | 525 | 0.77 |
| 16 | KQIYDWKSKFESLSLE | 756 | 0.76 |
| 16 | HQSQQRDAESYTKQIE | 638 | 0.76 |
| 16 | PVAVPVTEPVSAPVTA | 564 | 0.76 |
| 16 | PLTNHSMSVYKNKVYV | 265 | 0.76 |
| 17 | VASYEELPKPKKPAAS | 444 | 0.75 |
| 17 | SANRKSTSRFSSILHS | 31 | 0.75 |
| 17 | KVYVFGGVYNNEKVSN | 277 | 0.75 |
| 17 | DGSVFGDTWKIVPQIN | 111 | 0.75 |

| Start | End | Max_score_pos | Sequence |
|---|---|---|---|
| 559 | 593 | 566 | APEVAPVAVPVTEPVSAPVTAPVAEPVVAPAVAPD |
| 339 | 353 | 345 | GIIYSSLYVLDLQTL |
| 828 | 858 | 845 | SRDLSAPPVINPESHPVVAKLQSQVDELLKI |
| 148 | 167 | 155 | PARVGHAAVLCGNAFIVYGG |
| 270 | 283 | 281 | SMSVYKNKVYVFGG |
| 414 | 450 | 432 | GTMVYELDLNIIDHFLAASAPVNAPTIIPPVASYEEL |
| 504 | 520 | 507 | FVEVDLPSTTISQVDDD |
| 730 | 739 | 733 | FQKVIKVQEE |
| 1002 | 1015 | 1011 | KDNVTSLQKQLYLA |
| 314 | 334 | 323 | PPPVNEHSSCVADDRMYVYGG |
| 607 | 614 | 612 | NELVQLKA |
| 809 | 819 | 815 | SSQLGNLVSLW |
| 209 | 215 | 212 | TIGVISL |
| 696 | 707 | 701 | NNKLLYLEQENV |
| 251 | 267 | 255 | TWQLVEPLNDVKPPPLT |
| 291 | 299 | 297 | SNDLWVFDA |
| 234 | 243 | 236 | FNDLYYFELN |
| 750 | 758 | 756 | DQEALHKQI |
| 988 | 995 | 992 | EADLYILK |
| 633 | 640 | 639 | QNSLLHQS |
| 40 | 52 | 46 | FSSILHSSAAPMP |
| 60 | 73 | 69 | HTSRPPPHANLSVT |
| 221 | 227 | 222 | RLYLFGG |
| 119 | 125 | 124 | WKIVPQI |
| 946 | 952 | 950 | TPVVNEY |
| 654 | 661 | 660 | EKDVLINE |
| 132 | 142 | 133 | INYVAENIEVV |
| 81 | 97 | 94 | LFDSPFPRYRHAAASIA |
| 930 | 935 | 932 | KLYKKA |
| 595 | 604 | 600 | SGKVKKIISE |
| 797 | 802 | 800 | SILSSA |
| 368 | 384 | 376 | GPRCGHSMTLLPKFNKI |
| 192 | 200 | 196 | YTIPNHILN |
| 765 | 771 | 767 | FESLSLE |
| 663 | 669 | 665 | KSSLDPS |
| 355 | 361 | 359 | WSSLQSS |
| 20 | 26 | 25 | DTTVSSS |
| 4 | 9 | 5 | FKLGGK |
| 742 | 747 | 746 | DKLSNQ |
| 452 | 458 | 453 | KPKKPAA |

56. GAP1
MAIKIGINGFGRIGRLVLRVALGRKDIEVVAVNDPFIAPDYAAYMFKYDSTHGRYKGEVTASGDDLVIDGHK
IKVFQERDPANIPWGKSGVDYVIESTGVFTKLEGAQKHIDAGAKKVIITAPSADAPMFVVGVNEDKYTPDLK
IISNASCTTNCLAPLAKVVNDTFGIEEGLMTTVHSITATQKTVDGPSHKDWRGGRTASGNIIPSSTGAAKAV
GKVIPELNGKLTGMSLRVPTTDVSVVDLTVRLKKAASYEEIAQAIKKASEGPLKGVLGYTEDAVVSTDFLGS
SYSSIFDEKAGILLSPTFVKLISWYDNEYGYSTRVVDLLEHVAKASA

| Rank | Sequence | Start position | Score |
|---|---|---|---|
| 1 | TAPSADAPMFVVGVNE | 121 | 0.94 |
| 2 | VGVNEDKYTPDLKIIS | 132 | 0.93 |
| 3 | KKASEGPLKGVLGYTE | 262 | 0.91 |
| 4 | PSHKDWRGGRTASGNI | 190 | 0.89 |
| 4 | QKTVDGPSHKDWRGGR | 184 | 0.89 |
| 5 | DYVIESTGVFTKLEGA | 92 | 0.85 |
| 5 | PLKGVLGYTEDAVVST | 268 | 0.85 |
| 5 | KYTPDLKIISNASCTT | 138 | 0.85 |
| 5 | TKLEGAQKHIDAGAKK | 102 | 0.85 |
| 6 | TGMSLRVPTTDVSVVD | 228 | 0.84 |
| 6 | QKHIDAGAKKVIITAP | 108 | 0.84 |
| 7 | IPELNGKLTGMSLRVP | 220 | 0.83 |
| 7 | IEEGLMTTVHSITATQ | 169 | 0.83 |
| 8 | YAAYMFKYDSTHGRYK | 41 | 0.82 |
| 9 | GHKIKVFQERDPANIP | 70 | 0.81 |
| 10 | LISWYDNEYGYSTRVV | 309 | 0.80 |
| 11 | KAGILLSPTFVKLISW | 297 | 0.79 |
| 11 | YSSIFDEKAGILLSPT | 290 | 0.79 |
| 12 | PFIAPDYAAYMFKYDS | 35 | 0.78 |
| 13 | STHGRYKGEVTASGDD | 50 | 0.77 |
| 14 | IPWGKSGVDYVIESTG | 84 | 0.75 |
| 14 | ASCTTNCLAPLAKVVN | 149 | 0.75 |

| Start | End | Max_score_pos | Sequence |
|---|---|---|---|
| 14 | 24 | 19 | GRLVLRVALGR |
| 231 | 252 | 242 | SLRVPTTDVSVVDLTVRLKKAA |
| 152 | 166 | 160 | TTNCLAPLAKVVNDT |
| 320 | 332 | 327 | STRVVDLLEHVAK |
| 27 | 47 | 29 | IEVVAVNDPFIAPDYAAYMFK |
| 128 | 135 | 131 | PMFVVGVN |
| 299 | 312 | 304 | GILLSPTFVKLISW |
| 277 | 293 | 283 | EDAVVSTDFLGSSYSSI |
| 213 | 222 | 218 | AKAVGKVIPE |
| 90 | 103 | 92 | GVDYVIESTGVFTK |
| 115 | 125 | 121 | AKKVIITAPSA |
| 269 | 275 | 272 | LKGVLGY |
| 64 | 78 | 74 | DDLVIDGHKIKVFQE |
| 140 | 150 | 148 | TPDLKIISNAS |
| 174 | 181 | 179 | MTTVHSIT |
| 254 | 263 | 260 | YEEIAQAIKK |
| 204 | 209 | 208 | NIIPSS |

57. IRS4_CANAL
MSGNSAANAAALAAFNGIGKKKKESTTKLNGTDNNTNHLGVIGSTSNQTKQHQQQQQQPQALRTPLPAHPSR
KKSNKFSQLKRLNTAPAMASLQPALQIASPSISPTQPSAPASALDSDPDYFTLSPHTIPSKNEIAKSPQTPQ
DMIRNVRQSIELKAIPNNAQAKRLSVDYSPQEMLKNLRHSLHSRTKTSPMLTTSDKMGQTMLAEMRDRLENT
RRIASNSVASLSLSPNLDFNKSTSDVSNLSHHYDVDTVSTDSFASFSSSINDRHLPHGISIDVTNHDSDEDE
IDDREREEDHEPLDNGELKSTNNKVTVSSRLRRKPPPGEDFQMQLNDKSRDTISSGSYSLNPDEVYSFTDPD
SYENLVSEVEVGETTRLFPQFPDANHYHQHSSKFRKKHQKVKPINGIYYRDMDSSNTSDTEESTSNLPSRST
TPLLGQPQQQVHFRSTMRKANTKKDKKSRFNELKPWKNHNDLNYLTDQEKKRYEGIWASNKGNYMSQVVIKL
HGVNYETQKDPKEEAKMEHSRTAALLSAAAVEDSNYNGNNSLHNLDSVEINQLICGPVVKRIWKRSRLPSDT
LEKIWNLIDFRRDGTLNKNEFLVGMWLVDQCLYGRKLPKKVDNVVWDSLGGIGVNVTVKKKK*

| Rank | Sequence | Start position | Score |
|---|---|---|---|
| 1 | PINGIYYRDMDSSNTS | 403 | 0.94 |
| 2 | DKSRDTISSGSYSLNP | 335 | 0.93 |
| 3 | PDEVYSFTDPDSYENL | 350 | 0.90 |
| 4 | ASPSISPTQPSAPASA | 100 | 0.89 |
| 5 | AVEDSNYNGNNSLHNL | 534 | 0.88 |
| 5 | AKSPQTPQDMIRNVRQ | 137 | 0.88 |
| 6 | LEKIWNLIDFRRDGTL | 577 | 0.87 |
| 6 | KQHQQQQQQPQALRTP | 50 | 0.87 |
| 7 | TKTSPMLTTSDKMGQT | 189 | 0.86 |
| 7 | NGIGKKKKESTTKLNG | 16 | 0.86 |
| 8 | DTEESTSNLPSRSTTP | 419 | 0.85 |
| 8 | YRDMDSSNTSDTEEST | 409 | 0.85 |
| 8 | PHTIPSKNEIAKSPQT | 127 | 0.85 |
| 9 | YETQKDPKEEAKMEHS | 509 | 0.84 |
| 9 | HGISIDVTNHDSDEDE | 273 | 0.84 |
| 10 | NHYHQHSSKFRKKHQK | 385 | 0.83 |
| 10 | NSVASLSLSPNLDFNK | 222 | 0.83 |
| 10 | TTSDKMGQTMLAEMRD | 196 | 0.83 |
| 11 | KKRYEGIWASNKGNYM | 482 | 0.82 |
| 11 | ANTKKDKKSRFNELKP | 452 | 0.82 |
| 12 | PGEDFQMQLNDKSRDT | 325 | 0.81 |
| 12 | REREEDHEPLDNGELK | 292 | 0.81 |
| 13 | QQQVHFRSTMRKANTK | 440 | 0.80 |
| 13 | KVTVSSRLRRKPPPGE | 312 | 0.80 |
| 13 | DEDEIDDREREEDHEP | 285 | 0.80 |
| 14 | GSYSLNPDEVYSFTDP | 344 | 0.79 |
| 14 | SSSINDRHLPHGISID | 263 | 0.79 |
| 14 | PSAPASALDSDPDYFT | 109 | 0.79 |
| 15 | LRTPLPAHPSRKKSNK | 62 | 0.78 |
| 15 | LPSRSTTPLLGQPQQQ | 427 | 0.78 |
| 15 | SEVEVGETTRLFPQFP | 367 | 0.78 |
| 15 | KKESTTKLNGTDNNTN | 22 | 0.78 |
| 16 | CGPVVKRIWKRSRLPS | 559 | 0.77 |
| 16 | AALLSAAAVEDSNYNG | 527 | 0.77 |
| 16 | LGVIGSTSNQTKQHQQ | 39 | 0.77 |
| 17 | MWLVDQCLYGRKLPKK | 601 | 0.76 |
| 17 | KKSRFNELKPWKNHND | 458 | 0.76 |
| 18 | NVVWDSLGGIGVNVTV | 619 | 0.75 |
| 18 | KSTSDVSNLSHHYDVD | 237 | 0.75 |
| 18 | RQSIELKAIPNNAQAK | 151 | 0.75 |

| Start | End | Max_score_pos | Sequence |
|---|---|---|---|
| 547 | 566 | 560 | HNLDSVEINQLICGPVVKRI |
| 596 | 612 | 606 | EFLVGMWLVDQCLYGRK |
| 497 | 508 | 502 | MSQVVIKLHGVN |
| 363 | 372 | 368 | ENLVSEVEVG |
| 526 | 537 | 532 | TAALLSAAAVED |
| 221 | 232 | 226 | SNSVASLSLSPN |
| 242 | 254 | 248 | VSNLSHHYDVDTV |
| 627 | 635 | 631 | GIGVNVTVK |
| 312 | 318 | 316 | KVTVSSR |
| 89 | 117 | 96 | AMASLQPALQIASPSISPTQPSAPASALD |
| 166 | 173 | 171 | KRLSVDYS |
| 432 | 446 | 442 | TTPLLGQPQQQVHFR |
| 8 | 15 | 12 | NAAALAAF |
| 614 | 624 | 623 | PKKVDNVVWDS |
| 38 | 44 | 41 | HLGVIGS |
| 51 | 70 | 68 | QHQQQQQQPQALRTPLPAHP |
| 268 | 280 | 274 | DRHLPHGISIDVT |
| 180 | 187 | 184 | NLRHSLHS |
| 122 | 130 | 125 | YFTLSPHTI |
| 398 | 407 | 401 | HQKVKPINGI |
| 150 | 159 | 153 | VRQSIELKAI |
| 384 | 392 | 389 | ANHYHQHSS |
| 350 | 356 | 356 | PDEVYSF |
| 78 | 84 | 81 | FSQLKRL |
| 259 | 264 | 263 | FASFSS |
| 343 | 348 | 345 | SGSYSL |

58. INP51
MRLYLIEKPRTFVITTNTHALIIRHPSPTYKHSGIKGLVSGHSKDKDQNKDTKVLVEFVLKEYLDLSLYRDI
TPKHGGLLGLLGLLNVKGKTFIGFITRDEWTASATVTDRIYKITDTEFYCINNDEYDYLLDKEYENMSHQER
ERLRYPAASVQRLLSSGAFYYSKQFDMTSNIQERGFVSSDYKLIADSSFFKSFMWNGFMTEELIETRKRMSP
AEQKIIDKSGLLIIVIRGYAKTVNTTVGGCEALMTLISKQSCAKEGPLFGDWGSDGDGYVSNYLESEIIIYT
EKFCLSYVIVRGNVPMYWELENNFSTKTILAANGKQIAFPRSFEASQEALVRHFDRLSSQYGDIHVLNTLSD
KSYKGVLNSAYEEQLKYFLQNRESTDIGYKVLYTRIPIASSRIKKIGYSGQNPYDIVSLLSNSIIDFGALFY
DSKPNSFIGKQLGVFRINSFDSLNKANFLSKIISQEVIDLAFRDIGLELDRELYVKHAQLWEENDLWISKLT
LNFASTSDKLHTSHNSIKSSFVKSHITKKYFGGVVESKPNEIAMLKLLGRLQDQSPVTMFNPIHNYVNKELN
KRAKDFTSKLDLSVYASTFNVNGSVYEGDIDKWIYPEENDYDLIFIGLQEIVVLNAGQMVNTDFRNKTQWER
KILGVLQKRNKYMVMWSGQLGGVALYFFVKESQVKYVSNVECSFKKTGLGGVSANKGGIAVSFKFSDTTICF
VSAHLAAGLSNIEERHQNYKALIKGIQFSKNRRIQNHDAVIWLGDFNYRIDLTNDQVKPMILQKLYAKIFEC
DQLNKQMANGESFPFFSEQEINFPPTYKFDKGTKVYDTSEKQRIPAWTDRILFLSRQNLIEPLSYNSCQNLT
FSDHRPVYATFKITVKIINHTIKKNLSDEIYKNYKDSHNGIFDILVKSFDNKELNEGKDASLPAPSSDKHKW
WLEGGKAAKIIIPGLEDDNMVMNPWRPINPFEKSNEPEFVSKNDLEAIQN

| Rank | Sequence | Start position | Score |
|---|---|---|---|
| 1 | LYLIEKPRTFVITTNT | 3 | 0.96 |
| 2 | EQEINFPPTYKFDKGT | 810 | 0.95 |
| 3 | EGDIDKWIYPEENDYD | 603 | 0.93 |
| 4 | PGLEDDNMVMNPWRPI | 949 | 0.92 |
| 5 | EWTASATVTDRIYKIT | 101 | 0.91 |
| 6 | HPSPTYKHSGIKGLVS | 25 | 0.90 |
| 6 | GFMTEELIETRKRMSP | 201 | 0.90 |
| 7 | PFEKSNEPEFVSKNDL | 966 | 0.89 |
| 7 | IPAWTDRILFLSRQNL | 836 | 0.89 |
| 7 | SEIIIYTEKFCLSYVI | 282 | 0.89 |
| 7 | HALIIRHPSPTYKHSG | 19 | 0.89 |
| 8 | SCAKEGPLFGDWGSDG | 257 | 0.88 |
| 9 | LVSGHSKDKDQNKDTK | 38 | 0.87 |
| 9 | HSGIKGLVSGHSKDKD | 32 | 0.87 |
| 9 | EFYCINNDEYDYLLDK | 119 | 0.87 |
| 9 | RIYKITDTEFYCINND | 111 | 0.87 |
| 10 | AQLWEENDLWISKLTL | 490 | 0.86 |
| 10 | PLFGDWGSDGDGYVSN | 263 | 0.86 |
| 10 | GLLIIVIRGYAKTVNT | 226 | 0.86 |
| 11 | MVMWSGQLGGVALYFF | 661 | 0.85 |
| 11 | QSPVTMFNPIHNYVNK | 558 | 0.85 |
| 11 | SGAFYYSKQFDMTSNI | 160 | 0.85 |
| 12 | YKLIADSSFFKSFMWN | 185 | 0.84 |
| 13 | APSSDKHKWWLEGGKA | 928 | 0.83 |
| 13 | TKVYDTSEKQRIPAWT | 825 | 0.83 |
| 13 | KELNKRAKDFTSKLDL | 573 | 0.83 |
| 13 | GGVVESKPNEIAMLKL | 536 | 0.83 |
| 13 | KSHITKKYFGGVVESK | 527 | 0.83 |
| 13 | EYENMSHQERERLRYP | 135 | 0.83 |
| 14 | HDAVIWLGDFNYRIDL | 757 | 0.82 |
| 14 | GYSGQNPYDIVSLLSN | 407 | 0.82 |
| 15 | DEIYKNYKDSHNGIFD | 892 | 0.81 |
| 15 | ANKGGIAVSFKFSDTT | 702 | 0.81 |
| 15 | NAGQMVNTDFRNKTQW | 631 | 0.81 |
| 15 | HVLNTLSDKSYKGVLN | 353 | 0.81 |
| 15 | GKQIAFPRSFEASQEA | 322 | 0.81 |
| 16 | MNPWRPINPFEKSNEP | 958 | 0.80 |
| 16 | FIGFITRDEWTASATV | 93 | 0.80 |
| 16 | NRRIQNHDAVIWLGDF | 751 | 0.80 |
| 16 | ECSFKKTGLGGVSANK | 689 | 0.80 |
| 16 | IGLELDRELYVKHAQL | 477 | 0.80 |
| 17 | LNEGKDASLPAPSSDK | 918 | 0.79 |
| 17 | TVKIINHTIKKNLSDE | 878 | 0.79 |
| 17 | YVIVRGNVPMYWELEN | 295 | 0.79 |
| 17 | TFVITTNTHALIIRHP | 11 | 0.79 |
| 18 | RQNLIEPLSYNSCQNL | 848 | 0.78 |
| 18 | GVFRINSFDSLNKANF | 445 | 0.78 |
| 19 | KGIQFSKNRRIQNHDA | 744 | 0.77 |
| 19 | AASVQRLLSSGAFYYS | 151 | 0.77 |
| 20 | HKWWLEGGKAAKIIIP | 934 | 0.76 |
| 20 | IFDILVKSFDNKELNE | 905 | 0.76 |
| 20 | SSRIKKIGYSGQNPYD | 400 | 0.76 |
| 20 | FEASQEALVRHFDRLS | 331 | 0.76 |
| 20 | HQERERLRYPAASVQR | 141 | 0.76 |
| 21 | TFSDHRPVYATFKITV | 864 | 0.75 |
| 21 | NDLWISKLTLNFASTS | 496 | 0.75 |
| 21 | VGGCEALMTLISKQSC | 243 | 0.75 |
| 21 | TRKRMSPAEQKIIDKS | 210 | 0.75 |

| Start | End | Max_score_pos | Sequence |
|---|---|---|---|
| 281 | 301 | 295 | ESEIIIYTEKFCLSYVIVRGN |
| 52 | 71 | 57 | TKVLVEFVLKEYLDLSLYRD |
| 668 | 693 | 674 | LGGVALYFFVKESQVKYVSNVECSFK |
| 716 | 729 | 722 | TTICFVSAHLAAGL |
| 618 | 633 | 627 | DLIFIGLQEIVVLNAG |
| 224 | 234 | 229 | KSGLLIIVIRG |
| 414 | 424 | 417 | YDIVSLLSNSI |
| 76 | 89 | 85 | HGGLLGLLGLLNVK |
| 585 | 595 | 589 | KLDLSVYASTF |
| 388 | 401 | 390 | GYKVLYTRIPIASS |
| 482 | 492 | 489 | DRELYVKHAQL |
| 650 | 656 | 652 | ILGVLQK |
| 148 | 167 | 155 | RYPAASVQRLLSSGAFYYSK |
| 335 | 357 | 355 | QEALVRHFDRLSSQYGDIHVLNT |
| 905 | 912 | 908 | IFDILVKS |
| 778 | 795 | 784 | KPMILQKLYAKIFECDQL |
| 240 | 262 | 255 | NTTVGGCEALMTLISKQSCAKEG |
| 852 | 863 | 854 | IEPLSYNSCQNL |
| 706 | 712 | 710 | GIAVSFK |
| 548 | 555 | 549 | MLKLLGRL |
| 460 | 476 | 467 | FLSKIISQEVIDLAFRD |
| 757 | 763 | 760 | HDAVIWL |
| 372 | 380 | 377 | EEQLKYFLQ |
| 19 | 33 | 22 | HALIIRHPSPTYKHS |
| 535 | 541 | 536 | FGGVVES |
| 868 | 884 | 874 | HRPVYATFKITVKIINH |
| 523 | 529 | 527 | SSFVKSH |
| 841 | 849 | 846 | DRILFLSRQ |
| 441 | 449 | 446 | GKQLGVFRI |
| 178 | 195 | 184 | RGFVSSDYKLLADSSFFK |
| 557 | 564 | 558 | DQSPVTMF |
| 944 | 950 | 946 | AKIIIPG |
| 35 | 42 | 41 | IKGLVSGH |
| 363 | 369 | 364 | YKGVLNS |
| 924 | 931 | 927 | ASLPAPSS |
| 129 | 134 | 131 | DYLLDK |
| 739 | 747 | 745 | YKALIKGIQ |
| 426 | 433 | 427 | DFGALFYD |
| 10 | 16 | 11 | RTFVITT |
| 500 | 508 | 502 | ISKLTLNFA |
| 325 | 330 | 328 | IAFPRS |
| 315 | 320 | 317 | KTILAA |
| 105 | 115 | 111 | SATVTDRIYKI |
| 825 | 830 | 828 | TKVYDT |

59. SET2
MSNNNFQESSNNTSSPSKRSTPMLFLDAENKTQEALTTFELLNACTYQNKYVGSANVTTTATTSTKTSNSTS
TKSHQQQHRRKLEYMTCDCEEEWDSELQMNLACGPDSNCINRITCVECVNRNCLCGDDCQNQRFQNRQYSKV
KVIQTELKGYGLIAEQDIEENQFIYEYIGEVIDEISFRQRMIEYDLRHLKHFYFMMLSNDSFIDATEKGSLG
RFINHSCNPNAFVDKWHVGDRLRMGIFAKRKISRGEEITFDYNVDRYGAQSQPCYCGEPNCIKFMGGKTQTD
AALLLPQMIAEALGVTPRQEKAWLKENKSIRNQQQNDESNINEEFVNSIEIEPIENQDGVTKVMSALMKTQH
PLIIKKLIERIFLSNDQDDINVMFVRFHGYKTISTILQDLLVAKNSGKESETTDNNDIDNSTGDDDQDKDEL
IIKILKILVSWPAVTKNKIASANLEEVVKDIQTNNENSNNNDEINQLCTSLLDRWSKLEMAYRIPKQESVPT
NNAAAAATTTATATGTTTSASPFERISSHTPEVGGTNTPSSTSQQQQQQNSRDAGLPENWRSAFDKNTGGYY
YYNLVTKETTWERPLGSLPLGPKPPSGPGLKGRINKYNEIDLAKREELRIQKEKEMKFIEMQNRDRKLKELI
EMSKKSMNNIGGSSGTTITAATINGLSDNGGNNNGNITGIYGDDKHSKHHHHHHDKHLKNGPRNTSTSSSSG
NNVEKIWKRIFAKYIPNIIKKYESEIGRDNVKGCAKELVNILTQSEIKHGNSLPSSSSSNGYSMELSDKKLK
KIKEYSHGYMDKFLIKFNNSKKHKSTMGSKGSDNHKRKHNGDGDNGVKRSKV*

| Rank | Sequence | Start position | Score |
|---|---|---|---|
| 1 | ACTYQNKYVGSANVTT | 44 | 0.94 |
| 2 | VKDIQTNNENSNNNDE | 460 | 0.93 |
| 2 | QRMIEYDLRHLKHFYF | 183 | 0.93 |
| 3 | HHHHDKHLKNGPRNTS | 699 | 0.92 |
| 3 | FERISSHTPEVGGTNT | 527 | 0.92 |
| 3 | KISRGEEITFDYNVDR | 247 | 0.92 |
| 4 | EEEWDSELQMNLACGP | 92 | 0.91 |
| 4 | KIKEYSHGYMDKFLIK | 793 | 0.91 |
| 4 | QESSNNTSSPSKRSTP | 7 | 0.91 |
| 4 | TGGYYYYNLVTKETTW | 572 | 0.91 |
| 4 | KFMGGKTQTDAALLLP | 279 | 0.91 |
| 4 | TEKGSLGRFINHSCNP | 210 | 0.91 |
| 5 | TINGLSDNGGNNNGNI | 670 | 0.90 |
| 5 | TPEVGGTNTPSSTSQQ | 534 | 0.90 |
| 5 | SGKESETTDNNDIDNS | 406 | 0.90 |
| 5 | GRFINHSCNPNAFVDK | 216 | 0.90 |
| 5 | DSFIDATEKGSLGRFI | 204 | 0.90 |
| 6 | YMTCDCEEEWDSELQM | 86 | 0.89 |
| 6 | SSNGYSMELSDKKLKK | 778 | 0.89 |
| 6 | MKFIEMQNRDRKLKEL | 632 | 0.89 |
| 6 | ANVTTTATTSTKTSNS | 55 | 0.89 |
| 6 | SQQQQQQNSRDAGLPE | 547 | 0.89 |
| 7 | LVTKETTWERPLGSLP | 580 | 0.87 |
| 7 | YGAQSQPCYCGEPNCI | 263 | 0.87 |
| 7 | KWHVGDRLRMGIFAKR | 231 | 0.87 |
| 8 | KYESEIGRDNVKGCAK | 741 | 0.86 |
| 8 | KHSKHHHHHHDKHLKN | 693 | 0.86 |
| 8 | SKLEMAYRIPKQESVP | 488 | 0.86 |
| 8 | IGEVIDEISFRQRMIE | 172 | 0.86 |
| 9 | IPNIIKKYESEIGRDN | 735 | 0.85 |
| 9 | KGRINKYNEIDLAKRE | 607 | 0.85 |
| 9 | NSIEIEPIENQDGVTK | 335 | 0.85 |
| 9 | EALGVTPRQEKAWLKE | 299 | 0.85 |
| 10 | NHKRKHNGDGDNGVKR | 826 | 0.84 |
| 10 | NGPRNTSTSSSSGNNV | 708 | 0.84 |
| 10 | TSTKTSNSTSTKSHQQ | 63 | 0.84 |
| 10 | PLIIKKLIERIFLSND | 361 | 0.84 |
| 11 | MGSKGSDNHKRKHNGD | 819 | 0.83 |
| 11 | AWLKENKSIRNQQQND | 310 | 0.83 |
| 11 | MNLACGPDSNCINRIT | 101 | 0.83 |
| 12 | NSTGDDDQDKDELIIK | 420 | 0.82 |
| 13 | TSTKSHQQQHRRKLEY | 71 | 0.81 |
| 13 | SGTTITAATINGLSDN | 662 | 0.81 |
| 13 | KPPSGPGLKGRINKYN | 599 | 0.81 |
| 13 | RPLGSLPLGPKPPSGP | 589 | 0.81 |
| 13 | TATGTTTSASPFERIS | 516 | 0.81 |
| 13 | IPKQESVPTNNAAAAA | 496 | 0.81 |
| 13 | NCINRITCVECVNRNC | 110 | 0.81 |
| 14 | NNSKKHKSTMGSKGSD | 810 | 0.80 |
| 15 | NGNITGIYGDDKHSKH | 682 | 0.79 |
| 15 | ECVNRNCLCGDDCQNQ | 119 | 0.79 |
| 16 | PCYCGEPNCIKFMGGK | 269 | 0.78 |
| 17 | QNSRDAGLPENWRSAF | 553 | 0.77 |
| 17 | MSALMKTQHPLIIKKL | 352 | 0.77 |
| 18 | KELIEMSKKSMNNIGG | 645 | 0.76 |
| 18 | YKTISTILQDLLVAKN | 390 | 0.76 |
| 18 | SKRSTPMLFLDAENKT | 17 | 0.76 |
| 18 | IQTELKGYGLIAEQDI | 147 | 0.76 |
| 19 | NILTQSEIKHGNSLPS | 760 | 0.75 |
| 19 | NAAAAATTTATATGTT | 506 | 0.75 |
| 19 | NKTQEALTTFELLNAC | 30 | 0.75 |

| Start | End | Max_score_pos | Sequence |
|---|---|---|---|
| 112 | 130 | 118 | INRITCVECVNRNCLCGDD |
| 393 | 405 | 399 | ISTILQDLLVAKN |
| 431 | 449 | 439 | ELIIKILKILVSWPAVTKN |
| 477 | 487 | 481 | NQLCTSLLDRW |
| 265 | 280 | 269 | AQSQPCYCGEPNCIKF |
| 575 | 583 | 578 | YYYYNLVTK |
| 288 | 306 | 292 | DAALLLPQMIAEALGVTPR |
| 141 | 151 | 144 | YSKVKVIQTEL |
| 38 | 55 | 44 | TFELLNACTYQNKYVGSA |
| 356 | 374 | 365 | MKTQHPLIIKKLIERIFLS |
| 591 | 599 | 593 | LGSLPLGPK |
| 751 | 766 | 756 | VKGCAKELVNILTQSE |
| 382 | 388 | 385 | VMFVRFH |
| 187 | 199 | 195 | EYDLRHLKHFYFM |
| 224 | 232 | 231 | NPNAFVDKW |
| 456 | 463 | 462 | LEEVVKDI |
| 731 | 739 | 733 | FAKYIPNII |
| 100 | 110 | 102 | QMNLACGPDSN |
| 168 | 179 | 169 | IYEYIGEVIDEI |
| 695 | 705 | 699 | SKHHHHHHDKH |
| 348 | 354 | 351 | VTKVMSA |
| 153 | 159 | 154 | GYGLIAE |
| 86 | 92 | 88 | YMTCDCE |
| 22 | 27 | 25 | PMLFLD |
| 770 | 776 | 775 | GNSLPSS |
| 257 | 263 | 257 | DYNVDRY |
| 803 | 809 | 806 | DKFLIKF |
| 791 | 800 | 794 | LKKIKEYSHG |
| 524 | 530 | 527 | ASPFERI |

60. DOT1
MISGHLQTPDSSDHSGDEAKLTKPSGLESKTNELWSSDLEEELESRIMQPATFSSILKQFPSISEETIISKI
MSNKNCDKNNWKKKIYCYRYFLQPSKEEPDVGNMKSLVEKLEKLKLKKWSASEIEQLFCNYLEDLTTSAVKV
SIPGKTLDEVAAAVNNIHPRPRWTRKEIECLIKNENDFKKLEKDLFVRDLDSIKKKIRRDNLSIQNEIQDSE
KKSPQSTDSNNKDSSRDLSRKERDQLKKLLSKPICFSELLAKFPGHSWEYIAREIIQLDSSEDNTIWLKKIY
YYCVVYSISVDKEITKYIGGGNRIYEKVRSDWSKIKTLDFFKDWSLQEFERLYCFAFHDLTKSALTKNFPSK
NLNDICKVVNISFPKVPYTDREMKYLERHLDTPMQTLENNLPFRSRGSIHKKLEALKALTETTEQKQPPTKT
RPKNEEEKNVENAAYMKELIMFDLTLEAIENTFPSEPIEEIIKEIKNSEIFDPLSFTRGEKELMAKLVKKGN
LIDDCFDYFPLREEEFIRSKYAEAEYVSGRKMKFNTPEERLAYEAKWTLFNMGKQEYGRGNRRSTKRYCE ID
ELSKLEQEASVKRSKKKIELTEEELEQRRKRSEHFRLCRLKKLEEKREKYRIEKAKRLEKIAAGLIKPSTSG
YELKDIVTSAEYFQSIVGDKQKVQEGQKRKRIQTEYFAPEFIEKPKAVKLKTTKRQAEKNKIKKQLKREAQL
KIKKKKTIAPKKGKRRVKTNNGIIEEIKDVYKLSSEPYVESEVEEEDEEEDYISPYDPPDIISDSQVKLNGR
HLYISSFYKELPEIPELKFVSLPHMEMSGNDITVAKQIMTTANDDILYDDCLAYEIVAQHIKSYRDLFISVP
PVLDPITHELNSANIVRIRFFLYPEHYESFMLASPKSNELDPVHEIAKLFMIQYSLYFSHSDTLKKIITEDY
CHKLEHSVEENDFGEFMFVVDKWNQLVMKLSPNLASVQNILGLKEDINEAPRAYLNQQEVSIPTNSDLKIET
FYDEIMYESASPLFNPINSNLEIDSESAPIPLGEVEIPNNVIEEINEKMPDNYIPDFFRRLKEKTEVSRYAM
QQILLRVYSRVVSTDSRKLRSYKAFTAETYGELLPSFTSEVLEKLNLLPTQKFYDLGSGVGNTTFQAALEFG
ACSSGGCEIMEHASKLTELQAGLIQKHLAVLGLQKLNLDFALHESFVGNEKVRASCLDCDVLIINNYLFDGQ
LNDEVGKLLYGLRPGTKIVSLRNFISPRYRATFDTVFDFLSVEKHEMSDIMSVSWTANKVPYYISTVEETIP
REYLSREETKETSGKSKSVSPVGEIENVAAAMMTPPTDSSESEIIKN*

| Rank | Sequence | Start position | Score |
|---|---|---|---|
| 1 | EETIISKIMSNKNCDK | 65 | 0.95 |
| 1 | SGHLQTPDSSDHSGDE | 3 | 0.95 |
| 2 | KELIMFDLTLEAIENT | 449 | 0.94 |
| 2 | DVLIINNYLFDGQLND | 1212 | 0.94 |
| 3 | SVEENDFGEFMFVVDK | 943 | 0.93 |
| 4 | LFMIQYSLYFSHSDTL | 913 | 0.92 |
| 4 | KSYRDLPISFPPVLDP | 854 | 0.92 |
| 5 | AAGLIKPSTSGYELKD | 638 | 0.91 |
| 5 | TTEQKQPPTKTRPKNE | 422 | 0.91 |
| 5 | DKEITKYIGGGNRIYE | 299 | 0.91 |
| 6 | TEDYCHKLEHSVEEND | 933 | 0.89 |
| 6 | DEEEDYISPYDPPDII | 767 | 0.89 |
| 7 | KQEYGRGNRRSTKRYC | 558 | 0.88 |
| 7 | ESRIMQPATFSSILKQ | 44 | 0.88 |
| 7 | RGSIHKKLEALKALTE | 406 | 0.88 |
| 8 | YFLQPSKEEPDVGNMK | 92 | 0.87 |
| 8 | NSEIFDPLSFTRGEKE | 479 | 0.87 |
| 8 | IMYESASPLFNPINSN | 1013 | 0.87 |
| 9 | GLKEDINEAPRAYLNQ | 978 | 0.86 |
| 9 | DILYDDCLAYEIVAQH | 837 | 0.86 |
| 9 | SGRKMKFNTPEERLAY | 532 | 0.86 |
| 9 | YTDREMKYLERHLDTP | 378 | 0.86 |
| 9 | TKSALTKNFPSKNLND | 349 | 0.86 |
| 9 | PGHSWEYIAREIIQLD | 260 | 0.86 |
| 10 | FFLYPEHYESFMLASP | 884 | 0.85 |
| 10 | APEFIEKPKAVKLKTT | 686 | 0.85 |
| 10 | AEAEYVSGRKMKFNTP | 526 | 0.85 |
| 10 | GNRIYEKVRSDWSKIK | 309 | 0.85 |
| 10 | TVEETIPREYLSREET | 1290 | 0.85 |
| 11 | YESFMLASPKSNELDP | 891 | 0.84 |
| 11 | AGLIQKHLAVLGLQKL | 1173 | 0.84 |
| 11 | FNPINSNLEIDSESAP | 1022 | 0.84 |
| 12 | KKKTIAPKKGKRRVKT | 724 | 0.83 |
| 12 | KRIQTEYFAPEFIEKP | 678 | 0.83 |
| 12 | FNTPEERLAYEAKWTL | 538 | 0.83 |
| 12 | IEEIIKEIKNSEIFDP | 470 | 0.83 |
| 12 | MQTLENNLPFRSRGSI | 394 | 0.83 |
| 12 | REIIQLDSSEDNTIWL | 269 | 0.83 |
| 12 | QNEIQDSEKKSPQSTD | 209 | 0.83 |
| 12 | SVSWTANKVPYYISTV | 1276 | 0.83 |
| 12 | EMSDIMSVSWTANKVP | 1270 | 0.83 |
| 12 | MQQILLRVYSRVVSTD | 1080 | 0.83 |
| 13 | AKQIMTTANDDILYDD | 827 | 0.82 |
| 13 | SEVEEEDEEEDYISPY | 761 | 0.82 |
| 13 | KDIVTSAEYFQSIVGD | 652 | 0.82 |
| 13 | KKLEEKREKYRIEKAK | 617 | 0.82 |
| 13 | SFTRGEKELMAKLVKK | 487 | 0.82 |
| 13 | KKIYYYCVVYSISVDK | 285 | 0.82 |
| 13 | KKSPQSTDSNNKDSSR | 217 | 0.82 |
| 13 | ECLIKNENDFKKLEKD | 173 | 0.82 |
| 13 | AFTAETYGELLPSFTS | 1104 | 0.82 |
| 13 | VIEEINEKMPDNYIPD | 1049 | 0.82 |
| 14 | YCEIDELSKLEQEASV | 572 | 0.81 |
| 14 | AAVNNIHPRPRWTRKE | 156 | 0.81 |
| 14 | SLRNFISPRYRATFDT | 1244 | 0.81 |
| 14 | LGSGVGNTTFQAALEF | 1136 | 0.81 |
| 15 | EMSGNDITVAKQIMTT | 818 | 0.80 |
| 15 | HSGDEAKLTKPSGLES | 14 | 0.80 |
| 15 | SPVGEIENVAAAMMTP | 1316 | 0.80 |
| 15 | RYRATFDTVFDFLSVE | 1252 | 0.80 |
| 15 | GCEIMEHASKLTELQA | 1158 | 0.80 |
| 15 | GACSSGGCEIMEHASK | 1152 | 0.80 |
| 16 | NQLVMKLSPNLASVQN | 960 | 0.79 |
| 16 | NNWKKKIYCYRYFLQP | 81 | 0.79 |
| 16 | PDIISDSQVKLNGRHL | 779 | 0.79 |
| 16 | KLEQEASVKRSKKKIE | 580 | 0.79 |
| 16 | GNLIDDCFDYFPLREE | 503 | 0.79 |
| 16 | DWSKIKTLDFFKDWSL | 319 | 0.79 |
| 16 | LGEVEIPNNVIEEINE | 1040 | 0.79 |
| 17 | MFVVDKWNQLVMKLSP | 953 | 0.78 |
| 17 | FPPVLDPITHELNSAN | 863 | 0.78 |
| 17 | KQKVQEGQKRKRIQTE | 668 | 0.78 |
| 17 | IELTEEELEQRRKRSE | 594 | 0.78 |
| 17 | VAAAMMTPPTDSSESE | 1324 | 0.78 |
| 17 | EETKETSGKSKSVSPV | 1303 | 0.78 |
| 18 | NGIIEEIKDVYKLSSE | 741 | 0.77 |
| 18 | KAVKLKTTKRQAEKNK | 694 | 0.77 |
| 18 | NFPSKNLNDICKVVNI | 356 | 0.77 |
| 18 | RVVSTDSRKLRSYKAF | 1090 | 0.77 |
| 19 | KSNELDPVHEIAKLFM | 900 | 0.76 |
| 19 | KNKIKKQLKREAQLKI | 707 | 0.76 |
| 19 | KYLERHLDTPMQTLEN | 384 | 0.76 |
| 19 | YCFAFHDLTKSALTKN | 341 | 0.76 |
| 19 | SIPGKTLDEVAAAVNN | 145 | 0.76 |
| 20 | NGRHLYISSFYKELPE | 790 | 0.75 |
| 20 | KNEEEKNVENAAYMKE | 435 | 0.75 |
| 20 | NNKDSSRDLSRKERDQ | 226 | 0.75 |
| 20 | NTTFQAALEFGACSSG | 1142 | 0.75 |

| Start | End | Max_score_pos | Sequence |
|---|---|---|---|
| 284 | 301 | 291 | LKKIYYYCVVYSISVDKE |
| 1205 | 1219 | 1211 | RASCLDCDVLIINNY |
| 839 | 873 | 846 | LYDDCLAYEIVAQHIKSYRDLPISFPPVLDPITHE |
| 1080 | 1094 | 1090 | MQQILLRVYSRVVST |
| 363 | 379 | 368 | NDICKVVNISFPKVPYT |
| 1165 | 1198 | 1182 | ASKLTELQAGLIQKHLAVLGLQKLNLDFALHESF |
| 337 | 352 | 343 | FERLYCFAFHDLTKSA |
| 86 | 96 | 91 | KIYCYRYFLQP |
| 1257 | 1267 | 1263 | FDTVFDFLSVE |
| 242 | 260 | 248 | LKKLLSKPICFSELLAKFP |
| 139 | 149 | 144 | TSAVKVSIPGK |
| 773 | 815 | 812 | ISPYDPPDIISDSQVKLNGRHLYISSFYKELPEIPEL |
| | | | KFVSLP |
| 126 | 137 | 131 | IEQLFCNYLEDL |
| 506 | 515 | 512 | IDDCFDYFPL |
| 610 | 617 | 616 | HFRLCRLK |
| 1282 | 1294 | 1286 | NKVPYYISTVEET |
| 904 | 931 | 919 | LDPVHEIAKLFMIQYSLYFSHSDTLKKI |
| 933 | 943 | 939 | TEDYCHKLEHS |
| 1312 | 1319 | 1315 | SKSVSPVG |
| 747 | 762 | 756 | IKDVYKLSSEPYVESE |
| 1230 | 1237 | 1232 | GKLLYGLR |
| 952 | 980 | 965 | FMFVVDKWNQLVMKLSPNLASVQNILGLK |
| 877 | 891 | 883 | ANIVRIRFFLYPEHY |
| 1111 | 1139 | 1124 | GELLPSFTSEVLEKLNLLPTQKFYDLGSG |
| 651 | 669 | 663 | LKDIVTSAEYFQSIVGDKQ |
| 151 | 161 | 155 | LDEVAAAVNNI |
| 1146 | 1159 | 1156 | QAALEFGACSSGGC |
| 410 | 419 | 416 | HKKLEALKAL |
| 1034 | 1043 | 1040 | ESAPIPLGEV |
| 496 | 503 | 498 | MAKLVKKG |
| 186 | 194 | 188 | EKDLFVRDL |
| 106 | 113 | 110 | MKSLVEKL |
| 824 | 829 | 827 | ITVAKQ |
| 172 | 177 | 173 | IECLIK |
| 692 | 699 | 695 | KPKAVKLK |
| 570 | 576 | 575 | KRYCEID |
| 1239 | 1248 | 1245 | GTKIVSLRNF |
| 527 | 533 | 528 | EAEYVSG |
| 483 | 488 | 485 | FDPLSF |
| 52 | 63 | 59 | TFSSILKQFPSI |
| 988 | 999 | 996 | RAYLNQQEVSIP |
| 634 | 644 | 639 | LEKIAAGLIKP |
| 268 | 275 | 274 | AREIIQLD |
| 584 | 589 | 584 | EASVKR |
| 1018 | 1024 | 1018 | ASPLFNP |
| 115 | 121 | 115 | KLKLKKW |
| 1099 | 1105 | 1102 | LRSYKAF |
| 453 | 459 | 454 | MFDLTLE |
| 893 | 899 | 896 | SFMLASP |
| 4 | 9 | 5 | GHLQTP |
| 323 | 328 | 326 | IKTLDF |
| 1274 | 1279 | 1275 | IMSVSW |

61. ENO1
MSYATKIHARYVYDSRGNPTVEVDFTTDKGLFRSIVPSGASTGVHEALELRDGDKSKWLGKGVLKAVANVND
IIAPALIKAKIDVVDQAKIDEFLLSLDGTPNKSKLGANAILGVSLAAANAAAAAQGIPLYKHIANISNAKKG
KFVLPVPFQNVLNGGSHAGGALAFQEFMIAPTGVSTFSEALRIGSEVYHNLKSLTKKKYGQSAGNVGDEGGV
APDIKTPKEALDLIMDAIDKAGYKGKVGIAMDVASSEFYKDGKYDLDFKNPESDPSKWLSGPQLADLYEQLI
SEYPIVSIEDPFAEDDWDAWVHFFERVGDKIQIVGDDLTVTNPTRIKTAIEKKAANALLLKVNQIGTLTESI
QAANDSYAAGWGVMVSHRSGETEDTFIADLSVGLRSGQIKTGAPARSERLAKLNQILRIEEELGSEAIYAGK
DFQKASQL

| Rank | Sequence | Start position | Score |
|---|---|---|---|
| 1 | ATKIHARYVYDSRGNP | 4 | 0.97 |
| 2 | TESIQAANDSYAAGWG | 357 | 0.94 |
| 3 | HRSGETEDTFIADLSV | 377 | 0.93 |
| 4 | AAAAQGIPLYKHIANI | 123 | 0.90 |
| 5 | IVSIEDPFAEDDWDAW | 293 | 0.89 |
| 6 | SEAIYAGKDFQKASQL | 425 | 0.88 |
| 6 | EDDWDAWVHFFERVGD | 302 | 0.88 |
| 7 | GGVAPDIKTPKEALDL | 214 | 0.87 |
| 8 | LSLDGTPNKSKLGANA | 96 | 0.85 |
| 8 | KWLGKGVLKAVANVND | 57 | 0.85 |
| 8 | VDFTTDKGLFRSIVPS | 23 | 0.85 |
| 9 | AAGWGVMVSHRSGETE | 368 | 0.84 |
| 9 | GNVGDEGGVAPDIKTP | 208 | 0.84 |
| 10 | PALIKAKIDVVDQAKI | 76 | 0.81 |
| 10 | YKDGKYDLDFKNPESD | 255 | 0.81 |
| 11 | VHEALELRDGDKSKWL | 44 | 0.79 |
| 11 | GQIKTGAPARSERLAK | 397 | 0.79 |
| 11 | LDLIMDAIDKAGYKGK | 227 | 0.79 |
| 11 | KYGQSAGNVGDEGGVA | 202 | 0.79 |
| 11 | RYVYDSRGNPTVEVDF | 10 | 0.79 |
| 12 | VNQIGTLTESIQAAND | 350 | 0.78 |
| 13 | PTRIKTAIEKKAANAL | 331 | 0.76 |
| 13 | KGKFVLPVPFQNVLNG | 143 | 0.76 |
| 14 | VNDIIAPALIKAKIDV | 70 | 0.75 |
| 14 | GKVGIAMDVASSEFYK | 241 | 0.75 |
| 14 | ALRIGSEVYHNLKSLT | 184 | 0.75 |

| Start | End | Max_score_pos | Sequence |
|---|---|---|---|
| 144 | 155 | 149 | GKFVLPVPFQNV |
| 109 | 137 | 114 | ANAILGVSLAAANAAAAAQGIPLYKHIAN |
| 60 | 89 | 65 | GKGVLKAVANVNDIIAPALIKAKIDVVDQA |
| 343 | 353 | 347 | ANALLLKVNQI |
| 4 | 14 | 10 | ATKIHARYVYD |
| 32 | 39 | 34 | FRSIVPSG |
| 387 | 397 | 389 | IADLSVGLRSG |
| 307 | 314 | 312 | AWVHFFER |
| 272 | 298 | 291 | SKWLSGPQLADLYEQLISEYPIVSIED |
| 41 | 50 | 47 | STGVHEALEL |
| 175 | 198 | 194 | PTGVSTFSEALRIGSEVYHNLKSL |
| 372 | 378 | 376 | GVMVSHR |
| 92 | 99 | 97 | DEFLLSLD |
| 240 | 253 | 252 | KGKVGIAMDVASSE |
| 317 | 326 | 325 | DKIQIVGDDL |
| 412 | 418 | 416 | KLNQILR |
| 163 | 172 | 168 | GGALAFQEFM |
| 425 | 432 | 426 | SEAIYAGK |
| 226 | 232 | 227 | ALDLIMD |

62. BGL2
MQIKFLTTLATVLTSVAAMGDLAFNLGVKNDDGTCKDVSTFEGDLDFLKSHSKIIKTYAVSDCNTLQNLGPA
AEAEGFQIQLGIWPNDDAHFEAEKEALQNYLPKISVSTIKIFLVGSEALYREDLTASELASKINDIKDLVKG
IKDKNGKSYSSVPVGTVDSWNVLVDGASKPAIDAADVVYSNSFSYWQKNSQANASYSLFDDVMQALQTLQTA
KGSTDIEFWVGETGWPTDGSSYGDSVPSVENAADQWQKGICALRAWGINVAVYEAFDEAWKPDTSGTSSVEK
HWGVWQSDKTLKYSIDCKF

| Rank | Sequence | Start position | Score |
|---|---|---|---|
| 1 | SVAAMGDLAFNLGVKN | 15 | 0.93 |
| 2 | DGTCKDVSTFEGDLDF | 32 | 0.91 |
| 3 | SKIIKTYAVSDCNTLQ | 52 | 0.89 |
| 4 | AWKPDTSGTSSVEKHW | 275 | 0.88 |
| 5 | PSVENAADQWQKGICA | 243 | 0.87 |
| 5 | VGETGWPTDGSSYGDS | 226 | 0.87 |
| 6 | GFQIQLGIWPNDDAHF | 77 | 0.86 |
| 7 | YEAFDEAWKPDTSGTS | 269 | 0.82 |
| 7 | TAKGSTDIEFWVGETG | 215 | 0.82 |
| 8 | KPAIDAADVVYSNSFS | 173 | 0.81 |
| 9 | VGTVDSWNVLVDGASK | 158 | 0.80 |
| 9 | VKGIKDKNGKSYSSVP | 142 | 0.80 |
| 10 | QKGICALRAWGINVAV | 253 | 0.79 |
| 10 | ASKINDIKDLVKGIKD | 132 | 0.79 |
| 11 | ASYSLFDDVMQALQTL | 198 | 0.78 |
| 11 | LVGSEALYREDLTASE | 115 | 0.78 |
| 12 | SGTSSVEKHWGVWQSD | 281 | 0.77 |
| 13 | GKSYSSVPVGTVDSWN | 150 | 0.76 |
| 14 | WGVWQSDKTLKYSIDC | 290 | 0.75 |
| 14 | LGVKNDDGTCKDVSTF | 26 | 0.75 |
| 14 | NVLVDGASKPAIDAAD | 165 | 0.75 |
| 14 | REDLTASELASKINDI | 123 | 0.75 |

| Start | End | Max_score_pos | Sequence |
|---|---|---|---|
| 4 | 21 | 15 | KFLTTLATVLTSVAAMGD |
| 153 | 161 | 155 | YSSVPVGTV |
| 263 | 271 | 269 | GINVAVYEA |
| 99 | 121 | 105 | LQNYLPKISVSTIKIFLVGSEAL |
| 173 | 186 | 181 | KPAIDAADVVYSNS |
| 163 | 171 | 169 | SWNVLVDGA |
| 45 | 66 | 60 | LDFLKSHSKIIKTYAVSDCNTL |
| 298 | 304 | 302 | TLKYSID |
| 254 | 261 | 259 | KGICALRA |
| 197 | 214 | 203 | NASYSLFDDVMQALQTLQ |
| 239 | 248 | 242 | GDSVPSVENA |
| 78 | 84 | 81 | FQIQLGI |
| 128 | 134 | 129 | ASELASK |
| 139 | 145 | 141 | KDLVKGI |

63. FBA1
MAPPAVLSKSGVIYGKDVKDLFDYAQEKGFAIPAINVTSSSTVVAALEAARDNKAPIILQTSQGGAAYFAGK
GVDNKDQAASIAGSIAAAHYIRAIAPTYGIPVVLHTDHCAKKLLPWFDGMLKADEEFFAKTGTPLFSSHMLD
LSEETDDENIATCAKYFERMAKMGQWLEMEIGITGGEEDGVNNEHVEKDALYTSPETVFAVYESLHKISPNF
SIAAAFGNVHGVYKPGNVQLRPEILGDHQVYAKKQIGTDAKHPLYLVFHGGSGSTQEEFNTAIKNGVVKVNL
DTDCQYAYLTGIRDYVTNKIEYLKAPVGNPEGADKPNKKYFDPRVWVREGEKTMSKRIAEALDIFHTKGQL*

| Rank | Sequence | Start position | Score |
|---|---|---|---|
| 1 | TSQGGAAYFAGKGVDN | 61 | 0.92 |
| 2 | PETVFAVYESLHKISP | 199 | 0.90 |
| 2 | DGVNNEHVEKDALYTS | 183 | 0.90 |
| 3 | AGSIAAAHYIRAIAPT | 84 | 0.89 |
| 3 | EMEIGITGGEEDGVNN | 172 | 0.89 |
| 3 | DGMLKADEEFFAKTGT | 120 | 0.89 |
| 4 | GGSGSTQEEFNTAIKN | 266 | 0.87 |
| 5 | TDCQYAYLTGIRDYVT | 290 | 0.84 |
| 5 | HCAKKLLPWFDGMLKA | 110 | 0.84 |
| 6 | VGNPEGADKPNKKYFD | 315 | 0.83 |
| 6 | DLSEETDDENIATCAK | 144 | 0.83 |
| 7 | KKQIGTDAKHPLYLVF | 249 | 0.82 |
| 8 | AVLSKSGVIYGKDVKD | 5 | 0.81 |
| 9 | YIRAIAPTYGIPVVLH | 92 | 0.80 |
| 9 | NKKYFDPRVWVREGEK | 325 | 0.80 |
| 10 | AAFGNVHGVYKPGNVQ | 220 | 0.79 |
| 10 | ATCAKYFERMAKMGQW | 155 | 0.79 |
| 11 | EGEKTMSKRIAEALDI | 337 | 0.78 |
| 11 | RPEILGDHQVYAKKQI | 237 | 0.78 |
| 12 | NTAIKNGVVKVNLDTD | 276 | 0.77 |
| 12 | HVEKDALYTSPETVFA | 189 | 0.77 |

| Start | End | Max_score_pos | Sequence |
|---|---|---|---|
| 257 | 266 | 262 | KHPLYLVFHG |
| 80 | 118 | 103 | AASIAGSIAAAHYIRAIAPTYGIPVVLHTDHCAKKLLPW |
| 30 | 49 | 44 | FAIPAINVTSSSTVVAALEA |
| 280 | 287 | 285 | KNGVVKVN |
| 289 | 303 | 295 | DTDCQYAYLTGIRDY |
| 191 | 239 | 205 | EKDALYTSPETVFAVYESLHKISPNFSIAAAFGNVHGVYKPG |
| | | | NVQLRPE |
| 4 | 24 | 5 | PAVLSKSGVIYGKDVKDLFDY |
| 305 | 316 | 313 | TNKIEYLKAPVG |
| 154 | 160 | 157 | IATCAKY |
| 241 | 250 | 244 | LGDHQVYAKK |
| 55 | 61 | 57 | APIILQT |
| 330 | 336 | 332 | DPRVWVR |
| 134 | 144 | 140 | GTPLFSSHMLD |
| 347 | 353 | 352 | AEALDIF |
| 65 | 72 | 71 | GAAYFAGK |

64. IPF9162
MKKRLVLFDDSDDNSETESDKSKLKSRKKQFKIPEYPQPPSFPVNEQNEDYMKYQLQDDKHEEPTESAIDKK
DCSLFSNPSTSIGLSIMERMGFKIGNALGNSATAIKEPIEVSLKSGRQGLGGGFKPLQYKQEDVENLKLNLA
NSNKQRIELRDLKKIMKLCFELSGEYDKYLEGEDITEVNSLWQPYVKIYVSKQQSAAVGSVKAKFNAVETLQ
LFEREVENTEQTLSDLLNYLRGTHNYCWYCGLKYNDQNNLLANCPGKTRDIHLTI*

| Rank | Sequence | Start position | Score |
|---|---|---|---|
| 1 | PEYPQPPSFPVNEQNE | 34 | 0.92 |
| 1 | TESDKSKLKSRKKQFK | 17 | 0.92 |
| 2 | YLEGEDITEVNSLWQP | 173 | 0.89 |
| 3 | TESAIDKKDCSLFSNP | 65 | 0.87 |
| 3 | RGTHNYCWYCGLKYND | 237 | 0.87 |
| 3 | SGRQGLGGGFKPLQYK | 117 | 0.87 |
| 4 | GLSIMERMGFKIGNAL | 85 | 0.84 |
| 5 | TEVNSLWQPYVKIYVS | 180 | 0.83 |
| 6 | EVENTEQTLSDLLNYL | 221 | 0.80 |
| 6 | SGEYDKYLEGEDITEV | 167 | 0.80 |
| 7 | GFKIGNALGNSATAIK | 93 | 0.78 |
| 7 | ATAIKEPIEVSLKSGR | 104 | 0.78 |
| 8 | KRLVLFDDSDDNSETE | 3 | 0.76 |
| 8 | LLANCPGKTRDIHLTI | 256 | 0.76 |

| Start | End | Max_score_pos | Sequence |
|---|---|---|---|
| 182 | 220 | 191 | VNSLWQPYVKIYVSKQQSAAVGSVKAKFNAVETLQLFER |
| 240 | 249 | 245 | HNYCWYCGLK |
| 159 | 167 | 164 | IMKLCFELS |
| 4 | 9 | 8 | RLVLFD |
| 31 | 44 | 41 | FKIPEYPQPPSFPV |
| 107 | 116 | 113 | IKEPIEVSLK |
| 71 | 79 | 77 | KKDCSLFSN |
| 229 | 236 | 232 | LSDLLNYL |
| 127 | 134 | 129 | KPLQYKQE |
| 53 | 58 | 54 | KYQLQD |
| 138 | 143 | 138 | NLKLNL |

65. PGK1
MSLSNKLSVKDLDVAGKRVFIRVDFNVPLDGKTITNNQRIVAALPTIKYVEEHKPKYIVLASHLGRPNGERN
DKYSLAPVATELEKLLGQKVTFLNDCVGPEVTKAVENAKDGEIFLLENLRYHIEEEGSSKDKDGKKVKADPE
AVKKFRQELTSLADVYINDAFGTAHRAHSSMVGLEVPQRAAGFLMSKELEYFAKALENPERPFLAILGGAKV
SDKIQLIDNLLDKVDMLIVGGGMAFTFKKILNKMPIGDSLFDEAGAKNVEHLVEKAKKNNVELILPVDFVTA
DKFDKDAKTSSATDAEGIPDNWMGLDCGPKSVELFQQAVAKAKTIVWNGPPGVFEFEKFANGTKSLLDAAVK
SAENGNIVIIGGGDTATVAKKYGVVEKLSHVSTGGGASLELLEGKDLPGVVALSNKN*

| Rank | Sequence | Start position | Score |
|---|---|---|---|
| 1 | DAEGIPDNWMGLDCGP | 302 | 0.95 |
| 2 | IVLASHLGRPNGERND | 58 | 0.89 |
| 2 | IVGGGMAFTFKKILNK | 234 | 0.89 |
| 2 | EGSSKDKDGKKVKADP | 128 | 0.89 |
| 3 | KMPIGDSLFDEAGAKN | 249 | 0.88 |
| 4 | DKDAKTSSATDAEGIP | 292 | 0.86 |
| 4 | PVDFVTADKFDKDAKT | 282 | 0.86 |
| 5 | GAKNVEHLVEKAKKNN | 261 | 0.85 |
| 6 | GGGDTATVAKKYGVVE | 371 | 0.82 |
| 6 | GFLMSKELEYFAKALE | 186 | 0.82 |
| 7 | GKTITNNQRIVAALPT | 31 | 0.81 |
| 7 | PERPFLAILGGAKVSD | 203 | 0.81 |
| 8 | AKTIVWNGPPGVFEFE | 330 | 0.80 |
| 9 | VGPEVTKAVENAKDGE | 99 | 0.79 |
| 9 | DAFGTAHRAHSSMVGL | 163 | 0.79 |
| 9 | GKRVFIRVDFNVPLDG | 16 | 0.79 |
| 9 | KFRQELTSLADVYIND | 148 | 0.79 |
| 10 | KADPEAVKKFRQELTS | 140 | 0.77 |
| 11 | NGERNDKYSLAPVATE | 68 | 0.76 |
| 11 | LELLEGKDLPGVVALS | 399 | 0.76 |
| 11 | KSLLDAAVKSAENGNI | 352 | 0.76 |
| 12 | KLSVKDLDVAGKRVFI | 6 | 0.75 |
| 12 | LPTIKYVEEHKPKYIV | 44 | 0.75 |
| 12 | PPGVFEFEKFANGTKS | 338 | 0.75 |

| Start | End | Max_score_pos | Sequence |
|---|---|---|---|
| 277 | 289 | 280 | VELILPVDFVTAD |
| 407 | 414 | 410 | LPGVVALS |
| 55 | 64 | 61 | PKYIVLASHL |
| 75 | 106 | 78 | YSLAPVATELEKLLGQKVTFLNDCVGPEVTKA |
| 376 | 393 | 387 | ATVAKKYGVVEKLSHVST |
| 352 | 361 | 356 | KSLLDAAVKS |
| 152 | 162 | 158 | ELTSLADVYIN |
| 39 | 53 | 43 | RIVAALPTIKYVEEH |
| 314 | 333 | 325 | DCGPKSVELFQQAVAKAKTI |
| 4 | 31 | 22 | SNKLSVKDLDVAGKRVFIRVDFNVPLDG |
| 173 | 188 | 179 | SSMVGLEVPQRAAGFL |
| 206 | 237 | 232 | PFLAILGGAKVSDKIQLIDNLLDKVDMLIVGG |
| 265 | 271 | 268 | VEHLVEK |
| 114 | 124 | 118 | EIFLLENLRYH |
| 339 | 344 | 341 | PGVFEF |
| 193 | 199 | 196 | LEYFAKA |
| 140 | 149 | 146 | KADPEAVKKF |

66. Aspergillus fumigatus Afu2g10620
MSWKLTKKLKDTHLAPLTNTFTRSSSTSTIKNESGEETPVVSQTPSISSTNSNGINASESLVSPPVDPVKPG
ILIVTLHEGRGFALSPHFQQVFTSHFQNNNYSSSVRPSSSSSHSTHGQTASFAQSGRPQSTSGGINAAPTIH
GRYSTKYLPYALLDFEKNQVFVDAVSGTPENPLWAGDNTAFKFDVSRKTELNVQLYLRNPSARPGAGRSEDI
FLGAVRVLPRFEEAQPYVDDPKLSKKDNQKAAAAHANNERHLGQLGAEWLDLQFGTGSIKIGVSFVENKQRS
LKLEDFDLLKVVGKGSFGKVMQVMKKDTGRIYALKTIRKAHIISRSEVTHTLAERSVLAQINNPFIVPLKFS
FQSPEKLYLVLAFVNGGELFHHLQREQRFDINRARFYTAELLCALECLHGFKVIYRDLKPENILLDYTGHIA
LCDFGLCKLDMKDEDRTNTFCGTPEYLAPELLLGNGYTKTVDWWTLGVLLYEMLTGLPPFYDENTNDMYRKI
LQEPLTFPSSDIVPPAARDLLTRLLDRDPQRRLGANGAAEIKSHHFFANIDWRKLLQRKYEPSFRPNVMGAS
DTTNFDTEFTSEAPQDSYVDGPVLSQTMQQQFAGWSYNRPVAGLGDAGGSVKDPSFGSIPE

| Rank | Sequence | Start position | Score |
|---|---|---|---|
| 1 | YEPSFRPNVMGASDTT | 564 | 0.95 |
| 2 | EMLTGLPPFYDENTND | 484 | 0.93 |
| 2 | HGRYSTKYLPYALLDF | 144 | 0.93 |
| 2 | SGGINAAPTIHGRYST | 134 | 0.93 |
| 3 | TPSISSTNSNGINASE | 44 | 0.91 |
| 4 | SEAPQDSYVDGPVLSQ | 587 | 0.88 |
| 4 | TGSIKIGVSFVENKQR | 272 | 0.88 |
| 4 | AQPYVDDPKLSKKDNQ | 230 | 0.88 |
| 5 | QFAGWSYNRPVAGLGD | 607 | 0.87 |
| 5 | LDMKDEDRTNTFCGTP | 441 | 0.87 |
| 6 | TRLLDRDPQRRLGANG | 526 | 0.86 |
| 6 | AVSGTPENPLWAGDNT | 168 | 0.86 |
| 7 | FKVIYRDLKPENILLD | 411 | 0.85 |
| 8 | RPVAGLGDAGGSVKDP | 615 | 0.84 |
| 8 | PPAARDLLTRLLDRDP | 518 | 0.84 |
| 8 | QVMKKDTGRIYALKTI | 310 | 0.84 |
| 8 | PSSSSSHSTHGQTASF | 109 | 0.84 |
| 9 | VMGASDTTNFDTEFTS | 572 | 0.83 |
| 9 | RKILQEPLTFPSSDIV | 502 | 0.83 |
| 9 | KYLPYALLDFEKNQVF | 150 | 0.83 |
| 10 | RKLLQRKYEPSFRPNV | 557 | 0.82 |
| 10 | AAEIKSHHFFANIDWR | 542 | 0.82 |
| 10 | GGELFHHLQREQRFDI | 376 | 0.82 |
| 10 | RSEVTHTLAERSVLAQ | 333 | 0.82 |
| 11 | DRTNTFCGTPEYLAPE | 447 | 0.81 |
| 11 | TGHIALCDFGLCKLDM | 428 | 0.81 |
| 11 | RFDINRARFYTAELLC | 388 | 0.81 |
| 11 | GQTASFAQSGRPQSTS | 119 | 0.81 |
| 12 | KAHIISRSEVTHTLAE | 327 | 0.80 |
| 12 | TGRIYALKTIRKAHII | 316 | 0.80 |
| 12 | IKNESGEETPVVSQTP | 30 | 0.80 |
| 12 | NPLWAGDNTAFKFDVS | 175 | 0.80 |
| 13 | VSPPVDPVKPGILIVT | 62 | 0.79 |
| 13 | PENILLDYTGHIALCD | 420 | 0.79 |
| 13 | KLTKKLKDTHLAPLTN | 4 | 0.79 |
| 13 | THLAPLTNTFTRSSST | 12 | 0.79 |
| 14 | SGRPQSTSGGINAAPT | 127 | 0.78 |
| 15 | VGKGSFGKVMQVMKKD | 300 | 0.76 |
| 15 | LGAEWLDLQFGTGSIK | 261 | 0.76 |
| 15 | VSRKTELNVQLYLRNP | 189 | 0.76 |
| 16 | GILIVTLHEGRGFALS | 72 | 0.75 |
| 16 | FYDENTNDMYRKILQE | 492 | 0.75 |

| Start | End | Max_score_pos | Sequence |
|---|---|---|---|
| 364 | 374 | 371 | PEKLYLVLAFV |
| 396 | 417 | 404 | FYTAELLCALECLHGFKVIYRD |
| 58 | 79 | 76 | SESLVSPPVDPVKPGILIVTLH |
| 149 | 159 | 154 | TKYLPYALLDF |
| 162 | 171 | 166 | NQVFVDAVSG |
| 421 | 442 | 436 | ENILLDYTGHIALCDFGLCKLD |
| 288 | 303 | 297 | SLKLEDFDLLKVVGKG |
| 195 | 203 | 198 | LNVQLYLRN |
| 475 | 492 | 480 | WWTLGVLLYEMLTGLPPF |
| 215 | 227 | 221 | DIFLGAVRVLPRF |
| 591 | 603 | 597 | QDSYVDGPVLSQT |
| 453 | 467 | 462 | CGTPEYLAPELLLGN |
| 351 | 362 | 356 | NPFIVPLKFSFQ |
| 38 | 47 | 42 | TPVVSQTPSI |
| 276 | 283 | 279 | KIGVSFVE |
| 84 | 96 | 91 | FALSPHFQQVFTS |
| 341 | 349 | 346 | AERSVLAQI |
| 379 | 384 | 382 | LFHHLQ |
| 502 | 528 | 519 | RKILQEPLTFPSSDIVPPAARDLLTRL |
| 319 | 325 | 322 | IYALKTI |
| 11 | 18 | 15 | DTHLAPLT |
| 230 | 237 | 236 | AQPYVDDP |
| 104 | 110 | 106 | SSSVRPS |
| 545 | 552 | 548 | IKSHHFFA |
| 327 | 339 | 338 | KAHIISRSEVTHT |
| 557 | 563 | 561 | RKLLQRK |
| 614 | 620 | 618 | NRPVAGL |
| 625 | 630 | 629 | GSVKDP |
| 263 | 269 | 269 | AEWLDLQ |
| 140 | 146 | 142 | APTIHGR |
| 112 | 117 | 112 | SSSHST |

67. Aspergillus nidulans AN8970.2
MTIFRRVALIGRGSLGTVLLDELLNSNFTVTVLTRSASSASSLPPGADIKQVDYSSAESLKTALAGHDIVIS
TLSPSAIPLQKQVIDAAIAVGVKRFIPAEYGAMTSDPVGRKLPFHKDAIEIHEFLRETVASGLIEYTVFGVG
VLTELLFTTTLVVDLEHREVKLFDGGIHSFSTSRLETVARAVVASLHKPDETRNRVIRVHDAVLTQRQVLDM
AKGWTPTLEWREVYVDAQAEVDRGLKQLEKEFSPALVPGVFAAALMSGRYGAEYKEVDNELLGLGFMDKREI
NDFGKKFTK

| Rank | Sequence | Start position | Score |
|---|---|---|---|
| 1 | ASGLIEYTVFGVGVLT | 132 | 0.93 |
| 2 | VALIGRGSLGTVLLDE | 7 | 0.87 |
| 2 | EVYVDAQAEVDRGLKQ | 228 | 0.87 |
| 3 | FMDKREINDFGKKFTK | 282 | 0.86 |
| 3 | LMSGRYGAEYKEVDNE | 261 | 0.86 |
| 3 | VLTQRQVLDMAKGWTP | 207 | 0.86 |
| 4 | AEYGAMTSDPVGRKLP | 100 | 0.84 |
| 5 | RGLKQLEKEFSPALVP | 239 | 0.83 |
| 5 | HSFSTSRLETVARAVV | 172 | 0.83 |
| 6 | TVTVLTRSASSASSLP | 29 | 0.82 |
| 7 | GVKRFIPAEYGAMTSD | 93 | 0.81 |
| 7 | KQVIDAAIAVGVKRFI | 83 | 0.81 |
| 8 | SSASSLPPGADIKQVD | 38 | 0.80 |
| 9 | MAKGWTPTLEWREVYV | 216 | 0.79 |
| 10 | HDIVISTLSPSAIPLQ | 67 | 0.76 |
| 11 | ETRNRVIRVHDAVLTQ | 195 | 0.75 |
| 11 | TVLLDELLNSNFTVTV | 17 | 0.75 |

| Start | End | Max_score_pos | Sequence |
|---|---|---|---|
| 179 | 192 | 188 | LETVARAVVASLHK |
| 132 | 169 | 143 | ASGLIEYTVFGVGVLTELLFTTTLVVDLE |
| | | | HREVKLFDG |
| 249 | 261 | 254 | SPALVPGVFAAAL |
| 15 | 23 | 21 | LGTVLLDEL |
| 57 | 101 | 91 | AESLKTALAGHDIVISTLSPSAIPLQ |
| | | | KQVIDAAIAVGVKRFIPAE |
| 200 | 216 | 206 | VIRVHDAVLTQRQVLDM |
| 226 | 236 | 232 | WREVYVDAQAE |
| 29 | 36 | 31 | TVTVLTRS |
| 4 | 12 | 6 | FRRVALIGR |
| 49 | 55 | 52 | IKQVDYS |
| 38 | 47 | 42 | SSASSLPPGA |
| 110 | 126 | 113 | VGRKLPFHKDAIEIHEF |
| 275 | 281 | 279 | NELLGLG |
| 238 | 244 | 240 | DRGLKQL |

68. Aspergillus nidulans AN2162.2
MDQAIYISSSSEDGFNDDPPLFDEGDNFQEQLPDEERFAAYFDRETPEELFPDRFPKRQRIHGPGDVALDQM
LSSPLAFRGPDSPQSSMAAAADGANTLFLQILEIFPGISHTYVNDLIAQKTVAFRLGADLKARGFQLAILRD
SIYEEILGQKSYPKQDSENGKRKREESEEADISWERTLQNATNSPEYFEAASAFLGPEFPWVPMSHIKKVLI
DKGRLYHAFVALYSDDNLLEQRKYQYVRLKSQRSTNSPKKYTPLRDTLIREINAARKHVEELQITLRKKKEE
EEAEKANEEEHIRTGSLIECHCCYADVPSNRCIPCDGDDLHFFCFTCIRRSADNQIGMMKYILQCFDVSGCQ
ASFNRQQLREILGPVVMDKLDSLQQEDEIRKAGLEGLEDCPFCSYKAVLPPVEEDREFRCENSQCKVVSCRL
CKEKSHIPQTCEEYRKDKGLSERHQVEEAMSNALIRKCPKCRLKIIKEYGCNKMQCTKCHTLMCYVCQKDIT
KEGYAHFGRGGCPQDDIHTQDRDDREIQRAERAAIDKILAENPDISEEQIRVGHEKTNAQTRGVRRDPRLQP
AIQMRDAMRVMRADMGGFYPQQHQHANTAAQRQLPVYPPPAYNVPYPMDYGTMFNPPFPGFNVLQRGLQPGN
LPAQPAVMQPMVVGLANPPANFHPQDIQNITAFPPQQSLPRNQNAAYRGVGFGPF

| Rank | Sequence | Start position | Score |
|---|---|---|---|
| 1 | DGFNDDPPLFDEGDNF | 13 | 0.97 |
| 2 | LREILGPVVMDKLDSL | 368 | 0.95 |
| 3 | GHEKTNAQTRGVRRDP | 557 | 0.94 |
| 4 | DQMLSSPLAFRGPDSP | 70 | 0.93 |
| 4 | AIYISSSSEDGFNDDP | 4 | 0.93 |
| 4 | ECHCCYADVPSNRCIP | 307 | 0.93 |
| 5 | YGTMFNPPFPGFNVLQ | 626 | 0.91 |
| 5 | KVLIDKGRLYHAFVAL | 213 | 0.91 |
| 6 | DITKEGYAHFGRGGCP | 502 | 0.90 |
| 6 | EDEIRKAGLEGLEDCP | 386 | 0.90 |
| 7 | IREINAARKHVEELQI | 265 | 0.89 |
| 7 | FPGISHTYVNDLIAQK | 107 | 0.89 |
| 8 | RQLPVYPPPAYNVPYP | 608 | 0.88 |
| 8 | FGRGGCPQDDIHTQDR | 511 | 0.88 |
| 9 | FRGPDSPQSSMAAAAD | 79 | 0.87 |
| 9 | ADMGGFYPQQHQHANT | 589 | 0.87 |
| 9 | QPAIQMRDAMRVMRAD | 575 | 0.87 |
| 10 | AYNVPYPMDYGTMFNP | 617 | 0.86 |
| 10 | KGLSERHQVEEAMSNA | 450 | 0.86 |
| 10 | YFDRETPEELFPDRFP | 41 | 0.86 |
| 10 | GMMKYILQCFDVSGCQ | 345 | 0.86 |
| 11 | RLKIIKEYGCNKMQCT | 474 | 0.85 |
| 12 | PDISEEQIRVGHEKTN | 547 | 0.84 |
| 12 | MQCTKCHTLMCYVCQK | 486 | 0.84 |
| 12 | SHIPQTCEEYRKDKGL | 437 | 0.84 |
| 12 | LPPVEEDREFRCENSQ | 409 | 0.84 |
| 13 | QDDIHTQDRDDREIQR | 518 | 0.83 |
| 13 | DLIAQKTVAFRLGADL | 117 | 0.83 |
| 14 | RRSADNQIGMMKYILQ | 337 | 0.82 |
| 15 | NALIRKCPKCRLKIIK | 464 | 0.81 |
| 15 | LKSQRSTNSPKKYTPL | 245 | 0.81 |
| 15 | PEFPWVPMSHIKKVLI | 201 | 0.81 |
| 16 | NITAFPPQQSLPRNQN | 677 | 0.80 |
| 16 | CYVCQKDITKEGYAHF | 496 | 0.80 |
| 16 | EGLEDCPFCSYKAVLP | 395 | 0.80 |
| 16 | RDSIYEEILGQKSYPK | 143 | 0.80 |
| 17 | NVLQRGLQPGNLPAQP | 638 | 0.79 |
| 17 | EEEAEKANEEEHIRTG | 288 | 0.79 |
| 17 | TNSPEYFEAASAFLGP | 186 | 0.79 |
| 17 | ADISWERTLQNATNSP | 174 | 0.79 |
| 18 | AQPAVMQPMVVGLANP | 651 | 0.78 |
| 18 | HQHANTAAQRQLPVYP | 599 | 0.78 |
| 18 | RQRIHGPGDVALDQML | 58 | 0.78 |
| 19 | PDEERFAAYFDRETPE | 33 | 0.77 |
| 19 | FEAASAFLGPEFPWVP | 192 | 0.77 |
| 20 | HQVEEAMSNALIRKCP | 456 | 0.76 |
| 20 | RCIPCDGDDLHFFCFT | 319 | 0.76 |
| 20 | QKSYPKQDSENGKRKR | 153 | 0.76 |
| 21 | LQPGNLPAQPAVMQPM | 644 | 0.75 |
| 21 | RAAIDKILAENPDISE | 536 | 0.75 |
| 21 | NFQEQLPDEERFAAYF | 27 | 0.75 |

| Start | End | Max_score_pos | Sequence |
|---|---|---|---|
| 421 | 444 | 430 | ENSQCKVVSCRLCKEKSHIPQTCE |
| 305 | 326 | 308 | LIECHCCYADVPSNRCIPCDGD |
| 488 | 502 | 497 | CTKCHTLMCYVCQKD |
| 397 | 412 | 406 | LEDCPFCSYKAVLPPV |
| 348 | 362 | 353 | KYILQCFDVSGCQAS |
| 328 | 338 | 333 | LHFFCFTCIRR |
| 191 | 229 | 224 | YFEAASAFLGPEFPWVPMSHIKKVLIDKGRLYHAFVALY |
| 607 | 624 | 613 | QRQLPVYPPPAYNVPYPM |
| 368 | 383 | 373 | LREILGPVVMDKLDSL |
| 98 | 131 | 101 | TLFLQILEIFPGISHTYVNDLIAQKTVAFRLGAD |
| 646 | 666 | 652 | PGNLPAQPAVMQPMVVGLANP |
| 466 | 481 | 472 | LIRKCPKCRLKIIKEY |
| 237 | 246 | 242 | QRKYQYVRLK |
| 64 | 80 | 76 | PGDVALDQMLSSPLAFR |
| 135 | 145 | 139 | RGFQLAILRDS |
| 633 | 643 | 642 | PFPGFNVLQRG |
| 272 | 282 | 277 | RKHVEELQITL |
| 4 | 9 | 6 | AIYISS |
| 678 | 688 | 684 | ITAFPPQQSLP |
| 573 | 578 | 575 | RLQPAI |
| 595 | 600 | 598 | YPQQHQ |
| 256 | 263 | 262 | KYTPLRDT |
| 695 | 700 | 697 | YRGVGF |

69. Aspergillus nidulans AN6709.2
MAEAENDPTNELNQTSVTQADKQNGVDVATEPHAPEVVAESKPALTDESERQEIPTIKENEDTMANNRLNDS
KNNLPHEPSVTSPDTTTDSNEPTDEPEQPHTEGDQLETLQQDQPPASDEQLNEAPDAPSTRDEQLAQDMRQR
SDSRSTTATFATNRSSVVSSTVFIVTALDAIGASREARKSKELEDAVKNALANVKQSDRQPIDPEILFYPLL
LASRTLSIPLQVTALDCIGKLITYSYFAFPSAQEAKPSEADATAEQPPLIERAIDAICDCFENEATPIEIQQ
QIIKSLLAAVLNDKIVVHGAGLLKAVRQIYNMFIYSKSSQNQQIAQGSLTQMVSTVFDRLRVRLDLRELRIR
EGEKAQAGSSESVTIEPVVSPPSAEDDQASDVASVAADQPVSKEPTEKLTLESFESNKDVTTVNDNVPTMVT
RANINQKRTQSYSGTSSEEKEAEDASSNEDDVDEIYVKDAFLVFRALCKLSHKVLSHEQQQDLKSQNMRSKL
LSLHLIHYLINNHVIIFTTPLLTLKNSSGNLEAMTFLQAIRPHLCLSLSRNGASSVPKVFEVCCEIFWLMLK
HMRVMMKKELEVFMKEIYLAILEKRNAPAFQKQYFMEILERLADEPRALVEMYLNYDCDRTALENIFQNIIE
QLSRYASIPTVVNPLQQQQYHELHVKASSVGNEWHQRGTLPPNLTSASIGNNQQPPTHSVPSEYILKHQAVE
CLVVILESLDNWASQRSVDPTAARTFSQKSVDNPRDSMDSSAPAFLASPRVDGADGSTGRSTPVPDDDPSQV
EKVKQRKIALTNVIQQFNFKPKRGVKLALQEGFIRSDSPEDIAAFILRNDRLDKAMIGEYLGEGDAENIATM
HAFVDMMDFSKRRFVDALRSFLQHFRLPGEAQKIDRFMLKFSERYVTQNPNAFANADTAYVLAYSVILLNTD
QHSSKMKGRRMTKEDFIKNNRGINDNQDLPDEYLGSIFDEIANNEIVLDTEREQAANAAHPAPVPSGLASRA
GQVFATVGRDIQGERYAQASEEMANKTEQLYRSLIRAQRKTAVKEALSRFIFATSVQHAGSMFNVTWMSFLS
GLSAPMQDTQNLKTIKLCMEGMKLAIRISCTFDLETPRVAFVTALAKFTNLGNVREMVAKNVEAVKILLDVA
LSEGNHLKSSWRDILTCVSQLDRLQLLSDGVDEGSLPDMSRAGVVPPSASDGPRRSMQAPRRPRPKSITGPT
PFRAEIAMESRSTEMVKGVDRIFTNTANLSHEAIIDFVRALSEVSWQEIQSSGQTASPRTYSLQKLVEISYY
NMTRVRIEWSKIWEVLGQHFNQVGCHSNTTVVFFALDSLRQLSMRFMEIEELPGFKFQKDFLKPFEHVMSNS
NAVTVKDMILRCLIQMIQARGDNIRSGWKTMFGVFSFAAREPYDTEGIVNMAFEHVTQIYNTRFGVVITQGA
FPDLVVCLTEFSKNTRFQKKSLQAIELLKSTVAKMLRTPECPLSHRSSTEAFHEDSTNLTQQLTKQSKEEQF
WYPILIAFQDILMTGDDLEARSRALTYLFDTLIRYGGSFPQEFWDVLWRQLLYPIFVVLQSKSEMSKVPNHE
ELSVWLSTTMIQALRHMITLFTHYFDALEYMLGRVLELLTLCICQENDTIARIGSNCLQQLILQNVEKFQKD
HWNKTVGAFIELFNKTTAYELFTAATTMATVTLKTPSAPTANGQLADTHDTVQDPTESSPAQETSTEPPKLN
GTQDTTAEHEDGDMPAASNTELEDYRPQSDTQQQPAAVTAARRRYFNRIITSCVLQLLMIETVHELFSNDKV
YAQIPSHELLRLMGLLKKSYQFAKKFNEDKELRMQLWRQGFMKSPPNLLKQESGSAATYVHILFRMYHDERE
ERKSSRSETEAALIPLCVDIISGFVRLDEDSQHRNIVAWRPVVVDVIEGYTNFPAEGFDKHIDTFYPLAVDL
LGRELNSEIRLAIQGLFQRIGEARLGLPVRPTPTPVSPRHSVSEHPSRKHSVGRR

| Rank | Sequence | Start position | Score |
|---|---|---|---|
| 1 | PKSITGPTPFRAEIAM | 1217 | 0.96 |
| 2 | TELEDYRPQSDTQQQP | 1748 | 0.94 |
| 2 | NEAPDAPSTRDEQLAQ | 124 | 0.94 |
| 2 | RRSMQAPRRPRPKSIT | 1206 | 0.94 |
| 3 | TANGQLADTHDTVQDP | 1696 | 0.93 |
| 4 | LGSIFDEIANNEIVLD | 970 | 0.91 |
| 4 | SVTSPDTTTDSNEPTD | 81 | 0.91 |
| 4 | VDEGSLPDMSRAGVVP | 1183 | 0.91 |
| 4 | TLQQDQPPASDEQLNE | 110 | 0.91 |
| 5 | HSSKMKGRRMTKEDFI | 938 | 0.90 |
| 5 | NWASQRSVDPTAARTF | 731 | 0.90 |
| 5 | HEDGDMPAASNTELED | 1737 | 0.90 |
| 5 | TRDEQLAQDMRQRSDS | 132 | 0.90 |
| 6 | RMTKEDFIKNNRGIND | 946 | 0.89 |
| 6 | PGEAQKIDRFMLKFSE | 892 | 0.89 |
| 6 | GSTGRSTPVPDDDPSQ | 776 | 0.89 |
| 6 | TIKENEDTMANNRLND | 56 | 0.89 |
| 6 | TPTPVSPRHSVSEHPS | 1976 | 0.89 |
| 7 | SPRVDGADGSTGRSTP | 768 | 0.88 |
| 7 | AVLNDKIVVHGAGLLK | 297 | 0.88 |
| 7 | ERAIDAICDCFENEAT | 267 | 0.88 |
| 7 | NGTQDTTAEHEDGDMP | 1728 | 0.88 |
| 7 | SPAQETSTEPPKLNGT | 1715 | 0.88 |
| 7 | HDTVQDPTESSPAQET | 1705 | 0.88 |
| 7 | LKTPSAPTANGQLADT | 1689 | 0.88 |
| 7 | GVVITQGAFPDLVVCL | 1433 | 0.88 |
| 8 | RGINDNQDLPDEYLGS | 957 | 0.87 |
| 8 | QKSVDNPRDSMDSSAP | 748 | 0.87 |
| 8 | EILERLADEPRALVEM | 613 | 0.87 |
| 8 | IYVKDAFLVFRALCKL | 467 | 0.87 |
| 8 | TTVNDNVPTMVTRANI | 421 | 0.87 |
| 8 | AYELFTAATTMATVTL | 1674 | 0.87 |
| 8 | HVTQIYNTRFGVVITQ | 1423 | 0.87 |
| 8 | AREPYDTEGIVNMAFE | 1407 | 0.87 |
| 8 | KSSWRDILTCVSQLDR | 1160 | 0.87 |
| 8 | KLAIRISCTFDLETPR | 1103 | 0.87 |
| 9 | DEPEQPHTEGDQLETL | 96 | 0.86 |
| 9 | SVDPTAARTFSQKSVD | 737 | 0.86 |
| 9 | RALVEMYLNYDCDRTA | 623 | 0.86 |
| 9 | VATEPHAPEVVAESKP | 28 | 0.86 |
| 9 | AVTAARRRYFNRIITS | 1765 | 0.86 |
| 9 | FTHYFDALEYMLGRVL | 1605 | 0.86 |
| 10 | EGFIRSDSPEDIAAFI | 823 | 0.85 |
| 10 | TPVPDDDPSQVEKVKQ | 782 | 0.85 |
| 10 | SHEQQQDLKSQNMRSK | 488 | 0.85 |
| 10 | SLTQMVSTVFDRLRVR | 336 | 0.85 |
| 10 | VDVIEGYTNFPAEGFD | 1916 | 0.85 |
| 10 | MGLLKKSYQFAKKFNE | 1813 | 0.85 |
| 10 | REARKSKELEDAVKNA | 179 | 0.85 |
| 10 | LDAIGASREARKSKEL | 172 | 0.85 |
| 10 | AKMLRTPECPLSHRSS | 1473 | 0.85 |
| 10 | QSSGQTASPRTYSLQK | 1274 | 0.85 |
| 10 | EVSWQEIQSSGQTASP | 1267 | 0.85 |
| 11 | PAPVPSGLASRAGQVF | 997 | 0.84 |
| 11 | DPSQVEKVKQRKIALT | 788 | 0.84 |
| 11 | TQSYSGTSSEEKEAED | 441 | 0.84 |
| 11 | DVASVAADQPVSKEPT | 391 | 0.84 |
| 11 | IGEARLGLPVRPTPTP | 1964 | 0.84 |
| 11 | EVLGQHFNQVGCHSNT | 1310 | 0.84 |
| 11 | IAMESRSTEMVKGVDR | 1230 | 0.84 |
| 11 | APMQDTQNLKTIKLCM | 1084 | 0.84 |
| 12 | FSERYVTQNPNAFANA | 905 | 0.83 |
| 12 | EKLTLESFESNKDVTT | 407 | 0.83 |
| 12 | ATAEQPPLIERAIDAI | 258 | 0.83 |
| 12 | FPSAQEAKPSEADATA | 245 | 0.83 |
| 12 | TTMIQALRHMITLFTH | 1592 | 0.83 |
| 12 | LSHRSSTEAFHEDSTN | 1483 | 0.83 |
| 12 | SDSRSTTATFATNRSS | 145 | 0.83 |
| 12 | FMEIEELPGFKFQKDF | 1342 | 0.83 |
| 12 | HEAIIDFVRALSEVSW | 1255 | 0.83 |
| 13 | PRDSMDSSAPAFLASP | 754 | 0.82 |
| 13 | EDDVDEIYVKDAFLVF | 461 | 0.82 |
| 13 | ELRIREGEKAQAGSSE | 356 | 0.82 |
| 13 | GKLITYSYFAFPSAQE | 235 | 0.82 |
| 13 | IQMIQARGDNIRSGWK | 1382 | 0.82 |
| 13 | ASDEQLNEAPDAPSTR | 118 | 0.82 |
| 13 | LKTIKLCMEGMKLAIR | 1092 | 0.82 |
| 13 | ERYAQASEEMANKTEQ | 1022 | 0.82 |
| 14 | ASSVGNEWHQRGTLPP | 675 | 0.81 |
| 14 | SEEKEAEDASSNEDDV | 449 | 0.81 |
| 14 | SFESNKDVTTVNDNVP | 413 | 0.81 |
| 14 | EPVVSPPSAEDDQASD | 376 | 0.81 |
| 14 | HILFRMYHDEREERKS | 1861 | 0.81 |
| 14 | GCHSNTTVVFFALDSL | 1320 | 0.81 |
| 14 | IFTNTANLSHEAIIDF | 1246 | 0.81 |
| 14 | VPPSASDGPRRSMQAP | 1197 | 0.81 |
| 15 | TDSNEPTDEPEQPHTE | 89 | 0.80 |
| 15 | EGFDKHIDTFYPLAVD | 1928 | 0.80 |
| 15 | RSSVVSSTVFIVTALD | 158 | 0.80 |
| 15 | FVVLQSKSEMSKVPNH | 1568 | 0.80 |
| 16 | DRFMLKFSERYVTQNP | 899 | 0.79 |
| 16 | PPTHSVPSEYILKHQA | 703 | 0.79 |
| 16 | SRYASIPTVVNPLQQQ | 651 | 0.79 |
| 16 | KPALTDESERQEIPTI | 42 | 0.79 |
| 16 | GIVNMAFEHVTQIYNT | 1415 | 0.79 |
| 16 | LVEISYYNMTRVRIEW | 1290 | 0.79 |
| 16 | SRFIFATSVQHAGSMF | 1056 | 0.79 |
| 16 | NELNQTSVTQADKQNG | 10 | 0.79 |
| 17 | NNEIVLDTEREQAANA | 979 | 0.78 |
| 17 | KAMIGEYLGEGDAENI | 846 | 0.78 |
| 17 | EWHQRGTLPPNLTSAS | 681 | 0.78 |
| 17 | VMMKKELEVFMKEIYL | 580 | 0.78 |
| 17 | HVIIFTTPLLTLKNSS | 517 | 0.78 |
| 17 | DPEILFYPLLLASRTL | 207 | 0.78 |
| 17 | AATTMATVTLKTPSAP | 1680 | 0.78 |
| 17 | DVLWRQLLYPIFVVLQ | 1557 | 0.78 |
| 17 | ILMTGDDLEARSRALT | 1523 | 0.78 |
| 17 | GWKTMFGVFSFAAREP | 1395 | 0.78 |
| 17 | TVGRDIQGERYAQASE | 1014 | 0.78 |
| 18 | PEDIAAFILRNDRLDK | 831 | 0.77 |
| 18 | LPPNLTSASIGNNQQP | 688 | 0.77 |
| 18 | EIFWLMLKHMRVMMKK | 569 | 0.77 |
| 18 | RHSVSEHPSRKHSVGR | 1983 | 0.77 |
| 18 | FVRLDEDSQHRNIVAW | 1896 | 0.77 |
| 18 | TQQQPAAVTAARRRYF | 1759 | 0.77 |
| 18 | DTLIRYGGSFPQEFWD | 1542 | 0.77 |
| 18 | HTEGDQLETLQQDQPP | 102 | 0.77 |
| 19 | SRTLSIPLQVTALDCI | 219 | 0.76 |
| 19 | QDMRQRSDSRSTTATF | 139 | 0.76 |
| 19 | MAEAENDPTNELNQTS | 1 | 0.76 |
| 20 | SASIGNNQQPPTHSVP | 694 | 0.75 |
| 20 | RQIYNMFIYSKSSQNQ | 315 | 0.75 |
| 20 | KQESGSAATYVHILFR | 1850 | 0.75 |

| Start | End | Max_score_pos | Sequence |
|---|---|---|---|
| 704 | 728 | 722 | PTHSVPSEYILKHQAVECLVVILES |
| 1595 | 1629 | 1625 | IQALRHMITLFTHYFDALEYMLGRVLELLTLCICQ |
| 1906 | 1921 | 1916 | RNIVAWRPVVVDVIEG |
| 1441 | 1450 | 1445 | FPDLVVCLTE |
| 1882 | 1900 | 1887 | EAALIPLCVDIISGFVRLD |
| 1778 | 1794 | 1783 | ITSCVLQLLMIETVHEL |
| 1555 | 1573 | 1567 | FWDVLWRQLLYPIFVVLQS |
| 558 | 578 | 565 | SSVPKVFEVCCEIFWLMLKHM |
| 922 | 935 | 927 | TAYVLAYSVILLNT |
| 1638 | 1651 | 1644 | GSNCLQQLILQNVE |
| 538 | 553 | 549 | MTFLQAIRPHLCLSLS |
| 465 | 490 | 477 | DEIYVKDAFLVFRALCKLSHKVLSHE |
| 207 | 248 | 214 | DPEILFYPLLLASRTLSIPLQVTALDCIGKLITYSYF |
| | | | AFPSA |
| 283 | 317 | 296 | PIEIQQQIIKSLLAAVLNDKIVVHGAGLLKAVRQI |
| 503 | 529 | 510 | KLLSLHLIHYLINNHVIIFTTPLLTLK |
| 158 | 175 | 169 | RSSVVSSTVFIVTALDAI |
| 1166 | 1182 | 1170 | ILTCVSQLDRLQLLSDG |
| 1139 | 1154 | 1150 | AKNVEAVKILLDVALS |
| 1324 | 1338 | 1329 | NTTVVFFALDSLRQL |
| 1116 | 1128 | 1122 | TPRVAFVTALAKF |
| 1935 | 1947 | 1942 | DTFYPLAVDLLGR |
| 370 | 382 | 376 | SESVTIEPVVSPP |
| 1370 | 1385 | 1380 | AVTVKDMILRCLIQMI |
| 1856 | 1866 | 1861 | AATYVHILFRM |
| 1283 | 1294 | 1288 | RTYSLQKLVEIS |
| 1194 | 1200 | 1199 | AGVVPPS |
| 644 | 677 | 657 | QNIIEQLSRYASIPTVVNPLQQQQYHELHVKASS |
| 1513 | 1523 | 1517 | WYPILIAFQDI |
| 261 | 277 | 273 | EQPPLIERAIDAICDCF |
| 1105 | 1113 | 1107 | AIRISCTFD |
| 34 | 44 | 35 | APEVVAESKPA |
| 585 | 598 | 595 | ELEVFMKEIYLAIL |
| 1797 | 1823 | 1803 | NDKVYAQIPSHELLRLMGLLKKSYQFA |
| 389 | 404 | 398 | ASDVASVAADQPVSKE |
| 1459 | 1475 | 1465 | KKSLQAIELLKSTVAKM |
| 1968 | 1989 | 1983 | RLGLPVRPTPTPVSPRHSVSEH |
| 1254 | 1271 | 1265 | SHEAIIDFVRALSEVSWQ |
| 993 | 1018 | 998 | NAAHPAPVPSGLASRAGQVFATVGRD |
| 1431 | 1439 | 1435 | RFGVVITQG |
| 621 | 629 | 627 | EPRALVEMY |
| 816 | 824 | 819 | GVKLALQEG |
| 1478 | 1486 | 1482 | TPECPLSHR |
| 338 | 357 | 351 | TQMVSTVFDRLRVRLDLREL |
| 762 | 771 | 768 | APAFLASPRV |
| 78 | 85 | 79 | HEPSVTSP |
| 1038 | 1045 | 1040 | LYRSLIRA |
| 1585 | 1591 | 1589 | ELSVWLS |
| 1051 | 1067 | 1061 | VKEALSRFIFATSVQHA |
| 1954 | 1963 | 1958 | RLAIQGLFQR |
| 802 | 809 | 803 | LTNVIQQF |
| 1535 | 1548 | 1542 | RALTYLFDTLIRYG |
| 876 | 892 | 888 | RRFVDALRSFLQHFRLP |
| 1354 | 1364 | 1361 | QKDFLKPFEHV |
| 1662 | 1668 | 1665 | VGAFIEL |
| 1308 | 1322 | 1318 | IWEVLGQHFNQVGCH |
| 833 | 839 | 837 | DIAAFIL |
| 789 | 795 | 792 | PSQVEKV |
| 1762 | 1769 | 1763 | QPAAVTAA |
| 1400 | 1408 | 1403 | FGVFSFAAR |
| 1418 | 1427 | 1425 | NMAFEHVTQI |
| 1079 | 1085 | 1081 | LSGLSAP |
| 192 | 199 | 193 | KNALANVK |
| 25 | 32 | 29 | GVDVATEP |
| 1240 | 1246 | 1243 | VKGVDRI |
| 1686 | 1692 | 1690 | TVTLKTP |
| 966 | 973 | 972 | PDEYLGSI |
| 1674 | 1680 | 1677 | AYELFTA |
| 735 | 741 | 740 | QRSVDPT |
| 1846 | 1851 | 1848 | PNLLKQ |
| 689 | 695 | 691 | PPNLTSA |
| 14 | 20 | 17 | QTSVTQA |
| 980 | 985 | 985 | NEIVLD |
| 1500 | 1505 | 1502 | TQQLTK |
| 781 | 787 | 782 | STPVPDD |
| 420 | 426 | 425 | VTTVNDN |

70. Aspergillus nidulans AN7704.2
MARSFVCHVSDSITTLLFRSLSFVYCMPEFKISAALEGHGDDVRAVAFPNSKAVFSASRDATVRLWKLVSSP
PPTFDYTIICHGSAFINALAYYPPTPDFPEGLVFSGGQDTIIEARQPGKTSNDNADAMLLGHAHTVCSLDVC
PEGEWIVSGSWDSTARLWRIGKWESEVVLEDHQGSVWAVLAYDKNTIITDSRDVVRALCKLPPTHPTGANFV
SASNDGVIRLFTLQGDLVGELHGHESFIYSLAVLPTGELVSSGEDRTVRIWNETQCVQTITHPAISVWGVAV
CPENGDIVTGASDRVTRVFTRAPERQASAEVLQQFETAVRESAIPAQQVGKINKEKLPGPEFLQQKSGTKDG
QVQMIREANGSVTAHTWSAALGRWESVGTVVDSAGSSGRKTEYLGQDYDFVFDVDVEDGKPPLKLPYNLSQN
PYEAATKFIGDNELPMSYLDQVAQFIVQNTQGATIGQPSQETAGGPDPWGQDRRYRPGDAPAQSTAIPESRP
KVLPQKTYLSIKSANLKVISKKLNELNGKLVSEGSKDLSLSPSELETIVSLCNELEASNTLKGPSAVEAVVI
LLFKVATVWPAANRLPGLDLLRLFAAATPVTATADYNGKDLVSGIIESGVFDAPVNVNNAMLSVRMFANLFE
TDAGRRLIIDRFDQVIAAIRTCLTNSGSSVNRNLTIAVATLYINIAVFSTSEARNLSIESNQRGLILLEELT
GMLRNEKDSEAVYRSLVALGTLVKELVSEVKAAAKEVYDLGAILQAISSSNLGKEPRIKGIVAEIKDSLP

| Rank | Sequence | Start position | Score |
|---|---|---|---|
| 1 | QDTIIEARQPGKTSND | 110 | 0.97 |
| 2 | VSGIIESGVFDAPVNV | 618 | 0.95 |
| 2 | PDPWGQDRRYRPGDAP | 478 | 0.95 |
| 3 | QPSQETAGGPDPWGQD | 469 | 0.92 |
| 3 | DGKPPLKLPYNLSQNP | 418 | 0.92 |
| 4 | TKFIGDNELPMSYLDQ | 438 | 0.91 |
| 5 | NALAYYPPTPDFPEGL | 89 | 0.90 |
| 5 | PAQSTAIPESRPKVLP | 493 | 0.90 |
| 6 | RALCKLPPTHPTGANF | 200 | 0.89 |
| 7 | VQTITHPAISVWGVAV | 273 | 0.88 |
| 8 | DGQVQMIREANGSVTA | 359 | 0.87 |
| 8 | AALEGHGDDVRAVAFP | 34 | 0.87 |
| 8 | QGSVWAVLAYDKNTII | 177 | 0.87 |
| 8 | EWIVSGSWDSTARLWR | 148 | 0.87 |
| 9 | EFLQQKSGTKDGQVQM | 349 | 0.86 |
| 10 | KLVSSPPPTFDYTIIC | 67 | 0.85 |
| 10 | NGSVTAHTWSAALGRW | 369 | 0.85 |
| 10 | GELVSSGEDRTVRIWN | 253 | 0.85 |
| 11 | TPDFPEGLVFSGGQDT | 97 | 0.84 |
| 11 | LLFKVATVWPAANRLP | 577 | 0.84 |
| 11 | KNTIITDSRDVVRALC | 188 | 0.84 |
| 12 | ELTGMLRNEKDSEAVY | 718 | 0.83 |
| 12 | MLLGHAHTVCSLDVCP | 130 | 0.83 |
| 13 | KVLPQKTYLSIKSANL | 505 | 0.82 |
| 13 | RKTEYLGQDYDFVFDV | 399 | 0.82 |
| 14 | YTIICHGSAFINALAY | 78 | 0.81 |
| 14 | EVKAAAKEVYDLGAIL | 749 | 0.81 |
| 14 | DQVIAAIRTCLTNSGS | 661 | 0.81 |
| 14 | AATPVTATADYNGKDL | 602 | 0.81 |
| 14 | SWDSTARLWRIGKWES | 154 | 0.81 |
| 15 | SASRDATVRLWKLVSS | 56 | 0.80 |
| 15 | EGSKDLSLSPSELETI | 537 | 0.80 |
| 15 | YRPGDAPAQSTAIPES | 487 | 0.80 |
| 16 | VTGASDRVTRVFTRAP | 296 | 0.79 |
| 16 | PPTHPTGANFVSASND | 206 | 0.79 |
| 17 | LQAISSSNLGKEPRIK | 764 | 0.78 |
| 17 | SLSPSELETIVSLCNE | 543 | 0.78 |
| 17 | RESAIPAQQVGKINKE | 328 | 0.78 |
| 18 | VEAVVILLFKVATVWP | 571 | 0.77 |
| 18 | VGKINKEKLPGPEFLQ | 337 | 0.77 |
| 19 | DQVAQFIVQNTQGATI | 452 | 0.76 |
| 20 | SGVFDAPVNVNNAMLS | 624 | 0.75 |
| 20 | RWESVGTVVDSAGSSG | 383 | 0.75 |
| 20 | DRTVRIWNETQCVQTI | 261 | 0.75 |

| Start | End | Max_score_pos | Sequence |
|---|---|---|---|
| 129 | 154 | 141 | AMLLGHAHTVCSLDVCPEGEWIVSGS |
| 567 | 587 | 576 | GPSAVEAVVILLFKVATVWPA |
| 4 | 29 | 7 | SFVCHVSDSITTLLFRSLSFVYCMPE |
| 269 | 291 | 286 | ETQCVQTITHPAISVWGVAVCPE |
| 195 | 208 | 201 | SRDVVRALCKLPPT |
| 239 | 258 | 247 | GHESFIYSLAVLPTGELVSS |
| 177 | 186 | 183 | QGSVWAVLAY |
| 730 | 770 | 734 | EAVYRSLVALGTLVKELVSEVKAAAKEVYDLGAILQAISSS |
| 448 | 462 | 456 | MSYLDQVAQFIVQNT |
| 405 | 417 | 413 | GQDYDFVFDVDVE |
| 683 | 698 | 688 | TIAVATLYINIAVFST |
| 549 | 559 | 553 | LETIVSLCNEL |
| 62 | 98 | 66 | TVRLWKLVSSPPPTFDYTIICHGSAFINALAYYPPTP |
| 42 | 57 | 46 | DVRAVAFPNSKAVFSA |
| 223 | 237 | 226 | VIRLFTLQGDLVGEL |
| 712 | 718 | 716 | GLILLEE |
| 169 | 175 | 174 | SEVVLED |
| 593 | 609 | 599 | GLDLLRLFAAATPVTAT |
| 387 | 394 | 389 | VGTVVDSA |
| 422 | 432 | 424 | PLKLPYNLSQN |
| 328 | 339 | 334 | RESAIPAQQVGK |
| 623 | 633 | 628 | ESGVFDAPVNV |
| 654 | 673 | 668 | RLIIDRFDQVIAAIRTCLTN |
| 503 | 525 | 509 | RPKVLPQKTYLSIKSANLKVISK |
| 615 | 621 | 619 | KDLVSGI |
| 316 | 326 | 320 | SAEVLQQFETA |
| 101 | 108 | 103 | PEGLVFSG |
| 31 | 37 | 33 | KISAALE |
| 636 | 643 | 641 | AMLSVRMF |
| 532 | 537 | 533 | GKLVSE |
| 341 | 547 | 544 | DLSLSPS |
| 347 | 354 | 348 | GPEFLQQK |
| 779 | 787 | 779 | KGIVAEIKD |
| 303 | 309 | 305 | VTRVFTR |
| 213 | 218 | 216 | ANFVSA |

71. Saccharomyces cerevisiae YBL024w
MARRKNFKKGNKKTFGARDDSRAQKNWSELVKENEKWEKYYKTLALFPEDQWEEFKKTCQAPLPLTFRITGS
RKHAGEVLNLFKERHLPNLTNVEFEGEKIKAPVELPWYPDHLAWQLDVPKTVIRKNEQFAKTQRFLVVENAV
GNISRQEAVSMIPPIVLEVKPHHTVLDMCAAPGSKTAQLIEALHKDTDEPSGFVVANDADARRSHMLVHQLK
RLNSANLMVVNHDAQFFPRIRLHGNSNNKNDVLKFDRILCDVPCSGDGTMRKNVNVWKDWNTQAGLGLHAVQ
LNILNRGLHLLKNNGRLVYSTCSLNPIENEAVVAEALRKWGDKIRLVNCDDKLPGLIRSKGVSKWPVYDRNL
TEKTKGDEGTLDSFFSPSEEEASKFNLQNCMRVYPHQQNTGGFFITVFEKVEDSTEAATEKLSSETPALESE
GPQTKKIKVEEVQKKERLPRDANEEPFVFVDPQHEALKVCWDFYGIDNIFDRNTCLVRNATGEPTRVVYTVC
PALKDVIQANDDRLKIIYSGVKLFVSQRSDIECSWRIQSESLPIMKHHMKSNRIVEANLEMLKHLLIESFPN
FDDIRSKNIDNDFVEKMTKLSSGCAFIDVSRNDPAKENLFLPVWKGNKCINLMVCKEDTHELLYRIFGIDAN
AKATPSAEEKEKEKETTESPAETTTGTSTEAPSAAN

| Rank | Sequence | Start position | Score |
|---|---|---|---|
| 1 | PGLIRSKGVSKWPVYD | 342 | 0.94 |
| 2 | HLLIESFPNFDDIRSK | 568 | 0.93 |
| 2 | DVPCSGDGTMRKNVNV | 257 | 0.93 |
| 3 | KEKEKETTESPAETTT | 658 | 0.90 |
| 3 | KKTFGARDDSRAQKNW | 12 | 0.90 |
| 3 | PVELPWYPDHLAWQLD | 104 | 0.90 |
| 4 | NFKKGNKKTFGARDDS | 6 | 0.88 |
| 5 | LLYRIFGIDANAKATP | 638 | 0.87 |
| 5 | IRSKNIDNDFVEKMTK | 580 | 0.87 |
| 6 | RLPRDANEEPFVFVDP | 449 | 0.86 |
| 6 | KGVSKWPVYDRNLTEK | 348 | 0.86 |
| 7 | EVQKKERLPRDANEEP | 443 | 0.85 |
| 7 | LRKWGDKIRLVNCDDK | 325 | 0.85 |
| 7 | GRLVYSTCSLNPIENE | 303 | 0.85 |
| 8 | RNATGEPTRVVYTVCP | 490 | 0.84 |
| 8 | HKDTDEPSGFVVANDA | 188 | 0.84 |
| 9 | GIDANAKATPSAEEKE | 644 | 0.83 |
| 9 | LPVWKGNKCINLMVCK | 617 | 0.83 |
| 9 | SPSEEEASKFNLQNCM | 376 | 0.83 |
| 10 | TESPAETTTGTSTEAP | 665 | 0.82 |
| 10 | ENEKWEKYYKTLALFP | 33 | 0.82 |
| 10 | ARRSHMLVHQLKRLNS | 205 | 0.82 |
| 10 | AKTQRFLVVENAVGNI | 132 | 0.82 |
| 10 | PKTVIRKNEQFAKTQR | 121 | 0.82 |
| 11 | KKTCQAPLPLTFRITG | 56 | 0.81 |
| 12 | FVSQRSDIECSWRIQS | 528 | 0.80 |
| 12 | EKTKGDEGTLDSFFSP | 362 | 0.80 |
| 13 | KDVIQANDDRLKIIYS | 508 | 0.79 |
| 13 | IDNIFDRNTCLVRNAT | 478 | 0.79 |
| 13 | LFPEDQWEEFKKTCQA | 46 | 0.79 |
| 13 | ALESEGPQTKKIKVEE | 428 | 0.79 |
| 13 | FPRIRLHGNSNNKNDV | 233 | 0.79 |
| 13 | PGSKTAQLIEALHKDT | 176 | 0.79 |
| 13 | QEAVSMIPPIVLEVKP | 150 | 0.79 |
| 14 | TFRITGSRKHAGEVLN | 66 | 0.78 |
| 14 | LSSGCAFIDVSRNDPA | 596 | 0.78 |
| 14 | NRIVEANLEMLKHLLI | 556 | 0.78 |
| 14 | EDSTEAATEKLSSETP | 412 | 0.78 |
| 15 | SWRIQSESLPIMKHHM | 538 | 0.77 |
| 15 | PFVFVDPQHEALKVCW | 458 | 0.77 |
| 15 | KLSSETPALESEGPQT | 421 | 0.77 |
| 16 | HEALKVCWDFYGIDNI | 466 | 0.76 |
| 16 | FVVANDADARRSHMLV | 197 | 0.76 |

| Start | End | Max_score_pos | Sequence |
|---|---|---|---|
| 497 | 513 | 502 | TRVVYTVCPALKDVIQA |
| 248 | 262 | 257 | VLKFDRILCDVPCSG |
| 625 | 633 | 628 | CINLMVCKE |
| 150 | 176 | 160 | QEAVSMIPPIVLEVKPHHTVLDMCAAP |
| 304 | 313 | 308 | RLVYSTCSLN |
| 57 | 68 | 62 | KTCQAPLPLTFR |
| 135 | 143 | 141 | QRFLVVENA |
| 518 | 532 | 528 | LKIIYSGVKLFVSQR |
| 317 | 324 | 322 | NEAVVAEA |
| 457 | 475 | 469 | EPFVFVDPQHEALKVCWDF |
| 597 | 605 | 603 | SSGCAFIDV |
| 281 | 291 | 285 | GLGLHAVQLNI |
| 208 | 218 | 212 | SHMLVHQLKRL |
| 195 | 202 | 197 | SGFVVAND |
| 613 | 620 | 618 | ENLFLPVW |
| 347 | 358 | 353 | SKGVSKWPVYDR |
| 390 | 397 | 393 | CMRVYPHQ |
| 566 | 574 | 569 | LKHLLIESF |
| 102 | 124 | 121 | KAPVELPWYPDHLAWQLDVPKTV |
| 332 | 345 | 334 | IRLVNCDDKLPGLI |
| 41 | 49 | 43 | YKTLALFPE |
| 404 | 410 | 408 | FITVFEK |
| 222 | 237 | 223 | NLMVVNHDAQFFPRIR |
| 180 | 187 | 185 | TAQLIEAL |
| 484 | 491 | 489 | RNTCLVRN |
| 636 | 644 | 641 | HELLYRIFG |
| 438 | 444 | 442 | KIKVEEV |
| 294 | 299 | 297 | RGLHLL |
| 76 | 83 | 81 | AGEVLNLF |
| 543 | 550 | 549 | SESLPIMK |
| 88 | 94 | 91 | LPNLTNV |
| 534 | 540 | 540 | DIECSWR |
| 424 | 429 | 425 | SETPAL |

72. Aspergillus fumigatus CAF32099
MATFARPVASSISGIEFGVYSDEDIKSISVKRIHNTPTLDSFNNPVPGGLYDPALGAWGDHLSLVIAYFGPF
WLTAFSCTTCRQNSWSCTGHPGHIELPVRVYNVTFFDQLYRLLRAQCVYCHRFQMARVQINAYVCKLRLLQY
GLVDEVEAIEAMGTGQGNKKKSAKDADDSGSEEEDDDDLVARRNAYVKKVIREAHAAGRLKGIMSGAKNPMA
AEQRRTLVKQFFKDLVSIKKCSSCSGYRRDRFSKIFRKPLPEKSRLAMVQAGFQAPNSLILLQQAKKFDMKT
KESMANGISDTANAVSESHGAEEEVARGNAVVAQAESKKSAAGDAGQYMPSPEVHAAMVLLFEKEKEILSLI
YNSRPLPKKEAKVSPDMFFIKNILVPPNKYRPAAPQGPGEIMEAQQNTPFTQILKNCDIINQISKERQNAGA
DSVTRMRDYRDLLHAIVQLQDTVNGLIDKERGASGPAAGQAANGIKQILEKKEGLFRKNMMGKRVNFAARSV
ISPDPNIETNEIGVPLVFAKKLTYPEPVTNHNFWEMKQAVINGPDKYPGATAIENELGQVTNLKFKSLDERT
ALANQLLAPSNWRMKGARNKKVYRHLTTGDVVLMNRQPTLHKPSIMGHKARVLANERVIRMHYANCNTYNAD
FDGDEMNMHFPQNELARAEAMMLADADHQYLVATSGKPLRGLIQDHISMGTWFTCRDTFFDEEDYHELLYSC
LRPENSHTVTERIQLVEPTLLKPKRLWTGKQVITTILKNIMPPGRAGLNLKSKSSTPGDRWGEGNEEGTVIF
KDGEMLCGILDKKQIGPTAGGLIDAIHEVYGHTIAGRLISILGRLLTRYLNMRAFTCGIDDLRLTKEGDRLR
KEKLSQAASIGREVALKYVTLDQTTVPDQDAELRRRLEDVLRDDDKQSGLDSVSNARTAKLSTEITQACLPK
GLAKPFPWNQMQSMTISGAKGSSVNANLISCNLGQQVLEGRRVPVMISGKTLPSFRAFDTHPMAGGYVCGRF
LTGIKPQEYYFHAMAGREGLIDTAVKTSRSGYLQRCLIKGMEGLRAEYDTSVRESSDGSIVQFLYGEDGLDI
TKQVHLKDFDFLTSNYVSIMQSVNLTSDFHNLEKDEVTAWHKDAMKKVRKTGKVDAMDPVLSVYHPGGNLGS
TSEAFSQALKKYEDTNPDKLLRDKKKNIDGLISKKAFNTLMNMKYLKSVVDPGEAVGIVASQSIGEPSTQMT
LNTFHLAGHSAKNVTLGIPRLREIVMTASAHIMTPTMTLILNEELSKEHSERFAKAISKLSIAEVIDKVKVK
ERIATAGSRFKVYDVEIALFPAEEYTKEYAITTKDVQNTLQNKFIPKLVKLTRAELKRRNDEKSLKSFSTAQ
PEIGVSVGVIEEGPRGPDREVEPAQDDDEDDEDDAKRARSGQNRSNQVSYEGPEQEEIDMVRQQDAVEDDED
EDESGEDRRQDSDVDMDDSDEETDEETKDTKLREEDIKGKYGEVTQFKFNPSKGTSCVVQLQYDISTPKLLL
LPLVEEAARSAVIQSIPGLGNCTYVEADPVKGEPAHVITEGVNLLAMRDYQDIIKPHSIYTNSIHHMLMLYG
VEAARASIVREMSDVFQGHSISVDNRHLNLIGDVMTQSGGFRAFNRNGLVKDSSSPLAKMSFETTVGFLKDA
VIERDFDNLKSPSSRIVAGRSGMVGTGAFDVLAPVA

| Rank | Sequence | Start position | Score |
|---|---|---|---|
| 1 | QDHISMGTWFTCRDTF | 692 | 0.96 |
| 2 | GVIEEGPRGPDREVEP | 1376 | 0.95 |
| 2 | EYAITTKDVQNTLQNK | 1324 | 0.95 |
| 3 | VEPAQDDDEDDEDDAK | 1389 | 0.93 |
| 3 | HRFQMARVQINAYVCK | 123 | 0.93 |
| 4 | PAAPQGPGEIMEAQQN | 392 | 0.92 |
| 4 | VKRIHNTPTLDSFNNP | 30 | 0.92 |
| 4 | GSEEEDDDDLVARRNA | 174 | 0.92 |
| 4 | MVRQQDAVEDDEDEDE | 1428 | 0.92 |
| 4 | SQSIGEPSTQMTLNTF | 1213 | 0.92 |
| 5 | ISGIEFGVYSDEDIKS | 12 | 0.91 |
| 5 | EDTNPDKLLRDKKKNI | 1165 | 0.91 |
| 6 | ERVIRMHYANCNTYNA | 632 | 0.90 |
| 6 | HKPSIMGHKARVLANE | 617 | 0.90 |
| 7 | STEITQACLPKGLAKP | 926 | 0.89 |
| 7 | QNSWSCTGHPGHIELP | 84 | 0.89 |
| 7 | TILKNIMPPGRAGLNL | 755 | 0.89 |
| 7 | TNEIGVPLVFAKKLTY | 513 | 0.89 |
| 7 | PGEIMEAQQNTPFTQI | 398 | 0.89 |
| 7 | ASAHIMTPTMTLILNE | 1252 | 0.89 |
| 7 | EVTAWHKDAMKKVRKT | 1116 | 0.89 |
| 8 | PSNWRMKGARNKKVYR | 585 | 0.88 |
| 9 | PVMISGKTLPSFRAFD | 980 | 0.87 |
| 9 | HPGHIELPVPVYNVTF | 92 | 0.87 |
| 9 | TVTERIQLVEPTLLKP | 728 | 0.87 |
| 9 | GIMSGAKNPMAAEQRR | 206 | 0.87 |
| 9 | NGLVKDSSSPLAKMSF | 1631 | 0.87 |
| 9 | AIEAMGTGQGNKKKSA | 152 | 0.87 |
| 10 | DEDIKSISVKRIHNTP | 22 | 0.86 |
| 10 | AVIQSIPGLGNCTYVE | 1523 | 0.86 |
| 10 | LKSVVDPGEAVGIVAS | 1198 | 0.86 |
| 11 | HPMAGGYVCGRFLTGI | 997 | 0.85 |
| 11 | NMRAFTCGIDDLRLTK | 843 | 0.85 |
| 11 | KQAVINGPDKYPGATA | 541 | 0.85 |
| 11 | PDPNIETNEIGVPLVF | 507 | 0.85 |
| 11 | KEGLFRKNMMGKRVNF | 484 | 0.85 |
| 11 | KCSSCSGYRRDRFSKI | 236 | 0.85 |
| 11 | HHMLMLYGVEAARASI | 1577 | 0.85 |
| 11 | PVLSVYHPGGNLGSTS | 1139 | 0.85 |
| 11 | QSVNLTSDFHNLEKDE | 1101 | 0.85 |
| 11 | RCLIKGMEGLRAEYDT | 1043 | 0.85 |
| 12 | SMTISGAKGSSVNANL | 949 | 0.84 |
| 12 | YVTLDQTTVPDQDAEL | 882 | 0.84 |
| 12 | AGGLIDAIHEVYGHTI | 811 | 0.84 |
| 12 | GFLKDAVIERDFDNLK | 1651 | 0.84 |
| 12 | EELSKEHSERFAKAIS | 1267 | 0.84 |
| 13 | TAFSCTTCRQNSWSCT | 75 | 0.83 |
| 13 | PGGLYDPALGAWGDHL | 47 | 0.83 |
| 13 | AHVITEGVNLLAMRDY | 1547 | 0.83 |
| 13 | DVDMDDSDEETDEETK | 1453 | 0.83 |
| 14 | KSKSSTPGDRWGEGNE | 771 | 0.82 |
| 14 | HGAEEEVARGNAVVAQ | 307 | 0.82 |
| 14 | KKSAKDADDSGSEEED | 164 | 0.82 |
| 14 | EDESGEDRRQDSDVDM | 1441 | 0.82 |
| 14 | TGIKPQEYYFHAMAGR | 1010 | 0.82 |
| 15 | ARTAKLSTEITQACLP | 920 | 0.81 |
| 15 | FAKKLTYPEPVTNHNF | 522 | 0.81 |
| 15 | TRMRDYRDLLHAIVQL | 436 | 0.81 |
| 15 | IINQISKERQNAGADS | 419 | 0.81 |
| 15 | TLDSFNNPVPGGLYDP | 38 | 0.81 |
| 15 | MFFIKNILVPPNKYRP | 377 | 0.81 |
| 15 | TQSGGFRAFNRNGLVK | 1620 | 0.81 |
| 15 | LFPAEEYTKEYAITTK | 1315 | 0.81 |
| 15 | KERIATAGSRFKVYDV | 1296 | 0.81 |
| 15 | PSTQMTLNTFHLAGHS | 1219 | 0.81 |
| 16 | YNADFDGDEMNMHFPQ | 645 | 0.80 |
| 16 | KKVIREAHAAGRLKGI | 192 | 0.80 |
| 16 | SSRIVAGRSGMVGTGA | 1669 | 0.80 |
| 16 | QVSYEGPEQEEIDMVR | 1415 | 0.80 |
| 16 | EDDEDDAKRARSGQNR | 1397 | 0.80 |
| 16 | DGSIVQFLYGEDGLDI | 1065 | 0.80 |
| 16 | YFHAMAGREGLIDTAV | 1018 | 0.80 |
| 17 | GRLLTRYLNMRAFTCG | 835 | 0.79 |
| 17 | KKVYRHLTTGDVVLMN | 596 | 0.79 |
| 17 | ARSVISPDPNIETNEI | 501 | 0.79 |
| 17 | DVFQGHSISVDNRHLN | 1598 | 0.79 |
| 17 | PVKGEPAHVITEGVNL | 1541 | 0.79 |
| 17 | CTYVEADPVKGEPAHV | 1534 | 0.79 |
| 17 | DRRQDSDVDMDDSDEE | 1447 | 0.79 |
| 17 | AQCVYCHRFQMARVQI | 117 | 0.79 |
| 17 | TGKVDAMDPVLSVYHP | 1131 | 0.79 |
| 18 | RPENSHTVTERIQLVE | 722 | 0.78 |
| 18 | TKESMANGISDTANAV | 288 | 0.78 |
| 18 | PKLVKLTRAELKRRND | 1342 | 0.78 |
| 19 | REVALKYVTLDQTTVP | 876 | 0.77 |
| 19 | DKKQIGPTAGGLIDAI | 803 | 0.77 |
| 19 | GTVIFKDGEMLCGILD | 788 | 0.77 |
| 19 | GDRWGEGNEEGTVIFK | 778 | 0.77 |
| 19 | HELLYSCLRPENSHTV | 714 | 0.77 |
| 19 | AGQYMPSPEVHAAMVL | 333 | 0.77 |
| 19 | SDEETDEETKDTKLRE | 1459 | 0.77 |
| 20 | DVLRDDDKQSGLDSVS | 903 | 0.76 |
| 20 | HEVYGHTIAGRLISIL | 819 | 0.76 |
| 20 | KSLDERTALANQLLAP | 570 | 0.76 |
| 20 | IKQILEKKEGLFRKNM | 477 | 0.76 |
| 20 | MRDYQDIIKPHSIYTN | 1559 | 0.76 |
| 20 | VQLQYDISTPKLLLLP | 1499 | 0.76 |
| 20 | HPGGNLGSTSEAFSQA | 1145 | 0.76 |
| 21 | SGLDSVSNARTAKLST | 912 | 0.75 |
| 21 | DAELRRRLEDVLRDDD | 894 | 0.75 |
| 21 | GAWGDHLSLVIAYFGP | 56 | 0.75 |
| 21 | FSKIFRKPLPEKSRLA | 248 | 0.75 |
| 21 | AEVIDKVKVKERIATA | 1287 | 0.75 |
| 21 | RAEYDTSVRESSDGSI | 1053 | 0.75 |

| Start | End | Max_score_pos | Sequence |
|---|---|---|---|
| 1494 | 1519 | 1513 | GTSCVVQLQYDISTPKLLLLPLVEEA |
| 89 | 125 | 120 | CTGHPGHIELPVRVYNVTFFDQLYRLLRAQCVYCHRF |
| 1137 | 1147 | 1142 | MDPVLSVYHPG |
| 127 | 153 | 139 | MARVQINAYVCKLRLLQYGLVDEVEAI |
| 60 | 82 | 65 | DHLSLVIAYFGPFWLTAFSCTTC |
| 442 | 458 | 447 | RDLLHAIVQLQDTVNGL |
| 714 | 723 | 719 | HELLYSCLRP |
| 516 | 532 | 519 | IGVPLVFAKKLTYPEPV |
| 1038 | 1047 | 1043 | SGYLQRCLIK |
| 1682 | 1689 | 1688 | TGAFDVLA |
| 875 | 893 | 881 | GREVALKYVTLDQTTVPDQ |
| 339 | 351 | 345 | SPEVHAAMVLLFE |
| 221 | 243 | 234 | RTLVKQFFKDLVSIKKCSSCSGY |
| 1066 | 1073 | 1070 | GSIVQFLY |
| 1370 | 1380 | 1374 | EIGVSVGVIEE |
| 1198 | 1205 | 1199 | LKSVVDPG |
| 1001 | 1012 | 1006 | GGYVCGRFLTGI |
| 1278 | 1298 | 1292 | AKAISKLSIAEVIDKVKVKER |
| 182 | 198 | 192 | DLVARRNAYVKKVIREA |
| 272 | 281 | 278 | PNSLILLQQA |
| 1341 | 1350 | 1344 | IPKLVKLTRA |
| 729 | 744 | 738 | VTERIQLVEPTLLKPK |
| 1207 | 1214 | 1210 | AVGIVASQ |
| 673 | 684 | 679 | DADHQYLVATSG |
| 930 | 941 | 932 | TQACLPKGLAKP |
| 1305 | 1318 | 1311 | RFKVYDVEIALFPA |
| 1521 | 1557 | 1526 | RSAVIQSIPGLGNCTYVEADPVKGEPAHVITEGVNLL |
| 356 | 364 | 358 | ILSLIYNSR |
| 316 | 323 | 321 | GNAVVAQA |
| 1093 | 1109 | 1100 | TSNYVSIMQSVNLTSDF |
| 749 | 757 | 754 | GKQVITTIL |
| 1081 | 1091 | 1083 | TKQVHLKDFDF |
| 1648 | 1660 | 1655 | TTVGFLKDAVIER |
| 380 | 388 | 382 | IKNILVPPN |
| 798 | 805 | 801 | LCGILDKK |
| 961 | 984 | 967 | NANLISCNLGQQVLEGRRVPVMIS |
| 5 | 22 | 10 | ARPVASSISGIEFGVYSD |
| 47 | 56 | 48 | PGGLYDPALG |
| 579 | 585 | 580 | ANQLLAP |
| 625 | 632 | 627 | KARVLANE |
| 827 | 841 | 835 | AGRLISILGRLLTRY |
| 814 | 825 | 824 | LIDAIHEVYGHT |
| 1577 | 1594 | 1585 | HHMLMLYGVEAARASIVR |
| 498 | 508 | 502 | NFAARSVISPD |
| 1598 | 1610 | 1602 | DVFQGHSISVDNR |
| 27 | 33 | 31 | SISVKRI |
| 1563 | 1575 | 1569 | QDIIKPHSIYTNS |
| 604 | 611 | 605 | TGDVVLMN |
| 868 | 873 | 871 | LSQAAS |
| 1667 | 1675 | 1671 | SPSSRIVAG |
| 1030 | 1035 | 1031 | DTAVKT |
| 687 | 694 | 693 | LRGLIQDH |
| 1612 | 1618 | 1615 | LNLIGDV |
| 411 | 422 | 417 | TQILKNCDIINQ |
| 262 | 269 | 268 | LAMVQAGF |
| 1235 | 1243 | 1241 | AKNVTLGIP |
| 1227 | 1233 | 1231 | TFHLAGH |
| 1014 | 1021 | 1020 | PQEYYFHA |
| 1634 | 1644 | 1636 | VKDSSSPLAKM |
| 561 | 567 | 564 | LGQVTNL |
| 1245 | 1257 | 1246 | LREIVMTASAHIM |
| 986 | 992 | 991 | KTLPSFR |
| 1181 | 1186 | 1186 | DGLISK |
| 1157 | 1163 | 1159 | FSQALKK |
| 249 | 255 | 254 | SKIFRKP |
| 1261 | 1267 | 1266 | MTLILNE |
| 847 | 854 | 848 | FTCGIDDL |
| 368 | 378 | 376 | KKEAKVSPDMF |
| 542 | 547 | 545 | QAVING |
| 300 | 306 | 305 | ANAVSES |
| 1361 | 1368 | 1364 | LKSFSTAQ |

73. Aspergillus nidulans AN7465.2
MFYSETLLSKTGPLARVWLSANLERKLSKSHILQSDIESSVSAIVDQGQAPMALRLSGQLLLGVVRIYSRKA
RYLLDDCNEALMKIKMAFRLTNNNDLTTSAVVAPGGITLPDVLTEADLFMNLDSSLLIPQPLSLEPEGKRPG
PSMDFGSQLFPDTGLRRSASQEPALLEDPGDLQLNLGLDDETNLSFSHDFSMEVGRDAPAPRPMEEDNFSDA
GKVIDVGDLGLNLGEDDTPLDAVNFDANEDNFLPLDEPMDLGDDTVVADGNDERFERESTLTEVSEDMIERL
NTEHEGDYMHDEEQDDETIQHAQRAKRRKQLPTIELDEAVEFKGNSYFRIQQEQLSETLKPASFLPRDPVLL
TLMNMQKNGDFVSNVMGGGRGRGWAPEIRDLLSFDTVRKAGELKRKRDSGISDMDVDAAAAPALEIEEEAIV
PVDEGVGMESTLHQRSEIDFPGDEEDHLRLSDDEGAQQPLEDFDDTITPVDSALVSVGTKRAVHVLRDCLGN
AEQKKAVKFQDLLPEKKATRADATKMFFEVLVLATKDAVQVEQRSNTVGGPIKISGKRSLWGQWAEEDATGE
VSQAQVAA

| Rank | Sequence | Start position | Score |
|---|---|---|---|
| 1 | EGAQQPLEDFDDTITP | 466 | 0.91 |
| 1 | DDTPLDAVNFDANEDN | 232 | 0.91 |
| 2 | RDSGISDMDVDAAAAP | 407 | 0.90 |
| 2 | DETIQHAQRAKRRKQL | 304 | 0.90 |
| 3 | APEIRDLLSFDTVRKA | 385 | 0.89 |
| 3 | GRDAPAPRPMEEDNFS | 199 | 0.89 |
| 4 | TVVADGNDERFEREST | 261 | 0.88 |
| 5 | PRPMEEDNFSDAGKVI | 205 | 0.87 |
| 5 | PDVLTEADLFMNLDSS | 112 | 0.87 |
| 6 | FVSNVMGGGRGRGWAP | 371 | 0.86 |
| 7 | RSEIDFPGDEEDHLRL | 447 | 0.85 |
| 7 | IVPVDEGVGMESTLHQ | 431 | 0.85 |
| 7 | TIELDEAVEFKGNSYF | 321 | 0.85 |
| 7 | TEHEGDYMHDEEQDDE | 290 | 0.85 |
| 7 | MDLGDDTVVADGNDER | 255 | 0.85 |
| 7 | SDAGKVIDVGDLGLNL | 214 | 0.85 |
| 7 | QLFPDTGLRRSASQEP | 152 | 0.85 |
| 7 | PEGKRPGPSMDFGSQL | 138 | 0.85 |
| 8 | GVVRIYSRKARYLLDD | 63 | 0.83 |
| 9 | AVHVLRDCLGNAEQKK | 494 | 0.82 |
| 9 | ARVWLSANLERKLSKS | 15 | 0.82 |
| 10 | KIKMAFRLTNNNDLTT | 85 | 0.81 |
| 10 | VGMESTLHQRSEIDFP | 438 | 0.81 |
| 10 | RKAGELKRKRDSGISD | 398 | 0.81 |
| 11 | SETLLSKTGPLARVWL | 4 | 0.80 |
| 12 | CNEALMKIKMAFRLTN | 79 | 0.78 |
| 12 | QWAEEDATGEVSQAQV | 567 | 0.78 |
| 13 | QVEQRSNTVGGPIKIS | 544 | 0.77 |
| 13 | NAEQKKAVKFQDLLPE | 504 | 0.77 |
| 13 | STLTEVSEDMIERLNT | 275 | 0.77 |
| 14 | GPIKISGKRSLWGQWA | 554 | 0.76 |
| 14 | SAIVDQGQAPMALRLS | 42 | 0.76 |
| 14 | GLRRSASQEPALLEDP | 158 | 0.76 |
| 15 | VSVGTKRAVHVLRDCL | 487 | 0.75 |
| 15 | DEEDHLRLSDDEGAQQ | 455 | 0.75 |

| Start | End | Max_score_pos | Sequence |
|---|---|---|---|
| 51 | 82 | 62 | PMALRLSGQLLLGVVRIYSRKARYLLDDCNEA |
| 531 | 546 | 534 | FFEVLVLATKDAVQVE |
| 479 | 504 | 498 | ITPVDSALVSVGTKRAVHVLRDCLGN |
| 126 | 137 | 131 | SSLLIPQPLSLE |
| 38 | 48 | 42 | ESSVSAIVDQG |
| 344 | 362 | 359 | SETLKPASFLPRDPVLLTL |
| 576 | 581 | 580 | EVSQAQ |
| 100 | 109 | 101 | TSAVVAPGGI |
| 111 | 118 | 112 | LPDVLTEA |
| 429 | 437 | 435 | EAIVPVDEG |
| 508 | 519 | 514 | KKAVKFQDLLPE |
| 14 | 22 | 16 | LARVWLSAN |
| 216 | 228 | 222 | AGKVIDVGDLGLN |
| 388 | 397 | 394 | IRDLLSFDTV |
| 28 | 36 | 30 | SKSHILQSD |
| 175 | 181 | 179 | DLQLNLG |
| 417 | 425 | 421 | DAAAAPALE |
| 259 | 265 | 260 | DDTVVAD |
| 4 | 12 | 5 | SETLLSKTG |
| 165 | 173 | 167 | QEPALLEDP |
| 236 | 241 | 238 | LDAVNF |
| 553 | 558 | 555 | GGPIKI |
| 151 | 156 | 152 | SQLFPD |
| 336 | 342 | 337 | FRIQQEQ |

74. Aspergillus nidulans AN4541.2
MSLHARLRPLPRRLATQPPSESAAPTPHFEDPPDGANAEDDAEDLFSSFLPHLFPDDAPQFHGDPGQYLLYS
SPRYGELQIMVPSYPSQSQSGARSKEIAEGLPRSDGQVNQVEEGRKLFAHFLWSAAMVVAEGLEQADTESGG
SEAEFWKVQNEKVLELGAGAGLPSIVSALANASMVTITDHPSSPALGPAGAIASNVKHNLSSSTSIVDIRPH
EWGTTLTTDPWALSNKGSYTRIIAADCYWMRSQHENLVRTMKWFLAPEGKIWVVAGFHTGREIVAGFFETAV
SLGLKIESIYERDLNSSAEEGGEVRRAWVSFREGEGPENRRRWCVVAVLGHAPAAAGTGADA

| Rank | Sequence | Start position | Score |
|---|---|---|---|
| 1 | GEVRRAWVSFREGEGP | 310 | 0.94 |
| 2 | PHEWGTTLTTDPWALS | 215 | 0.91 |
| 2 | PPSESAAPTPHFEDPP | 18 | 0.91 |
| 3 | TPHFEDPPDGANAEDD | 26 | 0.90 |
| 4 | TESGGSEAEFWKVQNE | 140 | 0.89 |
| 5 | KEIAEGLPRSDGQVNQ | 97 | 0.88 |
| 5 | GLKIESIYERDLNSSA | 291 | 0.88 |
| 5 | MVTITDHPSSPALGPA | 178 | 0.88 |
| 6 | STSIVDIRPHEWGTTL | 207 | 0.87 |
| 6 | EGLEQADTESGGSEAE | 133 | 0.87 |
| 7 | SFREGEGPENRRRWCV | 318 | 0.85 |
| 7 | SSAEEGGEVRRAWVSF | 304 | 0.85 |
| 8 | AVLGHAPAAAGTGADA | 335 | 0.83 |
| 9 | HPSSPALGPAGAIASN | 184 | 0.82 |
| 9 | LPSIVSALANASMVTI | 166 | 0.82 |
| 9 | HFLWSAAMVVAEGLEQ | 122 | 0.82 |
| 10 | PQFHGDPGQYLLYSSP | 59 | 0.80 |
| 11 | IMVPSYPSQSQSGARS | 81 | 0.78 |
| 11 | DCYWMRSQHENLVRTM | 242 | 0.78 |
| 11 | AGAIASNVKHNLSSST | 193 | 0.78 |
| 12 | HLFPDDAPQFHGDPGQ | 52 | 0.77 |
| 13 | LHARLRPLFRRLATQP | 3 | 0.76 |
| 13 | TMKWFLAPEGKIWVVA | 256 | 0.76 |
| 14 | QYLLYSSPRYGELQIM | 67 | 0.75 |
| 14 | TGREIVAGFFETAVSL | 275 | 0.75 |
| 14 | RRLATQPPSESAAPTP | 12 | 0.75 |

| Start | End | Max_score_pos | Sequence |
|---|---|---|---|
| 330 | 343 | 334 | RWCVVAVLGHAPAA |
| 163 | 205 | 169 | GAGLPSIVSALANASMVTITDHPSSPALGPAGAIASN |
| | | | VKHNLS |
| 44 | 62 | 52 | DLFSSFLPHLFPDDAPQFH |
| 66 | 74 | 71 | GQYLLYSSP |
| 236 | 246 | 241 | TRIIAADCYWM |
| 285 | 297 | 291 | ETAVSLGLKIESI |
| 266 | 273 | 270 | KIWVVAGF |
| 119 | 134 | 129 | LFAHFLWSAAMVVAEG |
| 76 | 91 | 86 | YGELQIMVPSYPSQSQ |
| 154 | 161 | 159 | NEKVLELG |
| 207 | 214 | 213 | STSIVDIR |
| 277 | 283 | 281 | REIVAGF |
| 4 | 17 | 6 | HARLRPLPRRLATQ |

75. Aspergillus nidulans AN3628.2
MPQQLSSKDASLFRQVVRHYENKQYKKGIKTADQVLRKNPNHGDTLAMKALIMSNQGEQQEAFALAKEALKN
DMKSHICWHVYGLLYRAEKNYEEAIKAYRFALRIEPDSQPIQRDLALLQMQMRDYQGYIQSRSTMLQAPGF
RQNWTALAIAHHLSGDLEEAEKVLTTYEETLKTPPPLSDMEHSEATLYKNMIIAESGNIQKALEHLESVGHR
CSDVLAVMEMKADYLLRLDKKEEAAAAYTALLERNSENSLYYDGLIKAKGISSDDHKALKALYDSWAEKYPR
GDAPRRIPLDFLEGDDFKQAADAYLQRMLKKGVPSLFANIKLLYTNSSKRDTVQELVEGYVSNPPANGAADG
SENTEFLSSAYYFLAQHYNYHLSRDLSKALQNVDKALELSPKAVEYQMTKARIWKHYGNLEKAAEEMENARK
MDEKDRHINSKAAKYQLRNNNNDKALDKMSKFTRNETVGGALGDLHEMQCVWYLTEDGEAYLRQKKLGLALK
RFHAVYNIFDVWHEDQFDFHSFSLRKGMIRAYVDMVRWEDRLREHPFYTRAALSAIKAYILLHDQPDLAHGP
LPEINGADGDDAERKKALKKAKKEQQRLEKLEQEKREAARKAAANPKSLDGEVKKEDPDPLGNKLAQTQEPL
KEALKFLTPLLEHSPKNIEAQCLGFEVHLRRGKYALALKCLAAAHSIDASNPTLHVQLLQFRQALNKLYEPL
PPQVAEVVDSEFEALLPKAQNLEEWNKSFLSAHKDSIPHKYAYLTCQQLLKPESKSENEKELAATLDAGIMS
LETALAGLDLLGEWGSDKAAKTAYAEKASSKWPESTAFRVN

| Rank | Sequence | Start position | Score |
|---|---|---|---|
| 1 | KGMIRAYVDMVRWEDR | 530 | 0.94 |
| 2 | STMLQARPGFRQNWTA | 135 | 0.92 |
| 3 | LPEINGADGDDAERKK | 577 | 0.89 |
| 3 | DSWAEKYPRGDAPRRI | 280 | 0.89 |
| 3 | HHLSGDLEEAEKVLTT | 155 | 0.89 |
| 4 | HKALKALYDSWAEKYP | 272 | 0.88 |
| 5 | EGYVSNPPANGAADGS | 346 | 0.86 |
| 5 | DAPRRIPLDFLEGDDF | 290 | 0.86 |
| 5 | YKKGIKTADQVLRKNP | 25 | 0.86 |
| 6 | AQTQEPLKEALKFLTP | 642 | 0.85 |
| 6 | MVRWEDRLREHPFYTR | 539 | 0.85 |
| 6 | KARIWKHYGNLEKAAE | 410 | 0.85 |
| 6 | AQHYNYHLSRDLSKAL | 375 | 0.85 |
| 6 | RDTVQELVEGYVSNPP | 338 | 0.85 |
| 7 | YEEAIKAYRFALRIEP | 93 | 0.84 |
| 7 | AAAAYTALLERNSENS | 240 | 0.84 |
| 7 | MRDYQGYIQSRSTMLQ | 124 | 0.84 |
| 8 | KSHICWHVYGLLYRAE | 75 | 0.83 |
| 8 | EVKKEDPDPLGNKLAQ | 628 | 0.83 |
| 8 | NSKAAKYQLRNNNNDK | 441 | 0.83 |
| 8 | QVLRKNPNHGDTLAMK | 34 | 0.83 |
| 8 | GISSDDHKALKALYDS | 266 | 0.83 |
| 8 | PLSDMEHSEATLYKNM | 180 | 0.83 |
| 9 | DAGIMSLETALAGLDL | 787 | 0.82 |
| 9 | SAHKDSIPHKYAYLTC | 751 | 0.82 |
| 9 | KKLGLALKRFHAVYNI | 497 | 0.82 |
| 9 | ARKMDEKDRHINSKAA | 430 | 0.82 |
| 9 | AEKVLTTYEETLKTPP | 164 | 0.82 |
| 10 | IPHKYAYLTCQQLLKP | 757 | 0.81 |
| 11 | PDLAHGPLPEINGADG | 570 | 0.80 |
| 11 | QGEQQEAFALAKEALK | 56 | 0.80 |
| 11 | GNLEKAAEEMENARKM | 418 | 0.80 |
| 11 | AVEYQMTKARIWKHYG | 403 | 0.80 |
| 12 | TEDGEAYLRQKKLGLA | 487 | 0.79 |
| 12 | GGALGDLHEMQCVWYL | 471 | 0.79 |
| 12 | SGNIQKALEHLESVGH | 200 | 0.79 |
| 12 | ETLKTPPPLSDMEHSE | 173 | 0.79 |
| 13 | WGSDKAAKTAYAEKAS | 806 | 0.78 |
| 13 | VDSEFEALLPKAQNLE | 728 | 0.78 |
| 13 | QCVWYLTEDGEAYLRQ | 481 | 0.78 |
| 13 | ALRIEPDSQPIQRDLA | 103 | 0.78 |
| 14 | KAYILLHDQPDLAHGP | 561 | 0.76 |
| 14 | TEFLSSAYYFLAQHYN | 364 | 0.76 |
| 14 | HLESVGHRCSDVLAVM | 209 | 0.76 |
| 15 | QEKREAARKAAANPKS | 609 | 0.75 |

| Start | End | Max_score_pos | Sequence |
|---|---|---|---|
| 204 | 224 | 220 | QKALEHLESVGHRCSDVLAVM |
| 700 | 711 | 705 | PTLHVQLLQFRQ |
| 667 | 695 | 686 | EAQCLGFEVHLRRGKYALALKCLAAAHSI |
| 76 | 88 | 80 | SHICWHVYGLLYR |
| 713 | 731 | 725 | LNKLYEPLPPQVAEVVDSE |
| 749 | 773 | 767 | FLSAHKDSIPHKYAYLTCQQLLKPE |
| 479 | 486 | 484 | EMQCVWYL |
| 4 | 20 | 14 | QLSSKDASLFRQVVRHY |
| 150 | 159 | 154 | ALAIAHHLSG |
| 548 | 580 | 563 | EHPFYTRAALSAIKAYILLHDQPDLAHGPLPEI |
| 341 | 352 | 347 | VQELVEGYVSNP |
| 367 | 407 | 374 | LSSAYYFLAQHYNYHLSRDLSKALQNVDKALELSPKAVEYQ |
| 493 | 516 | 512 | YLRQKKLGLALKRFHAVYNIFDVW |
| 793 | 803 | 800 | LETALAGLDLL |
| 241 | 247 | 245 | AAAYTAL |
| 648 | 661 | 655 | LKEALKFLTPLLEH |
| 318 | 334 | 323 | KKGVPSLFANIKLLYTN |
| 229 | 234 | 231 | DYLLRL |
| 255 | 265 | 259 | SLYYDGLIKAK |
| 114 | 122 | 120 | QRDLALLQM |
| 272 | 281 | 276 | HKALKALYDS |
| 733 | 739 | 737 | EALLPKA |
| 534 | 540 | 537 | RAYVDMV |
| 294 | 300 | 297 | RIPLDFL |
| 48 | 53 | 49 | MKALIM |
| 308 | 316 | 310 | AADAYLQRM |
| 62 | 68 | 67 | AFALAKE |
| 96 | 107 | 103 | AIKAYRFALRIE |
| 522 | 528 | 526 | DFHSFSL |
| 32 | 38 | 33 | ADQVLRK |
| 164 | 170 | 168 | AEKVLTT |
| 128 | 133 | 130 | QGYIQS |
| 471 | 477 | 475 | GGALGDL |

76. Aspergillus nidulans EAA58968
MARTQKNKNTSYHLGQLKAKLAKLKRELLTPSGGGGGGSGAGFDVARTGVASVGFIGFPSVGKSTLMSRLTG
QHSEAAAYEFTTLTTVPGQVLYNGAKIQILDLPGIIQGAKDGKGRGRQVIAVAKTCHLIFIVLDVNKPLVDK
KVIENELEGFGIRINKQPPNIMFKKKDKGGISITSTVPLTHIDNDEIKAVMSEYKISSADISIRCDATIDDL
IDVLEAKSRAYIPVVYALNKIDAITIEELDLLYRIPNAVPISSEHGWNIDELLEMMWEKLNLRRIYTKPKGK
APDYTAPVVLRANACTVEDFCNAIHRTIKDQFKQAIVYGRSVKHQPQRVGLTHELADEDIGSRPFCEAYGTI
GLTV

| Rank | Sequence | Start position | Score |
|---|---|---|---|
| 1 | APDYTAPVVLRANACT | 289 | 0.95 |
| 2 | TPSGGGGGGSGAGFDV | 30 | 0.92 |
| 3 | GWNIDELLEMMWEKLN | 262 | 0.91 |
| 4 | LRRIYTKPKGKAPDYT | 278 | 0.89 |
| 4 | PGIIQGAKDGKGRGRQ | 105 | 0.89 |
| 5 | HELADEDIGSRPFCEA | 341 | 0.88 |
| 6 | EMMWEKLNLRRIYTKP | 270 | 0.85 |
| 6 | KVIENELEGFGIRINK | 145 | 0.85 |
| 7 | GISITSTVPLTHIDND | 174 | 0.84 |
| 8 | QHSEAAAYEFTTLTTV | 73 | 0.82 |
| 9 | TIEELDLLYRIPNAVP | 241 | 0.80 |
| 10 | GAKIQILDLPGIIQGA | 96 | 0.78 |
| 10 | ISSADISIRCDATIDD | 200 | 0.78 |
| 11 | VGFIGFPSVGKSTLMS | 53 | 0.77 |
| 11 | GGSGAGFDVARTGVAS | 37 | 0.77 |
| 12 | QLKAKLAKLKRELLTP | 16 | 0.76 |
| 12 | DGKGRGRQVIAVAKTC | 113 | 0.76 |

| Start | End | Max_score_pos | Sequence |
|---|---|---|---|
| 119 | 145 | 131 | RQVIAVAKTCHLIFIVLDVNKPLVDKK |
| 225 | 237 | 231 | RAYIPVVYALNKI |
| 291 | 314 | 295 | DYTAPVVLRANACTVEDFCNAIHR |
| 85 | 96 | 91 | LTTVPGQVLYNG |
| 42 | 64 | 53 | GFDVARTGVASVGFIGFPSVGKS |
| 321 | 342 | 327 | KQAIVYGRSVKHQPQRVGLTHE |
| 214 | 223 | 219 | DDLIDVLEAK |
| 242 | 259 | 248 | IEELDLLYRIPNAVPISS |
| 177 | 185 | 183 | ITSTVPLTH |
| 99 | 110 | 102 | IQILDLPGIIQG |
| 11 | 31 | 14 | SYHLGQLKAKLAKLKRELLTP |
| 199 | 212 | 206 | KISSADISIRCDAT |
| 351 | 357 | 355 | RPFCEAY |
| 76 | 83 | 77 | EAAAYEFT |
| 191 | 197 | 197 | IKAVMSE |

77. Aspergillus nidulans AN2960.2
MAPSIATSEHVDLRAPIKTLLKTNAGHNKENVIGYGETYKHADELKGTVKQPPASFPHYLPVWDNETERYPP
LQPFEHYDHGKDADPAFPDLFPKDASFHRDDLTPTIGSEVSGIQLSQLSKEGKDQLALFVAQRKVVAFRDQD
FAHLPIDKALEFGGYFGRHHIHQASGAPRGYPEIHLVHRGADDTSGADFLAQHTNSITWHSDVTFEVQPPGT
TFLYLLDGPTTGGDTLFADMAQAYKRLSPEFRKRLHGLKAVHSGVEQVNNSLNKGGIARRDPIMTEHPIVET
HPVTGEKALFVNAQFTRYIVGYKKEESDFLLKFLYDHIALSQDIQTRVRWRPGTVVVWDNRVACHSALFDWA
DGQRRHLARITPQAERPYETPFEG

| Rank | Sequence | Start position | Score |
|---|---|---|---|
| 1 | TNSITWHSDVTFEVQP | 198 | 0.94 |
| 2 | PLQPFEHYDHGKDADP | 72 | 0.93 |
| 3 | RAPIKTLLKTNAGHNK | 14 | 0.91 |
| 4 | SQDIQTRVRWRPGTVV | 329 | 0.90 |
| 5 | APSIATSEHVDLRAPI | 2 | 0.89 |
| 5 | HLPIDKALEFGGYFGR | 147 | 0.89 |
| 6 | RRDPIMTEHPIVRTHP | 275 | 0.88 |
| 6 | LVHRGADDTSGADFLA | 180 | 0.88 |
| 7 | LPVWDNETERYPPLQP | 60 | 0.87 |
| 7 | IGYGETYKHADELKGT | 33 | 0.87 |
| 8 | YDHGKDADPAFPDLFP | 79 | 0.85 |
| 8 | KGTVKQPPASFPHYLP | 46 | 0.85 |
| 8 | RWRPGTVVVWDNRVAC | 337 | 0.85 |
| 8 | YIVGYKKEESDFLLKF | 306 | 0.85 |
| 9 | HLARITPQAERPYETP | 366 | 0.84 |
| 9 | HPVTGEKALFVNAQFT | 289 | 0.84 |
| 9 | VSGIQLSQLSKEGKDQ | 112 | 0.84 |
| 10 | TFEVQPPGTTFLYLLD | 208 | 0.82 |
| 10 | GYPEIHLVHRGADDTS | 174 | 0.82 |
| 11 | DLFPKDASFHRDDLTP | 91 | 0.81 |
| 11 | QAYKRLSPEFRKRLHG | 238 | 0.81 |
| 11 | HQASGAPRGYPEIHLV | 166 | 0.81 |
| 12 | HPIVRTHPVTGEKALF | 283 | 0.80 |
| 13 | NKGGIARRDPIMTEHP | 269 | 0.79 |
| 14 | FDWADGQRRHLARITP | 357 | 0.77 |
| 15 | NRVACHSALFDWADGQ | 348 | 0.75 |
| 15 | DGPTTGGDTLFADMAQ | 223 | 0.75 |
| 15 | TPTIGSEVSGIQLSQL | 105 | 0.75 |

| Start | End | Max_score_pos | Sequence |
|---|---|---|---|
| 341 | 358 | 353 | GTVVVWDNRVACHSALFD |
| 46 | 63 | 59 | KGTVKQPPASFPHYLPVW |
| 127 | 141 | 131 | QLALFVAQRKVVAFR |
| 316 | 331 | 320 | DFLLKFLYDHIALSQD |
| 175 | 184 | 178 | YPEIHLVHRG |
| 282 | 294 | 288 | EHPIVRTHPVTGE |
| 251 | 265 | 254 | LHGLKAVHSGVEQVN |
| 216 | 224 | 219 | TTFLYLLDG |
| 70 | 79 | 73 | YPPLQPFEHY |
| 109 | 120 | 118 | GSEVSGIQLSQL |
| 204 | 214 | 210 | HSDVTFEVQPP |
| 296 | 302 | 300 | ALFVNAQ |
| 4 | 20 | 14 | SIATSEHVDLRAPIKTL |
| 145 | 153 | 147 | FAHLPIDKA |
| 304 | 310 | 309 | TRYIVGY |
| 161 | 169 | 166 | GRHHIHQAS |
| 87 | 97 | 90 | PAFPDLFPKDA |
| 191 | 198 | 194 | ADFLAQHT |
| 365 | 371 | 369 | RHLARIT |
| 240 | 246 | 240 | YKRLSPE |

78. Aspergillus nidulans AN5922.2
MGQYSSTQREHRHQFPTESPLPRQRSHSRNRDEDMNNGQNPERAGAFSPQAGQEIDNNDVQMTHSTETNFLV
DPLQSLISHSSMLGSEEQMGNTQDETGSQDDASQQDYQSALFARVARRHSTMSRLGSRILPNSVIRGLLNSE
EETPAEGHAHRHGVVSRTIPRSEVNQSSARFSPFASLSSRGGSRRRSLRGPYFIPRSDAAINNNGFLGTPSG
PSTDGSAEPGWGWRRSLRIRRVGRVGHSLPTPIAQMFGPPSSDSTPAQDTENPPYSFHNSDPFSFIPHPGPL
DTQMDFDTPHELNSVEPALADSQPASPMLTSQSQSSTRHFPSLLRARPPRALRREEQTPLSRILQLAATAIA
TQLSGGAGPALPNIPSLGNDGFDGSLESFIQSLRNATSGQPSSGDSNNNSEDERPPGPVNFMRVFRFASDN
SRSSDAPNRASTDQNNAVSNGDNMETDHHAEGQEGRTVTLVVIGVRSVPSGNGPAGDQQTAGLPGLDFLRLP
FFPPGTLSPRPGPRPETTTSDHSASSSAPPANVDGSIQPGSPNVPRRLSDVGSRGTLSSLPSVVSESPPGPH
PPPSTPAEPGLSAVSSGASTPSRRLSTTSAVSPNIMHQLNESRPSHPTVDNRDESLPHNTTHQRRRSDSEFA
RHREQLGSGAARRNGVVEPDNHNAAPGRSWLIYVVGTNLSENHPAFAAPSLFTDNPTYEDMVLLSSLLGPVK
PPVATQEDLISAGGLYRVVKCGDSMSAAAVDGTRTIQISEGERCLICLSEYEVAEELRQLTKCEHLYHRDCI
DQHGAFPSSFTISSHLSLFASDIHCCIATFWFWLWGSRKIMKFITYRIKVDYLPTSHTTFITESSKQETKEQ
QMDSIRLTVLISGSGTNLQAVIDDTTLPAKIVRVISNRKDAFGLERARRANIPTQYHNLVKYKKQHPATPEG
VQRAREEYDAELARLVLEDKPDLVACLGFMHVLSEGFLGPLEAKGVRIVNLHPALPGEFNGANAERAHQAW
LDGKIERTGVMIHNVISEVDMGKPILVKEIPFVKGADEDLHAFEQKVHEIEWKVVIEGLQKTIEEIRTTKS

| Rank | Sequence | Start position | Score |
|---|---|---|---|
| 1 | EGVQRAREEYDAELAR | 935 | 0.97 |
| 2 | SHSRNRDEDMNNGQNP | 26 | 0.95 |
| 3 | PGTLSPRPGPRPETTT | 508 | 0.94 |
| 4 | AFSPQAGQEIDNNDVQ | 46 | 0.93 |
| 4 | PPSSDSTPAQDTENPP | 255 | 0.93 |
| 4 | DGKIERTGVMIHNVIS | 1010 | 0.93 |
| 5 | GSEEQMGNTQDETGSQ | 86 | 0.92 |
| 6 | HPTVDNRDESLPHNTT | 622 | 0.91 |
| 6 | HPPPSTPAEPGLSAVS | 576 | 0.91 |
| 6 | VVSESPPGPHPPPSTP | 567 | 0.91 |
| 6 | SRSSDAPNRASTDQNN | 433 | 0.91 |
| 7 | SRKIMKFITYRIKVDY | 829 | 0.90 |
| 7 | AQDTENPPYSFHNSDP | 263 | 0.90 |
| 8 | ACLGFMHVLSEGFLGP | 961 | 0.89 |
| 8 | GQEIDNNDVQMTHSTE | 52 | 0.89 |
| 8 | RGPYFIPRSDAAINNN | 193 | 0.89 |
| 9 | TRTIQISEGERCLICL | 753 | 0.88 |
| 9 | NESRPSHPTVDNRDES | 616 | 0.88 |
| 9 | SIQPGSPNVPRRLSDV | 540 | 0.88 |
| 9 | PGPRPETTTSDHSASS | 515 | 0.88 |
| 9 | NATSGQPSSGDSNNNS | 395 | 0.88 |
| 9 | PAEGHAHRHGVVSRTI | 148 | 0.88 |
| 10 | TGSQDDASQQDYQSAL | 98 | 0.87 |
| 10 | DYLPTSHTTFITESSK | 843 | 0.87 |
| 10 | LDTQMDFDTPHELNSV | 288 | 0.87 |
| 11 | EDLISAGGLYRVVKCG | 727 | 0.86 |
| 11 | SWLIYVVGTNLSENHP | 677 | 0.86 |
| 11 | NMETDHHAEGQEGRTV | 455 | 0.86 |
| 11 | TPIAQMFGPPSSDSTP | 247 | 0.86 |
| 11 | GQYSSTQREHRHQFPT | 2 | 0.86 |
| 12 | TVLISGSGTNLQAVID | 872 | 0.85 |
| 12 | QLTKCEHLYHRDCIDQ | 779 | 0.85 |
| 12 | RHGVVSRTIPRSEVNQ | 155 | 0.85 |
| 13 | QAVIDDTTLPAKIVRV | 883 | 0.84 |
| 13 | AVSSGASTPSRRLSTT | 589 | 0.84 |
| 13 | RRHSTMSRLGSRILPN | 119 | 0.84 |
| 14 | LGPLEAKGVRIVNLHP | 974 | 0.83 |
| 14 | CGDSMSAAAVDGTRTI | 741 | 0.83 |
| 14 | DVQMTHSTETNFLVDP | 59 | 0.83 |
| 14 | RREEQTPLSRILQLAA | 341 | 0.83 |
| 15 | HQRRRSDSEFARHREQ | 638 | 0.82 |
| 15 | SGNGPAGDQQTAGLPG | 482 | 0.82 |
| 15 | SFHNSDPFSFIPHPGP | 272 | 0.82 |
| 15 | PSGPSTDGSAEPGWGW | 214 | 0.82 |
| 15 | VHEIEWKVVIEGLQKT | 1055 | 0.82 |
| 16 | SNRKDAFGLERARRAN | 900 | 0.81 |
| 16 | TYRIKVDYLPTSHTTF | 837 | 0.81 |
| 16 | PSSGDSNNNSEDERPP | 401 | 0.81 |
| 17 | RDCIDQHGAFPSSFTI | 789 | 0.80 |
| 17 | EQLGSGAARRNGVVEP | 652 | 0.80 |
| 17 | ESFIQSLRNATSGQPS | 387 | 0.80 |
| 17 | SLSSRGGSRRRSLRGP | 180 | 0.80 |
| 17 | HQFPTESPLPRQRSHS | 13 | 0.80 |
| 18 | IPTQYHNLVKYKKQHP | 916 | 0.79 |
| 18 | HSASSSAPPANVDGSI | 526 | 0.79 |
| 18 | GPVNFMRVFRFANSDN | 417 | 0.79 |
| 18 | PNSVIRGLLNSEEETP | 133 | 0.79 |
| 19 | GNTQDETGSQDDASQQ | 92 | 0.78 |
| 19 | DESLPHNTTHQRRRSD | 629 | 0.78 |
| 19 | AATAIATQLSGGAGPA | 355 | 0.78 |
| 19 | SEVNQSSARFSPFASL | 166 | 0.78 |
| 19 | KPILVKEIPFVKGADE | 1031 | 0.78 |
| 20 | NPTYEDMVLLSSLLGP | 703 | 0.77 |
| 21 | HAEGQEGRTVTLVVIG | 461 | 0.76 |
| 21 | SEDERPPGPVNFMRVF | 410 | 0.76 |
| 21 | LRARPPRALRREEQTP | 332 | 0.76 |
| 21 | EDMNNGQNPERAGAFS | 33 | 0.76 |
| 21 | SQSQSSTRHFPSLLRA | 319 | 0.76 |
| 21 | PGWGWRRSLRIRRVGR | 225 | 0.76 |
| 22 | SGGAGPALPNIPSLGN | 364 | 0.75 |
| 22 | RVGRVGHSLPTPIAQM | 237 | 0.75 |

| Start | End | Max_score_pos | Sequence |
|---|---|---|---|
| 707 | 742 | 713 | EDMVLLSSLLGPVKPPVATQEDLISAGGLYRVVKCG |
| 469 | 480 | 472 | TVTLVVIGVRSV |
| 761 | 777 | 766 | GERCLICLSEYEVAEEL |
| 946 | 977 | 962 | AELARLVLEDKPDLVACLGFMHVLSEGFLGPL |
| 676 | 686 | 680 | RSWLIYVVGTN |
| 779 | 824 | 818 | QLTKCEHLYHRDCIDQHGAFPSSFTISSHLSLFASDIHC |
| | | | CIATFWF |
| 561 | 571 | 565 | LSSLPSVVSES |
| 870 | 877 | 872 | RLTVLISG |
| 892 | 899 | 896 | PAKIVRVI |
| 979 | 992 | 985 | AKGVRIVNLHPALP |
| 69 | 84 | 73 | NFLVDPLQSLISHSSM |
| 1047 | 1066 | 1065 | DLHAFEQKVHEIEWKVVIEG |
| 1032 | 1043 | 1038 | PILVKEIPFVKG |
| 837 | 848 | 843 | TYRIKVDYLPTS |
| 348 | 362 | 351 | LSRILQLAATAIATQ |
| 128 | 140 | 138 | GSRILPNSVIRGL |
| 107 | 120 | 115 | QDYQSALFARVARR |
| 154 | 162 | 156 | HRHGVVSRT |
| 1018 | 1026 | 1021 | VMIHNVISE |
| 917 | 932 | 923 | PTQYHNLVKYKKQHPA |
| 493 | 514 | 503 | AGLPGLDFLRLPFFPPGTLSPR |
| 300 | 320 | 305 | LNSVEPALADSQPASPMLTSQ |
| 584 | 593 | 588 | EPGLSAVSSG |
| 325 | 335 | 330 | TRHFPSLLRAR |
| 692 | 699 | 697 | PAFAAPSL |
| 238 | 252 | 244 | VGRVGHSLPTPIAQM |
| 882 | 889 | 885 | LQAVIDDT |
| 173 | 182 | 178 | ARFSPFASLS |
| 279 | 288 | 281 | FSFIPHPGPL |
| 368 | 375 | 374 | GPALPNIP |
| 603 | 610 | 604 | TTSAVSPN |
| 193 | 202 | 195 | RGPYFIPRSD |
| 662 | 667 | 667 | NGVVEP |
| 527 | 540 | 534 | SASSSAPPANVDGS |
| 387 | 393 | 391 | ESFIQSL |
| 545 | 555 | 551 | SPNVPRRLSDV |
| 573 | 582 | 575 | PGPHPPPSTP |
| 16 | 24 | 18 | PTESPLPRQ |

Patients with candidiasis mounted significant IgG titers against Candida proteins within 2 weeks of their diagnoses, whereas IgM titers did not differ from those of controls. This finding suggests that systemic candidiasis is well-established prior to the point at which the disease is diagnosed by current methods. Alternatively, a patient might have ongoing, low-level systemic exposure to Candida that predisposes them to systemic candidiasis.

Four C. albicans proteins (Set1p, Rbt4p, Met6p and Bgl2p) may serve as diagnostic targets, for serologic or antigen detection tests. Antibody profiling against candida proteins such as these may identify patients at high-risk to develop candidiasis, and could be a tool for targeted, pre-emptive therapy.

Antibody responses among patients with candidiasis due to other Candida spp. are being carried out. Trends of immunoglobulin responses during the course of candidiasis are being determined.

### Example 2

### Expression and Purification of Antigens

Fifteen antigens from 12 distinct proteins were expressed as 6× His-tagged polypeptides in E. coli and purified from cell-free supernatants by chromatography on Ni²⁺-NTA-agarose. Nine of the purified recombinant antigens were full-length (Table 9). MET1, PGK2 and MUC2 could not be efficiently expressed as full-length proteins, and were instead purified as two fragments (MET6-F1 and -F2; PGK1-F1 and -F2, and MUC1-F1 and -F2). The purified antigens appeared as single bands of expected sizes by SDS-PAGE and were detected by probing with anti-His antibodies (data not shown).

### Example 3

### Recognition of Antigens by Sera from Patients with Candidiasis and Un-Infected Controls

Sera from the patients with candidiasis and un-infected controls were tested against the 15 recombinant antigens using ELISA. The sera from 2 patients (one with systemic candidiasis and one control) were sufficient to test against only 14 antigens (all except PGK1). As anticipated, the IgM and IgG titers in the sera of the 8 premature or newborn infants were consistently low (at or below the control levels) against each of the recombinant antigens. As such, these sera were excluded from further data analysis.

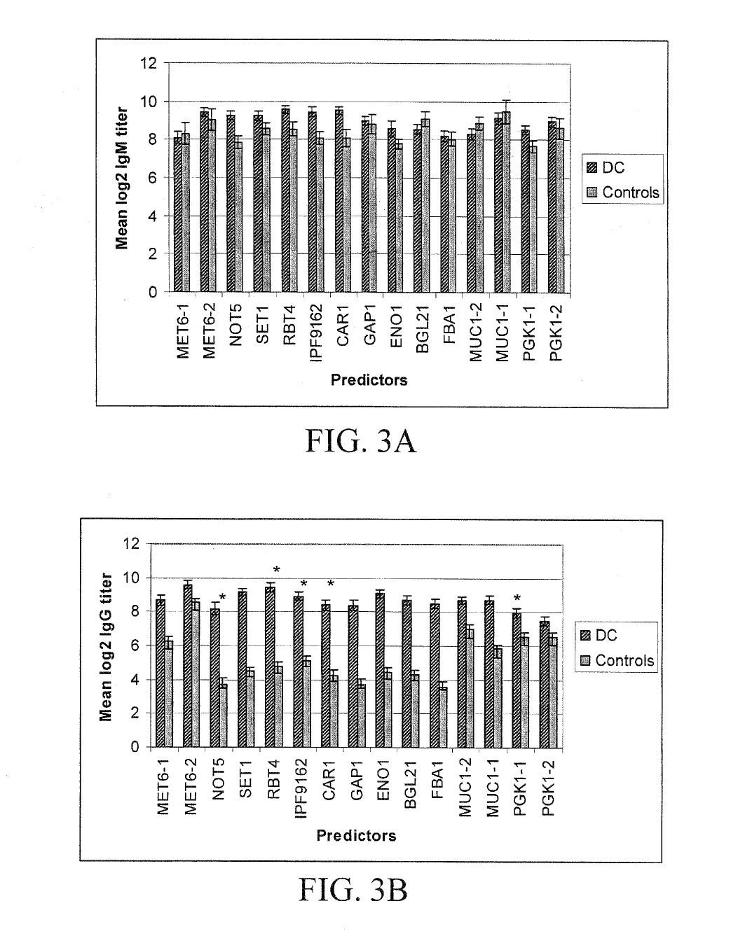

Overall, the IgG titers from the sera of patients enrolled in this study better differentiated patients with systemic candidiasis and controls than IgM (FIGS. 3A and 3B). For this reason, only IgG response was selected for further analysis. There were no significant differences in titers between immunocompromised hosts, burn victims and other patients with candidiasis, between patients infected with C. albicans vs. non-C. albicans spp., or based on portal of entry.

### Example 4

### Identification of Antibody Responses that Discriminate Patients with Systemic Candidiasis from Controls

For each of the antigens, cut-off antibody titers were assigned that best discriminated patients from controls. The sensitivity and specificity of the antibody responses against individual antigens in identifying patients with systemic candidiasis are presented in Table 12.

Having shown that IgG titers against specific antigens diagnosed systemic candidiasis with reasonable sensitivity and specificity, the inventors sought to develop a predictive model that considered antibody responses against a panel of antigens. Collinearity was assessed by determining the tolerance of each of the 15 predictor variables (i.e., antibodies to specific antigens) on the others. Since collinearity diagnostics revealed no collinearity problems, all 15 antigens were included in the analysis.

Backwards elimination and canonical correlation analyses were then used to identify variables that might best predict systemic candidiasis. The two methods demonstrated excellent overall agreement in identifying predictors likely to be significant. The rank order in which the predictor variables were identified using canonical correlation analysis is presented in Table 13. Based on these results, a panel of seven predictors (SET1, MUC1-2, FBA1, PGK1-F1, PGK1-F2, and BCL2 and ENO1) were selected for discriminant analysis.

Discriminant analysis of the full set of 15 predictors yielded an outcome classification error rate of 3.7% (3/82) (Table 14). Among the 82 patients and controls enrolled in the study, only 3 were classified incorrectly: one patient with systemic candidiasis was predicted to be a control, and 2 controls were predicted to have systemic candidiasis. The sensitivity and specificity of the panel of 15 predictors were 96.6% and 95.6%, respectively. Discriminant analysis of the panel of 7 predictors yielded identical results. Using classification tables generated for smaller subsets of predictors, the inventors identified a panel of 4 predictors (SET1, ENO1, MUC1-2 and PGK1-2) that performed as well as the larger panels (Table 14). Canonical discriminate analysis was applied to assign weights to each of the 4 variables in the prediction of systemic candidiasis; the strongest correlations were with SET1 (the standardized canonical coefficients for SET1, ENO1, MUC1-2 and PGK1-2 were 1.29, 0.91, −0.39 and −0.20, respectively).

Using regression analysis, it was confirmed that the panel of 4 predictors performed as well as the 15 predictors in diagnosing systemic candidiasis. The analysis of the 4 predictors yielded an R² of 0.69, which was not significantly different from the R² of 0.73 for the full model (F=0.98, p=0.47).

### Example 5

### Testing a Prediction Model for Systemic Candidiasis

Based on the data, the equation that best predicted systemic candidiasis was: prediction score=(0.10*SET1±0.07*ENO−0.04*MUC1-F2−0.02*PGK1-F2−0.12), where SET1, ENO, MUC1-F2 and PGK1-F2 denote the log₂ of the specific antibody titers in individual patients. A score >0.5 for a given patient was predictive of systemic candidiasis.

To test the validity of this model, ELISAs were performed against the 15 recombinant antigens using sera that had been collected from 16 patients with systemic candidiasis and 16 un-infected controls prior to the present study. The panel of 4 predictors yielded sensitivity and specificity of 100% (16/16) and 87.5% (14/16), respectively. The only classification errors were two controls who were predicted to have systemic candidiasis.

To the inventors' knowledge, this is the first study to demonstrate that antibody responses against a panel of C. albicans antigens can accurately diagnose systemic candidiasis. Serum IgG responses were measured against 15 recombinant C. albicans antigens by ELISA and derived a prediction model that identified patients with systemic candidiasis with an error rate of only 3.7%, sensitivity of 96.6% and specificity of 95.6%. The performance of the prediction model was superior to antibody detection against any individual antigen. Using backwards elimination and canonical correlation analyses, a subset of 4 antigens that performed as accurately as the full panel was identified. The inventors further confirmed the validity of the 4 antigen panel by testing sera that were different from those used to derive the prediction model. Given the limitations of current diagnostic tests, measuring antibody responses against a panel such as ours might represent a significant advance in the diagnosis of systematic candidiasis.

These data refute several of the major concerns about the limitations of antibody detection as a diagnostic tool. First, the inventors demonstrated that patients with systemic candidiasis exhibited significant IgG titers against a wide range of antigens at the time of positive blood cultures. This observation corroborates a number of reports documenting significant IgG titers against individual proteins like Eno1p, Hwp1p, mannan and CAGTA before or at the time of the first positive blood culture (Lain, A et al. BMC Microbiol., 2007; 7:35; Lain, A et al. Clin Vaccine Immunol., 2007; 14:318-9; Pazos, C et al. Rev Iberoam Micol., 2006; 23:209-15). Such findings are consistent with the fact that blood cultures are often not positive until relatively late in the disease course (Morris, A J et al. J Clin Microbiol., 1996; 34:1583-5; Garey, K W et al. Clin Infect Dis., 2006 Jul. 1; 43:25-3; Morrell, M et al. Antimicrob Agents Chemother., 2005; 49:3640-5). Alternatively, it is possible that invasive diseases like candidemia are preceded in at least some patients by low-level systemic exposure to Candida spp., perhaps reflecting “leakage” from mucosal sites of colonization. It is striking that IgG responses were superior to IgM in discriminating patients with systemic candidiasis from controls. In fact, the majority of studies to date have looked at IgG antibodies (Quindos, G et al. Rev Iberoam Micol., 2004; 21(1)). Studies of IgM responses have yielded conflicting results, with results superior to IgG against blastospore cytoplasm and mannan in one study and inferior against hyphal cell wall proteins in another (Gutierrez, J et al. J Clin Microbiol., 1993; 31:2550-2; Torres-Rodriguez, J M et al. Mycoses., 1997; 40:439-44). We hypothesize that the potent IgG responses in the present study are consistent with amnestic responses against tissue invasion by a commensal organism to which the host has already been exposed.

Second, the inventors showed that the sensitivity of antibody detection was not attenuated among patients who were significantly immunocompromised, including stem cell and solid organ transplant recipients, patients with hematologic malignancies and a high-dose steroid recipient with lupus. Again, this observation is consistent with numerous reports assessing antibody responses against individual antigens. Third, it was found that antibody responses to the recombinant C. albicans antigens included in the panel diagnosed patients infected with non-C. albicans spp. as well as C. albicans, which likely reflected the inclusion of conserved proteins like glycolytic enzymes, SET1 (a histone methyltransferase) and NOT5 (a component of the CCR-NOT global regulatory complex). Indeed, IgG responses against C. albicans enolase have been reported to be effective in identifying patients with candidemia caused by diverse Candida spp. (Lain, A et al. BMC Microbial., 2007; 7:35). Interestingly, a recent study showed that the detection of antibodies to HWP1, a protein produced exclusively during hyphal growth by C. albicans, is also useful among patients infected with non-C. albicans spp. (Lain, A et al. BMC Microbial., 2007; 7:35), suggesting that epitopes within non-conserved proteins might also elicit cross-reactive responses.

The sensitivities and specificities that were observed with individual proteins are within the ranges previously reported for antibody detection against a variety of antigens. Studies of IgG responses to enolase, for example, have yielded sensitivities of 50-92% and specificities of 78-95% (Quindos, Get al. Rev Iberoam Micol., 2004; 21(1); Lain, A et al. Clin Vaccine Immunol., 2007; 14:318-9; van Deventer, A J et al. Microbial Immunol., 1996; 40:125-31; Mitsutake, K et al. J Clin Microbial., 1996; 1918-21), and similar performances have been reported for tests against SAP (Na B K and Song C Y Clin Diagn Lab Immunol., 1999; 6:924-9; Yang Q et al. Mycoses, 2007; 50:165-71), HWP1 (Lain, A et al. Clin Vaccine Immunol., 2007; 14:318-9), a 52 kDa metalloprotein (El Moudni, B et al. Clin Diagn Lab Immunol., 1998; 5:823-5), mannan (Sendid, B et al. J Clin Microbial., 1999; 37:1510-7; Yera, H et al. Eur J Clin Microbial Infect Dis., 2001; 20:864-70) and CTGTA (Quindos, G et al. Rev Iberoam Micol., 2004; 21(1); Lain, A et al. BMC Microbial., 2007; 7:35). In attempts to overcome the diagnostic limitations of existing antibody tests, investigators have used a number of them in combination with antigen and/or metabolite detection. In general, these strategies have improved the sensitivity and specificity of the individual tests, as well as resulted in earlier diagnoses of systemic candidiasis (Fisher, J F et al. Am J Med Sci., 1985; 290:135-42; Sendid, B et al. J Clin Microbial., 1999; 37:1510-7; Sendid, B et al. J Med Microbial., 2002; 51:433-42; Sendid, B et al. J Clin Microbial., 2004; 42:164-71; Yera, H et al. Eur J Clin Microbiol Infect Dis., 2001; 20:864-70; Platenkamp, G J et al. J Clin Pathol., 1987; 40:1162-7). It is quite possible, therefore, that a prediction model such as ours based on antibody responses to multiple antigens will ultimately be of greatest utility in combination with cultures and other diagnostic markers.

In conclusion, a highly accurate model can be derived using a few rationally selected antigens. Indeed, limiting the number of antigens is desirable since it simplifies large-scale testing by ELISA. Once a prediction model is validated, the precise roles of antibody profiling against candidal antigens in the management of systemic candidiasis can be further investigated in well-designed prospective studies. In addition to diagnosing systemic candidiasis, potential applications of antibody testing include tracking responses to antifungal therapy and identifying high-risk patients who could benefit from preventive or pre-emptive treatment.

It should be understood that the examples and embodiments described herein are for illustrative purposes only and that various modifications or changes in light thereof will be suggested to persons skilled in the art and are to be included within the spirit and purview of this application.

1-58. (canceled)

59. A composition of matter comprising:
(a) an amino acid sequence listed in. Tables 3, 4, 5, 6, 7, 8, 9, 12, 13, 14, 15, 16, 17 or 18 disclosed herein; or
(b) a fragment of (a); or
(c) an array comprising a combination of polypeptides selected from SET1 (chromatin regulatory protein), ENO1 (enolase I), PGK1-2 (phosphoglycerate kinease), or MUC 1-2 (cell surface glycoprotein); or
(d) an array comprising one or more polypeptides selected from MET6-1, METE-2, NOT5, RBT4, IPF9162, CAR1, GAP1, SET1, ENO1, FBA1, PGK1-1, PGK1-2, MUC1-1, MCU1-2, or BGL2; or
(e) an array comprising one or more polypeptides selected from among SET1, ENO1, FBA1, PGK1-1, PGK1-2, MUC1-2, and BGL2; or
(f) a variant of a polypeptide of (a), (b) or (c), wherein said variant polypeptide specifically binds to an antibody that specifically binds to a polypeptide of (a), (b) or (c); or
(g) a heterologous polypeptide fused, in frame, to a polypeptide comprising the polypeptide of (a) or (b); or
(h) a multimeric construct comprising a polypeptide of (a) or (b).

60. A composition comprising at least one isolated or purified polypeptide according to claim 59, or an isolated polynucleotide encoding the polypeptide; and an additional component.

61. The composition according to claim 60, wherein said additional component is a solid support, and wherein said polypeptide or said encoding polynucleotide is immobilized on said support.

62. The composition according to claim 61, wherein said solid support is selected from the group consisting of microtiter wells, magnetic beads, non-magnetic beads, agarose beads, glass, cellulose, plastics, polyethylene, polypropylene, polyester, nitrocellulose, nylon, and polysulfone.

63. The composition according to claim 60, wherein said additional component is a pharmaceutically acceptable excipient.

64. The composition according to claim 61, wherein said solid support provides an array of polypeptides or encoding polynucleotides, and wherein said array of polypeptides is selected from among the polypeptides listed in Tables 3, 4, 5, 6, 7, 8, 9, 12, 13, 14, 15, 16, 17 or 18 disclosed herein, or a fragment or variant thereof.

65. The composition according to claim 61, wherein said solid support provides an array of polypeptides or encoding polynucleotides, and wherein said array of polypeptides is selected from SET1 (chromatin regulatory protein), ENO1 (enolase I), PGK1-2 (phosphoglycerate kinease), and/or MUC1-2 (cell surface glycoprotein).

66. The composition according to claim 61, wherein said solid support provides an array of polypeptides or encoding polynucleotides, and wherein said array of is selected from among MET6-1, METE-2, NOT5, RBT4, IPF9162, CAR1, GAP1, SET1, ENO1, FBA1, PGK1-1, PGK1-2, MUC1-1, MUC1-2, and/or BGL2.

67. The composition according to claim 61, wherein said solid support provides an array of polypeptides or encoding polynucleotides, and wherein said array of polypeptides is selected from SET1, ENO1, FBA1, PGK1-1, PGK1-2, MUC1-2, and/or BGL2.

68. The composition according to claim 60, further comprising an additional antigen of interest.

69. A method of binding an antibody to a polypeptide comprising contacting a sample containing an antibody with a polypeptide under conditions that allow for the formation of an antibody-antigen complex, wherein said polypeptide is selected from the group consisting of the polypeptides listed in Tables 3, 4, 5, 6, 7, 8, 9, 12, 13, 14, 15, 16, 17 or 18 disclosed herein, or a fragment or variant thereof.

70. The method according to claim 69, further comprising the step of detecting the formation of said antibody-antigen complex.

71. The method according to claim 69, wherein said method is performed using an array of polypeptides.

72. The method according to claim 71, wherein said array comprises SET1 (chromatin regulatory protein), ENO1 (enolase I), PGK1-2 (phosphoglycerate kinease), and MUC1-2 (cell surface glycoprotein).

73. The method according to claim 71, wherein said array comprises MET6-1, MET6-2, NOT5, RBT4, IPF9162, CAR1, GAP1, SET1, ENO1, FBA1, PGK1-1, PGK1-2, MUC1-1, MUC1-2, and BGL2.

74. The method according to claim 71, wherein said array comprises SET1, ENO1, FBA1, PGK1-1, PGK1-2, MUC1-2, and BGL2.

75. An isolated antibody that specifically binds to a polypeptide of claim 59, or a fragment or variant thereof

76. A method of hybridizing polynucleotides comprising contacting a sample comprising a population of polynucleotides with a second population of polynucleotides under conditions that allow for the formation of a hybridization complex, wherein said second population of polynucleotides comprises polynucleotides that encode at least one polypeptide that is selected from among:
(a) an amino acid sequence listed in Tables 3, 4, 5, 6, 7, 8, 9, 12, 13, 14, 15, 16, 17 or 18 disclosed herein; or
(b) a fragment of (a); or
(c) a polypeptide listed in Tables 3, 4, 5, 6, 7, 8, 9, 12, 13, 14, 15, 16, 17 or 18 disclosed herein; or
(d) one or more polypeptides (e.g., one, two, three, or four or more polypeptides) selected from among SET1 (chromatin regulatory protein), ENO1 (enolase I), PGK1-2 (phosphoglycerate kinease), and MUC1-2 (cell surface glycoprotein); or
(e) one or more polypeptides (e.g., one, two, three, four, five, six, seven, eight, nine, ten, eleven, twelve, thirteen, fourteen, or fifteen or more polypeptides) selected from among MET6-1, MET6-2, NOT5, RBT4, IPF9162, CAR1, GAP1, SET1, ENO1, FBA1, PGK1-1, PGK1-2, MUC1-1, MUC1-2, and BGL2; or
(f) one or more polypeptides (e.g., one, two, three, four, five, six, or seven or more polypeptides) selected from among SET1, ENO1, FBA1, PGK1-1, PGK1-2, MUC1-2, and BGL2; or
(g) a variant of a polypeptide of (a), (b), (c), (d), (e), or (f), wherein said variant polypeptide specifically binds to an antibody that specifically binds to a polypeptide of (a), (b), (c), (d), (e), or (f); or
(h) a fragment of a polypeptide of (c), (d), (e), or (f), wherein said variant polypeptide fragment specifically binds to an antibody that specifically binds to a polypeptide of (c), (d), (e), or (f), or a fragment of (c), (d), (e), or (f).

77. The method according to claim 76, further comprising the step of detecting the hybridization complex.

78. A method for diagnosing or monitoring a Candida or Aspergillus infection in a subject, the method comprising:
(a) providing a gene expression profile obtained from a biological sample of the subject, wherein the expression profile comprises a plurality of Candida or Aspergillus genes that are expressed at the protein level; and
(b) comparing the subject's gene expression profile to a reference gene expression profile.

79. The method according to claim 78, wherein the reference gene expression profile is obtained from a normal, healthy individual, or from an infected individual.

80. The method according to claim 78, wherein said method further comprises:
(a) providing a gene expression profile obtained from a biological sample from the subject after the subject has undergone a treatment regimen for Candida or Aspergillus infection; and
(b) comparing the subject's post-treatment gene expression profile to the reference gene expression profile, to monitor the subject's response to the treatment regimen.

81. The method according to claim 78, wherein the Candida or Aspergillus genes are those listed in Tables 3, 4, 5, 6, 7, 8, 9, 12, 13, 14, 15, 16, 17 or 18 disclosed herein.
